(19) United States
(12) Patent Application Publication
(10) Pub. No.: US 2023/0310390 A1
(43) Pub. Date: Oct. 5, 2023

(54) INHIBITORS OF SHORT-CHAIN DEHYDROGENASE ACTIVITY FOR TREATING NEURODEGENERATION

(71) Applicants: CASE WESTERN RESERVE UNIVERSITY, Cleveland, OH (US); The United States Government as represented by the Department of Veterans Affairs, Washington, DC (US); UNIVERSITY OF TEXAS SOUTHWESTERN MEDICAL CENTER, Dallas, TX (US)

(72) Inventors: Sanford Markowitz, Pepper Pike, OH (US); Andrew Pieper, Washington, DC (US); Hongyun Li, Cleveland, OH (US); Edwin Vazquez Rosa, Cleveland, OH (US); Min-Kyoo Shin, Cleveland, OH (US); Yeojung Koh, Cleveland, OH (US); Joseph Ready, Dallas, TX (US)

(21) Appl. No.: 18/020,202

(22) PCT Filed: Aug. 9, 2021
(86) PCT No.: PCT/US2021/045231

§ 371 Date: Feb. 7, 2023

Related U.S. Application Data
(60) Provisional application No. 63/062,874, filed on Aug. 7, 2020.

(51) Int. Cl. A61K 31/4365; A61P 25/28

(52) U.S. Cl. CPC A61K 31/4365; A61P 25/28

(57) ABSTRACT

A method of treating neurodegeneration and/or neurodegenerative condition, disease, or disorder caused by and/or associated with elevated or aberrant 15-PGDH activity in a subject in need thereof includes administering to the subject a therapeutically effective amount of a 15-PGDH inhibitor.

### RELATED APPLICATION

This application claims priority from U.S. Provisional Application No. 63/062,874, filed Aug. 7, 2020, the subject matter of which are incorporated herein by reference in their entirety.

### GOVERNMENT FUNDING

This invention was made with government support under Grant No. CA150964 awarded by The National Institutes of Health. The United States government has certain rights in the invention.

### BACKGROUND

Prostaglandins, via their specific G protein coupled receptors, have a variety of physiological functions in the central nervous system. The major prostaglandin, prostaglandin E2 (PGE₂) can activate receptor types EP1, 2, 3, and 4. Activation of EP2 and EP4 receptors can regulates adenylate cyclase and the generation of 3, 5′-cyclic adenosine monophosphate (cAMP), whereas the activation of EP1 and EP3 receptors can regulates Ca²⁺ signaling. EP1 and EP2 receptors are expressed in cultured neurons and microglia as well as neurons of the cerebral cortex, striatum, and hippocampus. Also, activation of the EP2 receptor by PGE₂ is involved in long-term synaptic plasticity and cognitive function, as EP2^{-/-} mice showed impaired hippocampal synaptogenesis. (Chemtob et al. Semin Perinatol. 1994 February; 18(1):23-9; Yang et al., J Neurochem. 2009 January; 108(1):295-304). Following activation, different PGE₂ receptors can contribute or protect against N-methyl-D-aspartate (NMDA) neurotoxicity and ischemic stroke. For example, in a mouse model of focal cerebral ischemia, pretreatment with an EP2 receptor selective agonist was able to significantly decrease neurological deficits and deletion of EP2 receptors aggravated ischemic brain damage. (Ahmad et al., Exp Transl Stroke Med. 2010 Jul. 8; 2(1):12). Activation of the EP2 receptors with butaprost protected neurons from amyloid 0-peptide neurotoxicity in vitro. (Echeverria et al., Eur J Neurosci. 2005 November; 22(9):2199-206).

Several studies suggest that the mechanism by which PGE₂ affords neuroprotection is through EP2 or EP4 receptors, as they both increases cAMP, followed by a protein kinase A (PKA)-dependent pathway. (Echeverria et al. Eur J Neurosci. 2005 November; 22(9):2199-206; McCullough et al., J Neurosci. 2004 Jan. 7; 24(1):257-68). Administration of PGE₂ has not been shown to be therapeutically useful against the EP2 receptor as the half-life of PGE₂ is less than 1 min. following intravenous injection and approximately 30 sec. in the circulatory system. (Fitzpatrick et al., Prostaglandins. 1980 June; 19(6):917-31; Kimball et al. Prostaglandins. 1980 September; 20(3):559-69).

### SUMMARY

Embodiments described herein relate generally to compositions and methods of treating neurodegeneration and/or neurodegenerative conditions, diseases and/or disorders. In some embodiments, the neurodegeneration and/or neurodegenerative conditions, diseases and/or disorders can be caused by and/or associated with enhanced or aberrant 15-PGDH activity in a subject in need thereof. It was found that 15-PDGH activity is increased in certain neurodegenerative conditions, diseases, and/or disorders and that this activity could be inhibited, such as with a 15-PGDH inhibitor described herein, to provide or promote neuroprotection in a subject from axonal degeneration, neuronal cell death, and/or glia cell damage, augment neuronal signaling underlying learning and memory, ameliorate memory loss or cognitive decline, stimulate neuronal regeneration, attenuate or decrease blood brain barrier permeability and/or treat the neurodegenerative conditions, diseases and/or disorders.

In some embodiments, a method of treating neurodegeneration and/or neurodegenerative condition, disease, or disorder caused by and/or associated with elevated or aberrant 15-PGDH activity in a subject in need thereof includes administering to the subject a therapeutically effective amount of a 15-PGDH inhibitor.

In some embodiments, the neurodegenerative condition, disease, or disorder associated with aberrant or enhanced 15-PGDH activity can include at least one of subarachnoid hemorrhage, schizophrenia, major depression, bipolar disorder, normal aging, epilepsy, traumatic brain injury and/or a visual symptom associated therewith, post-traumatic stress disorder, Parkinson's disease, Parkinson Plus syndromes, Lewy Body Dementia, multiple system atrophy, corticobasal neurodegeneration, progressive supranuclear palsy, Alzheimer's disease, Alzheimer's disease related dementias, Down syndrome, spinocerebellar ataxia, amyotrophic lateral sclerosis, Huntington's disease, stroke, brain radiation therapy, chronic stress, abuse of a neuro-active drug, retinal degeneration, spinal cord injury, peripheral nerve injury, idiopathic peripheral neuropathy, cognitive decline and/or general frailty associated with normal aging and/or chemotherapy, chemotherapy induced neuropathy, concussive injury, peripheral nerve crush injury, peripheral neuropathy, diabetic neuropathy, post-traumatic headache, multiple sclerosis, retinal degeneration and dystrophy, Leber congenital amaurosis, retinitis pigmentosa, cone-rod dystrophy, microphthalmia, anophthalmia, myopia, and hyperopia, spinal cord injury, traumatic spinal cord injury, peripheral nerve injury, retinal neuronal death related diseases, retinal trauma, Autism, Stargardt disease, Kearns-Sayre syndrome, Pure neurosensory deafness, Hereditary hearing loss with retinal diseases, Hereditary hearing loss with system atrophies of the nervous system, Progressive spinal muscular atrophy, Progressive bulbar palsy, Primary lateral sclerosis, Hereditary forms of progressive muscular atrophy and spastic paraplegia, Frontotemporal dementia, Dementia with Lewy bodies, Corticobasal degeneration, Progressive supranuclear palsy, Prion disorders causing neurodegeneration, Multiple system atrophy, Hereditary spastic paraparesis, Friedreich ataxia, Non-Friedreich ataxia, Spinocerebellar atrophies, Amyloidoses, Metabolic-related neurodegenerative disorders, Toxin-related neurodegenerative disorders, Multiple sclerosis, Charcot Marie Tooth, Diabetic neuropathy, Metabolic neuropathies, Endocrine neuropathies, Creutzfeldt-Jacob Disease, Primary progressive aphasia, Frontotemporal Lobar Degeneration, Cortical blindness, Shy-Drager Syndrome, Diffuse cerebral cortical atrophy of non-Alzheimer type, Lewy-body dementia, Pick disease, Thalamic degeneration, Mesolimbocortical dementia of non-Alzheimer type, Nonhuntingtonian types of chorea and dementia, Cortical-striatal-spinal degeneration, Dementia-Parkinson-amyotrophic lateral sclerosis complex, Cerebrocerebellar degeneration, Cortico-basal ganglionic degeneration, Familial dementia with spastic paraparesis or myoclonus, Tourette syndrome, or viral infection.

In other embodiments, the neurodegeneration and/or neurodegenerative condition, disease, or disorder is associated with an aberrant level (e.g., decrease or increase in the level) of at least one eicosanoid selected from PGE2, 15-keto-PGE2, PGF2α, 6-keto-PGF1α, PGD2, PGJ2, TN-E, TXB2, LTB4, 15-HETE, 12-HETE, 8-HETE, 5-HETE, 17-HDA, 12, 13-DiHOME, 9,10-DiHOME, 14,15-DHET, or 11,12-DHET in neurotissue of the subject. The 15-PGDH inhibitor can be administered to the subject to modulate the level of the aberrant eicosanoid to a normal or healthy level in the neurotissue. For example, the neurodegeneration and/or neurodegenerative condition, disease, or disorder can be associated with an increase in the level of at least one of 15-keto-PGE2, TN-E, TXB2, LTB4, 15-HETE, 12-HETE, 8-HETE, 5-HETE, 17-HDA, 12, 13-DiHOME, 9,10-DiHOME, or 14,15-DHET in neurotissue (e.g., brain tissue) of the subject, and the 15-PGDH inhibitor can be administered to the subject at amount effective to decrease the level of the at least one of 15-keto-PGE2, TN-E, TXB2, LTB4, 15-HETE, 12-HETE, 8-HETE, 5-HETE, 17-HDA, 12, 13-DiHOME, 9,10-DiHOME, or 14,15-DHET in neurotissue of the subject. In another example, the neurodegeneration and/or neurodegenerative condition, disease, or disorder can be associated with a decrease in the level of at least one of PGJ2, TNE, 15-HETE, or 9,10-DiHOME in neurotissue (e.g., brain tissue) of the subject, and the 15-PGDH inhibitor can be administered to the subject at amount effective to increase the level of the at least one of PGJ2, TNE, 15-HETE, or 9,10-DiHOME in neurotissue (e.g., brain tissue) of the subject.

In some embodiments, the neurotissue can include brain tissue of the subject, such as the hippocampus. The 15-PGDH inhibitor can be administered at an amount effective to stimulate hippocampal neurogenesis.

Other embodiments described herein relate to a method of treating and/or inhibiting memory loss and/or cognitive decline in a subject in need thereof by administering to the subject a therapeutically effective amount of a 15-PGDH inhibitor. In some embodiments, the memory loss and/or cognitive decline can be caused by and/or associated with neurodegeneration and/or neurodegenerative conditions, diseases and/or disorders. The therapeutically effective amount of the administered 15-PGDH can be an amount effective to ameliorate memory loss and/or cognitive decline and/or improve memory and/or cognition.

In some embodiments, neurodegenerative condition, disease, or disorder can include at least one of Alzheimer's disease, Lewy body dementia, Vascular dementia, Age-related dementia, Frontotemporal dementia, mixed dementia, or traumatic brain injury.

In still other embodiments, the memory loss and/or cognitive decline is associated with an aberrant level (e.g., decrease or increase in the level) of at least one eicosanoid selected from PGE2, 15-keto-PGE2, PGF2α, 6-keto-PGF1α, PGD2, PGJ2, TN-E, TXB2, LTB4, 15-HETE, 12-HETE, 8-HETE, 5-HETE, 17-HDA, 12, 13-DiHOME, 9,10-DiHOME, 14,15-DHET, or 11,12-DHET in brain tissue (e.g., hippocampus) of the subject. The 15-PGDH inhibitor can be administered to the subject to modulate the level of the aberrant eicosanoid to a normal or healthy level in the brain tissue. For example, the memory loss and/or cognitive decline can be associated with an increase in the level of at least one of 15-keto-PGE2, TN-E, TXB2, LTB4, 15-HETE, 12-HETE, 8-HETE, 5-HETE, 17-HDA, 12, 13-DiHOME, 9,10-DiHOME, or 14,15-DHET in brain tissue of the subject, and the 15-PGDH inhibitor can be administered to the subject at amount effective to decrease the level of the at least one of 15-keto-PGE2, TN-E, TXB2, LTB4, 15-HETE, 12-HETE, 8-HETE, 5-HETE, 17-HDA, 12, 13-DiHOME, 9,10-DiHOME, or 14,15-DHET in brain tissue of the subject. In another example, the memory loss and/or cognitive decline can be associated with a decrease in the level of at least one of PGJ2, TNE, 15-HETE, or 9,10-DiHOME in brain tissue of the subject, and the 15-PGDH inhibitor can be administered to the subject at amount effective to increase the levels of the at least one of PGJ2, TNE, 15-HETE, or 9,10-DiHOME in e.g., brain tissue of the subject.

Still other embodiments described herein relate to methods of reducing blood brain barrier permeability in a subject in need thereof. The method includes administering to the subject a therapeutically effective amount of a 15-PGDH inhibitor.

In some embodiments, the subject with blood brain permeability has or is at risk of a neurodegenerative condition, disorder, or disease. For example, the subject can have or be at risk of mild cognitive impairment, Alzheimer's disease, Lewy body dementia, Vascular dementia, Age-related dementia, Frontotemporal dementia, mixed dementia, Parkinson's disease, Huntington's disease, multiple sclerosis, diabetic retinopathy, prion disorders, or amyotrophic lateral sclerosis.

In some embodiments, the subject has an aberrant level (e.g., decrease or increase in the level) of at least one eicosanoid selected from PGE2, 15-keto-PGE2, PGF2α, 6-keto-PGF1α, PGD2, PGJ2, TN-E, TXB2, LTB4, 15-HETE, 12-HETE, 8-HETE, 5-HETE, 17-HDA, 12, 13-DiHOME, 9,10-DiHOME, 14,15-DHET, or 11,12-DHET in brain tissue. The 15-PGDH inhibitor can be administered to the subject to modulate the level of the aberrant eicosanoid to a normal or healthy level in the brain tissue. For example, the subject can have an increase in the levels of at least one of 15-keto-PGE2, TN-E, TXB2, LTB4, 15-HETE, 12-HETE, 8-HETE, 5-HETE, 17-HDA, 12, 13-DiHOME, 9,10-DiHOME, or 14,15-DHET in neurotissue (e.g., brain tissue) of the subject, and the 15-PGDH inhibitor can be administered to the subject at amount effective to decrease the level of the at least one of 15-keto-PGE2, TN-E, TXB2, LTB4, 15-HETE, 12-HETE, 8-HETE, 5-HETE, 17-HDA, 12, 13-DiHOME, 9,10-DiHOME, or 14,15-DHET in the brain tissue. In another example, the subject can have a decrease in the level of at least one of PGJ2, TNE, 15-HETE, or 9,10-DiHOME in brain tissue, and the 15-PGDH inhibitor can be administered to the subject at amount effective to increase the levels of the at least one of PGJ2, TNE, 15-HETE, or 9,10-DiHOME in brain tissue of the subject.

In other embodiments, the 15-PGDH inhibitor can inhibit the enzymatic activity of recombinant 15-PGDH at an IC₅₀ of less than 1 μM, or preferably at an IC₅₀ of less than 250 nM, or more preferably at an IC₅₀ of less than 50 nM, or more preferably at an IC₅₀ of less than 10 nM, or more preferably at an IC₅₀ of less than 5 nM at a recombinant 15-PGDH concentration of about 5 nM to about 10 nM.

In some of the embodiments described herein, the 15-PGDH inhibitor has the following formula (V):

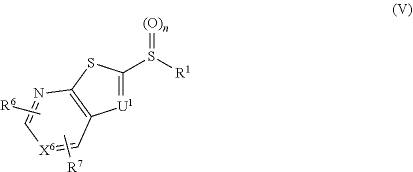

or a pharmaceutically acceptable salt, tautomer, or solvate thereof;
wherein n is 0-2
X⁶ is independently is N or CR^{c}
R¹, R⁶, R⁷, and R^{c} are the same or different each independently hydrogen or a substituted or unsubstituted group selected from C₁-C₂₄ alkyl, C₂-C₂₄ alkenyl, C₂-C₂₄ alkynyl, C₃-C₂₀ aryl, heteroaryl, heterocycloalkenyl containing from 5-6 ring atoms, C₆-C₂₄ alkaryl, C₆-C₂₄ aralkyl, halo, —Si(C₁-C₃ alkyl)₃, hydroxyl, sulfhydryl, C₁-C₂₄ alkoxy, C₂-C₂₄ alkenyloxy, C₂-C₂₄ alkynyloxy, C₅-C₂₀ aryloxy, acyl, acyloxy, C₂-C₂₄ alkoxycarbonyl, C₆-C₂₀ aryloxycarbonyl, C₂-C₂₄ alkylcarbonato, C₆-C₂₀ arylcarbonato, carboxy, carboxylato, carbamoyl, C₁-C₂₄ alkyl-carbamoyl, arylcarbamoyl, thiocarbamoyl, carbamido, cyano, isocyano, cyanato, isocyanato, isothiocyanato, azido, formyl, thioformyl, amino, C₁-C₂₄ alkyl amino, C₅-C₂₀ aryl amino, C₂-C₂₄ alkylamido, C₂-C₂₄ alkylamido substituted with a hydroxyl, C₆-C₂₀ arylamido, imino, alkylimino, arylimino, nitro, nitroso, sulfo, sulfonato, C₁-C₂₄ alkylsulfanyl, arylsulfanyl, C₁-C₂₄ alkylsulfinyl, C₅-C₂₀ arylsulfinyl, C₁-C₂₄ alkylsulfonyl, C₅-C₂₀ arylsulfonyl, sulfonamide, phosphono, phosphonato, phosphinato, phospho, phosphino, polyalkylethers, phosphates, and phosphate esters, groups incorporating amino acids or other moieties expected to bear positive or negative charge at physiological pH, and combinations thereof, and wherein R⁶ and R⁷ may be linked to form a cyclic or polycyclic ring, wherein the ring is a substituted or unsubstituted aryl, a substituted or unsubstituted heteroaryl, a substituted or unsubstituted cycloalkyl, and a substituted or unsubstituted heterocyclyl; and
U¹ is N, C—R², or C—NR³R⁴, wherein R² is selected from the group consisting of a H, a lower alkyl group, O, (CH₂)ₙ₁OR′ (wherein n1=1, 2, or 3), CF₃, CH₂—CH₂X, O—CH₂—CH₂X, CH₂—CH₂—CH₂X, O—CH₂—CH₂X, X, (wherein X=H, F, Cl, Br, or I), CN, (C═O)—R′, (C═O)N(R′)₂, O(CO)R′, COOR′ (wherein R′ is H or a lower alkyl group), and wherein R¹ and R² may be linked to form a cyclic or polycyclic ring, wherein R³ and R⁴ are the same or different and are each selected from the group consisting of H, a lower alkyl group, O, (CH₂)ₙ₁OR′ (wherein n1=1, 2, or 3), CF₃, CH₂—CH₂X, CH₂—CH₂—CH₂X, (wherein X=H, F, Cl, Br, or I), CN, (C═O)—R′, (C═O)N(R′)₂, COOR′ (wherein R′ is H or a lower alkyl group), and R³ or R⁴ may be absent.

### BRIEF DESCRIPTION OF THE DRAWINGS

FIG. 1 illustrates graphs showing comparisons of eicosanoid values in brains of 15-PGDH wild-type (Hpgd^{+/+}) versus 15-PGDH knockout (Hpgd^{-/-}) male mice.

FIG. 2 illustrates graphs showing comparison of eicosanoid values in brains of 6 month old male mice that are either wild-type or that are the 5×FAD strain of mice that model Alzheimer's disease.

FIG. 3 illustrates graphs showing comparison of eicosanoid values in brains of 6 month old male mice that are either wild-type or that are the 5×FAD strain of mice that model Alzheimer's disease.

FIG. 4 Illustrate graphs showing comparisons of eicosanoid values in brains of wild-type male mice who at 8 weeks of age were subjected to sham or actual traumatic brain injury (TBI) delivered by blast wave to the left skull.

FIG. 5 Illustrate graphs showing comparisons of eicosanoid values in brains of wild-type male mice who at 8 weeks of age were subjected to sham or actual traumatic brain injury (TBI) delivered by blast wave to the left skull.

FIG. 6 Illustrate graphs showing comparisons of eicosanoid values in brains of wild-type male mice who at 8 weeks of age were subjected to sham or actual traumatic brain injury (TBI) delivered by blast wave to the left skull.

FIG. 7 illustrate graphs that reprise the data of FIG. 4 highlighting comparison of post-TBI vehicle and (+)-SW033291 treated mice versus sham injury vehicle treated mice.

FIG. 8 illustrate graphs that reprise the data of FIG. 5 highlighting comparison of post-TBI vehicle and (+)-SW033291 treated mice versus sham injury vehicle treated mice.

FIG. 9 illustrate graphs that reprise the data of FIG. 6 highlighting comparison of post-TBI vehicle and (+)-SW033291 treated mice versus sham injury vehicle treated mice.

FIGS. 10(A-D) illustrate an immunoblot, images, and bar graphs showing that 15-PGDH activity is pathologically elevated in mouse TBI and AD, and human AD, and in mouse TBI and AD is returned to normal by treatment with (+)-SW033291.

FIG. 11 illustrates a bar graph showing treatment with (+)-SW033921 protects wild type mice from axon degeneration after TBI.

FIGS. 12(A-B) illustrates image and bar graphs showing treatment with (+)-SW033291 augments survival of newborn hippocampal neurons, thereby preserving hippocampal neurogenesis in 5×FAD mice, and does not affect accumulation of amyloid plaque in 5×FAD mice.

FIGS. 13(A-E) illustrate plots and bar graphs showing treatment with (+)-SW033921 protects wild type mice from cognitive impairment after TBI.

FIGS. 14(A-C) illustrate plots and a bar graph showing (+)-SW033921 protects cognitive function in 5×FAD mice.

FIGS. 15(A-D) illustrate images and bar graphs showing (+)-SW033921 protects the blood-brain barrier (BBB) in 5×FAD mice.

### DETAILED DESCRIPTION

For convenience, certain terms employed in the specification, examples, and appended claims are collected here. Unless defined otherwise, all technical and scientific terms used herein have the same meaning as commonly understood by one of ordinary skill in the art to which this application belongs.

The articles “a” and “an” are used herein to refer to one or to more than one (i.e., to at least one) of the grammatical object of the article. By way of example, “an element” means one element or more than one element.

The terms “comprise,” “comprising,” “include,” “including,” “have,” and “having” are used in the inclusive, open sense, meaning that additional elements may be included. The terms “such as”, “e.g.,”, as used herein are non-limiting and are for illustrative purposes only. “Including” and “including but not limited to” are used interchangeably.

The verb “comprise” as is used in this description and in the claims and its conjugations are used in its non-limiting sense to mean that items following the word are included, but items not specifically mentioned are not excluded. The present invention may suitably “comprise”, “consist of”, or “consist essentially of”, the steps, elements, and/or reagents described in the claims.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

Throughout the description, where compositions are described as having, including, or comprising, specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

The term “pharmaceutically acceptable” means suitable for use in contact with the tissues of humans and animals without undue toxicity, irritation, allergic response, and the like, commensurate with a reasonable benefit/risk ratio, and effective for their intended use within the scope of sound medical judgment.

The term “pharmaceutically acceptable salt” include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods. The term “pharmaceutically acceptable salts” also includes those obtained by reacting the active compound functioning as an acid, with an inorganic or organic base to form a salt, for example salts of ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris-(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, and the like. Non limiting examples of inorganic or metal salts include lithium, sodium, calcium, potassium, magnesium salts and the like.

Additionally, the salts of the compounds described herein, can exist in either hydrated or unhydrated (the anhydrous) form or as solvates with other solvent molecules. Non-limiting examples of hydrates include monohydrates, dihydrates, etc. Nonlimiting examples of solvates include ethanol solvates, acetone solvates, etc.

The term “solvates” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate, when the solvent is alcohol, the solvate formed is an alcoholate. Hydrates are formed by the combination of one or more molecules of water with one of the substances in which the water retains its molecular state as H₂O, such combination being able to form one or more hydrate.

The compounds and salts described herein can exist in several tautomeric forms, including the enol and imine form, and the keto and enamine form and geometric isomers and mixtures thereof. Tautomers exist as mixtures of a tautomeric set in solution. In solid form, usually one tautomer predominates. Even though one tautomer may be described, the present application includes all tautomers of the present compounds. A tautomer is one of two or more structural isomers that exist in equilibrium and are readily converted from one isomeric form to another. This reaction results in the formal migration of a hydrogen atom accompanied by a switch of adjacent conjugated double bonds. In solutions where tautomerization is possible, a chemical equilibrium of the tautomers will be reached. The exact ratio of the tautomers depends on several factors, including temperature, solvent, and pH. The concept of tautomers that are interconvertable by tautomerizations is called tautomerism.

Of the various types of tautomerism that are possible, two are commonly observed. In keto-enol tautomerism a simultaneous shift of electrons and a hydrogen atom occurs.

Tautomerizations can be catalyzed by: Base: 1. deprotonation; 2. formation of a delocalized anion (e.g., an enolate); 3. protonation at a different position of the anion; Acid: 1. protonation; 2. formation of a delocalized cation; 3. deprotonation at a different position adjacent to the cation.

The terms below, as used herein, have the following meanings, unless indicated otherwise:
“Amino” refers to the —NH₂ radical.
“Cyano” refers to the —CN radical.
“Halo” or “halogen” refers to bromo, chloro, fluoro or iodo radical.
“Hydroxy” or “hydroxyl” refers to the —OH radical.
“Imino” refers to the ═NH substituent.
“Nitro” refers to the —NO₂ radical.
“Oxo” refers to the ═O substituent.
“Thioxo” refers to the ═S substituent.

“Alkyl” or “alkyl group” refers to a fully saturated, straight or branched hydrocarbon chain radical having from one to twelve carbon atoms, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene” or “alkylene chain” refers to a fully saturated, straight or branched divalent hydrocarbon chain radical, and having from one to twelve carbon atoms. Non-limiting examples of C₁-C₁₂ alkylene include methylene, ethylene, propylene, n-butylene, ethenylene, propenylene, n-butenylene, propynylene, n-butynylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, an alkylene chain can be optionally substituted.

“Alkenyl” or “alkenyl group” refers to a straight or branched hydrocarbon chain radical having from two to twelve carbon atoms, and having one or more carbon-carbon double bonds. Each alkenyl group is attached to the rest of the molecule by a single bond. Alkenyl group comprising any number of carbon atoms from 2 to 12 are included. An alkenyl group comprising up to 12 carbon atoms is a C₂-C₁₂ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₀ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkenylene” or “alkenylene chain” refers to a straight or branched divalent hydrocarbon chain radical, having from two to twelve carbon atoms, and having one or more carbon-carbon double bonds. Non-limiting examples of C₂-C₁₂ alkenylene include ethene, propene, butene, and the like. The alkenylene chain is attached to the rest of the molecule through a single bond and to the radical group through a single bond. The points of attachment of the alkenylene chain to the rest of the molecule and to the radical group can be through one carbon or any two carbons within the chain. Unless stated otherwise specifically in the specification, an alkenylene chain can be optionally substituted.

“Alkynyl” or “alkynyl group” refers to a straight or branched hydrocarbon chain radical having from two to twelve carbon atoms, and having one or more carbon-carbon triple bonds. Each alkynyl group is attached to the rest of the molecule by a single bond. Alkynyl group comprising any number of carbon atoms from 2 to 12 are included. An alkynyl group comprising up to 12 carbon atoms is a C₂-C₁₂ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁₁ and C₁₂ alkynyls. Non-limiting examples of C₂-C₁₂ alkenyl include ethynyl, propynyl, butynyl, pentynyl and the like. Unless stated otherwise specifically in the specification, an alkyl group can be optionally substituted.

“Alkynylene” or “alkynylene chain” refers to a straight or branched divalent hydrocarbon chain radical, having from two to twelve carbon atoms, and having one or more carbon-carbon triple bonds. Non-limiting examples of C₂-C₁₂ alkynylene include ethynylene, propargylene and the like. The alkynylene chain is attached to the rest of the molecule through a single bond and to the radical group through a single bond. The points of attachment of the alkynylene chain to the rest of the molecule and to the radical group can be through one carbon or any two carbons within the chain. Unless stated otherwise specifically in the specification, an alkynylene chain can be optionally substituted.

“Alkoxy” refers to a radical of the formula —ORₐ where Rₐ is an alkyl, alkenyl or alkynl radical as defined above containing one to twelve carbon atoms. Unless stated otherwise specifically in the specification, an alkoxy group can be optionally substituted.

“Alkylamino” refers to a radical of the formula —NHRₐ or —NRₐRₐ where each Rₐ is, independently, an alkyl, alkenyl or alkynyl radical as defined above containing one to twelve carbon atoms. Unless stated otherwise specifically in the specification, an alkylamino group can be optionally substituted.

“Alkylcarbonyl” refers to the —C(═O)Rₐ moiety, wherein Rₐ is an alkyl, alkenyl or alkynyl radical as defined above. A non-limiting example of an alkyl carbonyl is the methyl carbonyl (“acetal”) moiety. Alkylcarbonyl groups can also be referred to as “C_{w}-C_{z} acyl” where w and z depicts the range of the number of carbon in Rₐ, as defined above. For example, “C₁-C₁₀ acyl” refers to alkylcarbonyl group as defined above, where Rₐ is C₁-C₁₀ alkyl, C₂-C₁₀ alkenyl, or C₂-C₁₀ alkynyl radical as defined above. Unless stated otherwise specifically in the specification, an alkyl carbonyl group can be optionally substituted.

“Aryl” refers to a hydrocarbon ring system radical comprising hydrogen, 6 to 18 carbon atoms and at least one aromatic ring. For purposes of this invention, the aryl radical can be a monocyclic, bicyclic, tricyclic or tetracyclic ring system, which can include fused or bridged ring systems. Aryl radicals include, but are not limited to, aryl radicals derived from phenyl (benzene), aceanthrylene, acenaphthylene, acephenanthrylene, anthracene, azulene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” is meant to include aryl radicals that are optionally substituted.

“Aralkyl” or “arylalkyl” refers to a radical of the formula —R_{b}—R_{c} where R_{b} is an alkylene group as defined above and R_{c} is one or more aryl radicals as defined above. Aralkyl radicals include, but are not limited to, benzyl, diphenylmethyl and the like. Unless stated otherwise specifically in the specification, an aralkyl group can be optionally substituted.

“Aralkenyl” or “arylalkenyl” refers to a radical of the formula —R_{b}—R_{c} where R_{b} is an alkenylene group as defined above and R_{c} is one or more aryl radicals as defined above. Unless stated otherwise specifically in the specification, an aralkenyl group can be optionally substituted.

“Aralkynyl” or “arylalkynyl” refers to a radical of the formula —R_{b}—R_{c} where R_{b} is an alkynylene group as defined above and R_{c} is one or more aryl radicals as defined above. Unless stated otherwise specifically in the specification, an aralkynyl group can be optionally substituted.

“Carbocyclyl,” “carbocyclic ring” or “carbocycle” refers to a ring structure, wherein the atoms which form the ring are each carbon. Carbocyclic rings can comprise from 3 to 20 carbon atoms in the ring. Carbocyclic rings include aryls and cycloalkyl. Cycloalkenyl and cycloalkynyl as defined herein. Unless stated otherwise specifically in the specification, a carbocyclyl group can be optionally substituted.

“Cycloalkyl” refers to a stable non-aromatic monocyclic or polycyclic fully saturated hydrocarbon radical consisting solely of carbon and hydrogen atoms, which can include fused, bridged, or spiral ring systems, having from three to twenty carbon atoms, preferably having from three to ten carbon atoms, and which is attached to the rest of the molecule by a single bond. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Cycloalkenyl” refers to a stable non-aromatic monocyclic or polycyclic hydrocarbon radical consisting solely of carbon and hydrogen atoms, having one or more carbon-carbon double bonds, which can include fused, bridged, or spiral ring systems, having from three to twenty carbon atoms, preferably having from three to ten carbon atoms, and which is attached to the rest of the molecule by a single bond. Monocyclic cycloalkenyl radicals include, for example, cyclopentenyl, cyclohexenyl, cycloheptenyl, cycloctenyl, and the like. Polycyclic cycloalkenyl radicals include, for example, bicyclo[2.2.1]hept-2-enyl and the like. Unless otherwise stated specifically in the specification, a cycloalkenyl group can be optionally substituted.

“Cycloalkynyl” refers to a stable non-aromatic monocyclic or polycyclic hydrocarbon radical consisting solely of carbon and hydrogen atoms, having one or more carbon-carbon triple bonds, which can include fused, bridged, or spiral ring systems, having from three to twenty carbon atoms, preferably having from three to ten carbon atoms, and which is attached to the rest of the molecule by a single bond. Monocyclic cycloalkynyl radicals include, for example, cycloheptynyl, cyclooctynyl, and the like. Unless otherwise stated specifically in the specification, a cycloalkynyl group can be optionally substituted.

“Cycloalkylalkyl” refers to a radical of the formula —R_{b}—R_{d} where R_{b} is an alkylene, alkenylene, or alkynylene group as defined above and R_{d} is a cycloalkyl, cycloalkenyl, cycloalkynyl radical as defined above. Unless stated otherwise specifically in the specification, a cycloalkylalkyl group can be optionally substituted.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group can be optionally substituted.

“Haloalkenyl” refers to an alkenyl radical, as defined above, that is substituted by one or more halo radicals, as defined above, e.g., 1-fluoropropenyl, 1,1-difluorobutenyl, and the like. Unless stated otherwise specifically in the specification, a haloalkenyl group can be optionally substituted.

“Haloalkynyl” refers to an alkynyl radical, as defined above, that is substituted by one or more halo radicals, as defined above, e.g., 1-fluoropropynyl, 1-fluorobutynyl, and the like. Unless stated otherwise specifically in the specification, a haloalkynyl group can be optionally substituted.

“Heterocyclyl,” “heterocyclic ring” or “heterocycle” refers to a stable 3- to 20-membered non-aromatic, partially aromatic, or aromatic ring radical which consists of two to twelve carbon atoms and from one to six heteroatoms selected from the group consisting of nitrogen, oxygen and sulfur. Heterocyclyl or heterocyclic rings include heteroaryls as defined below. Unless stated otherwise specifically in the specification, the heterocyclyl radical can be a monocyclic, bicyclic, tricyclic or tetracyclic ring system, which can include fused, bridged, and spiral ring systems; and the nitrogen, carbon or sulfur atoms in the heterocyclyl radical can be optionally oxidized; the nitrogen atom can be optionally quaternized; and the heterocyclyl radical can be partially or fully saturated. Examples of such heterocyclyl radicals include, but are not limited to, aziridinyl, oextanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, pyridine-one, and the like. The point of attachment of the heterocyclyl, heterocyclic ring, or heterocycle to the rest of the molecule by a single bond is through a ring member atom, which can be carbon or nitrogen. Unless stated otherwise specifically in the specification, a heterocyclyl group can be optionally substituted.

“Heterocyclylalkyl” refers to a radical of the formula —R_{b}—Rₑ where R_{b} is an alkylene group as defined above and Rₑ is a heterocyclyl radical as defined above. Unless stated otherwise specifically in the specification, a heterocyclylalkyl group can be optionally substituted.

“Heterocyclylalkenyl” refers to a radical of the formula —R_{b}—Rₑ where R_{b} is an alkenylene group as defined above and Rₑ is a heterocyclyl radical as defined above. Unless stated otherwise specifically in the specification, a heterocyclylalkenyl group can be optionally substituted.

“Heterocyclylalkynyl” refers to a radical of the formula —R_{b}—Rₑ where R_{b} is an alkynylene group as defined above and Rₑ is a heterocyclyl radical as defined above. Unless stated otherwise specifically in the specification, a heterocyclylalkynyl group can be optionally substituted.

“N-heterocyclyl” refers to a heterocyclyl radical as defined above containing at least one nitrogen and where the point of attachment of the heterocyclyl radical to the rest of the molecule is through a nitrogen atom in the heterocyclyl radical. Unless stated otherwise specifically in the specification, a N-heterocyclyl group can be optionally substituted.

“Heteroaryl” refers to a 5- to 20-membered ring system radical one to thirteen carbon atoms and one to six heteroatoms selected from the group consisting of nitrogen, oxygen and sulfur, as the ring member. For purposes of this invention, the heteroaryl radical can be a monocyclic, bicyclic, tricyclic or tetracyclic ring system, which can include fused or bridged ring systems, wherein at least one ring containing a heteroatom ring member is aromatic. The nitrogen, carbon or sulfur atoms in the heteroaryl radical can be optionally oxidized and the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, pyrazolopyridine, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl group can be optionally substituted.

“N-heteroaryl” refers to a heteroaryl radical as defined above containing at least one nitrogen and where the point of attachment of the heteroaryl radical to the rest of the molecule is through a nitrogen atom in the heteroaryl radical. Unless stated otherwise specifically in the specification, an N-heteroaryl group can be optionally substituted.

“Heteroarylalkyl” refers to a radical of the formula —R_{b}—R_{f} where R_{b} is an alkylene chain as defined above and R_{f} is a heteroaryl radical as defined above. Unless stated otherwise specifically in the specification, a heteroarylalkyl group can be optionally substituted.

“Heteroarylalkenyl” refers to a radical of the formula —R_{b}—R_{f} where R_{b} is an alkenylene, chain as defined above and R_{f} is a heteroaryl radical as defined above. Unless stated otherwise specifically in the specification, a heteroarylalkenyl group can be optionally substituted.

“Heteroarylalkynyl” refers to a radical of the formula —R_{b}—R_{f} where R_{b} is an alkynylene chain as defined above and R_{f} is a heteroaryl radical as defined above. Unless stated otherwise specifically in the specification, a heteroarylalkynyl group can be optionally substituted.

“Thioalkyl” refers to a radical of the formula —SRₐ where Rₐ is an alkyl, alkenyl, or alkynyl radical as defined above containing one to twelve carbon atoms. Unless stated otherwise specifically in the specification, a thioalkyl group can be optionally substituted.

The term “substituted” used herein means any of the above groups (e.g., alkyl, alkylene, alkenyl, alkenylene, alkynyl, alkynylene, alkoxy, alkylamino, alkylcarbonyl, thioalkyl, aryl, aralkyl, carbocyclyl, cycloalkyl, cycloalkenyl, cycloalkynyl, cycloalkylalkyl, haloalkyl, heterocyclyl, N-heterocyclyl, heterocyclylalkyl, heteroaryl, N-heteroaryl, heteroarylalkyl, heteroarylalkenyl, heteroarylalkynyl, etc.) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted” also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing substituents can also be optionally substituted with one or more of the above substituents.

As used herein, the symbol “ [symbol] ” (hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example, “ [symbol] ” indicates that the chemical entity “A” is bonded to another chemical entity via the point of attachment bond. Furthermore, the specific point of attachment to the non-depicted chemical entity can be specified by inference. For example, the compound

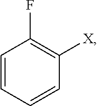

wherein X is “ [symbol] ” infers that the point of attachment bond is the bond by which X is depicted as being attached to the phenyl ring at the ortho position relative to fluorine.

The phrases “parenteral administration” and “administered parenterally” are art-recognized terms, and include modes of administration other than enteral and topical administration, such as injections, and include, without limitation, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrastemal injection and infusion.

The term “treating” is art-recognized and includes inhibiting a disease, disorder or condition in a subject, e.g., impeding its progress; and relieving the disease, disorder or condition, e.g., causing regression of the disease, disorder and/or condition. Treating the disease or condition includes ameliorating at least one symptom of the particular disease or condition, even if the underlying pathophysiology is not affected.

The term “preventing” is art-recognized and includes stopping a disease, disorder or condition from occurring in a subject, which may be predisposed to the disease, disorder and/or condition but has not yet been diagnosed as having it. Preventing a condition related to a disease includes stopping the condition from occurring after the disease has been diagnosed but before the condition has been diagnosed.

A “patient,” “subject,” or “host” to be treated by the subject method may mean either a human or non-human animal, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder.

The terms “prophylactic” or “therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if it is administered after manifestation of the unwanted condition, the treatment is therapeutic (i.e., it is intended to diminish, ameliorate, or stabilize the existing unwanted condition or side effects thereof).

The terms “therapeutic agent”, “drug”, “medicament” and “bioactive substance” are art-recognized and include molecules and other agents that are biologically, physiologically, or pharmacologically active substances that act locally or systemically in a patient or subject to treat a disease or condition. The terms include without limitation pharmaceutically acceptable salts thereof and prodrugs. Such agents may be acidic, basic, or salts; they may be neutral molecules, polar molecules, or molecular complexes capable of hydrogen bonding; they may be prodrugs in the form of ethers, esters, amides and the like that are biologically activated when administered into a patient or subject.

The phrase “therapeutically effective amount” or “pharmaceutically effective amount” is an art-recognized term. In certain embodiments, the term refers to an amount of a therapeutic agent that produces some desired effect at a reasonable benefit/risk ratio applicable to any medical treatment. In certain embodiments, the term refers to that amount necessary or sufficient to eliminate, reduce or maintain a target of a particular therapeutic regimen. The effective amount may vary depending on such factors as the disease or condition being treated, the particular targeted constructs being administered, the size of the subject or the severity of the disease or condition. One of ordinary skill in the art may empirically determine the effective amount of a particular compound without necessitating undue experimentation. In certain embodiments, a therapeutically effective amount of a therapeutic agent for in vivo use will likely depend on a number of factors, including: the rate of release of an agent from a polymer matrix, which will depend in part on the chemical and physical characteristics of the polymer; the identity of the agent; the mode and method of administration; and any other materials incorporated in the polymer matrix in addition to the agent.

The term “ED50” is art-recognized. In certain embodiments, ED50 means the dose of a drug, which produces 50% of its maximum response or effect, or alternatively, the dose, which produces a pre-determined response in 50% of test subjects or preparations. The term “LD50” is art-recognized. In certain embodiments, LD50 means the dose of a drug, which is lethal in 50% of test subjects. The term “therapeutic index” is an art-recognized term, which refers to the therapeutic index of a drug, defined as LD50/ED50.

The terms “IC₅₀,” or “half maximal inhibitory concentration” is intended to refer to the concentration of a substance (e.g., a compound or a drug) that is required for 50% inhibition of a biological process, or component of a process, including a protein, subunit, organelle, ribonucleoprotein, etc.

“Optional” or “optionally” means that the subsequently described circumstance may or may not occur, so that the description includes instances where the circumstance occurs and instances where it does not. For example, the phrase “optionally substituted” means that a non-hydrogen substituent may or may not be present on a given atom, and, thus, the description includes structures wherein a non-hydrogen substituent is present and structures wherein a non-hydrogen substituent is not present.

The term “or” as used herein should be understood to mean “and/or”, unless the context clearly indicates otherwise.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

All percentages and ratios used herein, unless otherwise indicated, are by weight.

The terms “healthy” and “normal” are used interchangeably herein to refer to a subject or particular cell or tissue that is devoid (at least to the limit of detection) of a disease condition.

The terms “neurodegenerative disease”, “neurodegenerative disorder”, or “neurodegenerative condition” are used interchangeably herein to refer to a varied assortment of central nervous system diseases, disorders, and conditions characterised by gradual and progressive loss of neural tissue and/or neural tissue function. A neurodegenerative disease is a class of neurological disorder or disease, and where the neurological disease is characterized by a gradual and progressive loss of neural tissue, and/or altered neurological function, typically reduced neurological function as a result of a gradual and progressive loss of neural tissue.

The term “vascular dementia” is also referred to as “multi-infarct dementia” in the art refers to a group of syndromes caused by different mechanisms all resulting in vascular lesions in the brain. The main subtypes of vascular dementia are, for example vascular mild cognitive impairment, multi-infarct dementia, vascular dementia due to a strategic single infarct (affecting the thalamus, the anterior cerebral artery, the parietal lobes or the cingulate gyrus), vascular dementia due to hemorrhagic lesions, small vessel disease (including, e.g., vascular dementia due to lacunar lesions and Binswanger disease), and mixed Alzheimer's Disease with vascular dementia.

The term “disease”, “disorder”, or “condition” is used interchangeably herein, and refers to any alteration in state of the body or of some of the organs, interrupting or disturbing the performance of the functions and/or causing symptoms such as discomfort, dysfunction, distress, or even death to the person afflicted or those in contact with a person. A disease or disorder can also relate to a distemper, ailing, ailment, malady, disorder, sickness, illness, complaint, inderdisposion or affectation.

The terms “blood-brain barrier” or “BBB” are used interchangeably herein, and are used to refer to the permeability barrier that exists in blood vessels as they travel through the brain tissue that severely restricts and closely regulates what is exchanged between the blood and the brain tissue. The blood brain barrier components include the endothelial cells that form the innermost lining of all blood vessels, the tight junctions between adjacent endothelial cells that are the structural correlate of the BBB, the basement membrane of endothelial cells and the expanded foot processes of nearby astrocytes which cover nearly all of the exposed outer surface of the blood vessel. The BBB prevents most substances in the blood from entering brain tissue, including most large molecules such as Ig, antibodies, complement, albumin and drugs and small molecules.

The term “abnormal BBB” is used to refer to a dysfunctional BBB, for example, where the BBB does not allow transit of molecules that normally transit a functional BBB, for example nutrients and sugars such as glucose. An abnormal BBB can also refer to when the BBB is permeable to molecules that a normally functioning BBB would typically exclude, which is typically referred to “BBB permeability” herein.

The terms “BBB permeability” or “permeable BBB” are commonly referred to by persons in the art as “leaky BBB”. The terms are used interchangeably herein to refer to impaired BBB integrity and increased vascular permeability. For example, a permeable BBB allows transit of molecules through the BBB that an intact BBB would normally exclude from the brain tissue, for example, Ig molecules, complement proteins, serum albumin and numerous other proteins. An assay to determine the presence of a permeable BBB can be, for example, to assess the presence of extravascular Ig in the brain tissue which is normally be restricted to the lumen of blood vessels when the BBB is functioning normally (i.e., when the BBB is not permeable).

Embodiments described herein relate generally to compositions and methods of treating neurodegeneration and/or neurodegenerative conditions, diseases and/or disorders. In some embodiments, the neurodegeneration and/or neurodegenerative conditions, diseases and/or disorders can be caused by and/or associated with enhanced or aberrant 15-PGDH activity in the subject in need thereof. It was found that 15-PDGH activity is increased in certain neurodegenerative conditions, diseases, and/or disorders and that this activity could be inhibited, such as with a 15-PGDH inhibitor described herein, to provide or promote neuroprotection in a subject from axonal degeneration, neuronal cell death, and/or glia cell damage after injury, augment neuronal signaling underlying learning and memory, ameliorate memory loss or cognitive decline, stimulate neuronal regeneration, attenuate or decrease blood brain barrier permeability and/or treat neurodegenerative conditions, diseases and/or disorders.

Accordingly, in one aspect, this disclosure provides a method of treating neurodegeneration and/or neurodegenerative conditions, diseases and/or disorders caused by and/or associated with enhanced or aberrant 15-PGDH activity. The method includes administering to the subject an amount of a 15-PGDH inhibitor effective to inhibit 15-PGDH activity.

In some embodiments, the neurodegenerative condition, disease, or disorder is associated with an increase in 15-PGDH activity in neurotissue, such as brain tissue, of at least about 5%, about 10%, about 20%, about 30%, about 40%, about 50%, about 60%, about 70%, about 80%, about 90%, about 100%, about 110%, about 120%, about 130%, about 140%, about 150%, about 160%, about 170%, about 180%, about 190%, about 200%, about 250%, about 300%, about 350%, about 400%, about 450%, about 500%, about 600%, about 700%, about 800%, about 900%, or about 1000% relative to normal or healthy neurotissue. In certain embodiments, administration of a 15-PGDH inhibitor can be used to decrease 15-PGDH activity in neurotissue of the subject from about 5% to about 200%, about 5% to about 180%, about 5% to about 160%, about 5% to about 140%, about 5% to about 120%, about 5% to about 100%, about 5% to about 80%, about 5% to about 60%, about 5% to about 40%, about 10% to about 200%, about 10% to about 180%, about 10% to about 160%, about 10% to about 140%, about 10% to about 120%, about 10% to about 100%, about 10% to about 80%, about 10% to about 60%, about 30% to about 200%, about 30% to about 180%, about 30% to about 160%, about 30% to about 140%, about 30% to about 120%, about 30% to about 100%, about 30% to about 80%, about 40% to about 200%, about 40% to about 180%, about 40% to about 160%, about 40% to about 140%, about 40% to about 120%, about 40% to about 100%, about 50% to about 200%, about 50% to about 180%, about 50% to about 160%, about 50% to about 140%, about 50% to about 120%, about 60% to about 200%, about 60% to about 180%, about 60% to about 160%, about 60% to about 140%, about 70% to about 200%, about 70% to about 180%, about 70% to about 160%, about 80% to about 200%, about 80% to about 180%, or about 80% to about 200%.

Neurodegenerative conditions, diseases, or disorders that can be associated with aberrant 15-PGDH activity include subarachnoid hemorrhage, schizophrenia, major depression, bipolar disorder, normal aging, epilepsy, traumatic brain injury and/or a visual symptom associated therewith, post-traumatic stress disorder, Parkinson's disease, Parkinson Plus syndromes, Lewy Body Dementia, multiple system atrophy, corticobasal neurodegeneration, progressive supranuclear palsy, Alzheimer's disease, Alzheimer's disease related dementias, Down syndrome, spinocerebellar ataxia, amyotrophic lateral sclerosis, Huntington's disease, stroke, brain radiation therapy, chronic stress, abuse of a neuro-active drug, retinal degeneration, spinal cord injury, peripheral nerve injury, idiopathic peripheral neuropathy, cognitive decline and/or general frailty associated with normal aging and/or chemotherapy, chemotherapy induced neuropathy, concussive injury, peripheral nerve crush injury, peripheral neuropathy, diabetic neuropathy, post-traumatic headache, multiple sclerosis, retinal degeneration and dystrophy, Leber congenital amaurosis, retinitis pigmentosa, cone-rod dystrophy, microphthalmia, anophthalmia, myopia, and hyperopia, spinal cord injury, traumatic spinal cord injury, peripheral nerve injury, retinal neuronal death related diseases, retinal trauma, Autism, Stargardt disease, Kearns-Sayre syndrome, Pure neurosensory deafness, Hereditary hearing loss with retinal diseases, Hereditary hearing loss with system atrophies of the nervous system, Progressive spinal muscular atrophy, Progressive bulbar palsy, Primary lateral sclerosis, Hereditary forms of progressive muscular atrophy and spastic paraplegia, Frontotemporal dementia, Dementia with Lewy bodies, Corticobasal degeneration, Progressive supranuclear palsy, Prion disorders causing neurodegeneration, Multiple system atrophy, Hereditary spastic paraparesis, Friedreich ataxia, Non-Friedreich ataxia, Spinocerebellar atrophies, Amyloidoses, Metabolic-related neurodegenerative disorders, Toxin-related neurodegenerative disorders, Multiple sclerosis, Charcot Marie Tooth, Diabetic neuropathy, Metabolic neuropathies, Endocrine neuropathies, Creutzfeldt-Jacob Disease, Primary progressive aphasia, Frontotemporal Lobar Degeneration, Cortical blindness, Shy-Drager Syndrome, Diffuse cerebral cortical atrophy of non-Alzheimer type, Lewy-body dementia, Pick disease, Thalamic degeneration, Mesolimbocortical dementia of non-Alzheimer type, Nonhuntingtonian types of chorea and dementia, Cortical-striatal-spinal degeneration, Dementia-Parkinson-amyotrophic lateral sclerosis complex, Cerebrocerebellar degeneration, Cortico-basal ganglionic degeneration, Familial dementia with spastic paraparesis or myoclonus, Tourette syndrome, or viral infection.

In some embodiments, the neurodegeneration and/or neurodegenerative condition, disease, or disorder is associated with an aberrant level (e.g., decrease or increase in the level) of at least one eicosanoid selected from PGE2, 15-keto-PGE2, PGF2α, 6-keto-PGF1α, PGD2, PGJ2, TN-E, TXB2, LTB4, 15-HETE, 12-HETE, 8-HETE, 5-HETE, 17-HDA, 12, 13-DiHOME, 9,10-DiHOME, 14,15-DHET, or 11,12-DHET in neurotissue of the subject. For example, the neurodegenerative condition, disease, or disorder can be associated with an increase or decrease in the level of at least one eicosanoid selected from PGE2, 15-keto-PGE2, PGF2α, 6-keto-PGF1α, PGD2, PGJ2, TN-E, TXB2, LTB4, 15-HETE, 12-HETE, 8-HETE, 5-HETE, 17-HDA, 12, 13-DiHOME, 9,10-DiHOME, 14,15-DHET, or 11,12-DHET of at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, or more in neurotissue of the subject relative to a normal or healthy subject.

In some embodiments, the 15-PGDH inhibitor can be administered to the subject to modulate the level of the aberrant eicosanoid to a normal or healthy level in the neurotissue. For example, the neurodegeneration and/or neurodegenerative condition, disease, or disorder can be associated with an increase in the level of at least one of 15-keto-PGE2, TN-E, TXB2, LTB4, 15-HETE, 12-HETE, 8-HETE, 5-HETE, 17-HDA, 12, 13-DiHOME, 9,10-DiHOME, or 14,15-DHET in neurotissue (e.g., brain tissue) of the subject, and the 15-PGDH inhibitor can be administered to the subject at amount effective to decrease the levels of the at least one of 15-keto-PGE2, TN-E, TXB2, LTB4, 15-HETE, 12-HETE, 8-HETE, 5-HETE, 17-HDA, 12, 13-DiHOME, 9,10-DiHOME, or 14,15-DHET in neurotissue of the subject. In another example, the neurodegeneration and/or neurodegenerative condition, disease, or disorder can be associated with decrease in the levels of at least one of PGJ2, TNE, 15-HETE, or 9,10-DiHOME in neurotissue (e.g., brain tissue) of the subject, and the 15-PGDH inhibitor can be administered to the subject at amount effective to increase the levels of the at least one of PGJ2, TNE, 15-HETE, or 9,10-DiHOME in neurotissue (e.g., brain tissue) of the subject.

In some embodiments, the neurotissue can include brain tissue of the subject, such as the hippocampus of the subject. The 15-PGDH inhibitor can be administered at an amount effective to stimulate hippocampal neurogenesis.

Other embodiments described herein relate to a method of treating and/or inhibiting memory loss and/or cognitive decline in a subject in need thereof by administering to the subject a therapeutically effective amount of a 15-PGDH inhibitor. In some embodiments, the memory loss and/or cognitive decline can be caused by and/or associated with neurodegeneration and/or neurodegenerative conditions, diseases and/or disorders. The therapeutically effective amount of the administered 15-PGDH can be an amount effective to ameliorate memory loss and/or cognitive decline and/or improve memory and/or cognition.

In some embodiments, neurodegenerative condition, disease, or disorder can include at least one of Alzheimer's disease, Lewy body dementia, Vascular dementia, Age-related dementia, Frontotemporal dementia, mixed dementia, or traumatic brain injury.

In still other embodiments, the memory loss and/or cognitive decline is associated with an aberrant level (e.g., decrease or increase in the level) of at least one eicosanoid selected from PGE2, 15-keto-PGE2, PGF2α, 6-keto-PGF1α, PGD2, PGJ2, TN-E, TXB2, LTB4, 15-HETE, 12-HETE, 8-HETE, 5-HETE, 17-HDA, 12, 13-DiHOME, 9,10-DiHOME, 14,15-DHET, or 11,12-DHET in brain tissue (e.g., hippocampus) of the subject. The 15-PGDH inhibitor can be administered to the subject to modulate the level of the aberrant eicosanoid to a normal or healthy level in the brain tissue. For example, the memory loss and/or cognitive decline can be associated with an increase in the level of at least one of 15-keto-PGE2, TN-E, TXB2, LTB4, 15-HETE, 12-HETE, 8-HETE, 5-HETE, 17-HDA, 12, 13-DiHOME, 9,10-DiHOME, or 14,15-DHET in brain tissue of the subject, and the 15-PGDH inhibitor can be administered to the subject at amount effective to decrease the level of the at least one of 15-keto-PGE2, TN-E, TXB2, LTB4, 15-HETE, 12-HETE, 8-HETE, 5-HETE, 17-HDA, 12, 13-DiHOME, 9,10-DiHOME, or 14,15-DHET in brain tissue of the subject. In another example, the memory loss and/or cognitive decline can be associated with a decrease in the level of at least one of PGJ2, TNE, 15-HETE, or 9,10-DiHOME in brain tissue of the subject, and the 15-PGDH inhibitor can be administered to the subject at amount effective to increase the levels of the at least one of PGJ2, TNE, 15-HETE, or 9,10-DiHOME in e.g., brain tissue of the subject.

Still other embodiments described herein relate to methods of reducing blood brain barrier permeability in a subject in need thereof. The method includes administering to the subject a therapeutically effective amount of a 15-PGDH inhibitor.

In some embodiments, the subject with blood brain permeability has or is at risk of neurodegenerative condition, disorder, or disease. For example, the subject can have or be at risk of mild cognitive impairment, Alzheimer's disease, Lewy body dementia, Vascular dementia, Age-related dementia, Frontotemporal dementia, mixed dementia, Parkinson's disease, Huntington's disease, multiple sclerosis, diabetic retinopathy, prion disorders, or amyotrophic lateral sclerosis.

In some embodiments, the subject has an aberrant level (e.g., decrease or increase in the level) of at least one eicosanoid selected from PGE2, 15-keto-PGE2, PGF2α, 6-keto-PGF1α, PGD2, PGJ2, TN-E, TXB2, LTB4, 15-HETE, 12-HETE, 8-HETE, 5-HETE, 17-HDA, 12, 13-DiHOME, 9,10-DiHOME, 14,15-DHET, or 11,12-DHET in brain tissue. The 15-PGDH inhibitor can be administered to the subject to modulate the level of the aberrant eicosanoid to a normal or healthy level in the brain tissue. For example, the subject can have an increase in the levels of at least one of 15-keto-PGE2, TN-E, TXB2, LTB4, 15-HETE, 12-HETE, 8-HETE, 5-HETE, 17-HDA, 12, 13-DiHOME, 9,10-DiHOME, or 14,15-DHET in neurotissue (e.g., brain tissue) of the subject, and the 15-PGDH inhibitor can be administered to the subject at amount effective to decrease the level of the at least one of 15-keto-PGE2, TN-E, TXB2, LTB4, 15-HETE, 12-HETE, 8-HETE, 5-HETE, 17-HDA, 12, 13-DiHOME, 9,10-DiHOME, or 14,15-DHET in the brain tissue. In another example, the subject can have a decrease in the level of at least one of PGJ2, TNE, 15-HETE, or 9,10-DiHOME in brain tissue, and the 15-PGDH inhibitor can be administered to the subject at amount effective to increase the levels of the at least one of PGJ2, TNE, 15-HETE, or 9,10-DiHOME in brain tissue of the subject.

In some embodiments, 15-PGDH inhibitors used to treat the neurodegenerative disease, disorder or condition can be identified using assays in which putative inhibitor compounds are applied to cells expressing 15-PGDH and then the functional effects on 15-PGDH activity are determined. Samples or assays comprising 15-PGDH that are treated with a potential inhibitor are compared to control samples without the inhibitor to examine the extent of effect. Control samples (untreated with modulators) are assigned a relative 15-PGDH activity value of 100%. Inhibition of 15-PGDH is achieved when the 15-PGDH activity value relative to the control is about 80%, optionally 50% or 25%, 10%, 5% or 1%.

Agents tested as 15-PGDH inhibitors can be any small chemical molecule or compound. Typically, test compounds will be small chemical molecules, natural products, or peptides. The assays are designed to screen large chemical libraries by automating the assay steps and providing compounds from any convenient source to assays, which are typically run in parallel (e.g., in microtiter formats on microtiter plates in robotic assays).

In some embodiments, the 15-PGDH inhibitor can include a compound having the following formula (I):

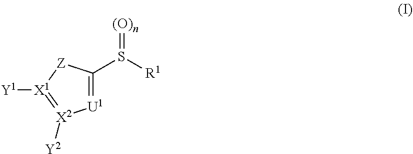

or a pharmaceutically acceptable salt, tautomer, or solvate thereof;
wherein n is 0-2;
Y¹, Y², and R¹ are the same or different and are independently hydrogen or a substituted or unsubstituted group selected from C₁-C₂₄ alkyl, C₂-C₂₄ alkenyl, C₂-C₂₄ alkynyl, C₃-C₂₀ aryl, heteroaryl, heterocycloalkenyl containing from 5-6 ring atoms, C₆-C₂₄ alkaryl, C₆-C₂₄ aralkyl, halo, —Si(C₁-C₃ alkyl)₃, hydroxyl, sulfhydryl, C₁-C₂₄ alkoxy, C₂-C₂₄ alkenyloxy, C₂-C₂₄ alkynyloxy, C₅-C₂₀ aryloxy, acyl, acyloxy, C₂-C₂₄ alkoxycarbonyl, C₆-C₂₀ aryloxycarbonyl, C₂-C₂₄ alkylcarbonato, C₆-C₂₀ arylcarbonato, carboxy, carboxylato, carbamoyl, C₁-C₂₄ alkyl-carbamoyl, arylcarbamoyl, thiocarbamoyl, carbamido, cyano, isocyano, cyanato, isocyanato, isothiocyanato, azido, formyl, thioformyl, amino, C₁-C₂₄ alkyl amino, C₅-C₂₀ aryl amino, C₂-C₂₄ alkylamido, C₆-C₂₀ arylamido, imino, alkylimino, arylimino, nitro, nitroso, sulfo, sulfonato, C₁-C₂₄ alkylsulfanyl, arylsulfanyl, C₁-C₂₄ alkylsulfinyl, C₅-C₂₀ arylsulfinyl, C₁-C₂₄ alkylsulfonyl, C₅-C₂₀ arylsulfonyl, sulfonamide, phosphono, phosphonato, phosphinato, phospho, phosphino, polyalkylethers, phosphates, and phosphate esters, groups incorporating amino acids or other moieties expected to bear positive or negative charge at physiological pH, and combinations thereof, and wherein Y¹ and Y² may be linked to form a cyclic or polycyclic ring, wherein the ring is a substituted or unsubstituted aryl, a substituted or unsubstituted heteroaryl, a substituted or unsubstituted cycloalkyl, and a substituted or unsubstituted heterocyclyl;
U¹ is N, C—R², or C—NR³R⁴, wherein R² is selected from the group consisting of a H, a lower alkyl group, O, (CH₂)˜₁OR′ (wherein n1=1, 2, or 3), CF₃, CH₂—CH₂X, O—CH₂—CH₂X, CH₂—CH₂—CH₂X, O—CH₂—CH₂X, X, (wherein X=H, F, Cl, Br, or I), CN, (C═O)—R′, (C═O)N(R′)₂, O(CO)R′, COOR′ (wherein R′ is H or a lower alkyl group), and wherein R¹ and R² may be linked to form a cyclic or polycyclic ring, wherein R³ and R⁴ are same or different and are each selected from the group consisting of H, a lower alkyl group, O, (CH₂)ₙ₁OR′ (wherein n1=1, 2, or 3), CF₃, CH₂—CH₂X, CH₂—CH₂—CH₂X, (wherein X=H, F, C₁, Br, or I), CN, (C═O)—R′, (C═O)N(R′)₂, COOR′ (wherein R′ is H or a lower alkyl group), and R³ or R⁴ may be absent;
X¹ and X² are independently N or C, and wherein when X¹ and/or X² are N, Y¹ and/or Y², respectively, are absent; and
Z¹ is O, S, CR^{a}R^{b} or NR^{a}, wherein R^{a} and R^{b} are independently H or a C₁₋₈ alkyl, which is linear, branched, or cyclic, and which is unsubstituted or substituted.

In other embodiments, the 15-PGDH inhibitor can include a compound having the following formula (II):

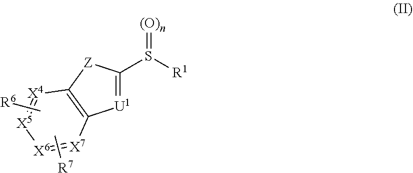

or a pharmaceutically acceptable salt, tautomer, or solvate thereof;
wherein n is 0-2
X⁴, X⁵, X⁶, and X⁷ are independently N or CR^{c};
R¹, R⁶, R⁷, and R^{c} are the same or different and independently hydrogen or a substituted or unsubstituted group selected from C₁-C₂₄ alkyl, C₂-C₂₄ alkenyl, C₂-C₂₄ alkynyl, C₃-C₂₀ aryl, heteroaryl, heterocycloalkenyl containing from 5-6 ring atoms, C₆-C₂₄ alkaryl, C₆-C₂₄ aralkyl, halo, —Si(C₁-C₃ alkyl)₃, hydroxyl, sulfhydryl, C₁-C₂₄ alkoxy, C₂-C₂₄ alkenyloxy, C₂-C₂₄ alkynyloxy, C₅-C₂₀ aryloxy, acyl, acyloxy, C₂-C₂₄ alkoxycarbonyl, C₆-C₂₀ aryloxycarbonyl, C₂-C₂₄ alkylcarbonato, C₆-C₂₀ arylcarbonato, carboxy, carboxylato, carbamoyl, C₁-C₂₄ alkyl-carbamoyl, arylcarbamoyl, thiocarbamoyl, carbamido, cyano, isocyano, cyanato, isocyanato, isothiocyanato, azido, formyl, thioformyl, amino, C₁-C₂₄ alkyl amino, C₅-C₂₀ aryl amino, C₂-C₂₄ alkylamido, C₂-C₂₄ alkylamido substituted with a hydroxyl, C₆-C₂₀ arylamido, imino, alkylimino, arylimino, nitro, nitroso, sulfo, sulfonato, C₁-C₂₄ alkylsulfanyl, arylsulfanyl, C₁-C₂₄ alkylsulfinyl, C₅-C₂₀ arylsulfinyl, C₁-C₂₄ alkylsulfonyl, C₅-C₂₀ arylsulfonyl, sulfonamide, phosphono, phosphonato, phosphinato, phospho, phosphino, polyalkylethers, phosphates, and phosphate esters, groups incorporating amino acids or other moieties expected to bear positive or negative charge at physiological pH, and combinations thereof, and wherein R⁶ and R⁷ may be linked to form a cyclic or polycyclic ring, wherein the ring is a substituted or unsubstituted aryl, a substituted or unsubstituted heteroaryl, a substituted or unsubstituted cycloalkyl, and a substituted or unsubstituted heterocyclyl;
U¹ is N, C—R², or C—NR³R⁴, wherein R² is selected from the group consisting of a H, a lower alkyl group, O, (CH₂)ₙ₁OR′ (wherein n1=1, 2, or 3), CF₃, CH₂—CH₂X, O—CH₂—CH₂X, CH₂—CH₂—CH₂X, O—CH₂—CH₂X, X, (wherein X=H, F, Cl, Br, or I), CN, (C═O)—R′, (C═O)N(R′)₂, O(CO)R′, COOR′ (wherein R′ is H or a lower alkyl group), and wherein R¹ and R² may be linked to form a cyclic or polycyclic ring, wherein R³ and R⁴ are the same or different and are each selected from the group consisting of H, a lower alkyl group, O, (CH₂)ₙ₁OR′ (wherein n1=1, 2, or 3), CF₃, CH₂—CH₂X, CH₂—CH₂—CH₂X, (wherein X=H, F, Cl, Br, or I), CN, (C═O)—R′, (C═O)N(R′)₂, COOR′ (wherein R′ is H or a lower alkyl group), and R³ or R⁴ may be absent; and
Z¹ is O, S, CR^{a}R^{b} or NR^{a}, wherein R^{a} and R^{b} are independently H or a C₁₋₈ alkyl, which is linear, branched, or cyclic, and which is unsubstituted or substituted.

In yet other embodiments, the 15-PGDH inhibitor can include a compound having the following formula (III) or (IV):

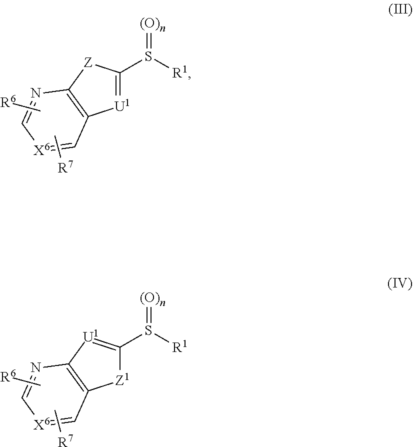

or a pharmaceutically acceptable salt, tautomer, or solvate thereof;
wherein n is 0-2
X⁶ is independently is N or CR^{c};
R¹, R⁶, R⁷, and R^{c} are the same or different and independently hydrogen or a substituted or unsubstituted group selected from C₁-C₂₄ alkyl, C₂-C₂₄ alkenyl, C₂-C₂₄ alkynyl, C₃-C₂₀ aryl, heteroaryl, heterocycloalkenyl containing from 5-6 ring atoms, C₆-C₂₄ alkaryl, C₆-C₂₄ aralkyl, halo, —Si(C₁-C₃ alkyl)₃, hydroxyl, sulfhydryl, C₁-C₂₄ alkoxy, C₂-C₂₄ alkenyloxy, C₂-C₂₄ alkynyloxy, C₅-C₂₀ aryloxy, acyl, acyloxy, C₂-C₂₄ alkoxycarbonyl, C₆-C₂₀ aryloxycarbonyl, C₂-C₂₄ alkylcarbonato, C₆-C₂₀ arylcarbonato, carboxy, carboxylato, carbamoyl, C₁-C₂₄ alkyl-carbamoyl, arylcarbamoyl, thiocarbamoyl, carbamido, cyano, isocyano, cyanato, isocyanato, isothiocyanato, azido, formyl, thioformyl, amino, C₁-C₂₄ alkyl amino, C₂-C₂₄ alkylamido substituted with a hydroxyl, C₅-C₂₀ aryl amino, C₂-C₂₄ alkylamido, C₆-C₂₀ arylamido, imino, alkylimino, arylimino, nitro, nitroso, sulfo, sulfonato, C₁-C₂₄ alkylsulfanyl, arylsulfanyl, C₁-C₂₄ alkylsulfinyl, C₅-C₂₀ arylsulfinyl, C₁-C₂₄ alkylsulfonyl, C₅-C₂₀ arylsulfonyl, sulfonamide, phosphono, phosphonato, phosphinato, phospho, phosphino, polyalkylethers, phosphates, and phosphate esters, groups incorporating amino acids or other moieties expected to bear positive or negative charge at physiological pH, and combinations thereof, and wherein R⁶ and R⁷ may be linked to form a cyclic or polycyclic ring, wherein the ring is a substituted or unsubstituted aryl, a substituted or unsubstituted heteroaryl, a substituted or unsubstituted cycloalkyl, and a substituted or unsubstituted heterocyclyl;
U¹ is N, C—R², or C—NR³R⁴, wherein R² is selected from the group consisting of a H, a lower alkyl group, O, (CH₂)ₙ₁OR′ (wherein n1=1, 2, or 3), CF₃, CH₂—CH₂X, O—CH₂—CH₂X, CH₂—CH₂—CH₂X, O—CH₂—CH₂X, X, (wherein X=H, F, Cl, Br, or I), CN, (C═O)—R′, (C═O)N(R′)₂, O(CO)R′, COOR′ (wherein R′ is H or a lower alkyl group), and wherein R¹ and R² may be linked to form a cyclic or polycyclic ring, wherein R³ and R⁴ are the same or different and are each selected from the group consisting of H, a lower alkyl group, O, (CH₂)ₙ₁OR′ (wherein n1=1, 2, or 3), CF₃, CH₂—CH₂X, CH₂—CH₂—CH₂X, (wherein X=H, F, Cl, Br, or I), CN, (C═O)—R′, (C═O)N(R′)₂, COOR′ (wherein R′ is H or a lower alkyl group), and R³ or R⁴ may be absent;
Z¹ is O, S, CR^{a}R^{b} or NR^{a}, wherein R^{a} and R^{b} are independently H or a C₁₋₈ alkyl, which is linear, branched, or cyclic, and which is unsubstituted or substituted.

In some embodiments, R¹ is selected from the group consisting of branched, linear, or cyclic alkyl,

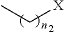

wherein n₂=0-6 and X is any of the following: CF_{y}H_{z} (y+z=3), CCl_{y}H_{z} (y+z=3), OH, OAc, OMe, R⁷¹, OR⁷², CN, N(R⁷³)₂,

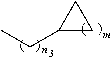

(n₃=0-5, m=1-5), and

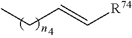

(n₄=0-5).

In other embodiments, R⁶ and R⁷ can each independently be one of the following:

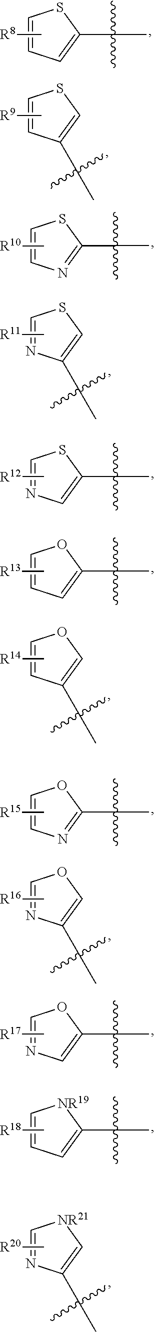

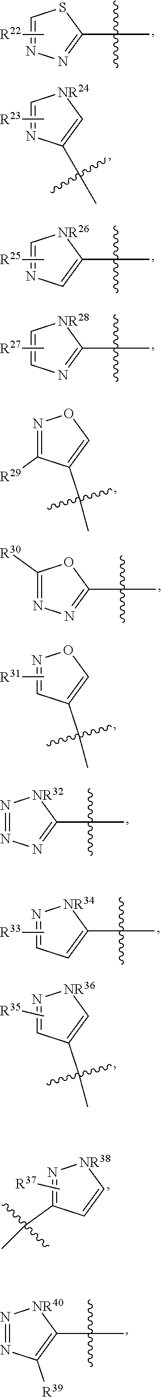

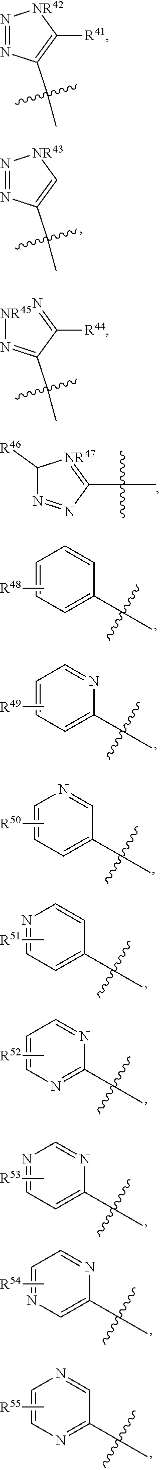

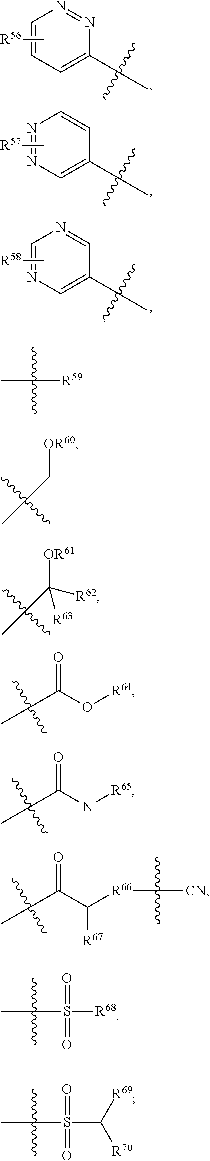

each R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R⁴⁸, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶¹, R⁶², R⁶³, R⁶⁴, R⁶⁵, R⁶⁶, R⁶⁷, R⁶⁸, R⁶⁹, R⁷⁰, R⁷¹, R⁷², R⁷³, and R⁷⁴ are the same or different and are independently selected from the group consisting of hydrogen, substituted or unsubstituted C₁-C₂₄ alkyl, C₂-C₂₄ alkenyl, C₂-C₂₄ alkynyl, C₃-C₂₀ aryl, heterocycloalkenyl containing from 5-6 ring atoms, (wherein from 1-3 of the ring atoms is independently selected from N, NH, N(C₁-C₆ alkyl), NC(O)(C₁-C₆ alkyl), O, and S), heteroaryl or heterocyclyl containing from 5-14 ring atoms, (wherein from 1-6 of the ring atoms is independently selected from N, NH, N(C₁-C₃ alkyl), O, and S), C₆-C₂₄ alkaryl, C₆-C₂₄ aralkyl, halo, silyl, hydroxyl, sulfhydryl, C₁-C₂₄ alkoxy, C₂-C₂₄ alkenyloxy, C₂-C₂₄ alkynyloxy, C₅-C₂₀ aryloxy, acyl (including C₂-C₂₄ alkylcarbonyl (—CO— alkyl) and C₆-C₂₀ arylcarbonyl (—CO-aryl)), acyloxy (—O-acyl), C₂-C₂₄ alkoxycarbonyl (—(CO)—O-alkyl), C₆-C₂₀ aryloxycarbonyl (—(CO)—O-aryl), C₂-C₂₄ alkylcarbonato (—O—(CO)—O-alkyl), C₆-C₂₀ arylcarbonato (—O—(CO)—O-aryl), carboxy (—COOH), carboxylato (—COO⁻), carbamoyl (—(CO)—NH₂), C₁-C₂₄ alkyl-carbamoyl (—(CO)—NH(C₁-C₂₄ alkyl)), arylcarbamoyl (—(CO)—NH-aryl), thiocarbamoyl (—(CS)—NH₂), carbamido (—NH—(CO)—NH₂), cyano (—CN), isocyano (—N⁺C⁻), cyanato (—O—CN), isocyanato (—O—N⁺═C⁻), isothiocyanato (—S—CN), azido (—N═N⁺═N⁻), formyl (—(CO)—H), thioformyl (—(CS)—H), amino (—NH₂), C₁-C₂₄ alkyl amino, C₁-C₂₄ alkyl amino substituted with hydroxyl, C₅-C₂₀ aryl amino, C₂-C₂₄ alkylamido (—NH—(CO)-alkyl), C₆-C₂₀ arylamido (—NH—(CO)-aryl), sulfanamido (—SO₂N(R)₂ where R is independently H, alkyl, aryl or heteroaryl), imino (—CR═NH where R is hydrogen, C₁-C₂₄ alkyl, C₅-C₂₀ aryl, C₆-C₂₄ alkaryl, C₆-C₂₄ aralkyl, etc.), alkylimino (—CR═N(alkyl), where R=hydrogen, alkyl, aryl, alkaryl, aralkyl, etc.), arylimino (—CR═N(aryl), where R=hydrogen, alkyl, aryl, alkaryl, etc.), nitro (—NO₂), nitroso (—NO), sulfo (—SO₂—OH), sulfonato (—SO₂—O⁻), C₁-C₂₄ alkylsulfanyl (—S-alkyl; also termed “alkylthio”), arylsulfanyl (—S-aryl; also termed “arylthio”), C₁-C₂₄ alkylsulfinyl (—(SO)-alkyl), C₅-C₂₀ arylsulfinyl (—(SO)-aryl), C₁-C₂₄ alkylsulfonyl (—SO₂-alkyl), C₅-C₂₀ arylsulfonyl (—SO₂-aryl), sulfonamide (—SO₂—NH₂, —SO₂NY₂ (wherein Y is independently H, aryl or alkyl), phosphono (—P(O)(OH)₂), phosphonato (—P(O)(O⁻)₂), phosphinato (—P(O)(O⁻)), phospho (—PO₂), phosphino (—PH₂), polyalkyl ethers (—[(CH₂)ₙO]ₘ), phosphates, phosphate esters [—OP(O)(OR)₂ where R=H, methyl or other alkyl], groups incorporating amino acids or other moieties expected to bear positive or negative charge at physiological pH, and combinations thereof, or a pharmaceutically acceptable salt, tautomer, or solvate thereof.

In still other embodiments, R⁶ and R⁷ can independently be a group that improves aqueous solubility, for example, a phosphate ester (—OPO₃H₂), a phenyl ring linked to a phosphate ester (—OPO₃H₂), a phenyl ring substituted with one or more methoxyethoxy groups, or a morpholine, or an aryl or heteroaryl ring substituted with such a group.

In other embodiments, the 15-PGDH inhibitor can include a compound having the following formula (V):

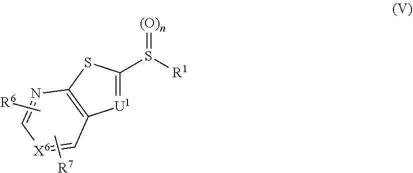

or a pharmaceutically acceptable salt, tautomer, or solvate thereof;
wherein n is 0-2
X⁶ is independently is N or CR^{c}
R¹, R⁶, R⁷, and R^{c} are the same or different each independently hydrogen or a substituted or unsubstituted group selected from C₁-C₂₄ alkyl, C₂-C₂₄ alkenyl, C₂-C₂₄ alkynyl, C₃-C₂₀ aryl, heteroaryl, heterocycloalkenyl containing from 5-6 ring atoms, C₆-C₂₄ alkaryl, C₆-C₂₄ aralkyl, halo, —Si(C₁-C₃ alkyl)₃, hydroxyl, sulfhydryl, C₁-C₂₄ alkoxy, C₂-C₂₄ alkenyloxy, C₂-C₂₄ alkynyloxy, C₅-C₂₀ aryloxy, acyl, acyloxy, C₂-C₂₄ alkoxycarbonyl, C₆-C₂₀ aryloxycarbonyl, C₂-C₂₄ alkylcarbonato, C₆-C₂₀ arylcarbonato, carboxy, carboxylato, carbamoyl, C₁-C₂₄ alkyl-carbamoyl, arylcarbamoyl, thiocarbamoyl, carbamido, cyano, isocyano, cyanato, isocyanato, isothiocyanato, azido, formyl, thioformyl, amino, C₁-C₂₄ alkyl amino, C₅-C₂₀ aryl amino, C₂-C₂₄ alkylamido, C₂-C₂₄ alkylamido substituted with a hydroxyl, C₆-C₂₀ arylamido, imino, alkylimino, arylimino, nitro, nitroso, sulfo, sulfonato, C₁-C₂₄ alkylsulfanyl, arylsulfanyl, C₁-C₂₄ alkylsulfinyl, C₅-C₂₀ arylsulfinyl, C₁-C₂₄ alkylsulfonyl, C₅-C₂₀ arylsulfonyl, sulfonamide, phosphono, phosphonato, phosphinato, phospho, phosphino, polyalkylethers, phosphates, and phosphate esters, groups incorporating amino acids or other moieties expected to bear positive or negative charge at physiological pH, and combinations thereof, and wherein R⁶ and R⁷ may be linked to form a cyclic or polycyclic ring, wherein the ring is a substituted or unsubstituted aryl, a substituted or unsubstituted heteroaryl, a substituted or unsubstituted cycloalkyl, and a substituted or unsubstituted heterocyclyl; and
U¹ is N, C—R², or C—NR³R⁴, wherein R² is selected from the group consisting of a H, a lower alkyl group, O, (CH₂)ₙ₁OR′ (wherein n1=1, 2, or 3), CF₃, CH₂—CH₂X, O—CH₂—CH₂X, CH₂—CH₂—CH₂X, O—CH₂—CH₂X, X, (wherein X=H, F, Cl, Br, or I), CN, (C═O)—R′, (C═O)N(R′)₂, O(CO)R′, COOR′ (wherein R′ is H or a lower alkyl group), and wherein R¹ and R² may be linked to form a cyclic or polycyclic ring, wherein R³ and R⁴ are the same or different and are each selected from the group consisting of H, a lower alkyl group, O, (CH₂)ₙ₁OR′ (wherein n1=1, 2, or 3), CF₃, CH₂—CH₂X, CH₂—CH₂—CH₂X, (wherein X=H, F, Cl, Br, or I), CN, (C═O)—R′, (C═O)N(R′)₂, COOR′ (wherein R′ is H or a lower alkyl group), and R³ or R⁴ may be absent.

In some embodiments, R¹ is selected from the group consisting of branched, linear, or cyclic alkyl,

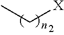

wherein n₂=0-6 and X is any of the following: CF_{y}H_{Z} (y+z=3), CCl_{y}H_{z} (+z=3), OH, OAc, OMe, R⁷¹, OR⁷², CN, N(R⁷³)₂,

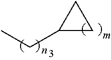

(n₃=0-5, m=1-5), and

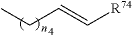

(n₄=0-5).

In other embodiments, R⁶ and R⁷ can each independently be one of the following:

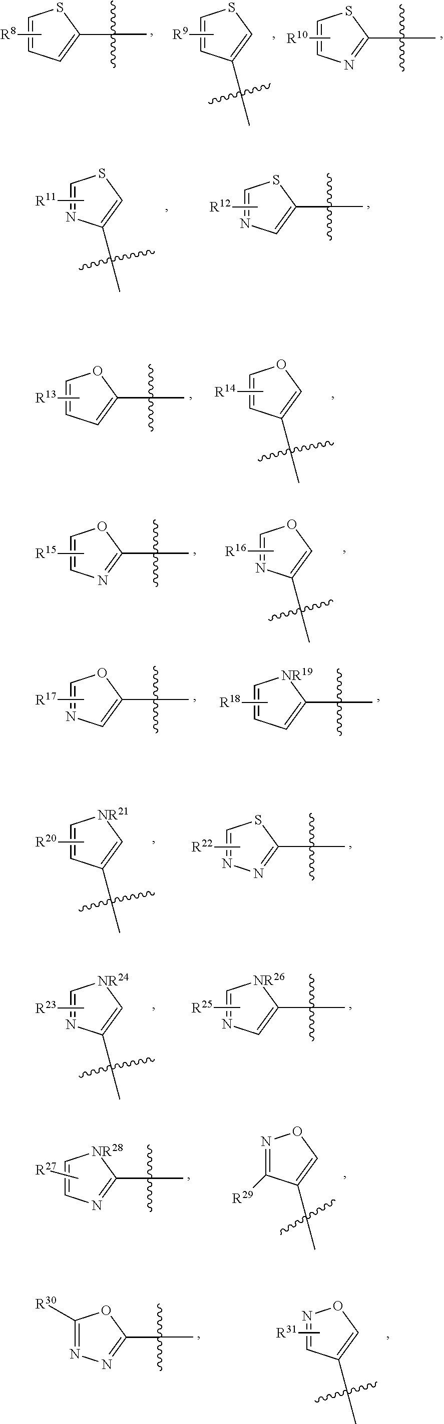

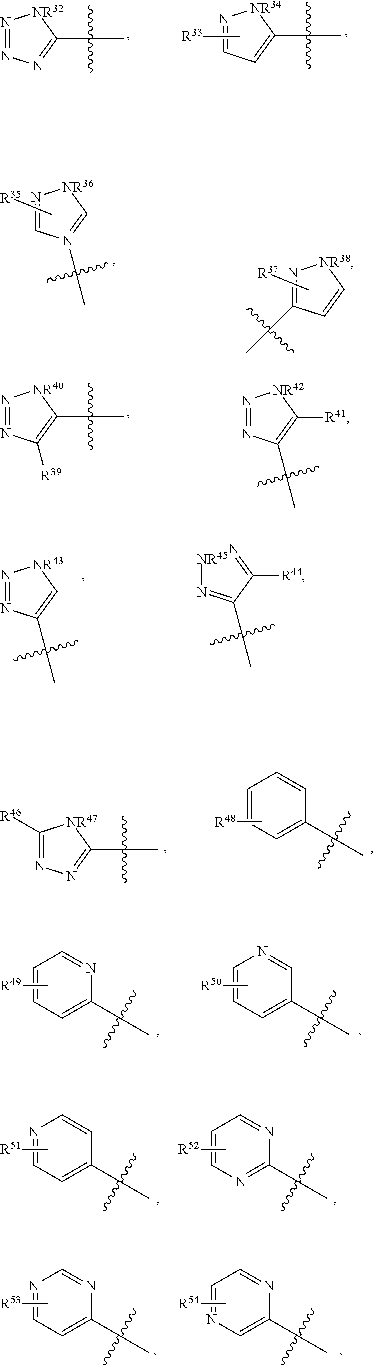

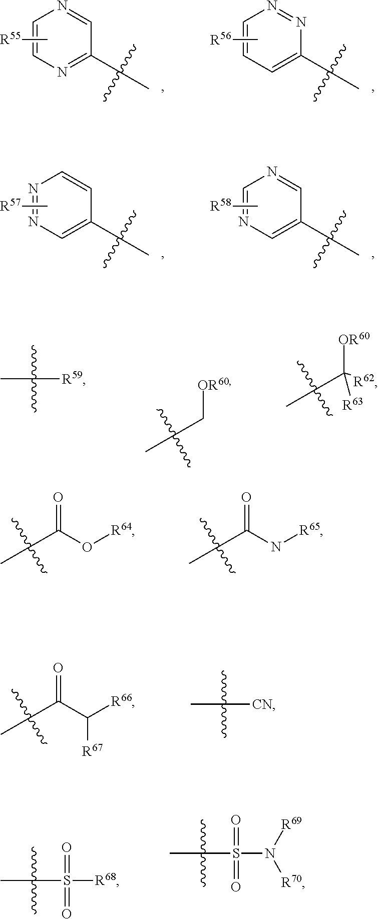

each R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, R²⁸, R²⁹, R³⁰, R³¹, R³², R³³, R³⁴, R³⁵, R³⁶, R³⁷, R³⁸, R³⁹, R⁴⁰, R⁴¹, R⁴², R⁴³, R⁴⁴, R⁴⁵, R⁴⁶, R⁴⁷, R⁴⁸, R⁴⁹, R⁵⁰, R⁵¹, R⁵², R⁵³, R⁵⁴, R⁵⁵, R⁵⁶, R⁵⁷, R⁵⁸, R⁵⁹, R⁶⁰, R⁶¹, R⁶², R⁶³, R⁶⁴, R⁶⁵, R⁶⁶, R⁶⁷, R⁶⁸, R⁶⁹, R⁷⁰, R⁷¹, R⁷², R⁷³, and R⁷⁴, are the same or different and are independently selected from the group consisting of hydrogen, substituted or unsubstituted C₁-C₂₄ alkyl, C₂-C₂₄ alkenyl, C₂-C₂₄ alkynyl, C₃-C₂₀ aryl, heterocycloalkenyl containing from 5-6 ring atoms, (wherein from 1-3 of the ring atoms is independently selected from N, NH, N(C₁-C₆ alkyl), NC(O)(C₁-C₆ alkyl), O, and S), heteroaryl or heterocyclyl containing from 5-14 ring atoms, (wherein from 1-6 of the ring atoms is independently selected from N, NH, N(C₁-C₃ alkyl), O, and S), C₆-C₂₄ alkaryl, C₆-C₂₄ aralkyl, halo, silyl, hydroxyl, sulfhydryl, C₁-C₂₄ alkoxy, C₂-C₂₄ alkenyloxy, C₂-C₂₄ alkynyloxy, C₅-C₂₀ aryloxy, acyl (including C₂-C₂₄ alkylcarbonyl (—CO— alkyl) and C₆-C₂₀ arylcarbonyl (—CO-aryl)), acyloxy (—O-acyl), C₂-C₂₄ alkoxycarbonyl (—(CO)—O-alkyl), C₆-C₂₀ aryloxycarbonyl (—(CO)—O-aryl), C₂-C₂₄ alkylcarbonato (—O—(CO)—O-alkyl), C₆-C₂₀ arylcarbonato (—O—(CO)—O-aryl), carboxy (—COOH), carboxylato (—COO⁻), carbamoyl (—(CO)—NH₂), C₁-C₂₄ alkyl-carbamoyl (—(CO)—NH(C₁-C₂₄ alkyl)), arylcarbamoyl (—(CO)—NH-aryl), thiocarbamoyl (—(CS)—NH₂), carbamido (—NH—(CO)—NH₂), cyano (—CN), isocyano (—N⁺C⁻), cyanato (—O—CN), isocyanato (—O—N⁺═C⁻), isothiocyanato (—S—CN), azido (—N═N⁺═N⁻), formyl (—(CO)—H), thioformyl (—(CS)—H), amino (—NH₂), C₁-C₂₄ alkyl amino, C₁-C₂₄ alkyl amino substituted with hydroxyl, C₅-C₂₀ aryl amino, C₂-C₂₄ alkylamido (—NH—(CO)-alkyl), C₆-C₂₀ arylamido (—NH—(CO)-aryl), sulfanamido (—SO₂N(R)₂ where R is independently H, alkyl, aryl or heteroaryl), imino (—CR═NH where R is hydrogen, C₁-C₂₄ alkyl, C₅-C₂₀ aryl, C₆-C₂₄ alkaryl, C₆-C₂₄ aralkyl, etc.), alkylimino (—CR═N(alkyl), where R=hydrogen, alkyl, aryl, alkaryl, aralkyl, etc.), arylimino (—CR═N(aryl), where R=hydrogen, alkyl, aryl, alkaryl, etc.), nitro (—NO₂), nitroso (—NO), sulfo (—SO₂—OH), sulfonato (—SO₂—O⁻), C₁-C₂₄ alkylsulfanyl (—S-alkyl; also termed “alkylthio”), arylsulfanyl (—S-aryl; also termed “arylthio”), C₁-C₂₄ alkylsulfinyl (—(SO)-alkyl), C₅-C₂₀ arylsulfinyl (—(SO)-aryl), C₁-C₂₄ alkylsulfonyl (—SO₂-alkyl), C₅-C₂₀ arylsulfonyl (—SO₂-aryl), sulfonamide (—SO₂—NH₂, —SO₂NY₂ (wherein Y is independently H, arlyl or alkyl), phosphono (—P(O)(OH)₂), phosphonato (—P(O)(O⁻)₂), phosphinato (—P(O)(O⁻)), phospho (—PO₂), phosphino (—PH₂), polyalkyl ethers (—[(CH₂)ₙO]ₘ), phosphates, phosphate esters [—OP(O)(OR)₂ where R=H, methyl or other alkyl], groups incorporating amino acids or other moieties expected to bear positive or negative charge at physiological pH, and combinations thereof, or a pharmaceutically acceptable salt, tautomer, or solvate thereof.

In still other embodiments, R⁶ and R⁷ can independently be a group that improves aqueous solubility, for example, a phosphate ester (—OPO₃H₂), a phenyl ring linked to a phosphate ester (—OPO₃H₂), a phenyl ring substituted with one or more methoxyethoxy groups, or a morpholine, or an aryl or heteroaryl ring substituted with such a group.

In other embodiments, the 15-PGDH inhibitor can include a compound having a structure of formula (IA):

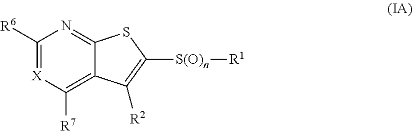

or a pharmaceutically acceptable salt, tautomer, or solvate thereof, wherein:
R¹ is alkyl, haloalkyl, cycloalkyl, alkylene-cycloalkyl, alkylene-alkoxy, heterocyclyl, or alkylene-heterocyclyl;
R² is —NH₂, CN, or —NHC(O)alkyl;
R⁶ is heterocyclyl or heteroaryl, each of which is optionally substituted with one or more R³;
R⁷ is alkyl, haloalkyl, cycloalkyl, aryl, heterocyclyl, heteroaryl, —C(O)-alkyl,
—C(O)O-alkyl, or —C(O)NR⁵-alkyl, each of which is optionally substituted with one or more R⁴;
R³ is oxo, —OH, —O-alkylene-OH, —O-alkylene-N(R⁵)₂, —N(R⁵)₂, —N(R⁵)(alkylene-OH), —N(R⁵)(alkylene-O-alkyl), alkyl, -alkylene-OH, haloalkyl, cycloalkyl, heterocyclyl, —C(O)N(R⁵)₂, —C(O)N(R⁵)(alkylene-OH), —C(O)-alkyl, —C(O)O-alkyl, or —S(O)ₘ-alkyl, wherein the cycloalkyl and the heterocyclyl is each optionally substituted with R¹⁰;
R⁴ is oxo, halogen, —CN, —N(R⁵)₂, —OH, —O-alkylene-OH, —S(O)ₘ-alkyl, —C(O)— alkyl, —C(O)-cycloalkyl, alkyl, -alkylene-O-alkyl, alkoxy, haloalkyl, cycloalkyl, heterocyclyl, or -alkylene-aryl optionally substituted with R⁸;
each R⁵ is independently, H, alkyl, -alkylene-OH optionally substituted with —OH, -alkylene-NH₂, -alkylene-N(R⁹)₂, -alkylene-O-alkylene-OH, -alkylene-O-alkylene-NH₂, —C(O)-alkyl, —C(O)O-alkyl, -alkylene-COOH, or —S(O)ₘ-alkyl;
or alternatively, two R⁵ together with the N atom to which they are attached can form a 4- to 7-membered heterocycle, optionally containing an additional heteroatom selected from O, S, or N, and wherein the heterocycle is optionally substituted with R⁸;
R⁸ is halogen, alkyl, or alkoxy;
R⁹ is H or alkyl, or two R⁹ together with the N atom to which they are attached can form a 4- to 7-membered heterocycle, optionally containing an additional heteroatom selected from 0, S(O)ₜ, or N;
R¹⁰ is —OH, halogen, alkyl, or alkoxy;
X is N or CH;
m is 0, 1, or 2;
n is 0, 1, or 2; and
t is 0, 1, or 2.

In some embodiment, the 15-PGDH inhibitor can include a compound having a structure of formula (IIA):

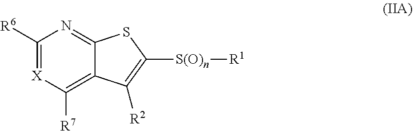

or a pharmaceutically acceptable salt, tautomer, or solvate thereof, wherein:
R¹ is alkyl, haloalkyl, cycloalkyl, alkylene-cycloalkyl, alkylene-alkoxy, heterocyclyl, or alkylene-heterocyclyl;
R² is —NH₂, CN, or —NHC(O)alkyl;
R⁶ is heterocyclyl or heteroaryl, each of which is optionally substituted with one or more R³; R⁷ is alkyl, haloalkyl, cycloalkyl, aryl, heterocyclyl, heteroaryl, —C(O)-alkyl, —C(O)O-alkyl, or —C(O)NR⁵-alkyl, each of which is optionally substituted with one or more R⁴;
R³ is oxo, —OH, —O-alkylene-N(R⁵)₂, —N(R⁵)₂, —N(R⁵)(alkylene-OH), alkyl, haloalkyl, cycloalkyl, heterocyclyl, —C(O)N(R⁵)₂, —C(O)N(R⁵)(alkylene-OH), —C(O)-alkyl, —C(O)O-alkyl, or —S(O)ₘ-alkyl;
R⁴ is oxo, halogen, —CN, —N(R⁵)₂, —OH, —O-alkylene-OH, —S(O)ₘ-alkyl, —C(O)— alkyl, —C(O)-cycloalkyl, alkyl, alkoxy, haloalkyl, cycloalkyl, heterocyclyl, or -alkylene-aryl optionally substituted with R⁸;
each R⁵ is independently, H, alkyl, -alkylene-OH optionally substituted with —OH, -alkylene-NH₂, -alkylene-N(R⁹)₂, -alkylene-O-alkylene-OH, -alkylene-O-alkylene-NH₂, —C(O)-alkyl, —C(O)O-alkyl, -alkylene-COOH, or —S(O)ₘ-alkyl;
or alternatively, two R⁵ together with the N atom to which they are attached can form a 4- to 7-membered heterocycle, optionally containing an additional heteroatom selected from O, S, or N, and wherein the heterocycle is optionally substituted with R⁸;
R⁸ is halogen, alkyl, or alkoxy;
R⁹ is H or alkyl, or two R⁹ together with the N atom to which they are attached can form a 4- to 7-membered heterocycle, optionally containing an additional heteroatom selected from O, S(O)ₜ, or N;
X is N or CH;
m is 0, 1, or 2;
n is 0, 1, or 2; and
t is 0, 1, or 2.

In some embodiments, R¹ is C₁-C₆ alkyl, C₁-C₆ haloalkyl, 3- to 6-membered cycloalkyl, —(C₁-C₆ alkylene)-(3- to 6-membered cycloalkyl), —(C₁-C₆ alkylene)-(C₁-C₆ alkoxy), 3- to 6-membered heterocyclyl, or —(C₁-C₆ alkylene)-(3- to 6-membered heterocyclyl).

In other embodiments, R¹ is cyclopropyl, cyclobutyl, cyclopentyl, cyclohexyl, —(CH₂)ₚ-cyclopropyl, —(CH₂)ₚ-cyclobutyl, —(CH₂)ₚ-cyclopentyl, or —(CH₂)ₚ-cyclohexyl; wherein p is 1, 2, or 3.

In still other embodiments, R² is —NH₂.

In some embodiments, R⁶ is 5- to 6-membered heterocyclyl or 5- to 10-membered heteroaryl, each of which is optionally substituted with one or more R³.

In other embodiments, R⁶ is 5- to 6-membered heteroaryl optionally substituted with one or more R³.

In still other embodiments, R⁶ is 8- to 10-membered bicyclic heteroaryl optionally substituted with one or more R³.

In some embodiments, R⁷ is C₁-C₆ alkyl, C₁-C₆ haloalkyl, 3- to 6-membered cycloalkyl, 6- to 10-membered aryl, 3- to 6-membered heterocyclyl, 5- to 10-membered heteroaryl, —C(O)(C₁-C₆ alkyl), —C(O)O(C₁-C₆ alkyl), or —C(O)NR⁵(C₁-C₆ alkyl), each of which is optionally substituted with one or more R⁴.

In other embodiments, R⁷ is C₁-C₆ alkyl, C₁-C₆ haloalkyl, 3- to 6-membered cycloalkyl, phenyl, 3- to 6-membered heterocyclyl, or 5- to 10-membered heteroaryl, each of which is optionally substituted with one or more R⁴.

In still other embodiments, R⁷ is C₁-C₆ haloalkyl, 3- to 6-membered cycloalkyl, phenyl, 5- to 10-membered heteroaryl each of which is optionally substituted with one or more R⁴.

In some embodiments, R³ is —O—(C₁-C₆ alkylene)-N(R⁵)₂, —N(R⁵)₂, —N(R⁵)(C₁-C₆ alkylene-OH), —C(O)N(R⁵)₂, —C(O)N(R⁵)(C₁-C₆ alkylene-OH), —C(O)(C₁-C₆ alkyl), —C(O)O(C₁-C₆ alkyl), or —S(O)ₘ(C₁-C₆ alkyl).

In other embodiments, R³ is —(C₁-C₃ alkyl)OH, —NH₂, —N(C₁-C₃ alkyl)₂, —NHCH₂CH₂OH, —N(C₁-C₃ alkyl)CH₂CH₂OH, N(CH₂CH₂OH)₂, —NHCH₂CH(CH₂OH)₂, —N(C₁-C₃ alkyl)CH₂CH(CH₂OH)₂, —NHCH₂CH₂OCH₂CH₂OH, —NHCH₂CH₂OCH₂CH₂NH₂, —NHCH₂CH₂NH₂, —N(C₁-C₃ alkyl)CH₂CH₂NH₂, —NHCH₂CH₂NH(C₁-C₃ alkyl), —NHCH₂CH₂N(C₁-C₃ alkyl)₂, —N(C₁-C₃ alkyl)CH₂CH₂NH(C₁-C₃ alkyl), —N(C₁-C₃ alkyl)CH₂CH₂N(C₁-C₃ alkyl)₂, —NHSO₂CH₃, —N(C₁-C₃ alkyl)SO₂CH₃, —OCH₂CH₂OH, —OCH₂CH₂NH₂, —OCH₂CH₂NH(C₁-C₃ alkyl), or —OCH₂CH₂N(C₁-C₃ alkyl)₂.

In other embodiments, R³ is —NH₂, —N(C₁-C₃ alkyl)₂, —NHCH₂CH₂OH, —N(C₁-C₃ alkyl)CH₂CH₂OH, N(CH₂CH₂OH)₂, —NHCH₂CH(CH₂OH)₂, —N(C₁-C₃ alkyl)CH₂CH(CH₂OH)₂, —NHCH₂CH₂OCH₂CH₂OH, —NHCH₂CH₂OCH₂CH₂NH₂, —NHCH₂CH₂NH₂, —N(C₁-C₃ alkyl)CH₂CH₂NH₂, —NHCH₂CH₂NH(C₁-C₃ alkyl), —NHCH₂CH₂N(C₁-C₃ alkyl)₂, —N(C₁-C₃ alkyl)CH₂CH₂NH(C₁-C₃ alkyl), —N(C₁-C₃ alkyl)CH₂CH₂N(C₁-C₃ alkyl)₂, —NHSO₂CH₃, —N(C₁-C₃ alkyl)SO₂CH₃, —OCH₂CH₂OH, —OCH₂CH₂NH₂, —OCH₂CH₂NH(C₁-C₃ alkyl), or —OCH₂CH₂N(C₁-C₃ alkyl)₂.

In still other embodiments, R³ is —NHCH₂CH₂OH or —N(CH₃)CH₂CH₂OH.

In some embodiments, R⁴ is halogen, —CN, —N(R⁵)₂, —OH, —O—(C₁-C₆ alkylene)-OH, —S(O)ₘ(C₁-C₆ alkyl), —C(O)(C₁-C₆ alkyl), —C(O)-(3- to 6-membered cycloalkyl), C₁-C₆ alkyl, C₁-C₆ haloalkyl, 3- to 6-membered cycloalkyl, or 3- to 6-membered heterocyclyl.

In some embodiments, n is 1.

In other embodiments, the compound has the structure of formula (IIIA):

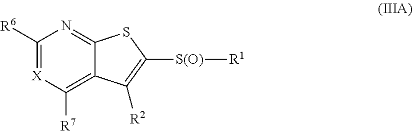

or a pharmaceutically acceptable salt, tautomer, or solvate thereof, wherein:
R¹ is 3- to 6-membered cycloalkyl, —(C₁-C₆ alkylene)-(3- to 6-membered cycloalkyl), 3- to 6-membered heterocyclyl, or —(C₁-C₆ alkylene)-(3- to 6-membered heterocyclyl);
R² is —NH₂, CN, or —NHC(O)alkyl;
R⁶ is heterocyclyl or heteroaryl, each of which is optionally substituted with one or more R³;
R⁷ is alkyl, haloalkyl, cycloalkyl, aryl, heterocyclyl, heteroaryl, —C(O)-alkyl, —C(O)O-alkyl, or —C(O)NR⁵-alkyl, each of which is optionally substituted with one or more R⁴;
R³ is oxo, —OH, —O-alkylene-N(R⁵)₂, —N(R⁵)₂, —N(R⁵)(alkylene-OH), alkyl, haloalkyl, cycloalkyl, heterocyclyl, —C(O)N(R⁵)₂, —C(O)N(R⁵)(alkylene-OH), —C(O)-alkyl, —C(O)O-alkyl, or —S(O)ₘ-alkyl,
R⁴ is oxo, halogen, —CN, —N(R⁵)₂, —OH, —O-alkylene-OH, —S(O)ₘ-alkyl, —C(O)— alkyl, —C(O)-cycloalkyl, alkyl, haloalkyl, cycloalkyl, heterocyclyl, or -alkylene-aryl optionally substituted with R⁸;
each R⁵ is independently, H, alkyl, -alkylene-OH optionally substituted with —OH, -alkylene-NH₂, -alkylene-N(R⁹)₂, -alkylene-O-alkylene-OH, -alkylene-O-alkylene-NH₂, —C(O)-alkyl, —C(O)O-alkyl, -alkylene-COOH, or —S(O)ₘ-alkyl;
or alternatively, two R⁵ together with the N atom to which they are attached can form a 4- to 7-membered heterocycle, optionally containing an additional heteroatom selected from O, S, or N, and wherein the heterocycle is optionally substituted with R⁸;
R⁸ is halogen, alkyl, or alkoxy;
R⁹ is H or alkyl, or two R⁹ together with the N atom to which they are attached can form a 4- to 7-membered heterocycle, optionally containing an additional heteroatom selected from 0, S(O)ₜ, or N;
X is N or CH;
m is 0, 1, or 2; and
t is 0, 1, or 2.

In some embodiments, R¹ is 3- to 5-membered cycloalkyl or —(C₁-C₆ alkylene)-(3- to 5-membered cycloalkyl).

In other embodiments, R¹ is cyclobutyl.

In still other embodiments, R¹ is a bicyclic 4- to 6-membered cycloalkyl.

In some embodiments, R⁷ is C₁-C₆ alkyl, C₁-C₆ haloalkyl, 3- to 6-membered cycloalkyl, 6- to 10-membered aryl, 3- to 6-membered heterocyclyl, 5- to 10-membered heteroaryl, —C(O)(C₁-C₆ alkyl), —C(O)O(C₁-C₆ alkyl), or —C(O)NR⁵(C₁-C₆ alkyl), each of which is optionally substituted with one or more R⁴.

In other embodiments, R⁷ is C₁-C₄ alkyl, C₁-C₆ haloalkyl, 3- to 6-membered cycloalkyl, phenyl, 3- to 6-membered heterocyclyl, or 5- to 10-membered heteroaryl, each of which is optionally substituted with one or more R⁴. In other embodiments, R⁷ is C₁-C₆ haloalkyl, 3- to 6-membered cycloalkyl, phenyl, 3- to 6-membered heterocyclyl, or 5- to 10-membered heteroaryl, each of which is optionally substituted with one or more R⁴.

In other embodiments, R⁷ is C₁-C₃ alkyl, C₁-C₃ haloalkyl, 3-membered cycloalkyl, phenyl, 4-membered heterocyclyl, or 5- to 6-membered heteroaryl, each of which is optionally substituted with one or more R⁴. In other embodiments, R⁷ is C₁-C₃ haloalkyl, 3-membered cycloalkyl, phenyl, 4-membered heterocyclyl, or 5- to 6-membered heteroaryl, each of which is optionally substituted with one or more R⁴.

In other embodiments, R⁷ is —CF₃, isopropyl, cyclopropyl, phenyl, pyridyl, pyrazole, or triazole, each of which is optionally substituted with one or more R⁴. In other embodiments, R⁷ is —CF₃, cyclopropyl, phenyl, pyridyl, pyrazole, or triazole, each of which is optionally substituted with one or more R⁴.

In some embodiments, R³ is —O—(C₁-C₆ alkylene)-N(R⁵)₂, —N(R⁵)₂, —N(R⁵)(C₁-C₆ alkylene-OH), —C(O)N(R⁵)₂, —C(O)N(R⁵)(C₁-C₆ alkylene-OH), —C(O)(C₁-C₆ alkyl), —C(O)O(C₁-C₆ alkyl), or —S(O)ₘ(C₁-C₆ alkyl).

In other embodiments, R³ is —NH₂, —N(C₁-C₃ alkyl)₂, —NHCH₂CH₂OH, —N(C₁-C₃ alkyl)CH₂CH₂OH, N(CH₂CH₂OH)₂, —NHCH₂CH(CH₂OH)₂, —N(C₁-C₃ alkyl)CH₂CH(CH₂OH)₂, —NHCH₂CH₂OCH₂CH₂OH, —NHCH₂CH₂OCH₂CH₂NH₂, —NHCH₂CH₂NH₂, —N(C₁-C₃ alkyl)CH₂CH₂NH₂, —NHCH₂CH₂NH(C₁-C₃ alkyl), —NHCH₂CH₂N(C₁-C₃ alkyl)₂, —N(C₁-C₃ alkyl)CH₂CH₂NH(C₁-C₃ alkyl), —N(C₁-C₃ alkyl)CH₂CH₂N(C₁-C₃ alkyl)₂, —NHSO₂CH₃, —N(C₁-C₃ alkyl)SO₂CH₃, —OCH₂CH₂OH, —OCH₂CH₂NH₂, —OCH₂CH₂NH(C₁-C₃ alkyl), or —OCH₂CH₂N(C₁-C₃ alkyl)₂.

In still other embodiments, R³ is —NHCH₂CH₂OH or —N(CH₃)CH₂CH₂OH.

In some embodiments and without being limited by theory, Applicants surprisingly and unexpectedly discovered that substituents at the R⁷ position could be modified to improve hERG activity, including hERG inhibition (IC₅₀), blockade, and efflux ratio. For example, in some embodiments, certain 6- to 10-membered aryls (e.g., optionally substituted phenyl) and 5- to 10-membered heteroaryls (e.g., optionally substituted pyridyls, pyrazoles, and triazoles) were observed to have beneficial hERG properties. In some embodiments, certain C₁-C₆ haloalkyls (e.g., —CF₃) exhibited improved hERG inhibition (IC50), while also improving half-life and solubility.

In other embodiments, the compound has the structure of formula (IVA):

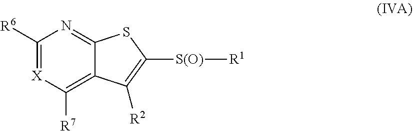

or a pharmaceutically acceptable salt, tautomer, or solvate thereof, wherein:
R¹ is cycloalkyl, alkylene-cycloalkyl, alkylene-alkoxy, heterocyclyl, or alkylene-heterocyclyl;
R² is —NH₂, CN, or —NHC(O)alkyl;
R⁶ is heterocyclyl or heteroaryl, each of which is optionally substituted with one or more R³;
R⁷ is C₁-C₆ haloalkyl, aryl or heteroaryl, each of which is optionally substituted with one or more R⁴;
R³ is oxo, —OH, —O-alkylene-N(R⁵)₂, —N(R⁵)₂, —N(R⁵)(alkylene-OH), alkyl, haloalkyl, cycloalkyl, heterocyclyl, —C(O)N(R⁵)₂, —C(O)N(R⁵)(alkylene-OH), —C(O)-alkyl, —C(O)O-alkyl, or —S(O)ₘ-alkyl;
each R⁵ is independently, H, alkyl, -alkylene-OH optionally substituted with —OH, -alkylene-NH₂, -alkylene-N(R⁹)₂, -alkylene-O-alkylene-OH, -alkylene-O-alkylene-NH₂, —C(O)-alkyl, -alkylene-COOH, —C(O)O-alkyl, or —S(O)ₘ-alkyl;
or alternative, two R⁵ together with the N atom to which they are attached can form a 4- to 7-membered heterocycle, optionally containing an additional heteroatom selected from O, S, or N, and wherein the heterocycle is optionally substituted with R⁸;
R⁴ is halogen, alkyl, or alkoxy;
X is N or CH; and
m is 0, 1, or 2.

In some embodiments, R⁷ is —CF₃, pyridyl, pyrazole, phenyl, or triazole, each of which is optionally substituted with R⁴.

In other embodiments, R⁷ is —CF₃, pyridyl, fluorophenyl, or a triazole optionally substituted with halogen or methyl.

In other embodiments, R⁷ is —CF₃,

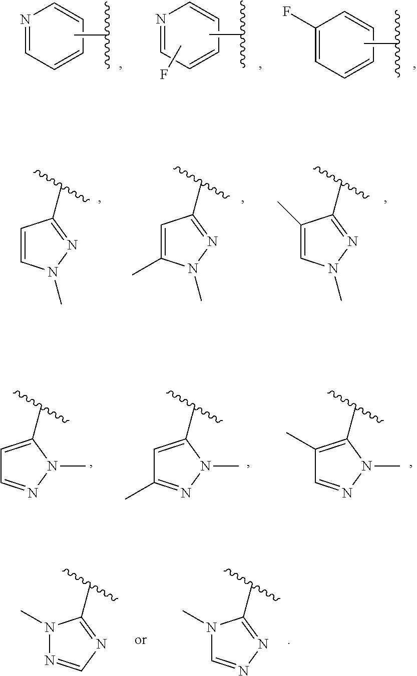

In still other embodiments, R⁷ is —CF₃.

In still other embodiments, R⁷ is

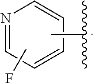

In some embodiments, R⁷ is

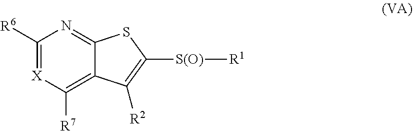

In some embodiments, R⁷ is

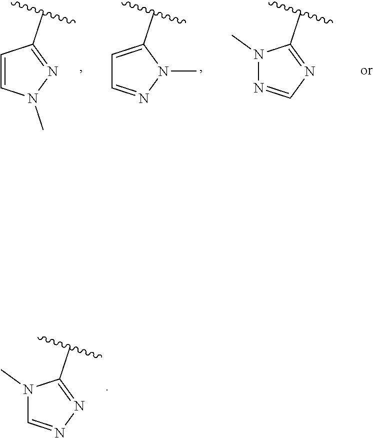

In other embodiments, R⁶ is 8- to 10-membered bicyclic heteroaryl optionally substituted with one or more R³.

In some embodiments and without being limited by theory, Applicants surprisingly and unexpectedly discovered that 3- to 6-membered cycloalkyls at the R⁷ position can improve solubility while maintaining PDGH activity.

In other embodiments, the compound has the structure of formula (VA):

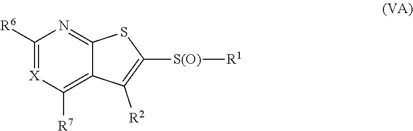

or a pharmaceutically acceptable salt, tautomer, or solvate thereof, wherein:
R¹ is cycloalkyl, -alkylene-cycloalkyl, -alkylene-alkoxy, heterocyclyl, or -alkylene-heterocyclyl;
R² is —NH₂, CN, or —NHC(O)alkyl;
R⁶ is heterocyclyl or heteroaryl, each of which is optionally substituted with one or more R³;
R⁷ is 3- to 6-membered cycloalkyl, optionally substituted with one or more R⁴;
R³ is oxo, —OH, —O-alkylene-N(R⁵)₂, —N(R⁵)₂, —N(R⁵)(alkylene-OH), alkyl, haloalkyl, cycloalkyl, heterocyclyl, —C(O)N(R⁵)₂, —C(O)N(R⁵)(alkylene-OH), —C(O)-alkyl, —C(O)O-alkyl, or —S(O)ₘ-alkyl;
R⁴ is halogen, —CN, —NH₂, —OH, or C₁-C₃ alkyl;
each R⁵ is independently, H, alkyl, -alkylene-OH optionally substituted with —OH, -alkylene-NH₂, -alkylene-N(R⁹)₂, -alkylene-O-alkylene-OH, -alkylene-O-alkylene-NH₂, —C(O)-alkyl, -alkylene-COOH, —C(O)O-alkyl, or —S(O)ₘ-alkyl;
or alternative, two R⁵ together with the N atom to which they are attached can form a 4- to 7-membered heterocycle, optionally containing an additional heteroatom selected from O, S, or N, and wherein the heterocycle is optionally substituted with R⁸;
R⁸ is halogen, alkyl, or alkoxy;
X is N or CH;
m is 0, 1, or 2.

In some embodiments, R⁷ is cyclopropyl.

In other embodiments, R¹ is 3- to 6-membered cycloalkyl, —(C₁-C₆ alkylene)-(3- to 6-membered cycloalkyl), —(C₁-C₆ alkylene)-(C₁-C₆ alkoxy), 3- to 6-membered heterocyclyl, or —(C₁-C₆ alkylene)-(3- to 6-membered heterocyclyl).

In some embodiments, R¹ is cyclopropyl, cyclobutyl, cyclopentyl, cyclohexyl, —(CH₂)ₚ-cyclopropyl, —(CH₂)ₚ-cyclobutyl, —(CH₂)ₚ-cyclopentyl, or —(CH₂)ₚ-cyclohexyl; wherein p is 1, 2, or 3.

In some embodiments, R³ is —O—(C₁-C₆ alkylene)-N(R⁵)₂, —N(R⁵)₂, —N(R⁵)(C₁-C₆ alkylene-OH), —C(O)N(R⁵)₂, —C(O)N(R⁵)(C₁-C₆ alkylene-OH), —C(O)(C₁-C₆ alkyl), —C(O)O(C₁-C₆ alkyl), or —S(O)ₘ(C₁-C₆ alkyl).

In some embodiments, R³ is —NH₂, —N(C₁-C₃ alkyl)₂, —NHCH₂CH₂OH, —N(C₁-C₃ alkyl)CH₂CH₂OH, N(CH₂CH₂OH)₂, —NHCH₂CH(CH₂OH)₂, —N(C₁-C₃ alkyl)CH₂CH(CH₂OH)₂, —NHCH₂CH₂OCH₂CH₂OH, —NHCH₂CH₂OCH₂CH₂NH₂, —NHCH₂CH₂NH₂, —N(C₁-C₃ alkyl)CH₂CH₂NH₂, —NHCH₂CH₂NH(C₁-C₃ alkyl), —NHCH₂CH₂N(C₁-C₃ alkyl)₂, —N(C₁-C₃ alkyl)CH₂CH₂NH(C₁-C₃ alkyl), —N(C₁-C₃ alkyl)CH₂CH₂N(C₁-C₃ alkyl)₂, —NHSO₂CH₃, —N(C₁-C₃ alkyl)SO₂CH₃, —OCH₂CH₂OH, —OCH₂CH₂NH₂, —OCH₂CH₂NH(C₁-C₃ alkyl), or —OCH₂CH₂N(C₁-C₃ alkyl)₂.

In other embodiments, R³ is —NHCH₂CH₂OH or —N(CH₃)CH₂CH₂OH.

In some embodiments and without being limited by theory, Applicants surprisingly and unexpectedly discovered that the R⁶ position can be substituted with certain R³ groups to improve solubility and activity.

In other embodiments, the compound has the structure of formula (VIA):

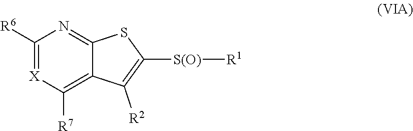

or a pharmaceutically acceptable salt, tautomer, or solvate thereof, wherein:
R¹ is cycloalkyl, -alkylene-cycloalkyl, -alkylene-alkoxy, heterocyclyl, or -alkylene-heterocyclyl;
R² is —NH₂, CN, or —NHC(O)alkyl;
R⁶ is heterocyclyl or heteroaryl, each of which is substituted with one or more R³;
R⁷ is haloalkyl, cycloalkyl, aryl, heterocyclyl, heteroaryl, —C(O)-alkyl, —C(O)O-alkyl, or —C(O)NR⁵-alkyl, each of which is optionally substituted with one or more R⁴;
R³ is —O—(C₁-C₆ alkylene)-N(R⁵)₂, —N(R⁵)₂, —N(R⁵)(C₁-C₆ alkylene-OH), —C(O)N(R⁵)₂, —C(O)N(R⁵)(C₁-C₆ alkylene-OH), —C(O)(C₁-C₆ alkyl), —C(O)O(C₁-C₆ alkyl), or —S(O)ₘ(C₁-C₆ alkyl);
R⁴ is oxo, halogen, —CN, —N(R⁵)₂, —OH, —O-alkylene-OH, —S(O)ₘ-alkyl, —C(O)— alkyl, —C(O)-cycloalkyl, alkyl, haloalkyl, cycloalkyl, heterocyclyl, or -alkylene-aryl optionally substituted with R⁸;
each R⁵ is independently H, C₁-C₆ alkyl, —(C₁-C₆ alkylene)-OH optionally substituted with —OH, -alkylene-NH₂, -alkylene-N(R⁹)₂, -alkylene-O-alkylene-OH, -alkylene-O-alkylene-NH₂, —C(O)(C₁-C₆ alkyl), —C(O)O(C₁-C₆ alkyl), alkylene-COOH, or —S(O)ₘ(C₁-C₆ alkyl);
or alternative, two R⁵ together with the N atom to which they are attached can form a 4- to 7-membered heterocycle, optionally containing an additional heteroatom selected from O, S, or N, and wherein the heterocycle is optionally substituted with R⁸;
R⁸ is halogen, alkyl, or alkoxy;
X is N or CH;
m is 0, 1, or 2.

In some embodiments, R³ is —O—(C₁-C₆ alkylene)-N(R⁵)₂, —N(R⁵)₂ or —N(R⁵)(C₁-C₆ alkylene-OH).

In other embodiments, R⁵ is H, C₁-C₆ alkyl, —(C₁-C₆ alkylene)-OH, or —S(O)₂(C₁-C₃ alkyl).

In some embodiments, R³ is —NH₂, —N(C₁-C₃ alkyl)₂, —NHCH₂CH₂OH, —N(C₁-C₃ alkyl)CH₂CH₂OH, N(CH₂CH₂OH)₂, —NHCH₂CH(CH₂OH)₂, —N(C₁-C₃ alkyl)CH₂CH(CH₂OH)₂, —NHCH₂CH₂OCH₂CH₂OH, —NHCH₂CH₂OCH₂CH₂NH₂, —NHCH₂CH₂NH₂, —N(C₁-C₃ alkyl)CH₂CH₂NH₂, —NHCH₂CH₂NH(C₁-C₃ alkyl), —NHCH₂CH₂N(C₁-C₃ alkyl)₂, —N(C₁-C₃ alkyl)CH₂CH₂NH(C₁-C₃ alkyl), —N(C₁-C₃ alkyl)CH₂CH₂N(C₁-C₃ alkyl)₂, —NHSO₂CH₃, —N(C₁-C₃ alkyl)SO₂CH₃, —OCH₂CH₂OH, —OCH₂CH₂NH₂, —OCH₂CH₂NH(C₁-C₃ alkyl), or —OCH₂CH₂N(C₁-C₃ alkyl)₂.

In other embodiments, R³ is —NHCH₂CH₂OH or —N(CH₃)CH₂CH₂OH.

In still other embodiments, R³ is —NHCH₂CH₂OH.

In some embodiments, R⁶ is 5- to 6-membered heterocyclyl or 5- to 10-membered heteroaryl, each of which is optionally substituted with one or more R³.

In other embodiments, R⁶ is 5- to 6-membered heteroaryl optionally substituted with one or more R³.

In some embodiments, R⁶ is furan, thiophene, pyrrole, thiazole, isothiazole, oxazole, isooxazole, pyrazole, imidazole, triazole, pyridine, pyrimidine, pyridazine, or pyrazine, each optionally substituted with one or more R³.

In other embodiments, R⁶ is thiazole, imidazole, oxazole, pyridine, or pyrimidine.

In some embodiments, R⁶ is 8- to 10-membered bicyclic heteroaryl optionally substituted with one or more R³.

In other embodiments, R⁶ is 5- to 6-membered heterocyclyl, optionally substituted with one or more R³, selected from morpholine, pyridine-one, or piperidine.

In some embodiments, R⁷ is C₁-C₃ haloalkyl, 3-membered cycloalkyl, phenyl, 4-membered heterocyclyl, or 5- to 6-membered heteroaryl, each of which is optionally substituted with one or more R⁴.

In other embodiments, R⁷ is —CF₃, cyclopropyl, phenyl, pyrazole, pyridyl, or triazole, each of which is optionally substituted with one or more R⁴.

In some embodiments, the compound has the structure of formula (VIIA):

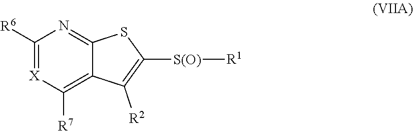

or a pharmaceutically acceptable salt, tautomer, or solvate thereof, wherein:
R¹ is cycloalkyl, alkylene-cycloalkyl, alkylene-alkoxy, heterocyclyl, or alkylene-heterocyclyl;
R² is —NH₂, CN, or —NHC(O)alkyl;
R⁶ is fused bicyclic heterocyclyl or fused bicyclic heteroaryl, each of which is optionally substituted with one or more R³;
R⁷ is alkyl, haloalkyl, cycloalkyl, aryl, heterocyclyl, heteroaryl, —C(O)-alkyl, —C(O)O-alkyl, or —C(O)NR⁵-alkyl, each of which is optionally substituted with one or more R⁴;
R³ is oxo, —OH, —O-alkylene-N(R⁵)₂, —N(R⁵)₂, —N(R⁵)(alkylene-OH), alkyl, haloalkyl, cycloalkyl, heterocyclyl, —C(O)N(R⁵)₂, —C(O)-alkyl, —C(O)O-alkyl, or —S(O)ₘ-alkyl;
R⁴ is oxo, halogen, —CN, —N(R⁵)₂, —OH, —O-alkylene-OH, —S(O)ₘ-alkyl, —C(O)— alkyl, —C(O)-cycloalkyl, alkyl, haloalkyl, cycloalkyl, heterocyclyl, or -alkylene-aryl optionally substituted with R⁸;
each R⁵ is independently, H, alkyl, -alkylene-OH optionally substituted with —OH, -alkylene-O-alkylene-OH, -alkylene-O-alkylene-NH₂, —C(O)-alkyl, —C(O)O-alkyl, or —S(O)ₘ-alkyl;
or alternatively, two R⁵ together with the N atom to which they are attached can form a 4- to 7-membered heterocycle, optionally containing an additional heteroatom selected from O, S, or N, and wherein the heterocycle is optionally substituted with R⁸;
R⁸ is halogen, alkyl, or alkoxy;
X is N or CH; and
m is 0, 1, or 2.

In some embodiments, R⁶ is 8- to 10-membered fused bicyclic heteroaryl, each of which is optionally substituted with one or more R³.

In some embodiments, R⁷ is C₁-C₆ alkyl, C₁-C₆ haloalkyl, 3- to 6-membered cycloalkyl, 6- to 10-membered aryl, 3- to 6-membered heterocyclyl, 5- to 10-membered heteroaryl, —C(O)(C₁-C₆ alkyl), —C(O)O(C₁-C₆ alkyl), or —C(O)NR⁵(C₁-C₆ alkyl), each of which is optionally substituted with one or more R⁴.

In other embodiments, R⁷ is C₁-C₄ alkyl, C₁-C₆ haloalkyl, 3- to 6-membered cycloalkyl, phenyl, 3- to 6-membered heterocyclyl, or 5- to 10-membered heteroaryl, each of which is optionally substituted with one or more R⁴. In other embodiments, R⁷ is C₁-C₆ haloalkyl, 3- to 6-membered cycloalkyl, phenyl, 3- to 6-membered heterocyclyl, or 5- to 10-membered heteroaryl, each of which is optionally substituted with one or more R⁴.

In still other embodiments, R⁷ is C₁-C₃ alkyl, C₁-C₆ haloalkyl, 3- to 6-membered cycloalkyl, phenyl, 3- to 6-membered heterocyclyl, or 5- to 6-membered heteroaryl, each of which is optionally substituted with one or more R⁴.

In other embodiments, R⁷ is C₁-C₃ alkyl, C₁-C₆ haloalkyl, 3- to 6-membered cycloalkyl, phenyl, pyrazole, pyridyl, or triazole, each of which is optionally substituted with one or more R⁴. In other embodiments, R⁷ is C₁-C₆ haloalkyl, 3- to 6-membered cycloalkyl, phenyl, pyrazole, pyridyl, or triazole, each of which is optionally substituted with one or more R⁴.

In other embodiments, R⁷ is —CF₃, isopropyl, cyclopropyl, phenyl, pyridyl, or triazole, each of which is optionally substituted with one or more R⁴. In other embodiments, R⁷ is —CF₃, cyclopropyl, phenyl, pyridyl, or triazole, each of which is optionally substituted with one or more R⁴.

In some embodiments, R¹ is C₁-C₆ alkyl, C₁-C₆ haloalkyl, 3- to 6-membered cycloalkyl, —(C₁-C₆ alkylene)-(3- to 6-membered cycloalkyl), —(C₁-C₆ alkylene)-(C₁-C₆ alkoxy), 3- to 6-membered heterocyclyl, or —(C₁-C₆ alkylene)-(3- to 6-membered heterocyclyl).

In other embodiments, R¹ is cyclopropyl, cyclobutyl, cyclopentyl, cyclohexyl, —(CH₂)ₚ-cyclopropyl, —(CH₂)ₚ-cyclobutyl, —(CH₂)ₚ-cyclopentyl, or —(CH₂)ₚ-cyclohexyl; wherein p is 1, 2, or 3.

In some embodiments, R³ is —O—(C₁-C₆ alkylene)-N(R⁵)₂, —N(R⁵)₂, —N(R⁵)(C₁-C₆ alkylene-OH), —C(O)N(R⁵)₂, —C(O)N(R⁵)(C₁-C₆ alkylene-OH), —C(O)(C₁-C₆ alkyl), —C(O)O(C₁-C₆ alkyl), or —S(O)ₘ(C₁-C₆ alkyl).

In other embodiments, R³ is —NH₂, —N(C₁-C₃ alkyl)₂, —NHCH₂CH₂OH, —N(C₁-C₃ alkyl)CH₂CH₂OH, N(CH₂CH₂OH)₂, —NHCH₂CH(CH₂OH)₂, —N(C₁-C₃ alkyl)CH₂CH(CH₂OH)₂, —NHCH₂CH₂OCH₂CH₂OH, —NHCH₂CH₂OCH₂CH₂NH₂, —NHCH₂CH₂NH₂, —N(C₁-C₃ alkyl)CH₂CH₂NH₂, —NHCH₂CH₂NH(C₁-C₃ alkyl), —NHCH₂CH₂N(C₁-C₃ alkyl)₂, —N(C₁-C₃ alkyl)CH₂CH₂NH(C₁-C₃ alkyl), —N(C₁-C₃ alkyl)CH₂CH₂N(C₁-C₃ alkyl)₂, —NHSO₂CH₃, —N(C₁-C₃ alkyl)SO₂CH₃, —OCH₂CH₂OH, —OCH₂CH₂NH₂, —OCH₂CH₂NH(C₁-C₃ alkyl), or —OCH₂CH₂N(C₁-C₃ alkyl)₂.

In some embodiments, R³ is —NHCH₂CH₂OH or —N(CH₃)CH₂CH₂OH.

In other embodiments, R⁴ is halogen, —CN, —N(R⁵)₂, —OH, —O—(C₁-C₆ alkylene)-OH, —S(O)ₘ(C₁-C₆ alkyl), —C(O)(C₁-C₆ alkyl), —C(O)-(3- to 6-membered cycloalkyl), C₁-C₆ alkyl, C₁-C₆ haloalkyl, 3- to 6-membered cycloalkyl, or 3- to 6-membered heterocyclyl.

In some embodiments, the compound has the structure of formula (VIIIA):

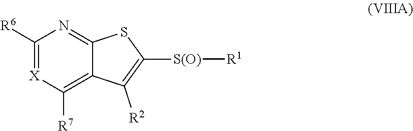

or a pharmaceutically acceptable salt, tautomer, or solvate thereof, wherein:
R¹ is cyclobutyl or —(C₁-C₄ alkylene)-(C₁-C₃ alkoxy);
R² is —NH₂, CN, or —NHC(O)alkyl;
R⁶ is heterocyclyl or heteroaryl, each of which is optionally substituted with one or more R³;
R⁷ is —CF₃, isopropyl,

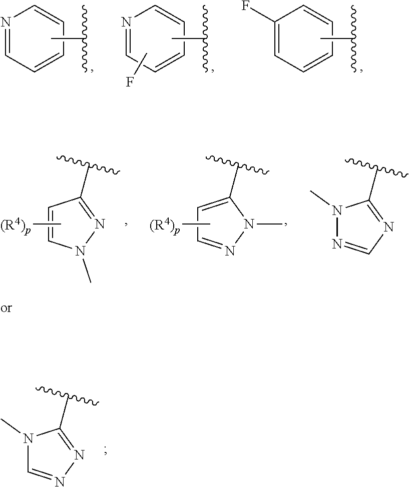

R³ is oxo, —OH, —O-alkylene-OH, —O-alkylene-N(R⁵)₂, —N(R⁵)₂, —N(R⁵)(alkylene-OH), —N(R⁵)(alkylene-O-alkyl), alkyl, -alkylene-OH, haloalkyl, cycloalkyl, heterocyclyl, —C(O)N(R⁵)₂, —C(O)N(R⁵)(alkylene-OH), —C(O)-alkyl, —C(O)O-alkyl, or —S(O)ₘ-alkyl, wherein the cycloalkyl and the heterocyclyl is each optionally substituted with R¹°;
R⁴ is C₁-C₃ alkyl;
each R⁵ is independently, H, alkyl, -alkylene-OH optionally substituted with —OH, -alkylene-NH₂, -alkylene-N(R⁹)₂, -alkylene-O-alkylene-OH, -alkylene-O-alkylene-NH₂, —C(O)-alkyl, —C(O)O-alkyl, -alkylene-COOH, or —S(O)ₘ-alkyl;
or alternatively, two R⁵ together with the N atom to which they are attached can form a 4- to 7-membered heterocycle, optionally containing an additional heteroatom selected from O, S, or N, and wherein the heterocycle is optionally substituted with R⁸;
R⁸ is halogen, alkyl, or alkoxy;
R⁹ is H or alkyl, or two R⁹ together with the N atom to which they are attached can form a 4- to 7-membered heterocycle, optionally containing an additional heteroatom selected from 0, S(O)ₜ, or N;
R¹⁰ is —OH, halogen, alkyl, or alkoxy;
X is N or CH;
m is 0, 1, or 2;
p is 0 or 1; and
t is 0, 1, or 2.

In still other embodiments, R² is —NH₂.

In some embodiments, R⁶ is 5- to 6-membered heterocyclyl or 5- to 10-membered heteroaryl, each of which is optionally substituted with one or more R³.

In other embodiments, R⁶ is 5- to 6-membered heteroaryl optionally substituted with one or more R³.

In still other embodiments, R⁶ is 8- to 10-membered bicyclic heteroaryl optionally substituted with one or more R³.

In some embodiments, R³ is —O—(C₁-C₆ alkylene)-N(R⁵)₂, —N(R⁵)₂, —N(R⁵)(C₁-C₆ alkylene-OH), —C(O)N(R⁵)₂, —C(O)N(R⁵)(C₁-C₆ alkylene-OH), —C(O)(C₁-C₆ alkyl), —C(O)O(C₁-C₆ alkyl), or —S(O)ₘ(C₁-C₆ alkyl).

In other embodiments, R³ is —(C₁-C₃ alkyl)OH, —NH₂, —N(C₁-C₃ alkyl)₂, —NHCH₂CH₂OH, —N(C₁-C₃ alkyl)CH₂CH₂OH, N(CH₂CH₂OH)₂, —NHCH₂CH(CH₂OH)₂, —N(C₁-C₃ alkyl)CH₂CH(CH₂OH)₂, —NHCH₂CH₂OCH₂CH₂OH, —NHCH₂CH₂OCH₂CH₂NH₂, —NHCH₂CH₂NH₂, —N(C₁-C₃ alkyl)CH₂CH₂NH₂, —NHCH₂CH₂NH(C₁-C₃ alkyl), —NHCH₂CH₂N(C₁-C₃ alkyl)₂, —N(C₁-C₃ alkyl)CH₂CH₂NH(C₁-C₃ alkyl), —N(C₁-C₃ alkyl)CH₂CH₂N(C₁-C₃ alkyl)₂, —NHSO₂CH₃, —N(C₁-C₃ alkyl)SO₂CH₃, —OCH₂CH₂OH, —OCH₂CH₂NH₂, —OCH₂CH₂NH(C₁-C₃ alkyl), or —OCH₂CH₂N(C₁-C₃ alkyl)₂.

In still other embodiments, R³ is —NHCH₂CH₂OH or —N(CH₃)CH₂CH₂OH.

In some embodiments, R⁴ is halogen, —CN, —N(R⁵)₂, —OH, —O—(C₁-C₆ alkylene)-OH, —S(O)ₘ(C₁-C₆ alkyl), —C(O)(C₁-C₆ alkyl), —C(O)-(3- to 6-membered cycloalkyl), C₁-C₆ alkyl, C₁-C₆ haloalkyl, 3- to 6-membered cycloalkyl, or 3- to 6-membered heterocyclyl.

In some embodiments, n is 1.

In some embodiments, the compound has the structure of formula (IXA):

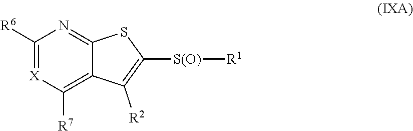

or a pharmaceutically acceptable salt, tautomer, or solvate thereof, wherein:
R¹ is cyclobutyl or —(C₁-C₄ alkylene)-(C₁-C₃ alkoxy);
R² is —NH₂, CN, or —NHC(O)alkyl;
R⁶ is

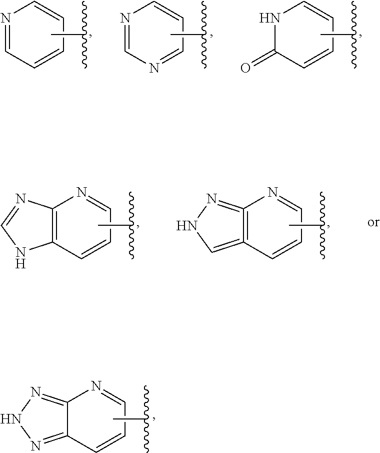

each of which is optionally substituted with one or more R³;
R⁷ is —CF₃, isopropyl, cyclopropyl, cyclobutyl

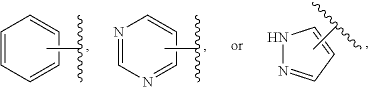

each of which is optionally substituted with one or more R⁴;
R³ is —NH₂, —NH(C₁-C₃ alkyl), —NH(C₁-C₄ alkylene)-OH, or C₁-C₃ alkyl;
R⁴ is C₁-C₃ alkyl; and
X is N or CH.

In some embodiments of Formula (IXA), R¹ is cyclobutyl. In some embodiments of Formula (IXA), R¹—(C₁-C₄ alkylene)-(C₁-C₃ alkoxy). In some embodiments of Formula (IXA), the R¹—(C₁-C₄ alkylene)-(C₁-C₃ alkoxy) is —(C₂-C₃ alkylene)-(C₁ alkoxy).

In some embodiments of Formula (IXA), R² is —NH₂.

In some embodiments of Formula (IXA), R⁶ is

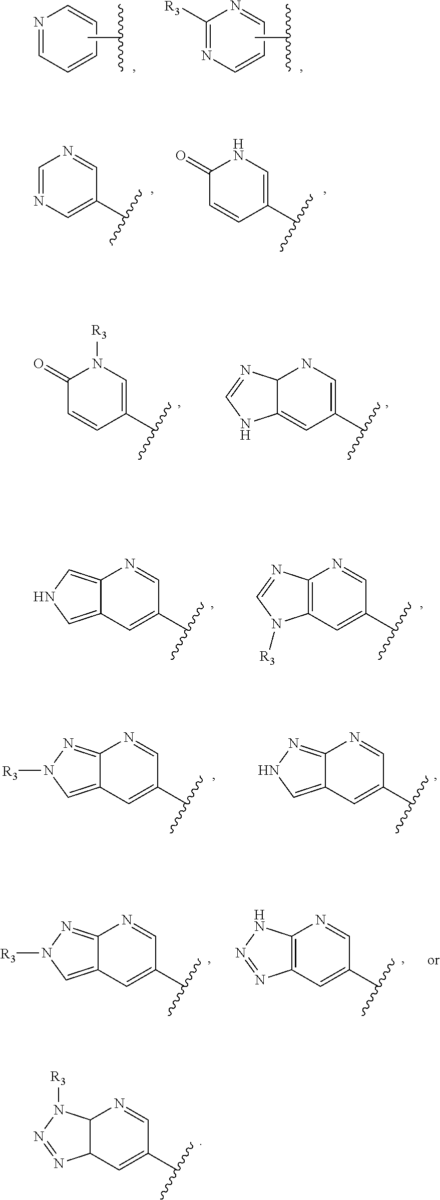

In some embodiments of Formula (IXA), R³ is —NH₂. In some embodiments of Formula (IXA), R³ is —NH(C₁-C₃ alkyl). In some embodiments of Formula (IXA), R³ is —NH(C₁-C₄ alkylene)-OH (e.g., —NH(C₂-C₄ alkylene)-OH). In some embodiments of Formula (IXA), R³ is C₁-C₃ alkyl (e.g., methyl or ethyl).

In some embodiments, of Formula (IXA), R⁷ is —CF₃, isopropyl, cyclopropyl, or cyclobutyl. In some embodiments, of Formula (IXA), R⁷ is isopropyl. In some embodiments of Formula (VII), R⁷ is

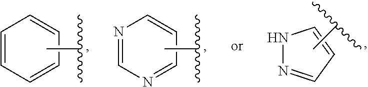

each of which is optionally substituted with one or more R⁴. In some embodiments, each R⁴ is independently selected from methyl or ethyl.

In some embodiments of Formula (IXA), X is —CH.

Examples of compounds having formulas (I), (II), (III), (IV), (V), (IA), (IIA), (IIIA), (IVA), (VA), (VIA), (VIIA), (VIIIA), and (IXA) are described in U.S. Patent Application Publication Nos. 2015/0072998, 2017/0165241, 2017/0173028, 2018/0118756, WO2018/218251, and WO2020/106998, all of which are incorporated by reference in their entirety.

For example, the 15-PGDH inhibitor can include a compound selected from the group consisting of:

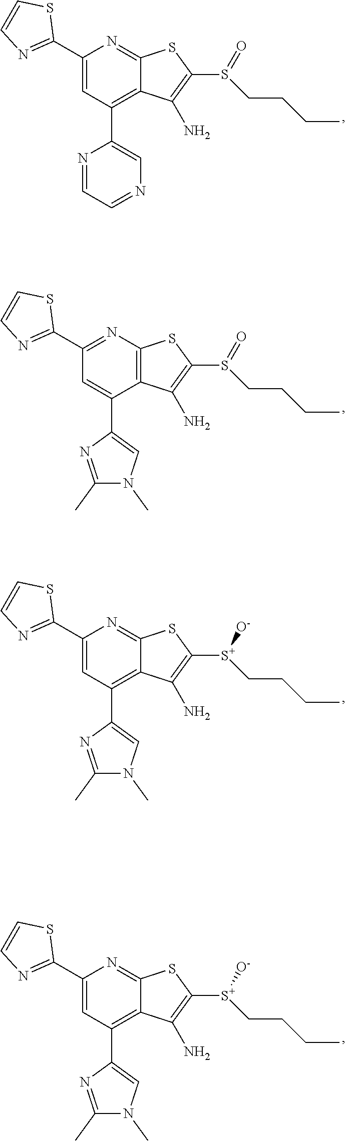 [SMILES: CCCCS(=O)c1sc2nc(-c3nccs3)cc(-c3cnccn3)c2c1N ; CCCCS(=O)c1sc2nc(-c3nccs3)cc(-c3cn(C)c(C)n3)c2c1N ; CCCC[S@+]([O-])c1sc2nc(-c3nccs3)cc(-c3cn(C)c(C)n3)c2c1N ; CCCC[S@@+]([O-])c1sc2nc(-c3nccs3)cc(-c3cn(C)c(C)n3)c2c1N]

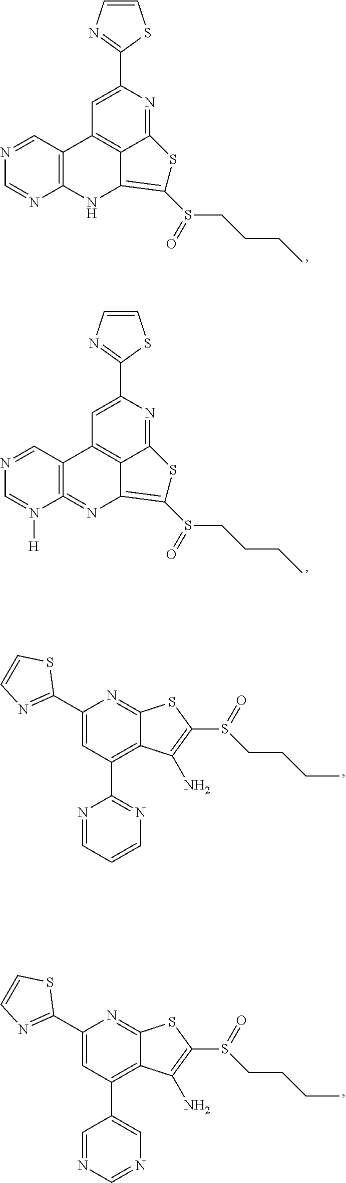

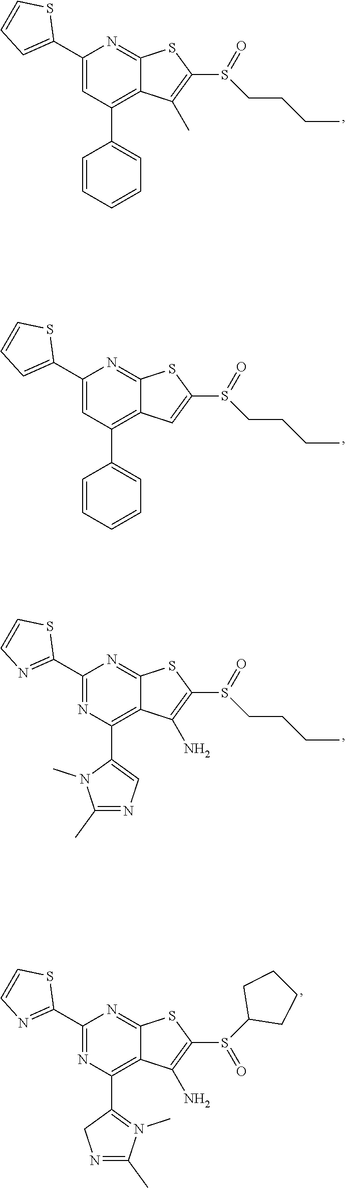

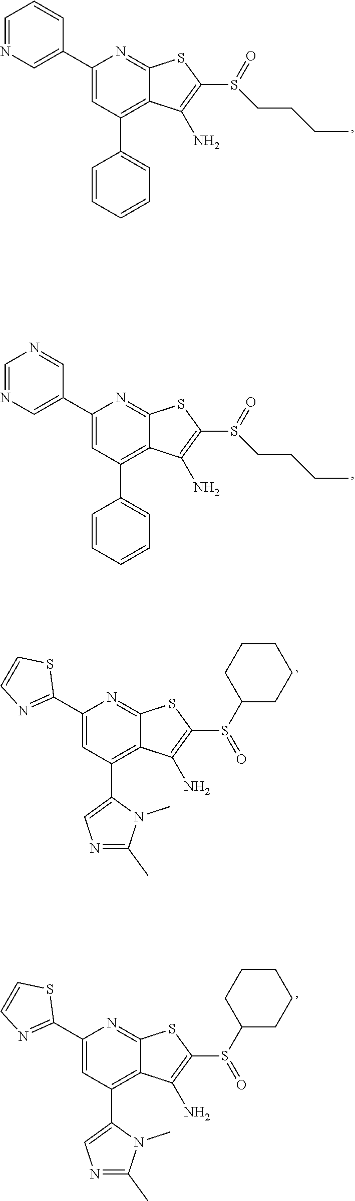 [SMILES: CCCCS(=O)c1sc2nc(-c3cccnc3)cc(-c3ccccc3)c2c1N ; CCCCS(=O)c1sc2nc(-c3cncnc3)cc(-c3ccccc3)c2c1N ; Cc1ncc(-c2cc(-c3nccs3)nc3sc(S(=O)C4CCCCC4)c(N)c23)n1C ; Cc1ncc(-c2cc(-c3nccs3)nc3sc(S(=O)C4CCCCC4)c(N)c23)n1C]

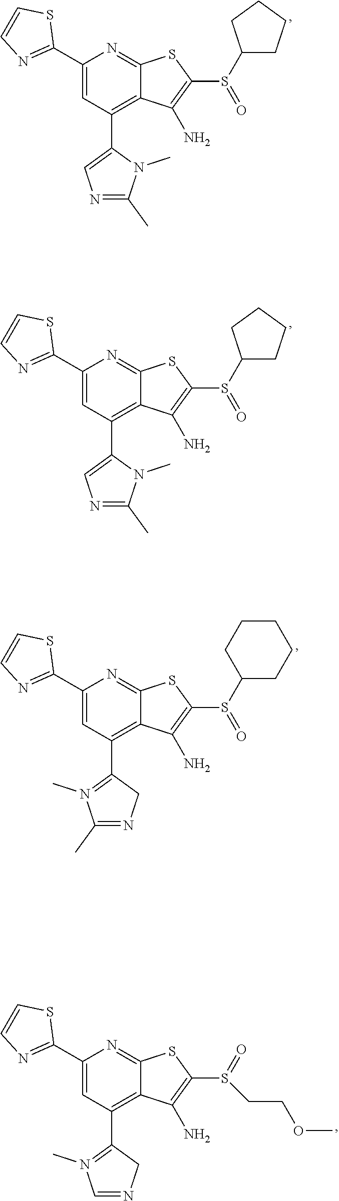

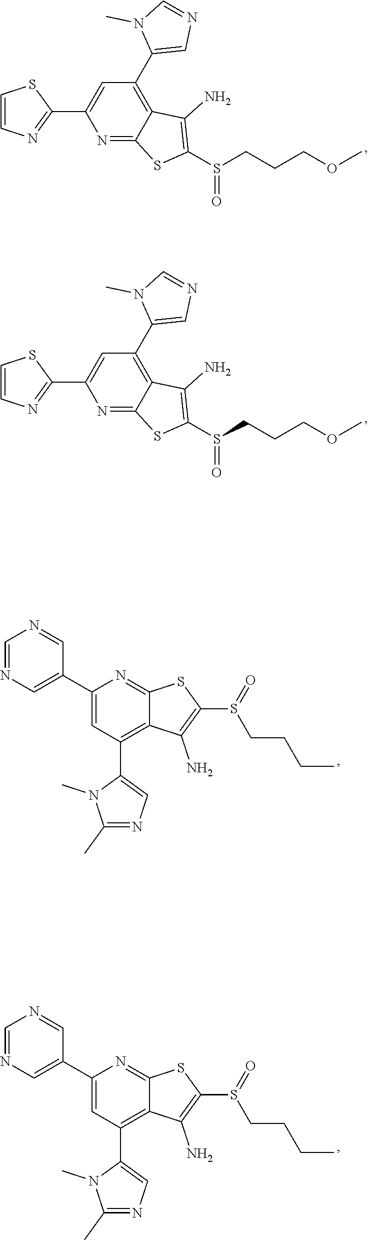 [SMILES: COCCCS(=O)c1sc2nc(-c3nccs3)cc(-c3cncn3C)c2c1N ; COCCC[S@@](=O)c1sc2nc(-c3nccs3)cc(-c3cncn3C)c2c1N ; CCCCS(=O)c1sc2nc(-c3cncnc3)cc(-c3cnc(C)n3C)c2c1N ; CCCCS(=O)c1sc2nc(-c3cncnc3)cc(-c3cnc(C)n3C)c2c1N]

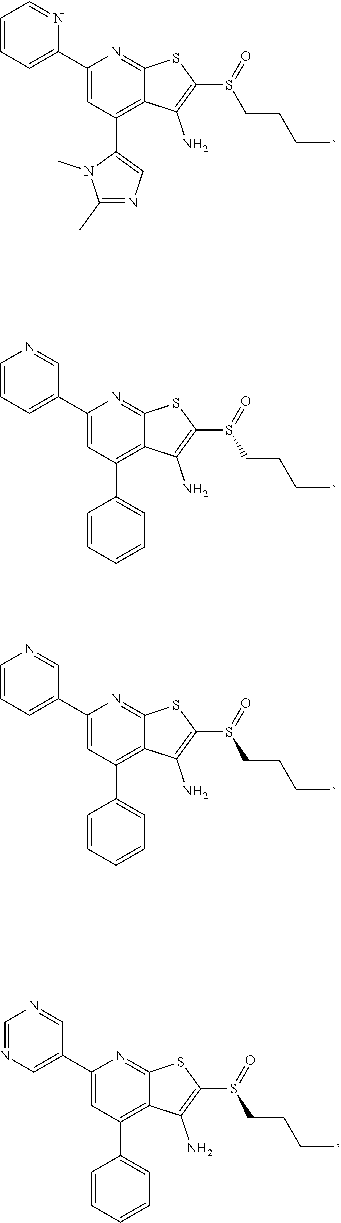 [SMILES: CCCCS(=O)c1sc2nc(-c3ccccn3)cc(-c3cnc(C)n3C)c2c1N ; CCCC[S@@](=O)c1sc2nc(-c3cccnc3)cc(-c3ccccc3)c2c1N ; CCCC[S@](=O)c1sc2nc(-c3cccnc3)cc(-c3ccccc3)c2c1N ; CCCC[S@](=O)c1sc2nc(-c3cncnc3)cc(-c3ccccc3)c2c1N]

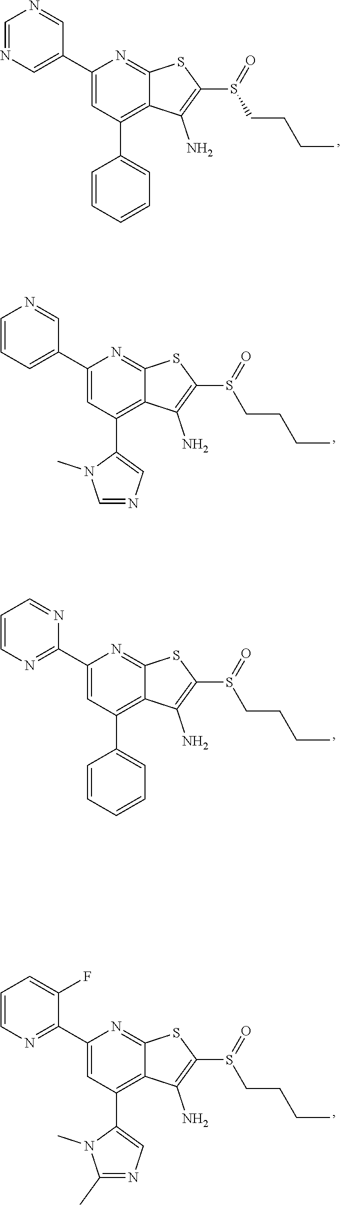 [SMILES: CCCC[S@@](=O)c1sc2nc(-c3cncnc3)cc(-c3ccccc3)c2c1N ; CCCCS(=O)c1sc2nc(-c3cccnc3)cc(-c3cncn3C)c2c1N ; CCCCS(=O)c1sc2nc(-c3ncccn3)cc(-c3ccccc3)c2c1N ; CCCCS(=O)c1sc2nc(-c3ncccc3F)cc(-c3cnc(C)n3C)c2c1N]

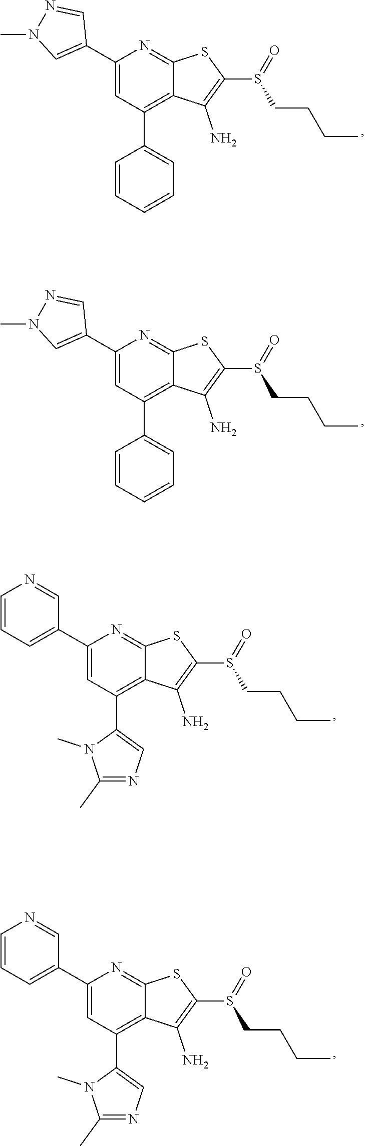

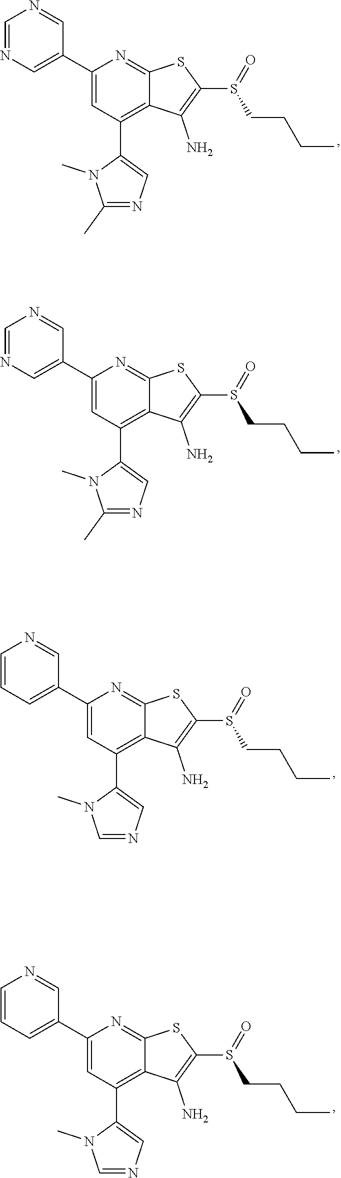 [SMILES: CCCC[S@@](=O)c1sc2nc(-c3cncnc3)cc(-c3cnc(C)n3C)c2c1N ; CCCC[S@](=O)c1sc2nc(-c3cncnc3)cc(-c3cnc(C)n3C)c2c1N ; CCCC[S@@](=O)c1sc2nc(-c3cccnc3)cc(-c3cncn3C)c2c1N ; CCCC[S@](=O)c1sc2nc(-c3cccnc3)cc(-c3cncn3C)c2c1N]

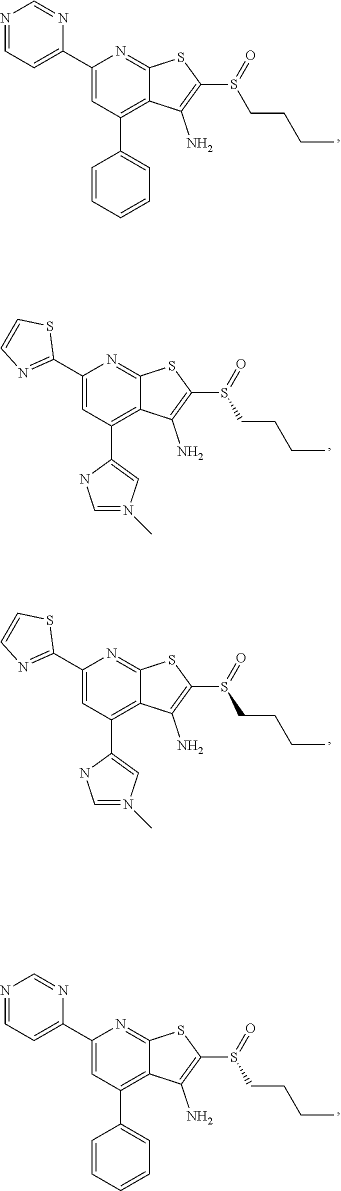

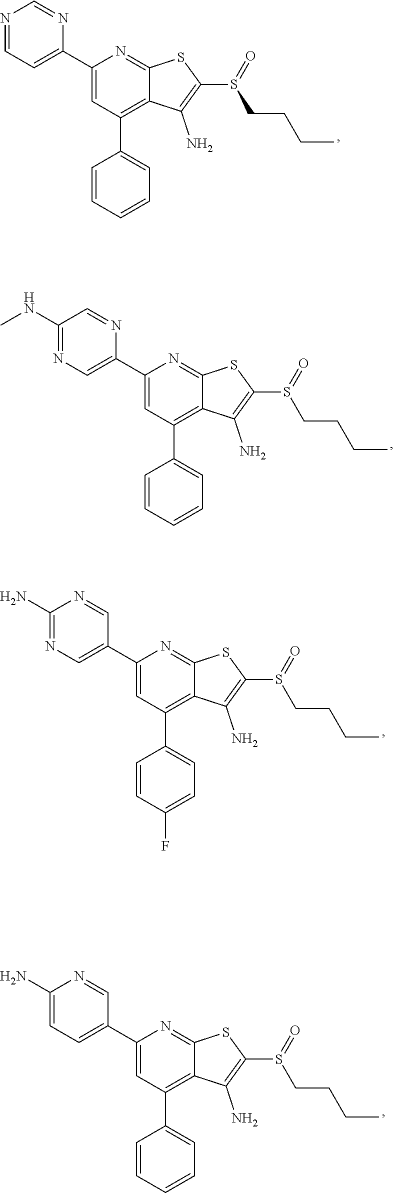

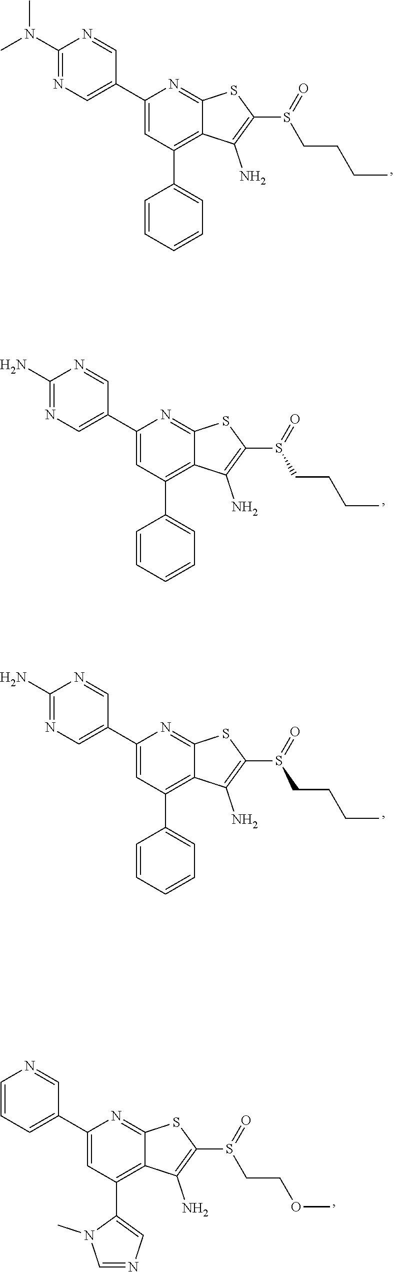 [SMILES: CCCCS(=O)c1sc2nc(-c3cnc(N(C)C)nc3)cc(-c3ccccc3)c2c1N ; CCCC[S@@](=O)c1sc2nc(-c3cnc(N)nc3)cc(-c3ccccc3)c2c1N ; CCCC[S@](=O)c1sc2nc(-c3cnc(N)nc3)cc(-c3ccccc3)c2c1N ; COCCS(=O)c1sc2nc(-c3cccnc3)cc(-c3cncn3C)c2c1N]

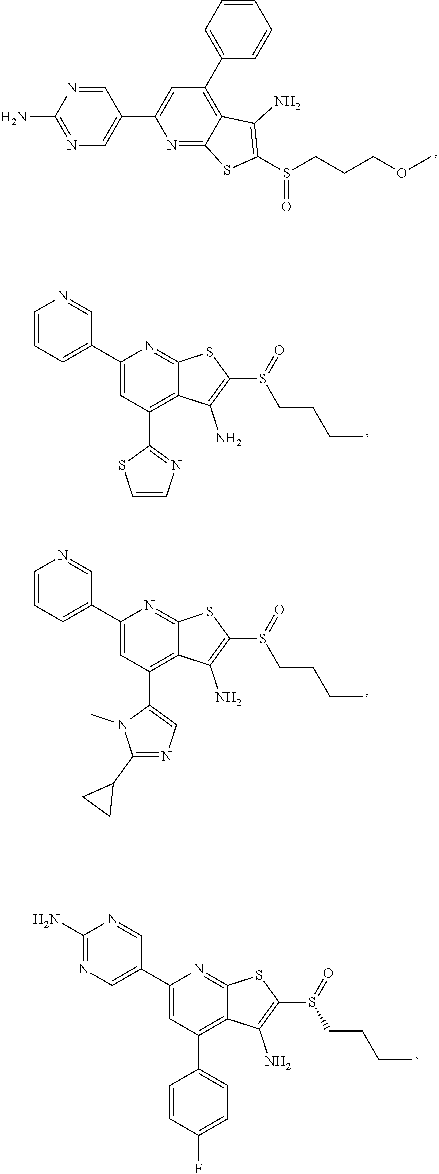 [SMILES: COCCCS(=O)c1sc2nc(-c3cnc(N)nc3)cc(-c3ccccc3)c2c1N ; CCCCS(=O)c1sc2nc(-c3cccnc3)cc(-c3nccs3)c2c1N ; CCCCS(=O)c1sc2nc(-c3cccnc3)cc(-c3cnc(C4CC4)n3C)c2c1N ; CCCC[S@@](=O)c1sc2nc(-c3cnc(N)nc3)cc(-c3ccc(F)cc3)c2c1N]

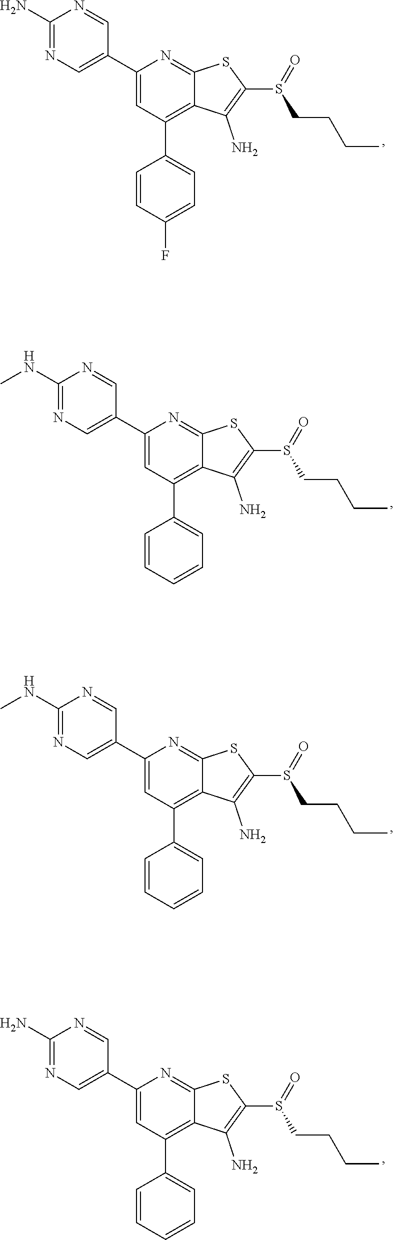

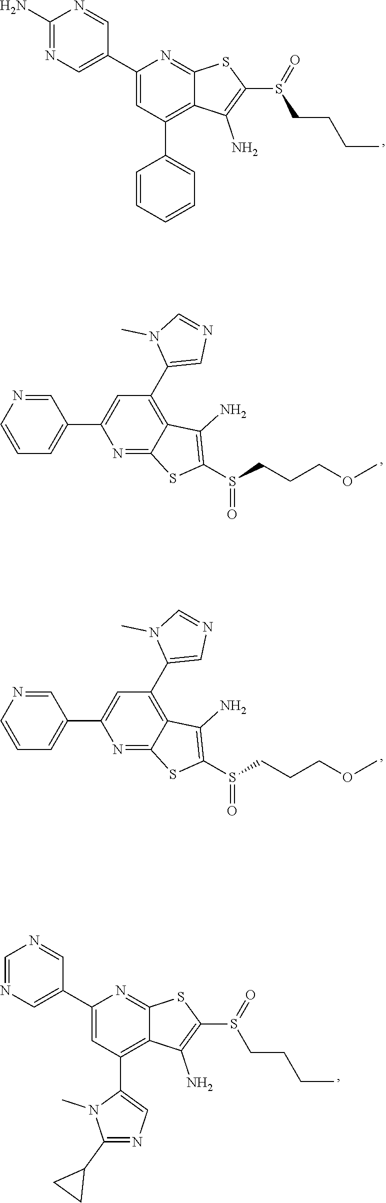 [SMILES: CCCC[S@](=O)c1sc2nc(-c3cnc(N)nc3)cc(-c3ccccc3)c2c1N ; COCCC[S@@](=O)c1sc2nc(-c3cccnc3)cc(-c3cncn3C)c2c1N ; COCCC[S@](=O)c1sc2nc(-c3cccnc3)cc(-c3cncn3C)c2c1N ; CCCCS(=O)c1sc2nc(-c3cncnc3)cc(-c3cnc(C4CC4)n3C)c2c1N]

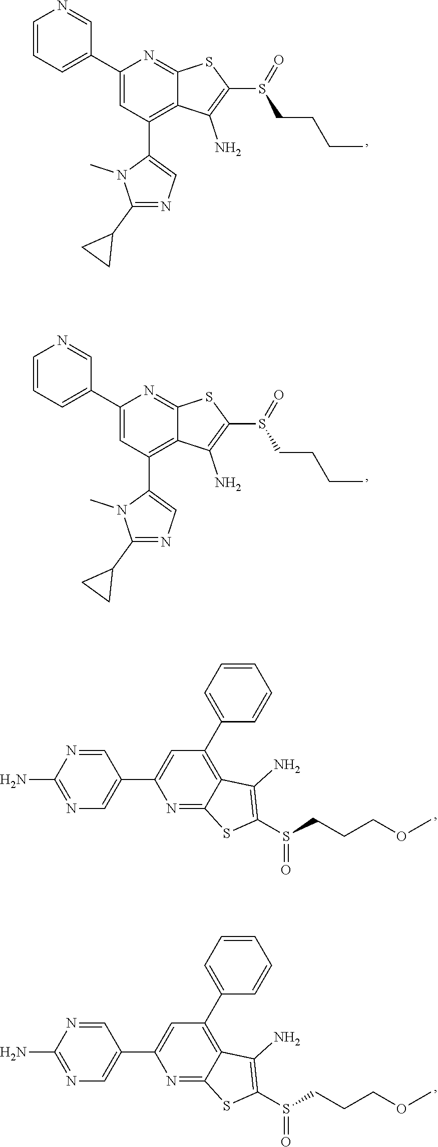 [SMILES: CCCC[S@](=O)c1sc2nc(-c3cccnc3)cc(-c3cnc(C4CC4)n3C)c2c1N ; CCCC[S@@](=O)c1sc2nc(-c3cccnc3)cc(-c3cnc(C4CC4)n3C)c2c1N ; COCCC[S@@](=O)c1sc2nc(-c3cnc(N)nc3)cc(-c3ccccc3)c2c1N ; COCCC[S@](=O)c1sc2nc(-c3cnc(N)nc3)cc(-c3ccccc3)c2c1N]

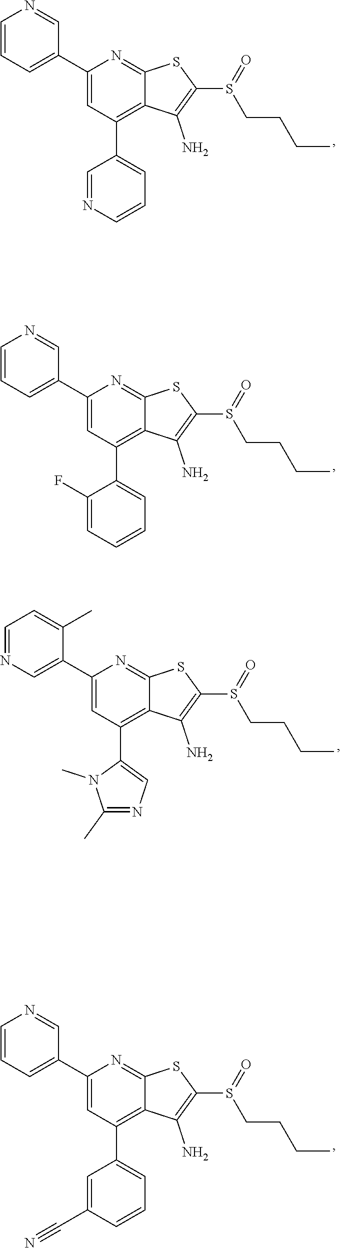 [SMILES: CCCCS(=O)c1sc2nc(-c3cccnc3)cc(-c3cccnc3)c2c1N ; CCCCS(=O)c1sc2nc(-c3cccnc3)cc(-c3ccccc3F)c2c1N ; CCCCS(=O)c1sc2nc(-c3cnccc3C)cc(-c3cnc(C)n3C)c2c1N ; CCCCS(=O)c1sc2nc(-c3cccnc3)cc(-c3cccc(C#N)c3)c2c1N]

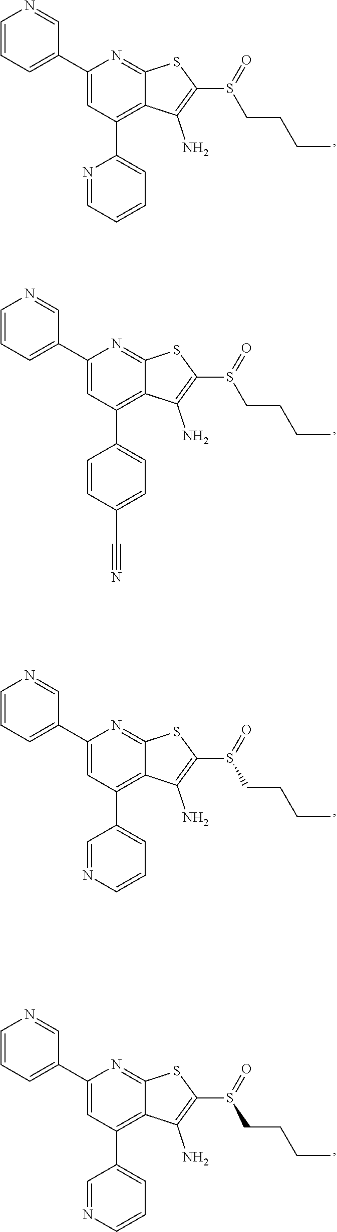 [SMILES: CCCCS(=O)c1sc2nc(-c3cccnc3)cc(-c3ccccn3)c2c1N ; CCCCS(=O)c1sc2nc(-c3cccnc3)cc(-c3ccc(C#N)cc3)c2c1N ; CCCC[S@@](=O)c1sc2nc(-c3cccnc3)cc(-c3cccnc3)c2c1N ; CCCC[S@](=O)c1sc2nc(-c3cccnc3)cc(-c3cccnc3)c2c1N]

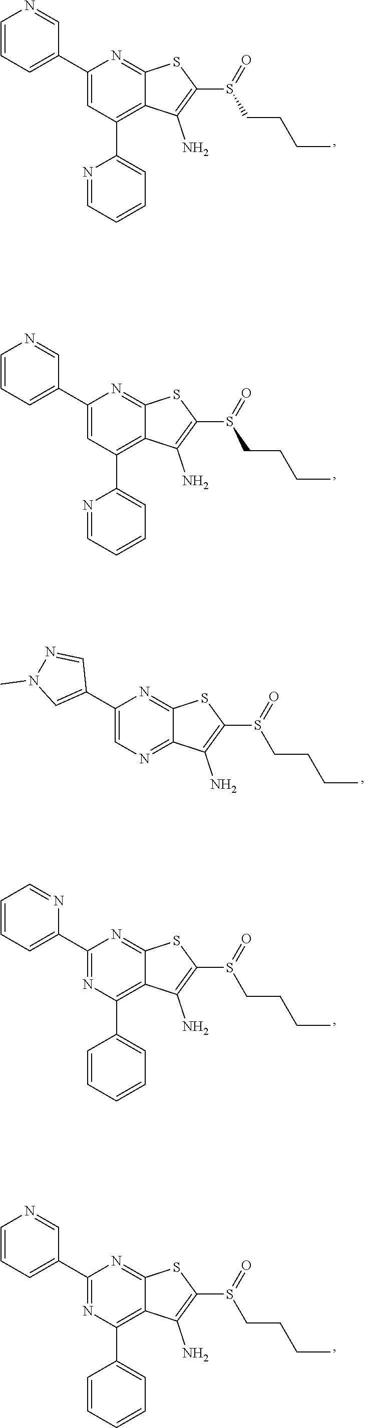

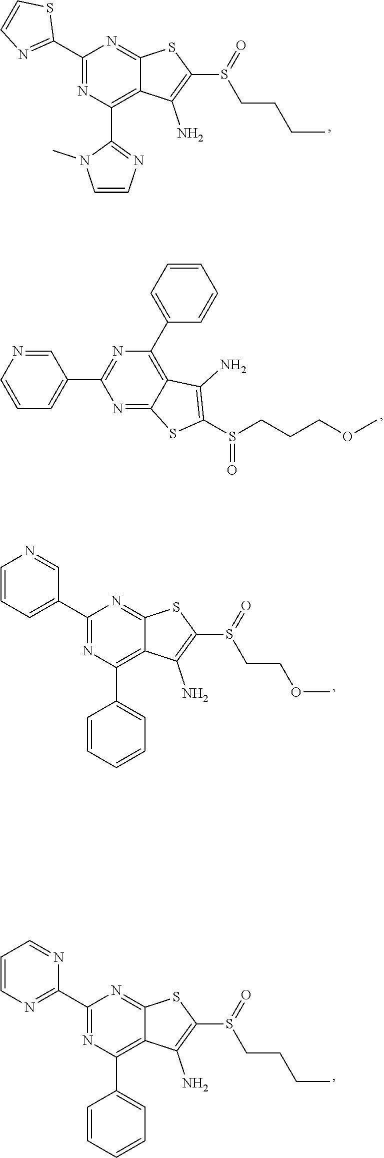 [SMILES: CCCCS(=O)c1sc2nc(-c3nccs3)nc(-c3nccn3C)c2c1N ; COCCCS(=O)c1sc2nc(-c3cccnc3)nc(-c3ccccc3)c2c1N ; COCCS(=O)c1sc2nc(-c3cccnc3)nc(-c3ccccc3)c2c1N ; CCCCS(=O)c1sc2nc(-c3ncccn3)nc(-c3ccccc3)c2c1N]

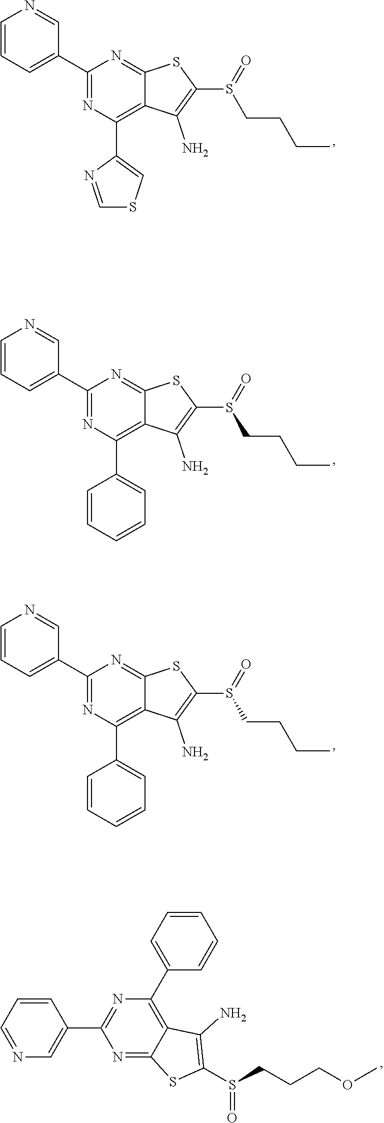 [SMILES: CCCCS(=O)c1sc2nc(-c3cccnc3)nc(-c3cscn3)c2c1N ; CCCC[S@](=O)c1sc2nc(-c3cccnc3)nc(-c3ccccc3)c2c1N ; CCCC[S@@](=O)c1sc2nc(-c3cccnc3)nc(-c3ccccc3)c2c1N ; COCCC[S@@](=O)c1sc2nc(-c3cccnc3)nc(-c3ccccc3)c2c1N]

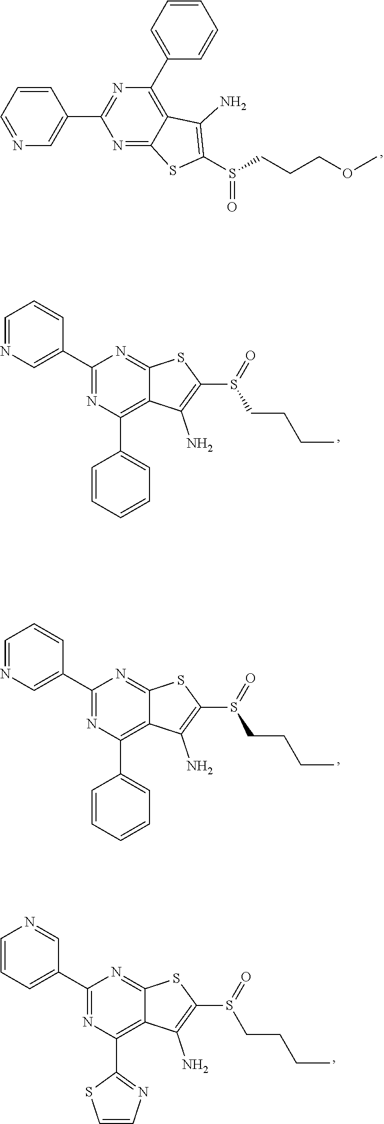 [SMILES: COCCC[S@](=O)c1sc2nc(-c3cccnc3)nc(-c3ccccc3)c2c1N ; CCCC[S@@](=O)c1sc2nc(-c3cccnc3)nc(-c3ccccc3)c2c1N ; CCCC[S@](=O)c1sc2nc(-c3cccnc3)nc(-c3ccccc3)c2c1N ; CCCCS(=O)c1sc2nc(-c3cccnc3)nc(-c3nccs3)c2c1N]

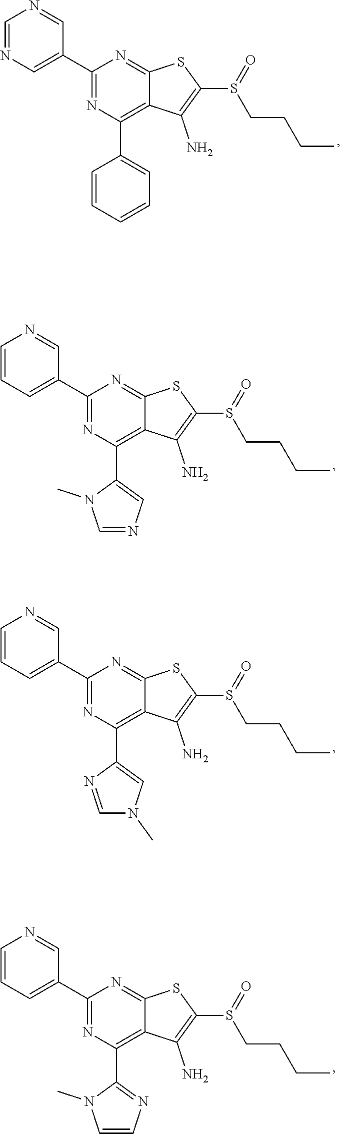 [SMILES: CCCCS(=O)c1sc2nc(-c3cncnc3)nc(-c3ccccc3)c2c1N ; CCCCS(=O)c1sc2nc(-c3cccnc3)nc(-c3cncn3C)c2c1N ; CCCCS(=O)c1sc2nc(-c3cccnc3)nc(-c3cn(C)cn3)c2c1N ; CCCCS(=O)c1sc2nc(-c3cccnc3)nc(-c3nccn3C)c2c1N]

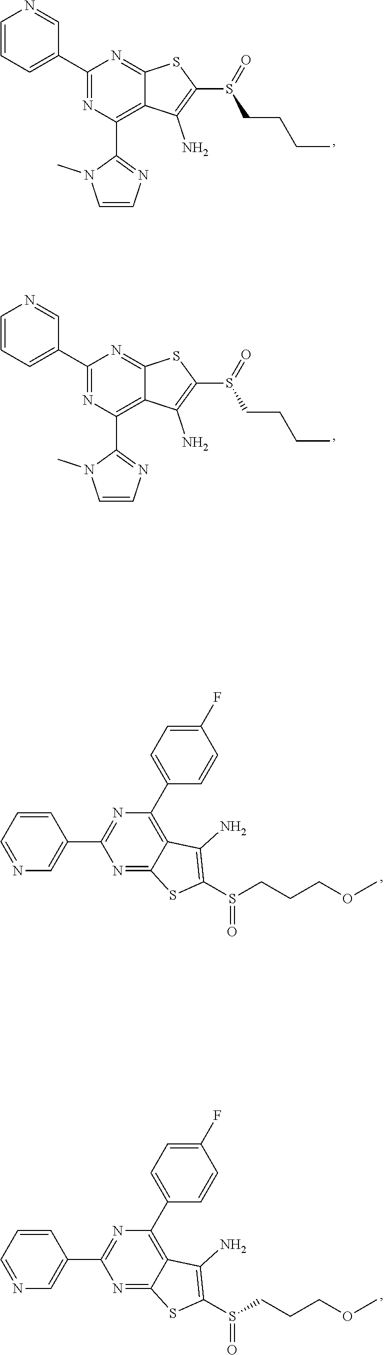 [SMILES: CCCC[S@](=O)c1sc2nc(-c3cccnc3)nc(-c3nccn3C)c2c1N ; CCCC[S@@](=O)c1sc2nc(-c3cccnc3)nc(-c3nccn3C)c2c1N ; COCCCS(=O)c1sc2nc(-c3cccnc3)nc(-c3ccc(F)cc3)c2c1N ; COCCC[S@](=O)c1sc2nc(-c3cccnc3)nc(-c3ccc(F)cc3)c2c1N]

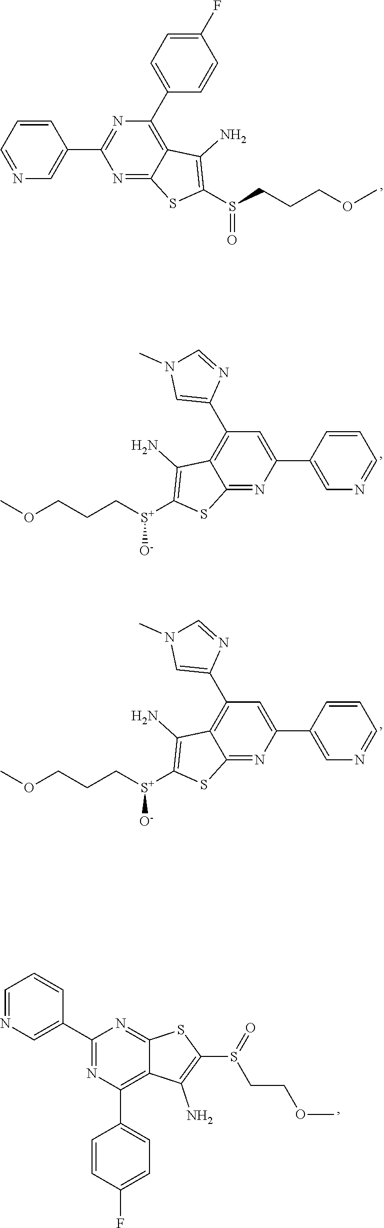

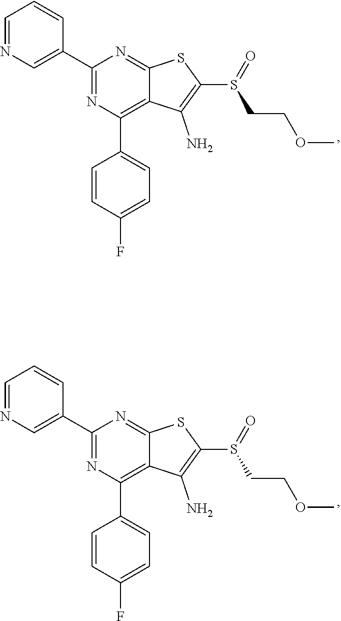 [SMILES: COCC[S@](=O)c1sc2nc(-c3cccnc3)nc(-c3ccc(F)cc3)c2c1N ; COCC[S@@](=O)c1sc2nc(-c3cccnc3)nc(-c3ccc(F)cc3)c2c1N]

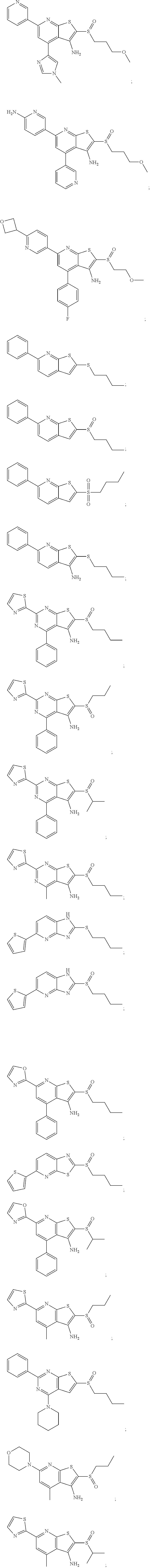

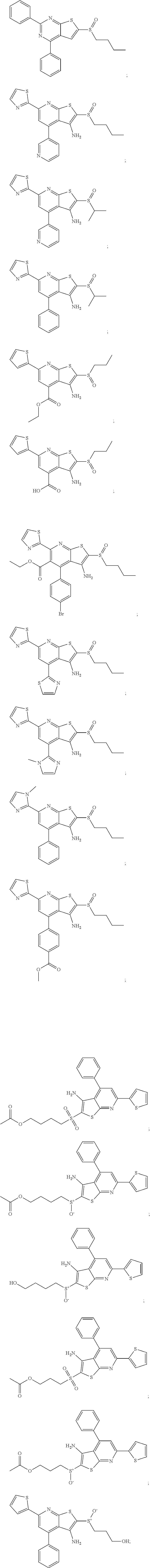

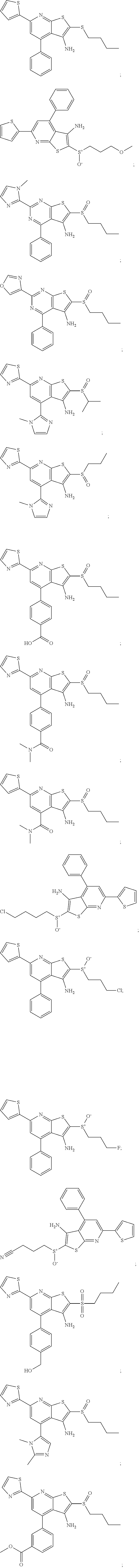

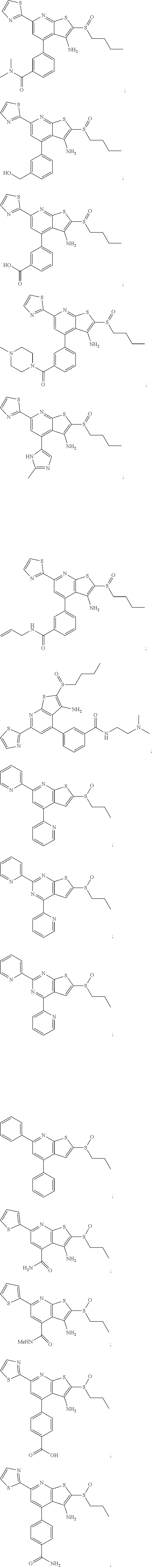

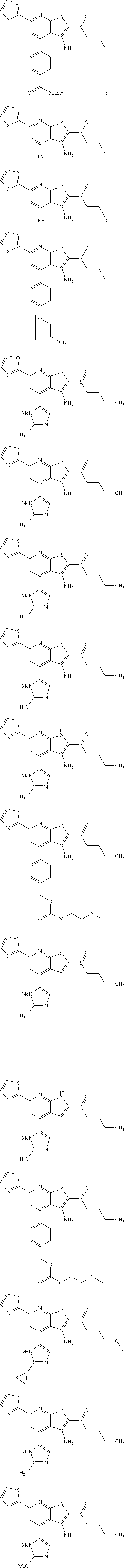

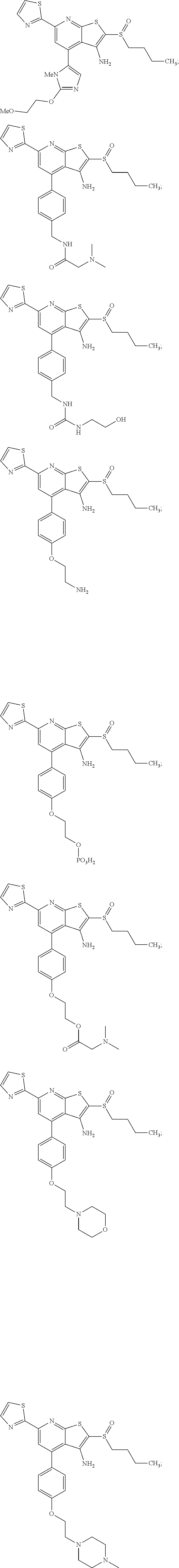

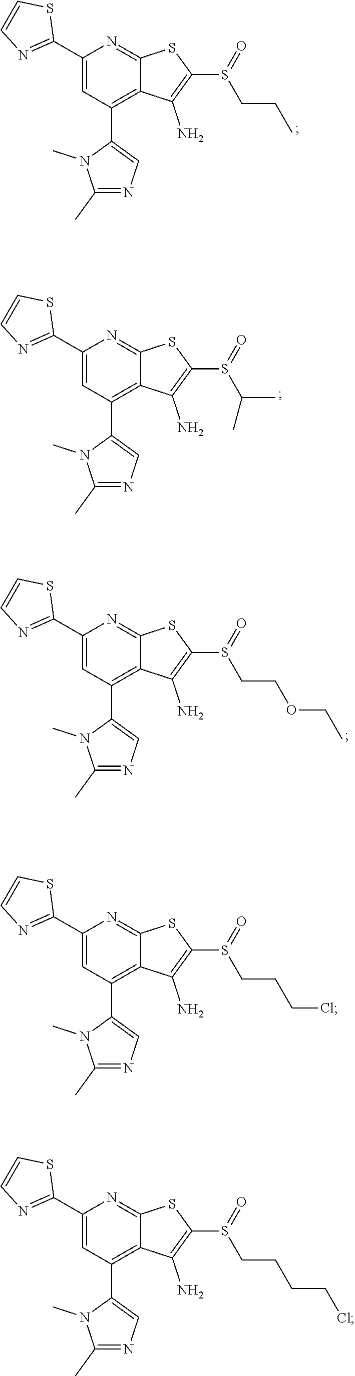 [SMILES: CCCS(=O)c1sc2nc(-c3nccs3)cc(-c3cnc(C)n3C)c2c1N ; Cc1ncc(-c2cc(-c3nccs3)nc3sc(S(=O)C(C)C)c(N)c23)n1C ; CCOCCS(=O)c1sc2nc(-c3nccs3)cc(-c3cnc(C)n3C)c2c1N ; Cc1ncc(-c2cc(-c3nccs3)nc3sc(S(=O)CCCCl)c(N)c23)n1C ; Cc1ncc(-c2cc(-c3nccs3)nc3sc(S(=O)CCCCCl)c(N)c23)n1C]

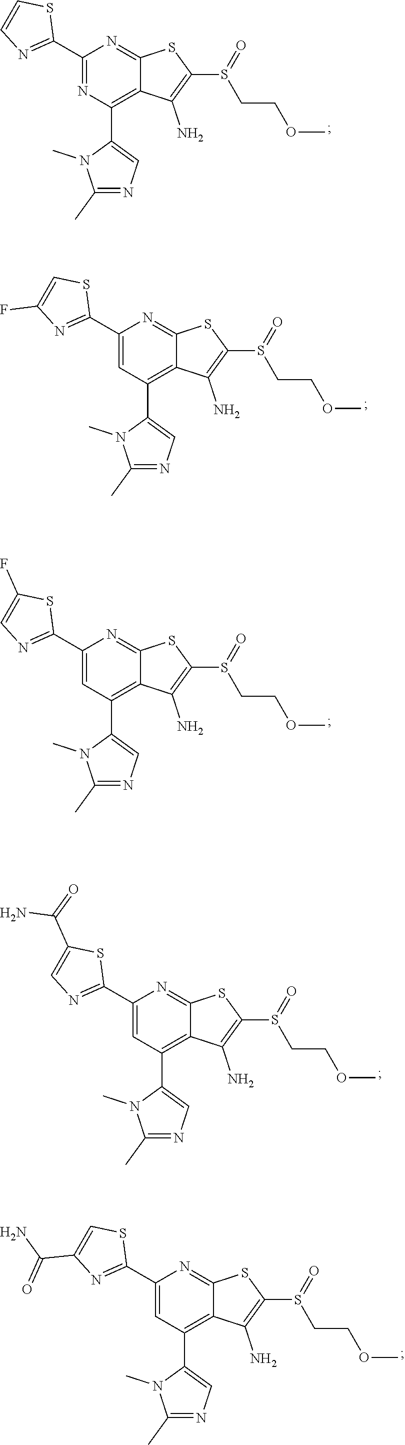 [SMILES: COCCS(=O)c1sc2nc(-c3nccs3)nc(-c3cnc(C)n3C)c2c1N ; COCCS(=O)c1sc2nc(-c3nc(F)cs3)cc(-c3cnc(C)n3C)c2c1N ; COCCS(=O)c1sc2nc(-c3ncc(F)s3)cc(-c3cnc(C)n3C)c2c1N ; COCCS(=O)c1sc2nc(-c3ncc(C(N)=O)s3)cc(-c3cnc(C)n3C)c2c1N ; COCCS(=O)c1sc2nc(-c3nc(C(N)=O)cs3)cc(-c3cnc(C)n3C)c2c1N]

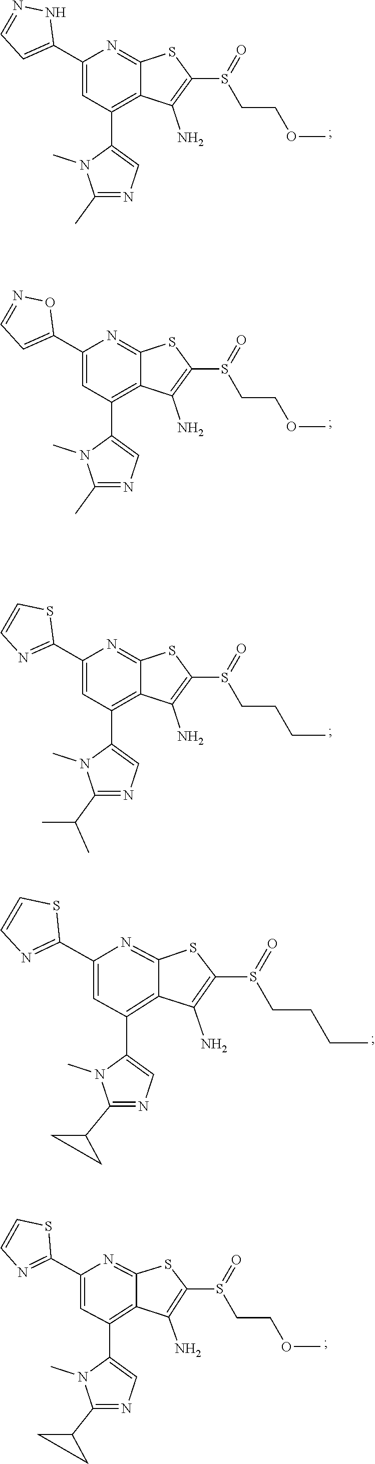 [SMILES: COCCS(=O)c1sc2nc(-c3ccn[nH]3)cc(-c3cnc(C)n3C)c2c1N ; COCCS(=O)c1sc2nc(-c3ccno3)cc(-c3cnc(C)n3C)c2c1N ; CCCCS(=O)c1sc2nc(-c3nccs3)cc(-c3cnc(C(C)C)n3C)c2c1N ; CCCCS(=O)c1sc2nc(-c3nccs3)cc(-c3cnc(C4CC4)n3C)c2c1N ; COCCS(=O)c1sc2nc(-c3nccs3)cc(-c3cnc(C4CC4)n3C)c2c1N]

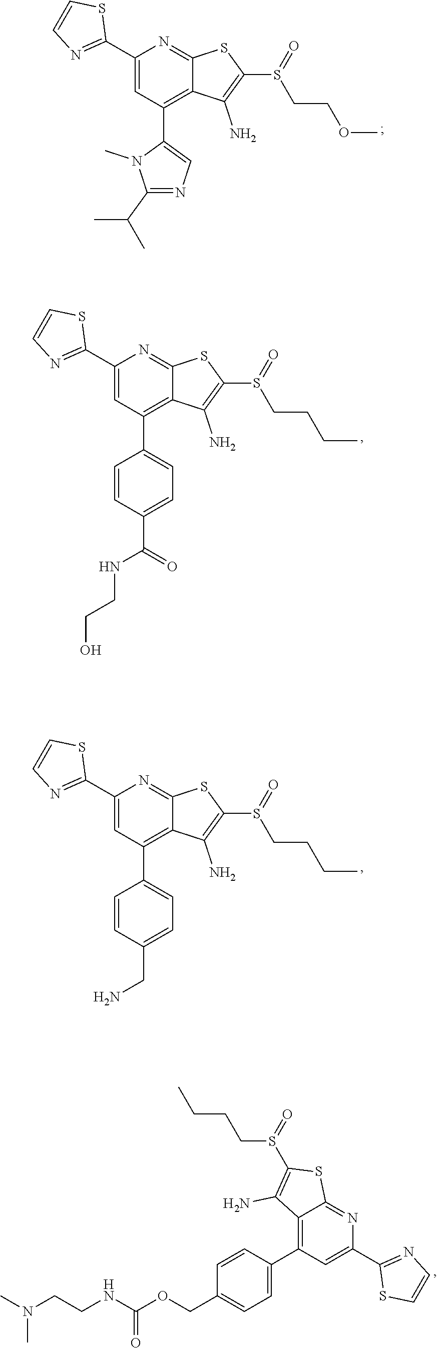

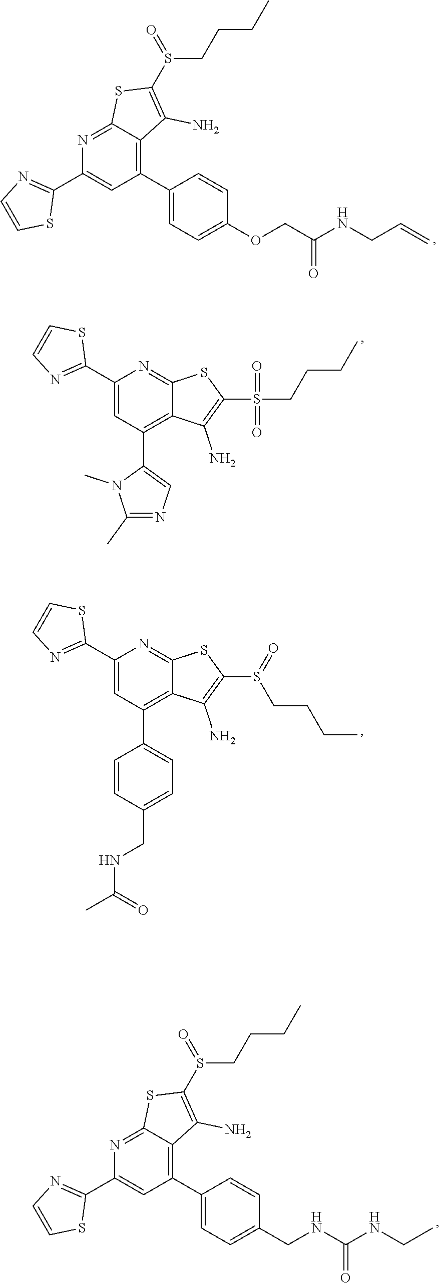 [SMILES: C=CCNC(=O)COc1ccc(-c2cc(-c3nccs3)nc3sc(S(=O)CCCC)c(N)c23)cc1 ; CCCCS(=O)(=O)c1sc2nc(-c3nccs3)cc(-c3cnc(C)n3C)c2c1N ; CCCCS(=O)c1sc2nc(-c3nccs3)cc(-c3ccc(CNC(C)=O)cc3)c2c1N ; CCCCS(=O)c1sc2nc(-c3nccs3)cc(-c3ccc(CNC(=O)NCC)cc3)c2c1N]

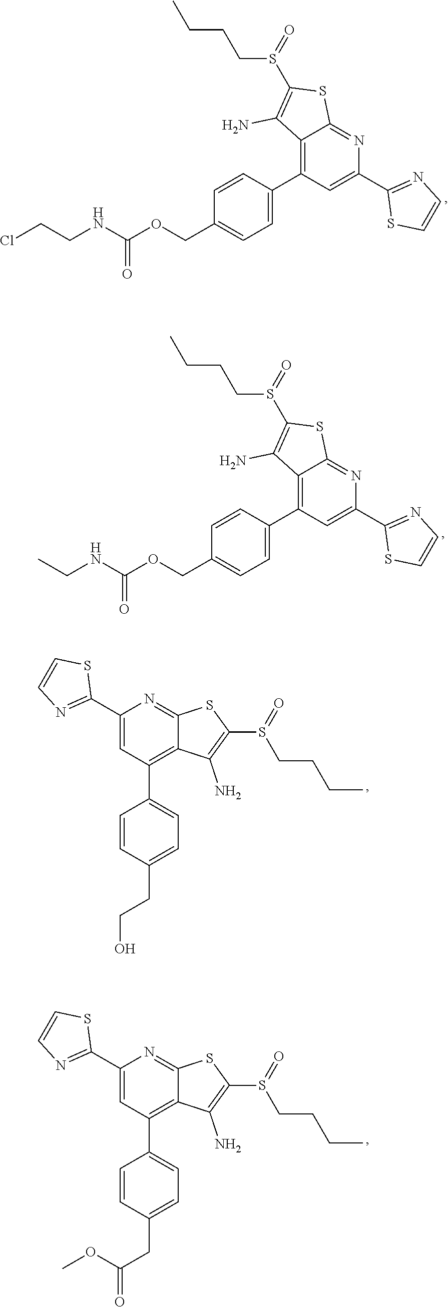 [SMILES: CCCCS(=O)c1sc2nc(-c3nccs3)cc(-c3ccc(COC(=O)NCCCl)cc3)c2c1N ; CCCCS(=O)c1sc2nc(-c3nccs3)cc(-c3ccc(COC(=O)NCC)cc3)c2c1N ; CCCCS(=O)c1sc2nc(-c3nccs3)cc(-c3ccc(CCO)cc3)c2c1N ; CCCCS(=O)c1sc2nc(-c3nccs3)cc(-c3ccc(CC(=O)OC)cc3)c2c1N]

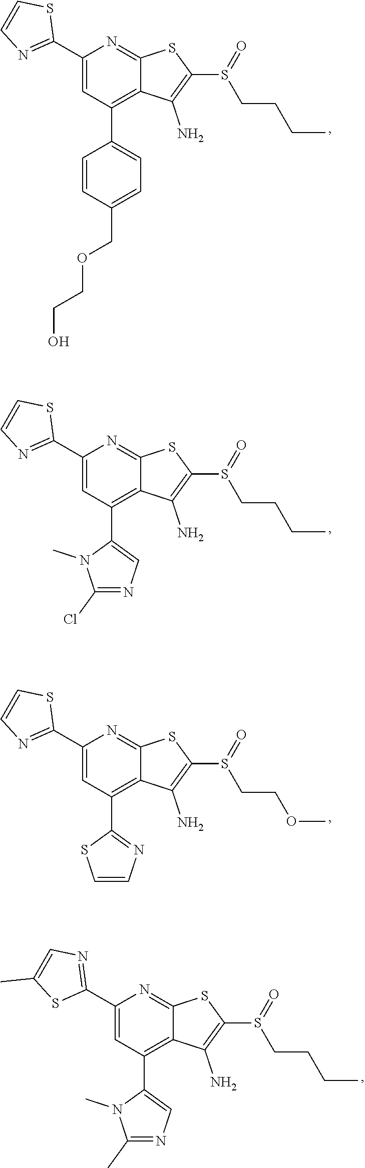

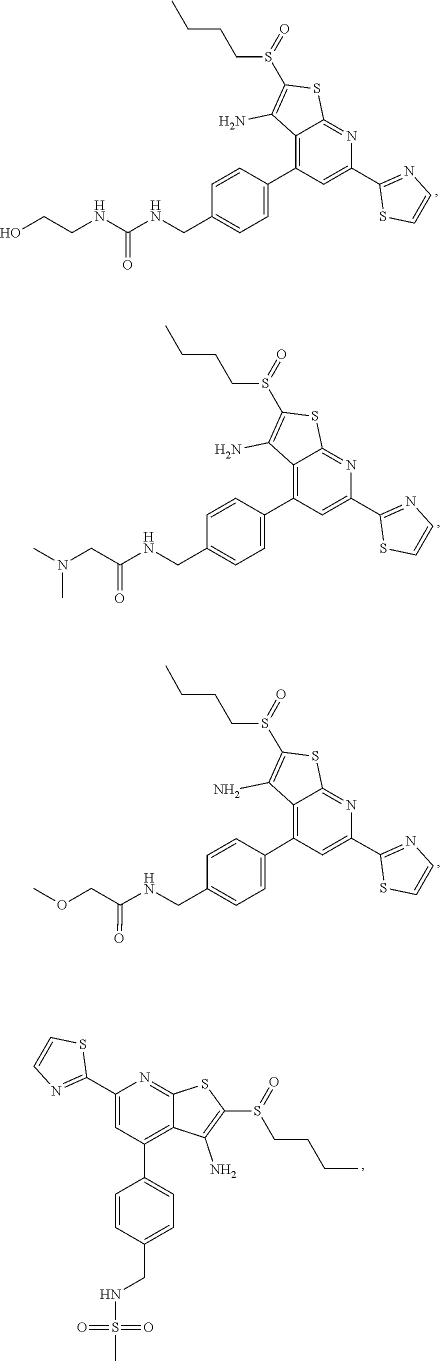 [SMILES: CCCCS(=O)c1sc2nc(-c3nccs3)cc(-c3ccc(CNC(=O)NCCO)cc3)c2c1N ; CCCCS(=O)c1sc2nc(-c3nccs3)cc(-c3ccc(CNC(=O)CN(C)C)cc3)c2c1N ; CCCCS(=O)c1sc2nc(-c3nccs3)cc(-c3ccc(CNC(=O)COC)cc3)c2c1N ; CCCCS(=O)c1sc2nc(-c3nccs3)cc(-c3ccc(CNS(C)(=O)=O)cc3)c2c1N]

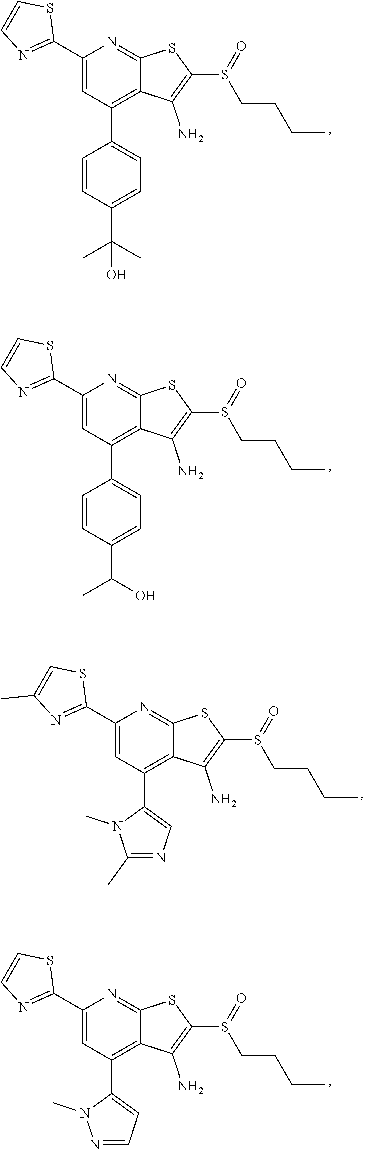

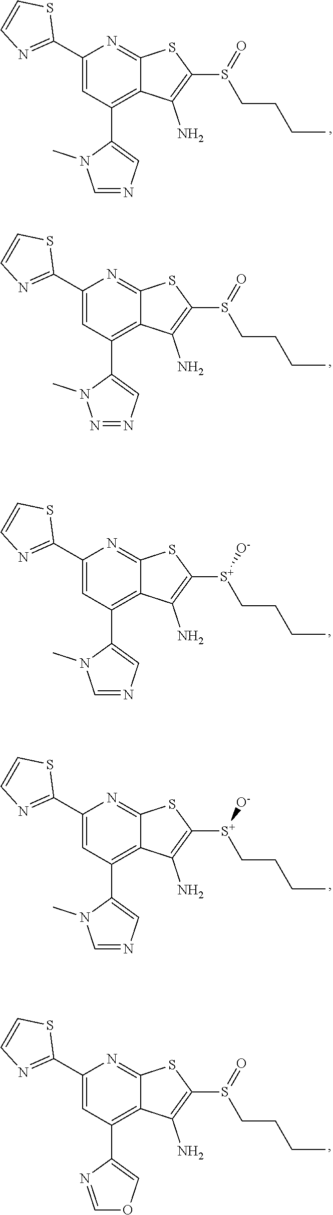

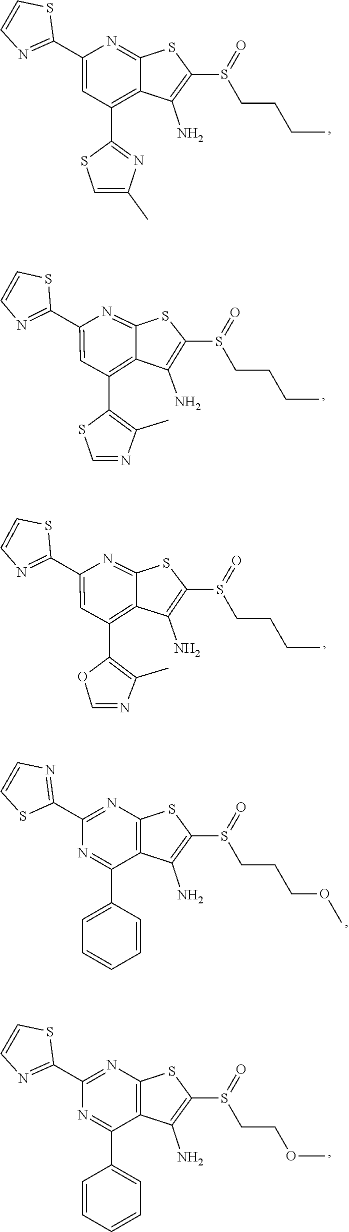 [SMILES: CCCCS(=O)c1sc2nc(-c3nccs3)cc(-c3nc(C)cs3)c2c1N ; CCCCS(=O)c1sc2nc(-c3nccs3)cc(-c3scnc3C)c2c1N ; CCCCS(=O)c1sc2nc(-c3nccs3)cc(-c3ocnc3C)c2c1N ; COCCCS(=O)c1sc2nc(-c3nccs3)nc(-c3ccccc3)c2c1N ; COCCS(=O)c1sc2nc(-c3nccs3)nc(-c3ccccc3)c2c1N]

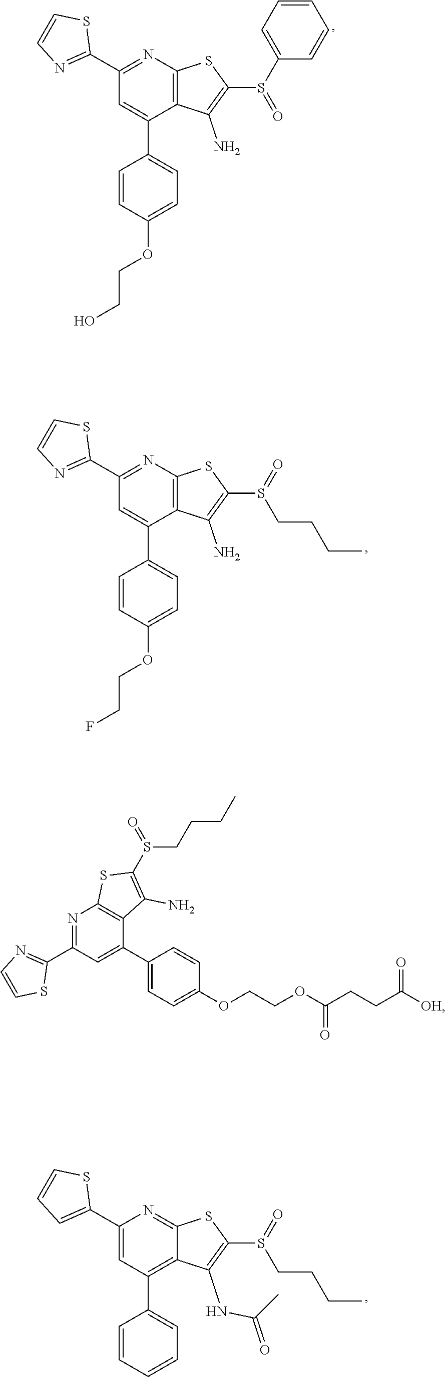 [SMILES: Nc1c(S(=O)c2ccccc2)sc2nc(-c3nccs3)cc(-c3ccc(OCCO)cc3)c12 ; CCCCS(=O)c1sc2nc(-c3nccs3)cc(-c3ccc(OCCF)cc3)c2c1N ; CCCCS(=O)c1sc2nc(-c3nccs3)cc(-c3ccc(OCCOC(=O)CCC(=O)O)cc3)c2c1N ; CCCCS(=O)c1sc2nc(-c3cccs3)cc(-c3ccccc3)c2c1NC(C)=O]

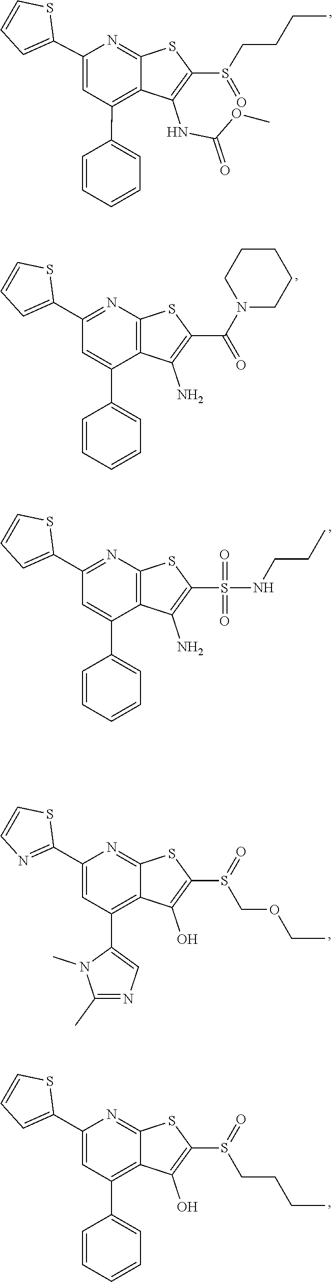 [SMILES: CCCCS(=O)c1sc2nc(-c3cccs3)cc(-c3ccccc3)c2c1NC(=O)OC ; Nc1c(C(=O)N2CCCCC2)sc2nc(-c3cccs3)cc(-c3ccccc3)c12 ; CCCNS(=O)(=O)c1sc2nc(-c3cccs3)cc(-c3ccccc3)c2c1N ; CCOCS(=O)c1sc2nc(-c3nccs3)cc(-c3cnc(C)n3C)c2c1O ; CCCCS(=O)c1sc2nc(-c3cccs3)cc(-c3ccccc3)c2c1O]

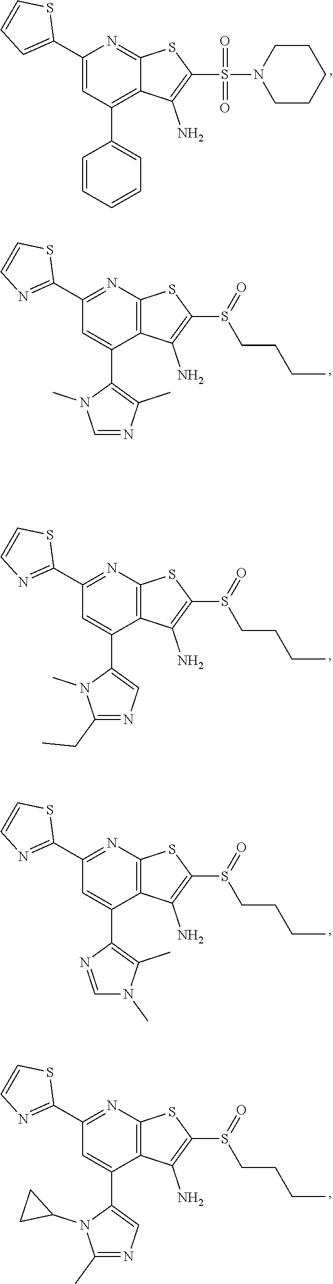 [SMILES: Nc1c(S(=O)(=O)N2CCCCC2)sc2nc(-c3cccs3)cc(-c3ccccc3)c12 ; CCCCS(=O)c1sc2nc(-c3nccs3)cc(-c3c(C)ncn3C)c2c1N ; CCCCS(=O)c1sc2nc(-c3nccs3)cc(-c3cnc(CC)n3C)c2c1N ; CCCCS(=O)c1sc2nc(-c3nccs3)cc(-c3ncn(C)c3C)c2c1N ; CCCCS(=O)c1sc2nc(-c3nccs3)cc(-c3cnc(C)n3C3CC3)c2c1N]

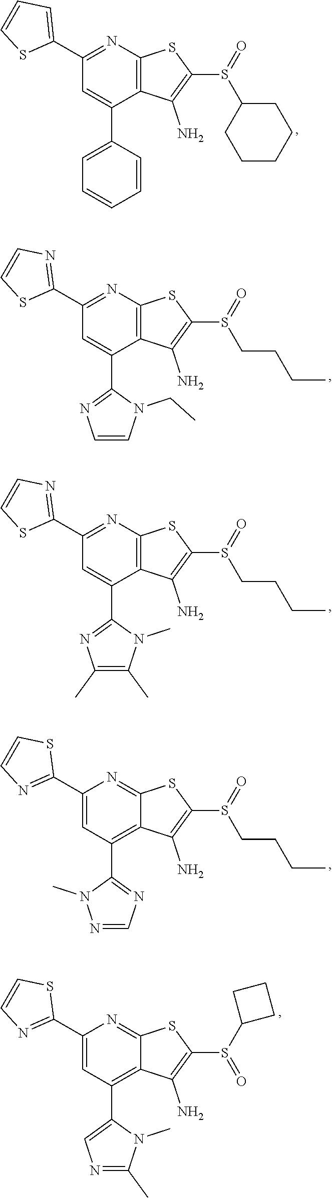 [SMILES: Nc1c(S(=O)C2CCCCC2)sc2nc(-c3cccs3)cc(-c3ccccc3)c12 ; CCCCS(=O)c1sc2nc(-c3nccs3)cc(-c3nccn3CC)c2c1N ; CCCCS(=O)c1sc2nc(-c3nccs3)cc(-c3nc(C)c(C)n3C)c2c1N ; CCCCS(=O)c1sc2nc(-c3nccs3)cc(-c3ncnn3C)c2c1N ; Cc1ncc(-c2cc(-c3nccs3)nc3sc(S(=O)C4CCC4)c(N)c23)n1C]

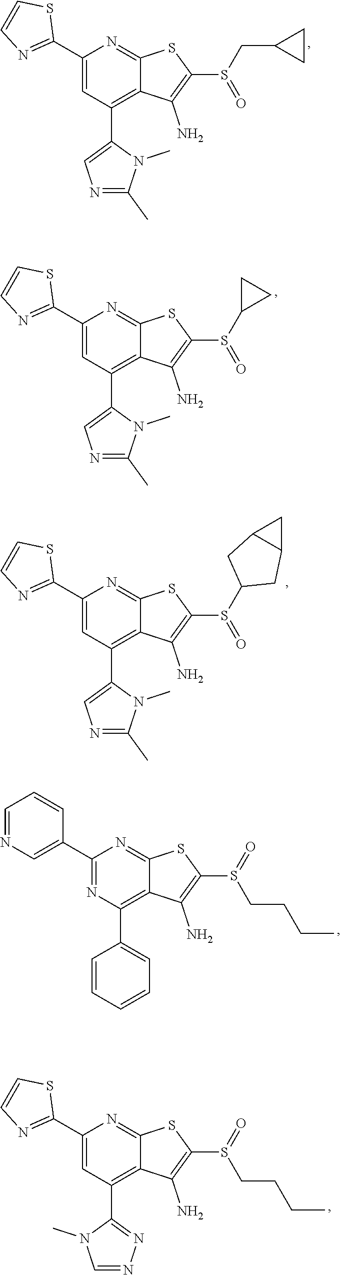 [SMILES: Cc1ncc(-c2cc(-c3nccs3)nc3sc(S(=O)CC4CC4)c(N)c23)n1C ; Cc1ncc(-c2cc(-c3nccs3)nc3sc(S(=O)C4CC4)c(N)c23)n1C ; Cc1ncc(-c2cc(-c3nccs3)nc3sc(S(=O)C4CC5CC5C4)c(N)c23)n1C ; CCCCS(=O)c1sc2nc(-c3cccnc3)nc(-c3ccccc3)c2c1N ; CCCCS(=O)c1sc2nc(-c3nccs3)cc(-c3nncn3C)c2c1N]

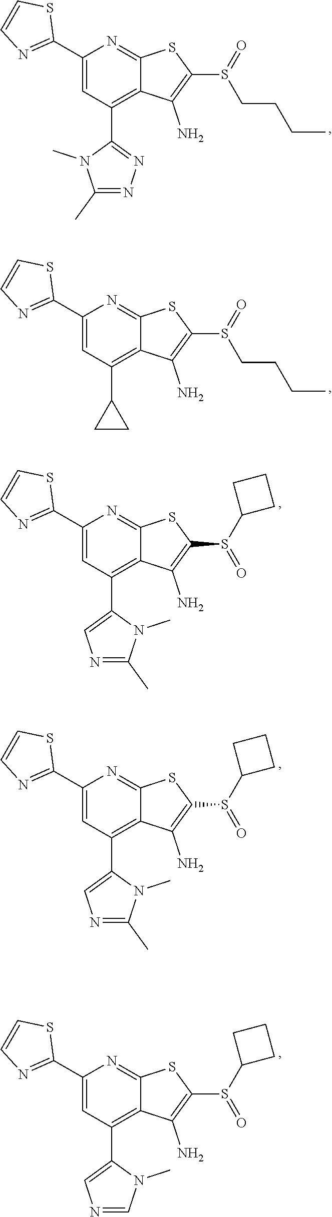 [SMILES: CCCCS(=O)c1sc2nc(-c3nccs3)cc(-c3nnc(C)n3C)c2c1N ; CCCCS(=O)c1sc2nc(-c3nccs3)cc(C3CC3)c2c1N ; Cc1ncc(-c2cc(-c3nccs3)nc3sc([S@](=O)C4CCC4)c(N)c23)n1C ; Cc1ncc(-c2cc(-c3nccs3)nc3sc([S@@](=O)C4CCC4)c(N)c23)n1C ; Cn1cncc1-c1cc(-c2nccs2)nc2sc(S(=O)C3CCC3)c(N)c12]

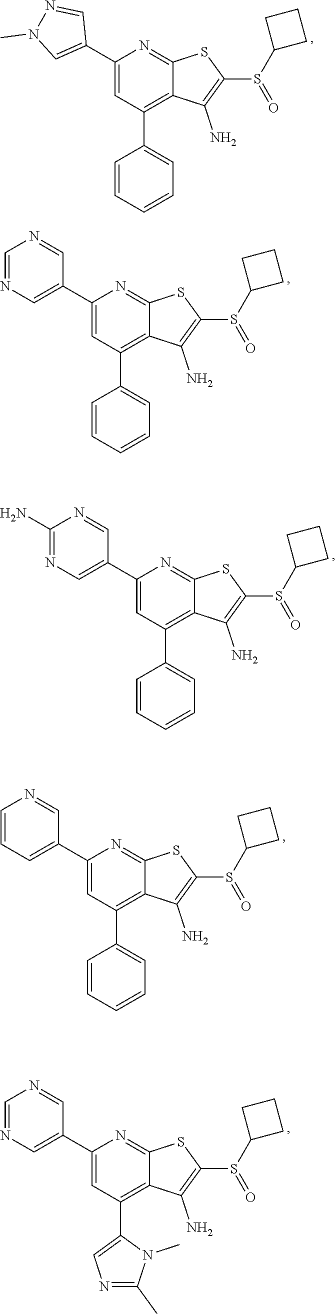 [SMILES: Cn1cc(-c2cc(-c3ccccc3)c3c(N)c(S(=O)C4CCC4)sc3n2)cn1 ; Nc1c(S(=O)C2CCC2)sc2nc(-c3cncnc3)cc(-c3ccccc3)c12 ; Nc1ncc(-c2cc(-c3ccccc3)c3c(N)c(S(=O)C4CCC4)sc3n2)cn1 ; Nc1c(S(=O)C2CCC2)sc2nc(-c3cccnc3)cc(-c3ccccc3)c12 ; Cc1ncc(-c2cc(-c3cncnc3)nc3sc(S(=O)C4CCC4)c(N)c23)n1C]

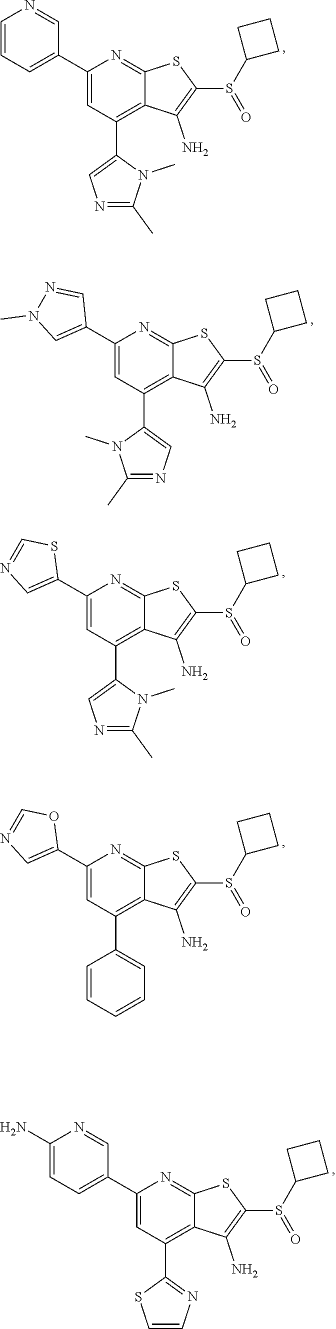 [SMILES: Cc1ncc(-c2cc(-c3cccnc3)nc3sc(S(=O)C4CCC4)c(N)c23)n1C ; Cc1ncc(-c2cc(-c3cnn(C)c3)nc3sc(S(=O)C4CCC4)c(N)c23)n1C ; Cc1ncc(-c2cc(-c3cncs3)nc3sc(S(=O)C4CCC4)c(N)c23)n1C ; Nc1c(S(=O)C2CCC2)sc2nc(-c3cnco3)cc(-c3ccccc3)c12 ; Nc1ccc(-c2cc(-c3nccs3)c3c(N)c(S(=O)C4CCC4)sc3n2)cn1]

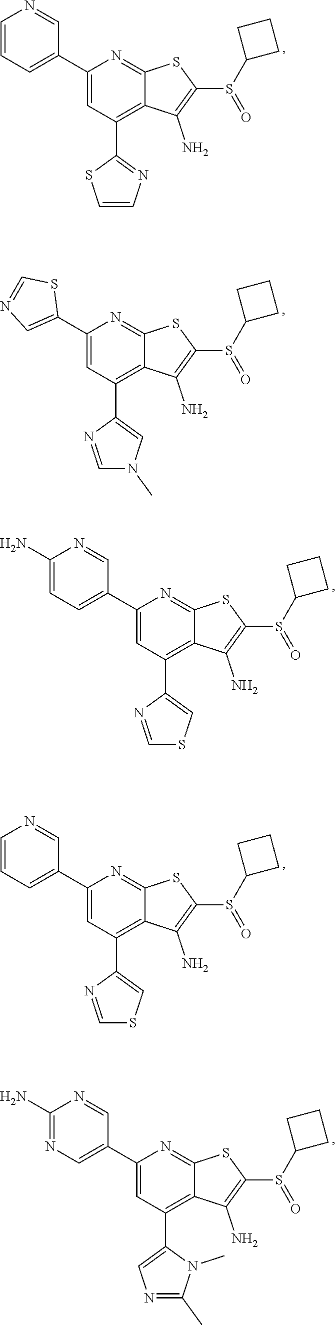 [SMILES: Nc1c(S(=O)C2CCC2)sc2nc(-c3cccnc3)cc(-c3nccs3)c12 ; Cn1cnc(-c2cc(-c3cncs3)nc3sc(S(=O)C4CCC4)c(N)c23)c1 ; Nc1ccc(-c2cc(-c3cscn3)c3c(N)c(S(=O)C4CCC4)sc3n2)cn1 ; Nc1c(S(=O)C2CCC2)sc2nc(-c3cccnc3)cc(-c3cscn3)c12 ; Cc1ncc(-c2cc(-c3cnc(N)nc3)nc3sc(S(=O)C4CCC4)c(N)c23)n1C]

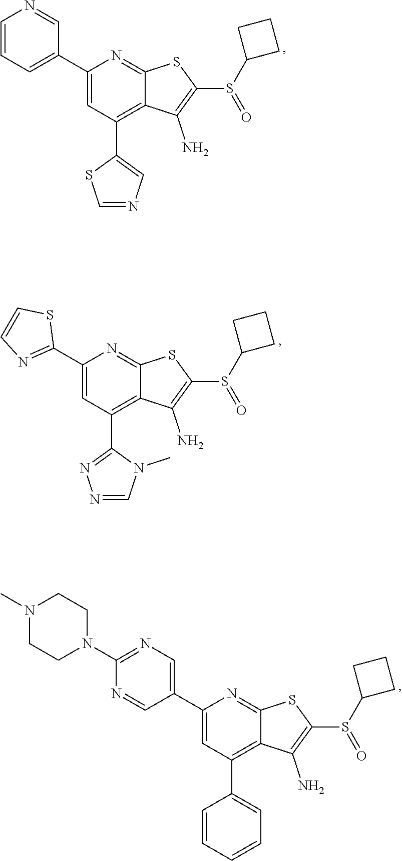 [SMILES: Nc1c(S(=O)C2CCC2)sc2nc(-c3cccnc3)cc(-c3cncs3)c12 ; Cn1cnnc1-c1cc(-c2nccs2)nc2sc(S(=O)C3CCC3)c(N)c12 ; CN1CCN(c2ncc(-c3cc(-c4ccccc4)c4c(N)c(S(=O)C5CCC5)sc4n3)cn2)CC1]

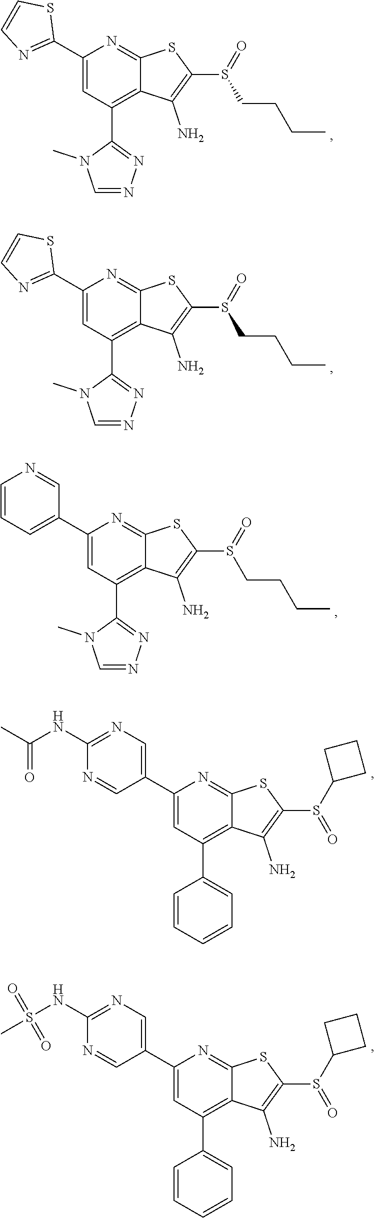

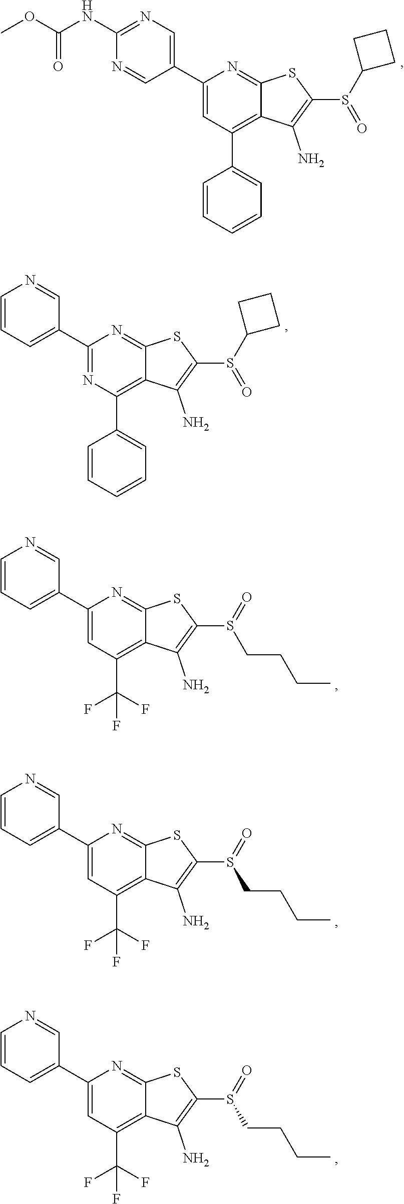 [SMILES: COC(=O)Nc1ncc(-c2cc(-c3ccccc3)c3c(N)c(S(=O)C4CCC4)sc3n2)cn1 ; Nc1c(S(=O)C2CCC2)sc2nc(-c3cccnc3)nc(-c3ccccc3)c12 ; CCCCS(=O)c1sc2nc(-c3cccnc3)cc(C(F)(F)F)c2c1N ; CCCC[S@](=O)c1sc2nc(-c3cccnc3)cc(C(F)(F)F)c2c1N ; CCCC[S@@](=O)c1sc2nc(-c3cccnc3)cc(C(F)(F)F)c2c1N]

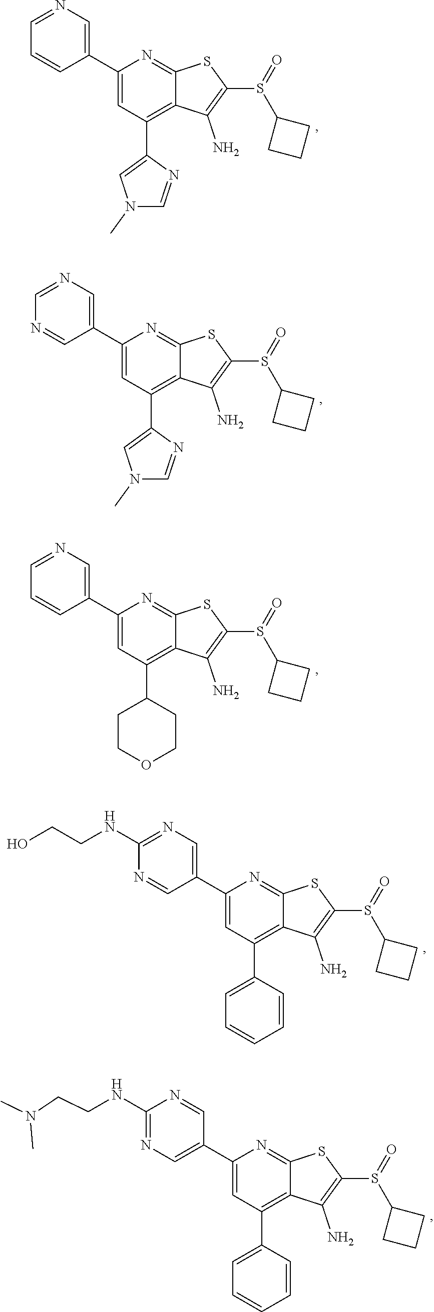 [SMILES: Cn1cnc(-c2cc(-c3cccnc3)nc3sc(S(=O)C4CCC4)c(N)c23)c1 ; Cn1cnc(-c2cc(-c3cncnc3)nc3sc(S(=O)C4CCC4)c(N)c23)c1 ; Nc1c(S(=O)C2CCC2)sc2nc(-c3cccnc3)cc(C3CCOCC3)c12 ; Nc1c(S(=O)C2CCC2)sc2nc(-c3cnc(NCCO)nc3)cc(-c3ccccc3)c12 ; CN(C)CCNc1ncc(-c2cc(-c3ccccc3)c3c(N)c(S(=O)C4CCC4)sc3n2)cn1]

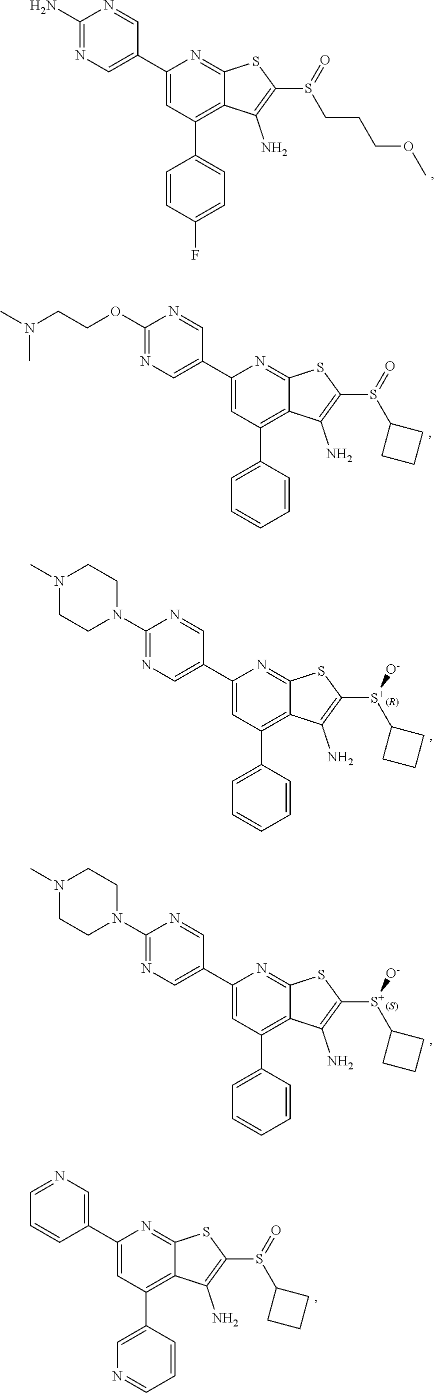

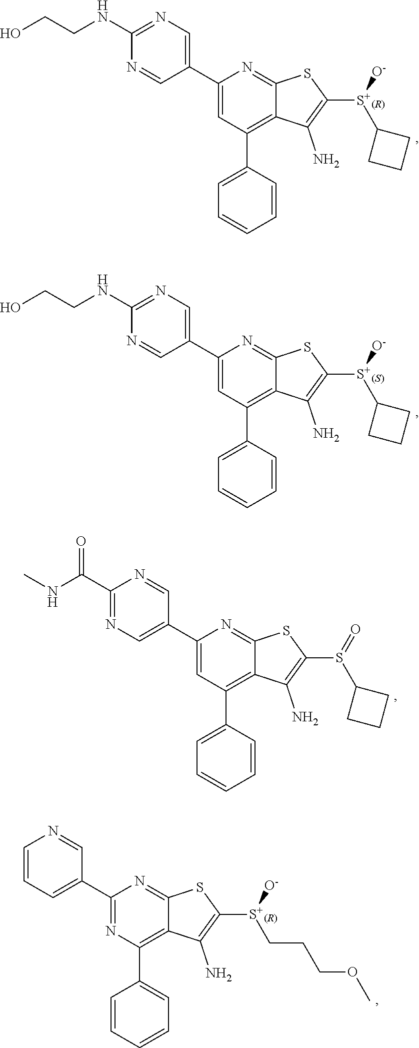

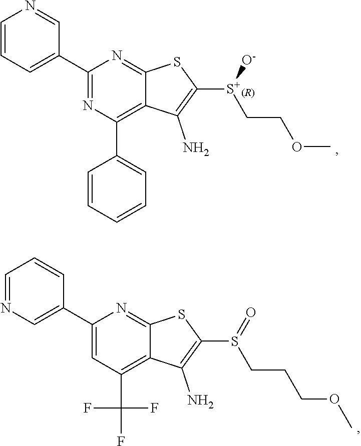

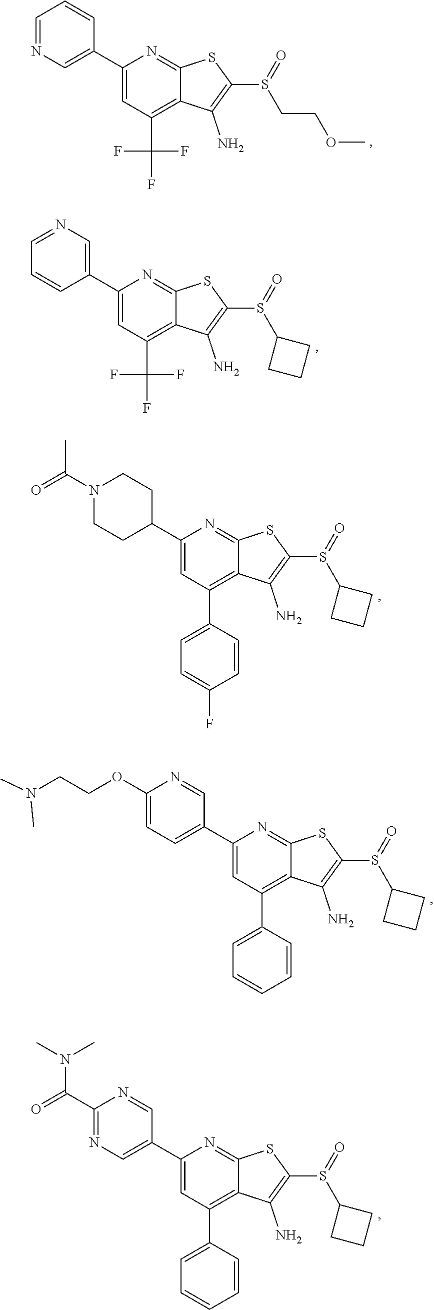

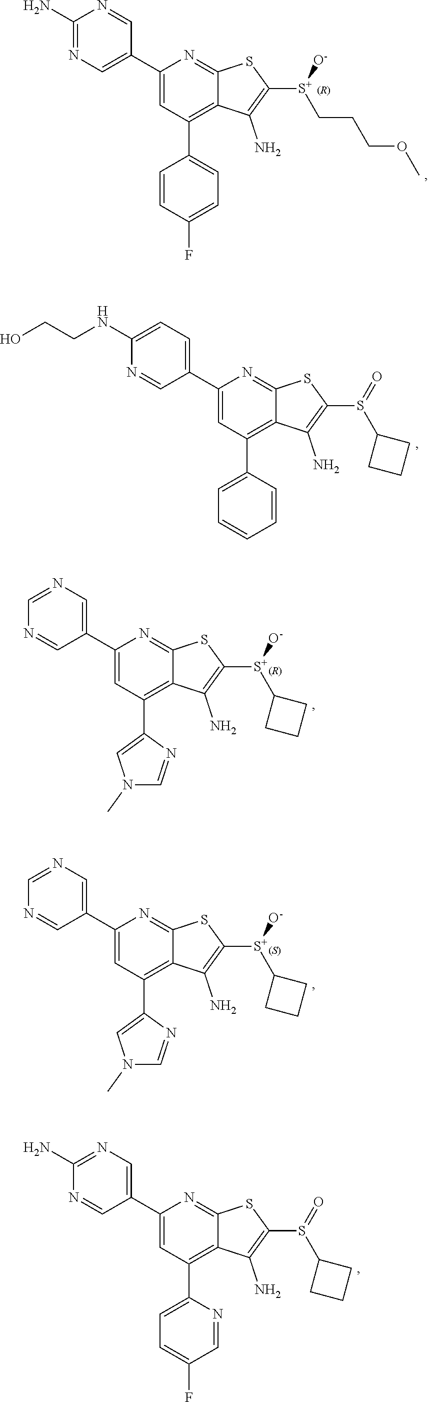

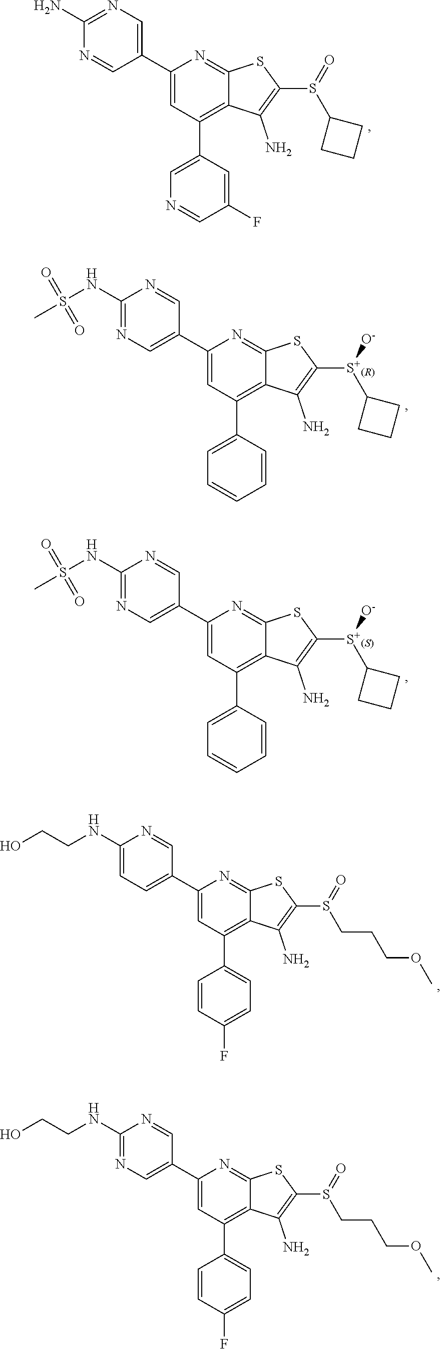

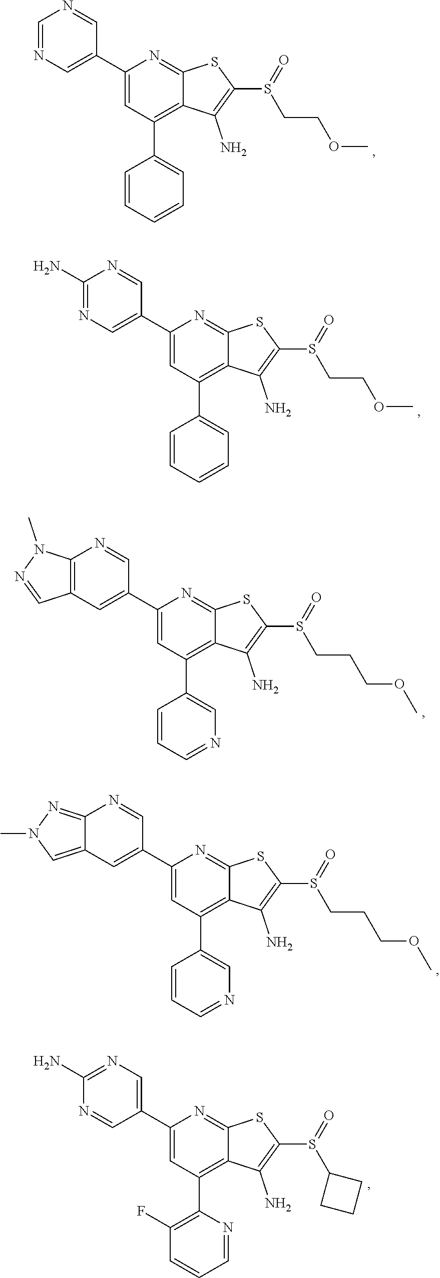

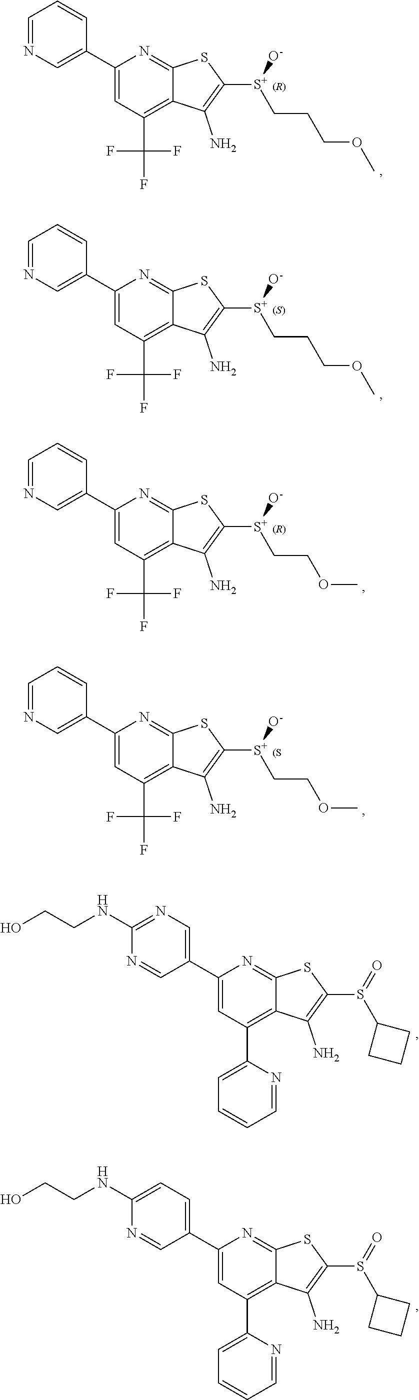

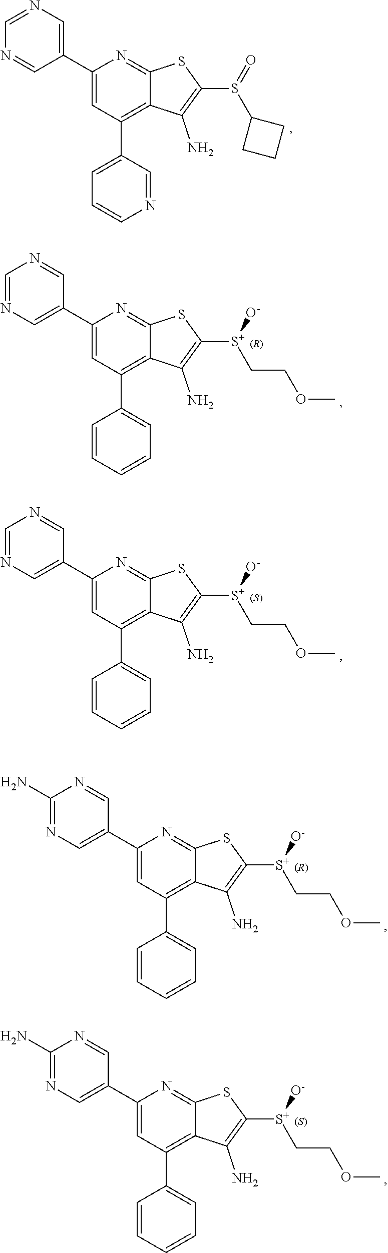

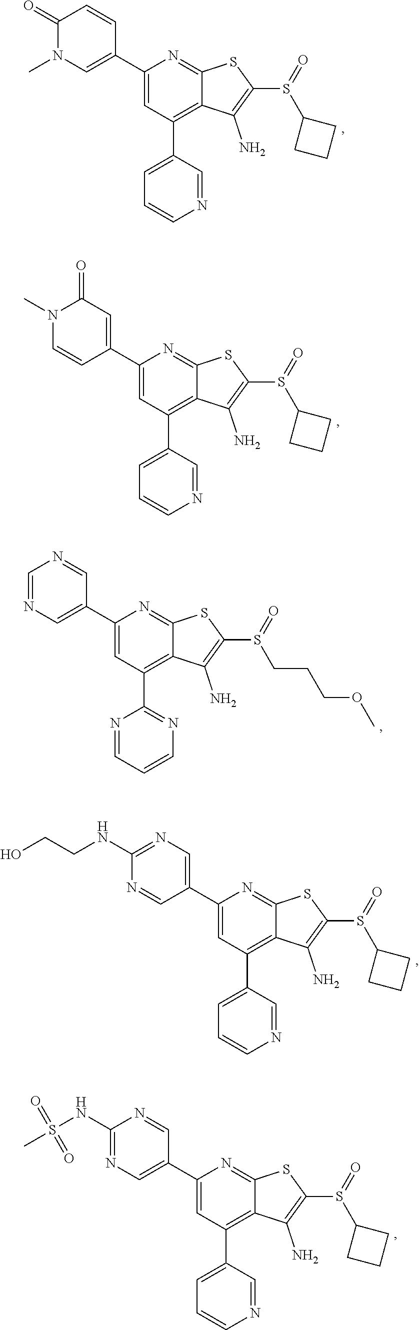

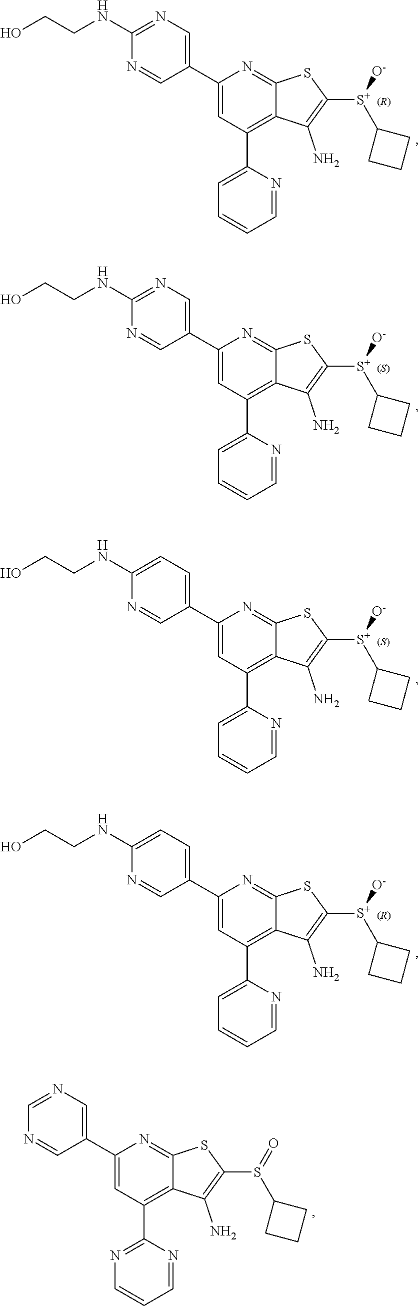

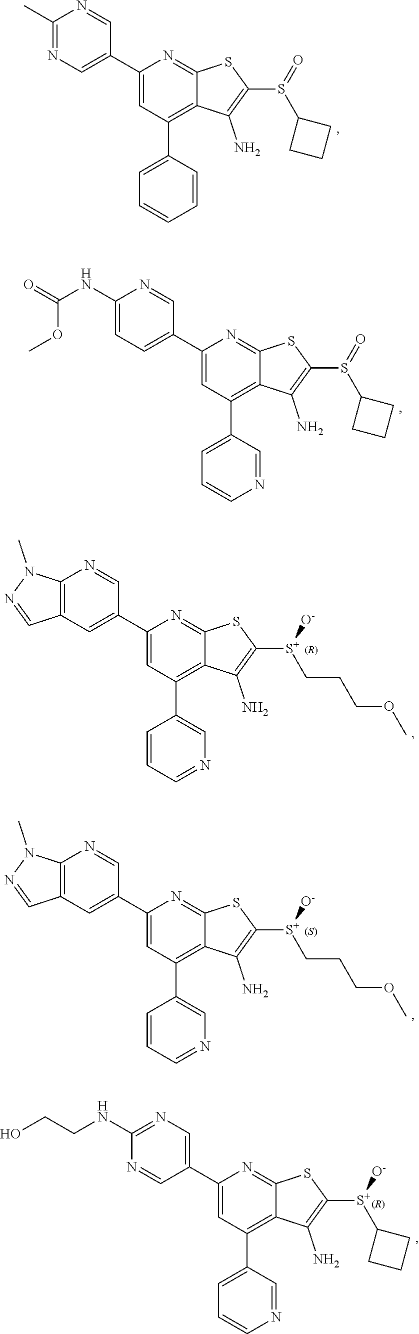

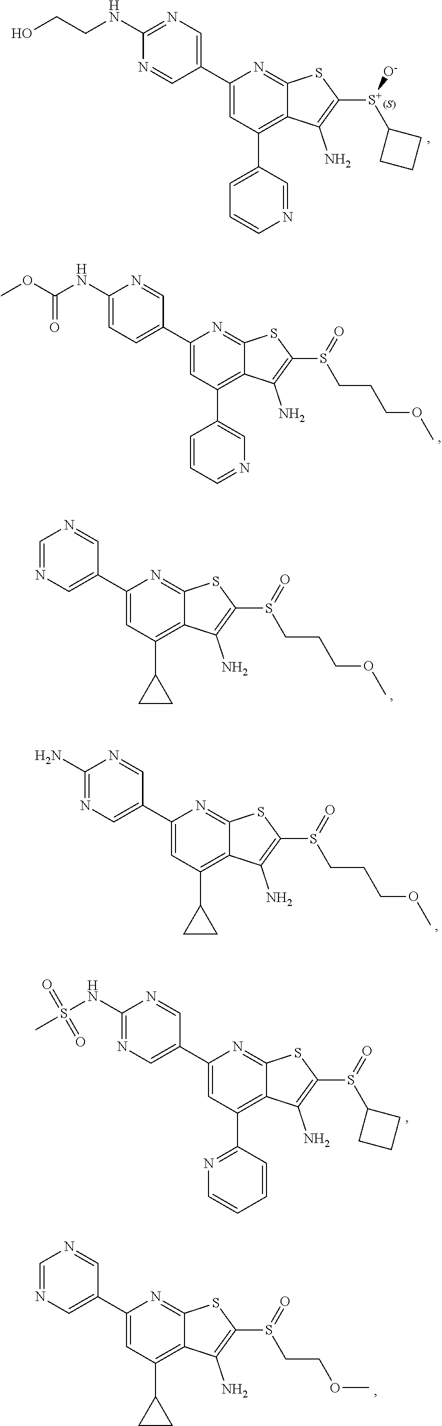

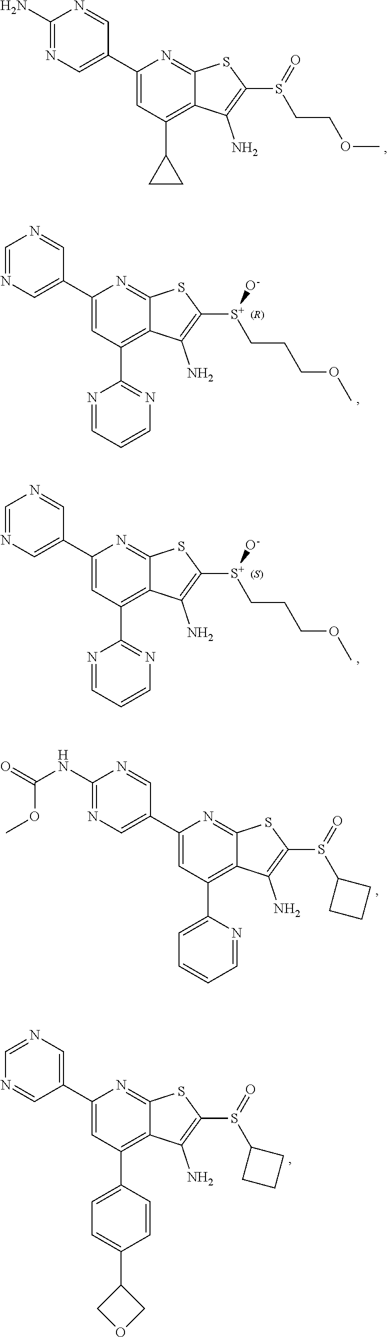

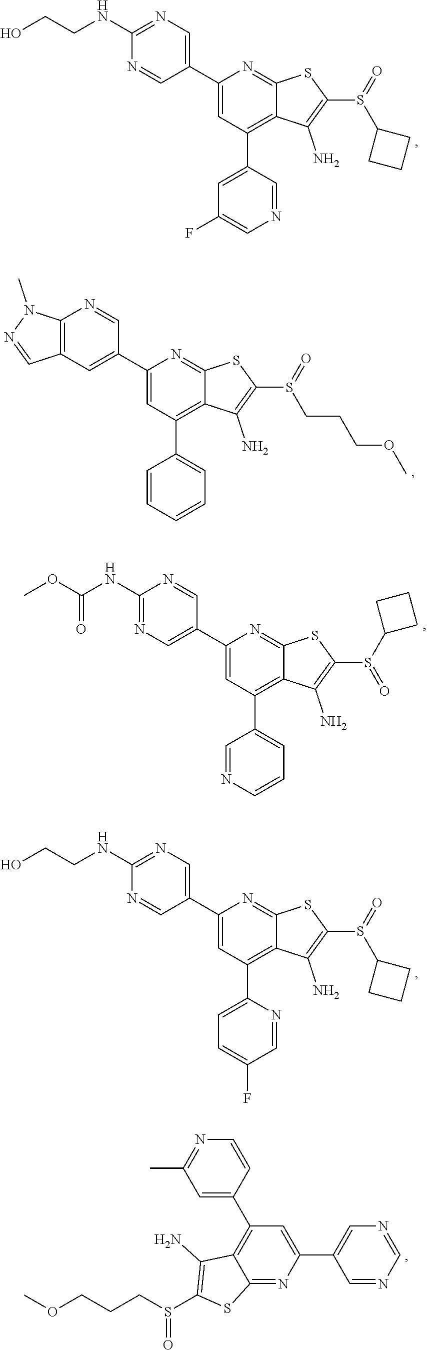

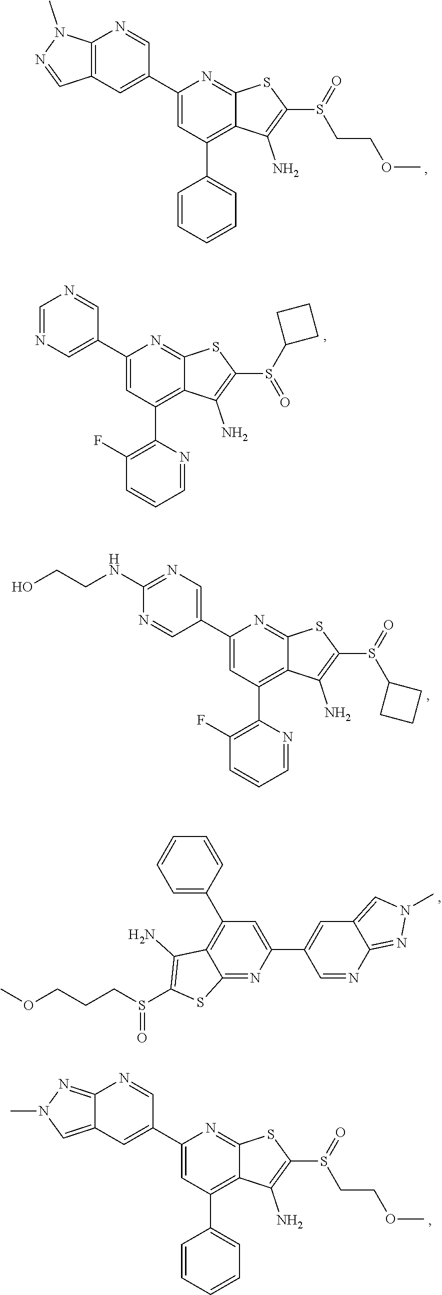

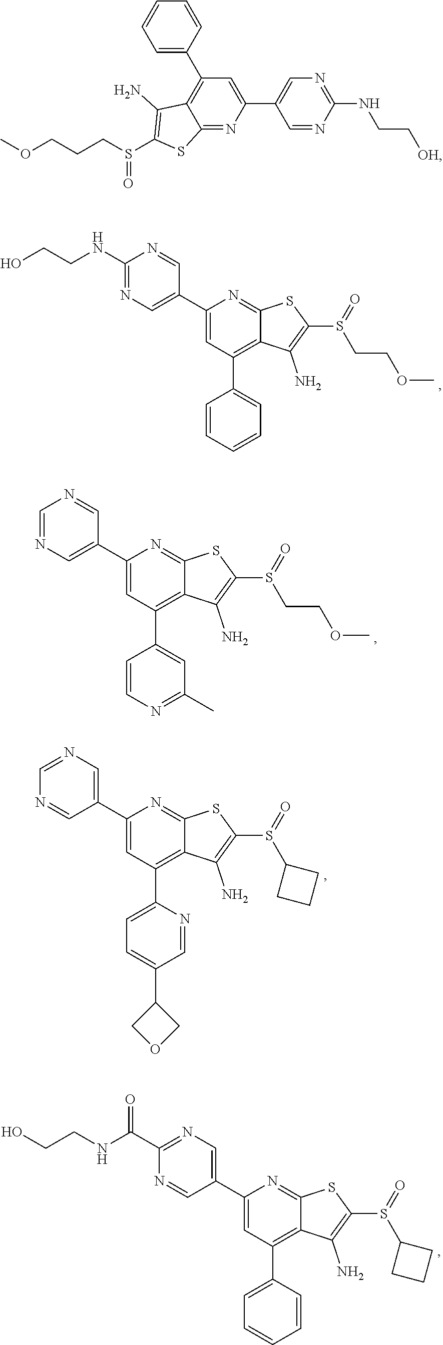 [SMILES: COCCCS(=O)c1sc2nc(-c3cnc(NCCO)nc3)cc(-c3ccccc3)c2c1N ; COCCS(=O)c1sc2nc(-c3cnc(NCCO)nc3)cc(-c3ccccc3)c2c1N ; COCCS(=O)c1sc2nc(-c3cncnc3)cc(-c3ccnc(C)c3)c2c1N ; Nc1c(S(=O)C2CCC2)sc2nc(-c3cncnc3)cc(-c3ccc(C4COC4)cn3)c12 ; Nc1c(S(=O)C2CCC2)sc2nc(-c3cnc(C(=O)NCCO)nc3)cc(-c3ccccc3)c12]

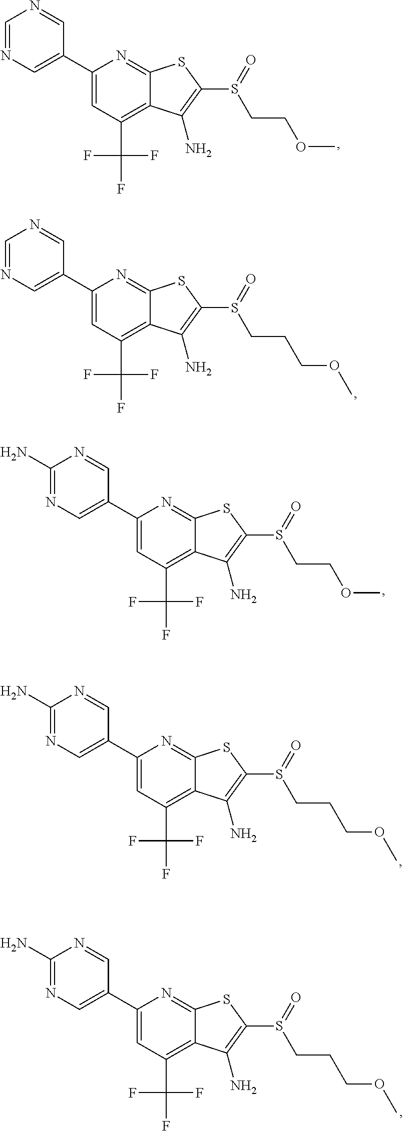 [SMILES: COCCS(=O)c1sc2nc(-c3cncnc3)cc(C(F)(F)F)c2c1N ; COCCCS(=O)c1sc2nc(-c3cncnc3)cc(C(F)(F)F)c2c1N ; COCCS(=O)c1sc2nc(-c3cnc(N)nc3)cc(C(F)(F)F)c2c1N ; COCCCS(=O)c1sc2nc(-c3cnc(N)nc3)cc(C(F)(F)F)c2c1N ; COCCCS(=O)c1sc2nc(-c3cnc(N)nc3)cc(C(F)(F)F)c2c1N]

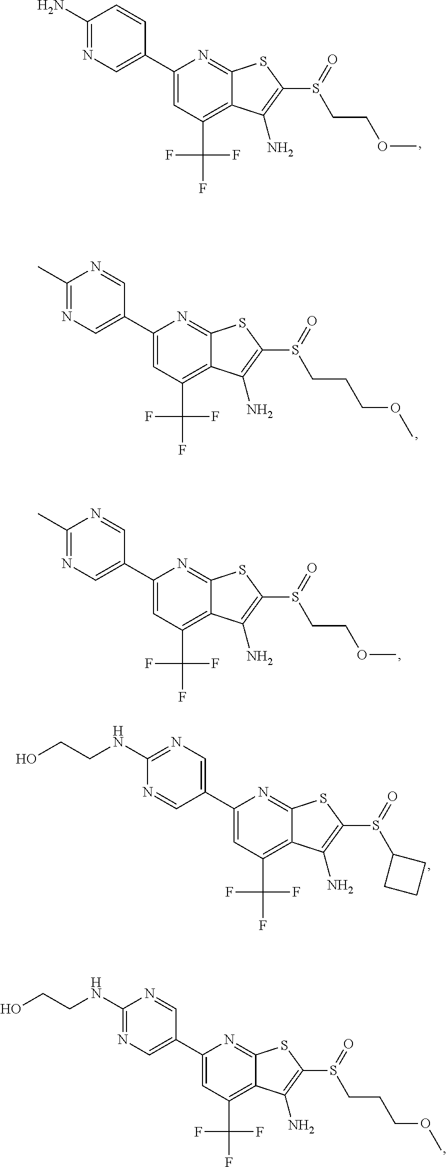

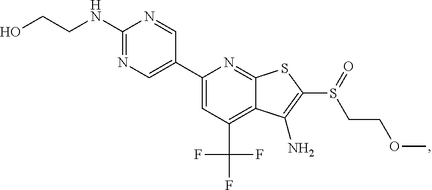 [SMILES: COCCS(=O)c1sc2nc(-c3cnc(NCCO)nc3)cc(C(F)(F)F)c2c1N]

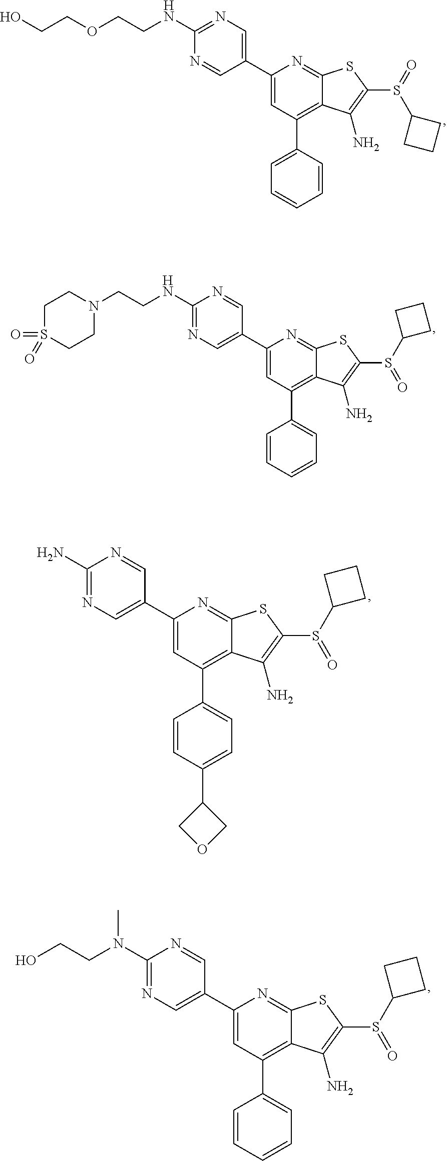 [SMILES: Nc1c(S(=O)C2CCC2)sc2nc(-c3cnc(NCCOCCO)nc3)cc(-c3ccccc3)c12 ; Nc1c(S(=O)C2CCC2)sc2nc(-c3cnc(NCCN4CCS(=O)(=O)CC4)nc3)cc(-c3ccccc3)c12 ; Nc1ncc(-c2cc(-c3ccc(C4COC4)cc3)c3c(N)c(S(=O)C4CCC4)sc3n2)cn1 ; CN(CCO)c1ncc(-c2cc(-c3ccccc3)c3c(N)c(S(=O)C4CCC4)sc3n2)cn1]

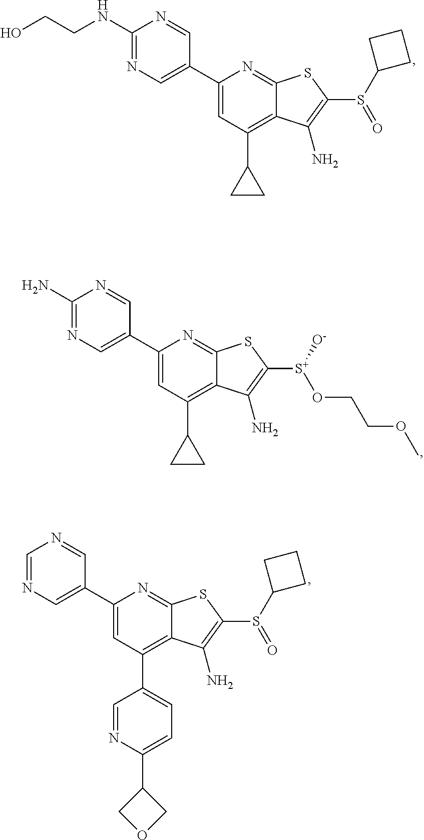 [SMILES: Nc1c(S(=O)C2CCC2)sc2nc(-c3cnc(NCCO)nc3)cc(C3CC3)c12 ; COCCO[S@@+]([O-])c1sc2nc(-c3cnc(N)nc3)cc(C3CC3)c2c1N ; Nc1c(S(=O)C2CCC2)sc2nc(-c3cncnc3)cc(-c3ccc(C4COC4)nc3)c12]

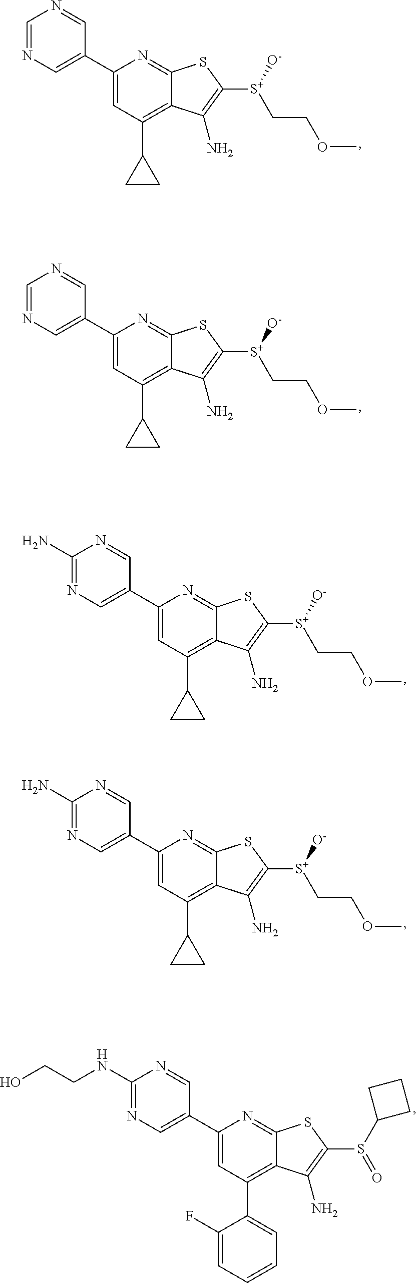 [SMILES: COCC[S@@+]([O-])c1sc2nc(-c3cncnc3)cc(C3CC3)c2c1N ; COCC[S@+]([O-])c1sc2nc(-c3cncnc3)cc(C3CC3)c2c1N ; COCC[S@@+]([O-])c1sc2nc(-c3cnc(N)nc3)cc(C3CC3)c2c1N ; COCC[S@+]([O-])c1sc2nc(-c3cnc(N)nc3)cc(C3CC3)c2c1N ; Nc1c(S(=O)C2CCC2)sc2nc(-c3cnc(NCCO)nc3)cc(-c3ccccc3F)c12]

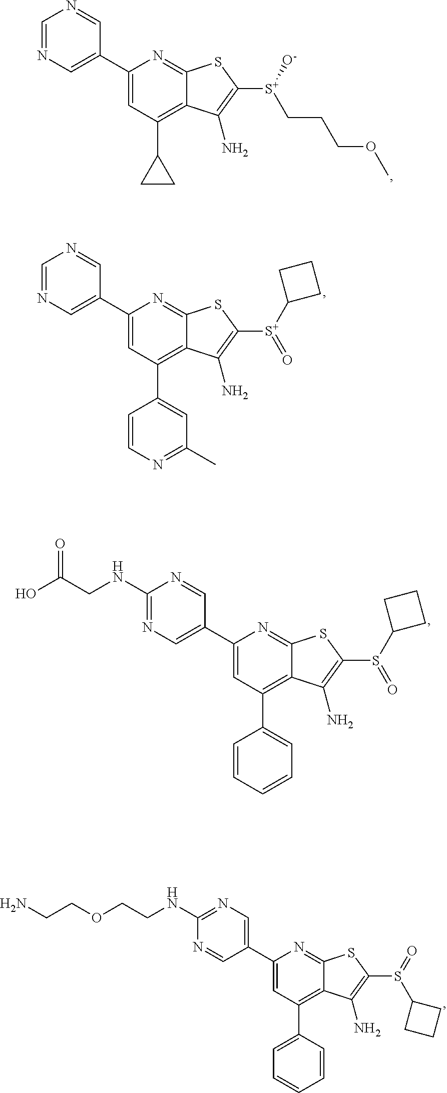

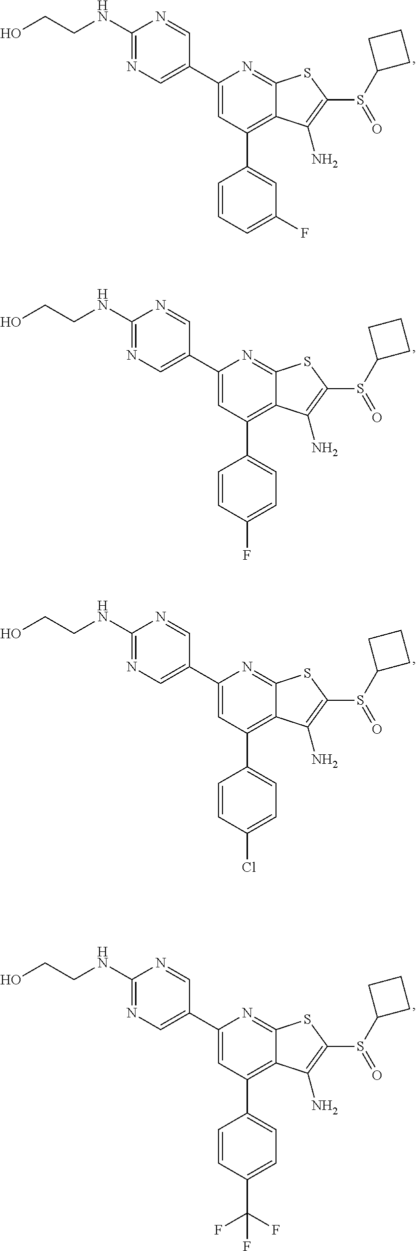 [SMILES: Nc1c(S(=O)C2CCC2)sc2nc(-c3cnc(NCCO)nc3)cc(-c3cccc(F)c3)c12 ; Nc1c(S(=O)C2CCC2)sc2nc(-c3cnc(NCCO)nc3)cc(-c3ccc(F)cc3)c12 ; Nc1c(S(=O)C2CCC2)sc2nc(-c3cnc(NCCO)nc3)cc(-c3ccc(Cl)cc3)c12 ; Nc1c(S(=O)C2CCC2)sc2nc(-c3cnc(NCCO)nc3)cc(-c3ccc(C(F)(F)F)cc3)c12]

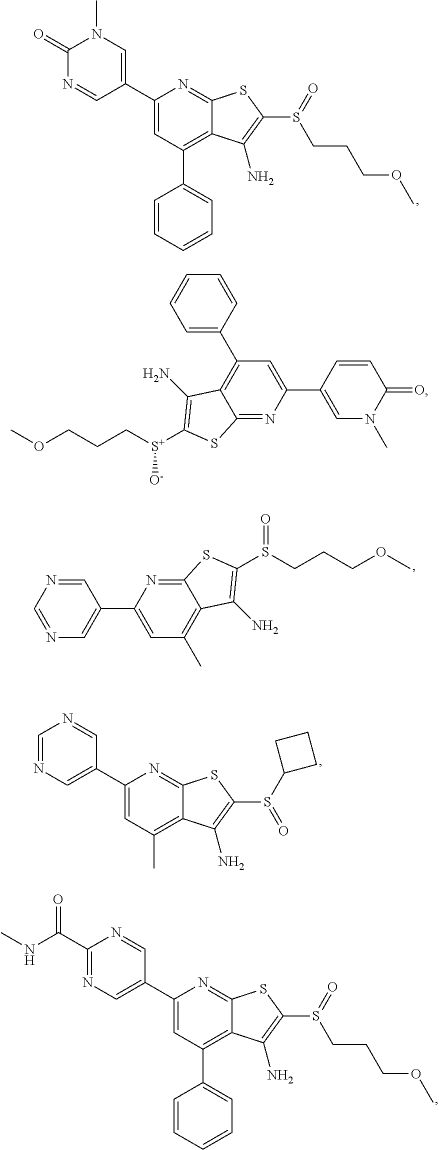

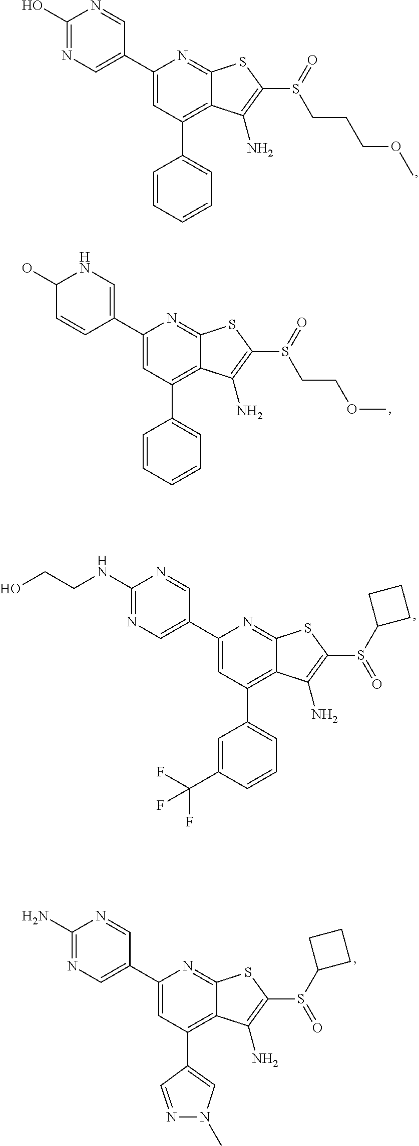 [SMILES: COCCCS(=O)c1sc2nc(-c3cnc(O)nc3)cc(-c3ccccc3)c2c1N ; COCCS(=O)c1sc2nc(C3=CNC(O)C=C3)cc(-c3ccccc3)c2c1N ; Nc1c(S(=O)C2CCC2)sc2nc(-c3cnc(NCCO)nc3)cc(-c3cccc(C(F)(F)F)c3)c12 ; Cn1cc(-c2cc(-c3cnc(N)nc3)nc3sc(S(=O)C4CCC4)c(N)c23)cn1]

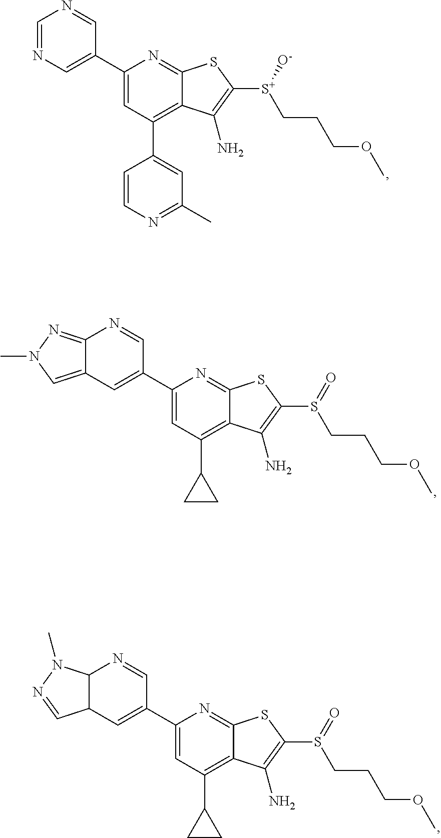

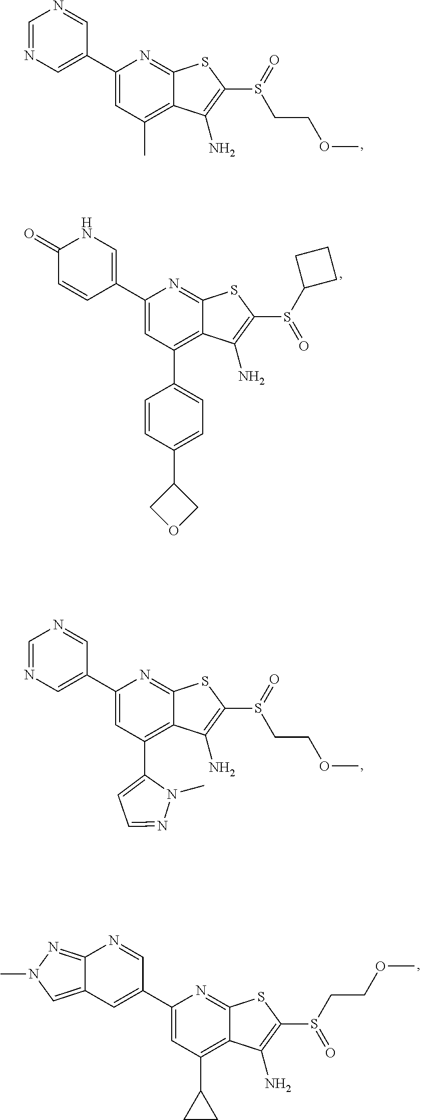

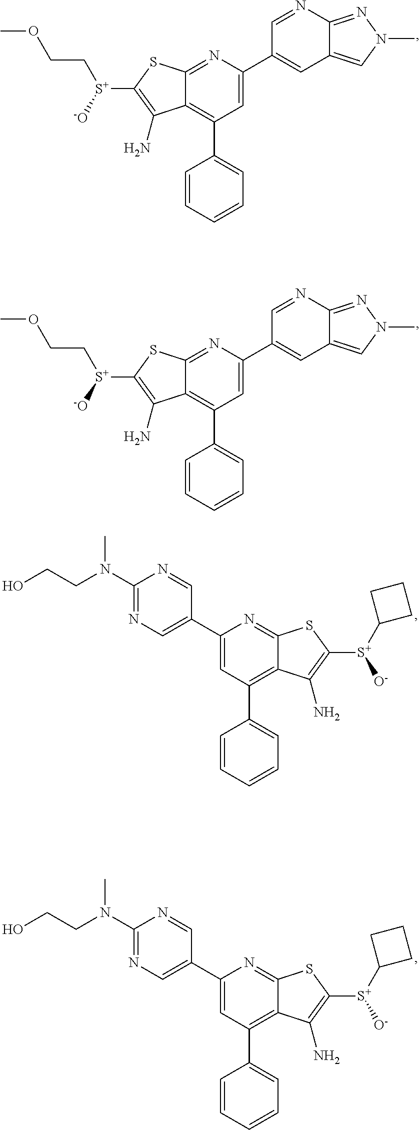

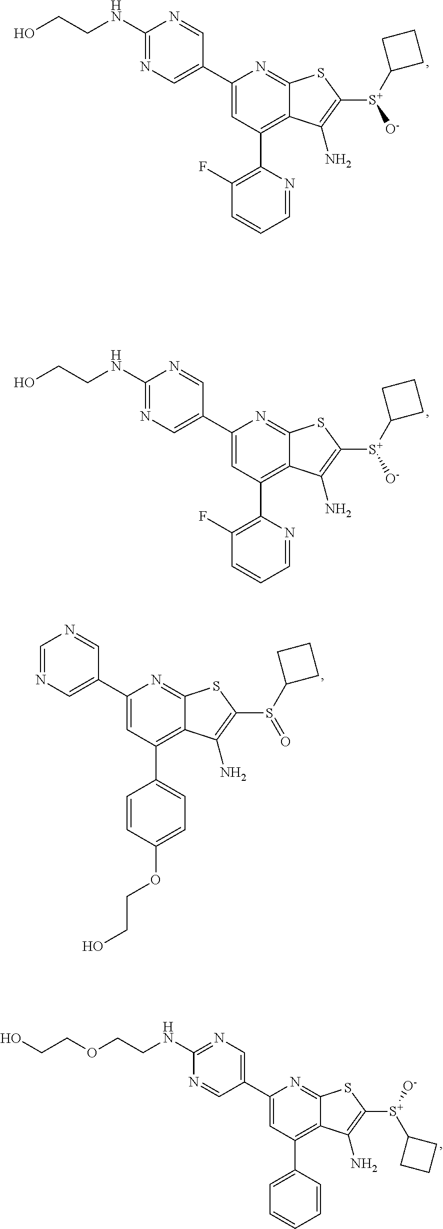 [SMILES: Nc1c([S@+]([O-])C2CCC2)sc2nc(-c3cnc(NCCO)nc3)cc(-c3ncccc3F)c12 ; Nc1c([S@@+]([O-])C2CCC2)sc2nc(-c3cnc(NCCO)nc3)cc(-c3ncccc3F)c12 ; Nc1c(S(=O)C2CCC2)sc2nc(-c3cncnc3)cc(-c3ccc(OCCO)cc3)c12 ; Nc1c([S@+]([O-])C2CCC2)sc2nc(-c3cnc(NCCOCCO)nc3)cc(-c3ccccc3)c12]

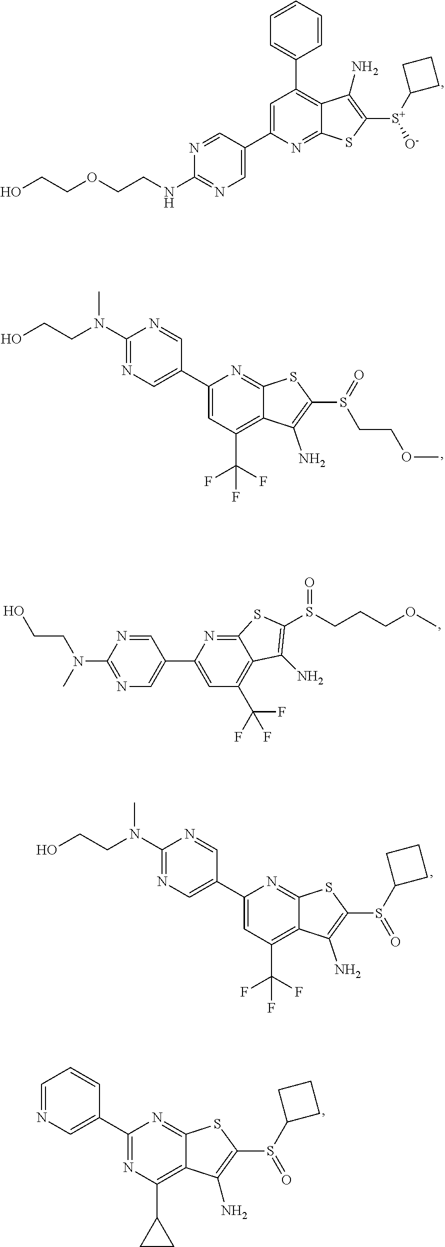 [SMILES: Nc1c([S@@+]([O-])C2CCC2)sc2nc(-c3cnc(NCCOCCO)nc3)cc(-c3ccccc3)c12 ; COCCS(=O)c1sc2nc(-c3cnc(N(C)CCO)nc3)cc(C(F)(F)F)c2c1N ; COCCCS(=O)c1sc2nc(-c3cnc(N(C)CCO)nc3)cc(C(F)(F)F)c2c1N ; CN(CCO)c1ncc(-c2cc(C(F)(F)F)c3c(N)c(S(=O)C4CCC4)sc3n2)cn1 ; Nc1c(S(=O)C2CCC2)sc2nc(-c3cccnc3)nc(C3CC3)c12]

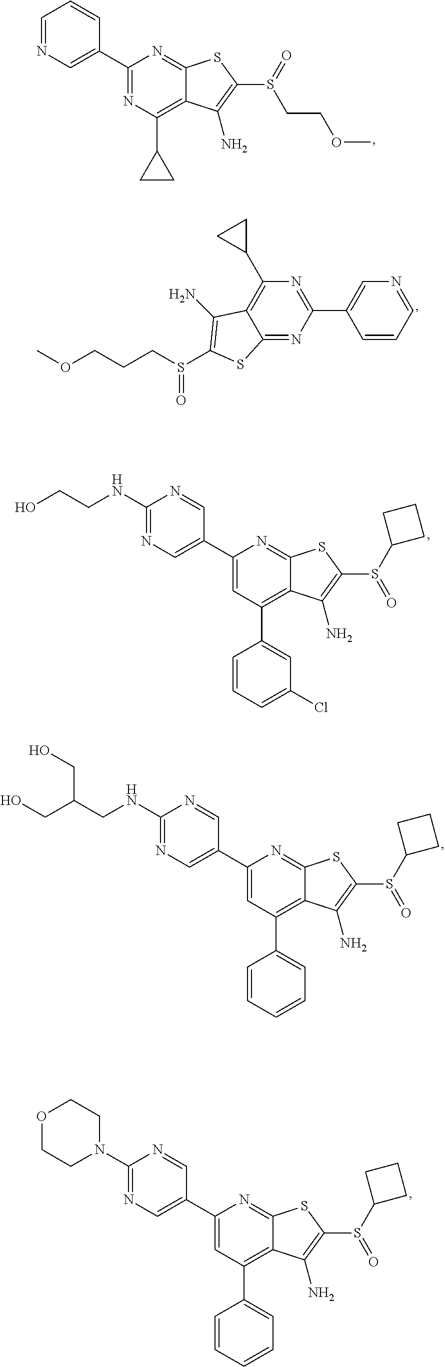

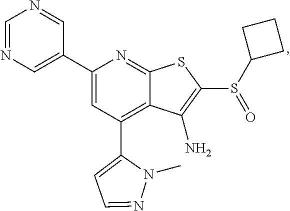 [SMILES: Cn1nccc1-c1cc(-c2cncnc2)nc2sc(S(=O)C3CCC3)c(N)c12]

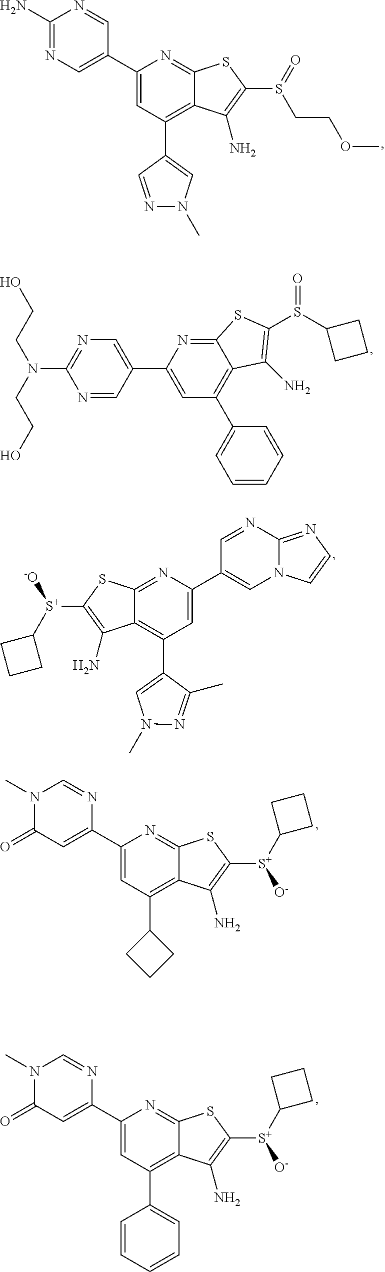

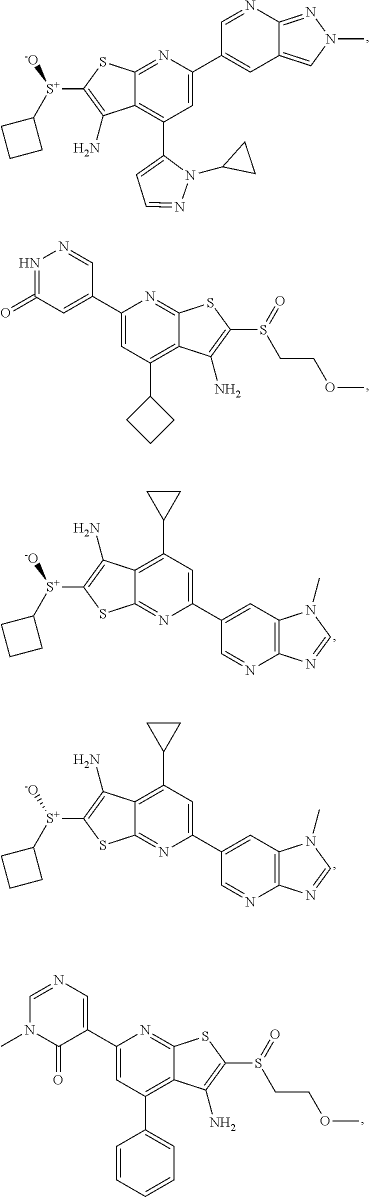

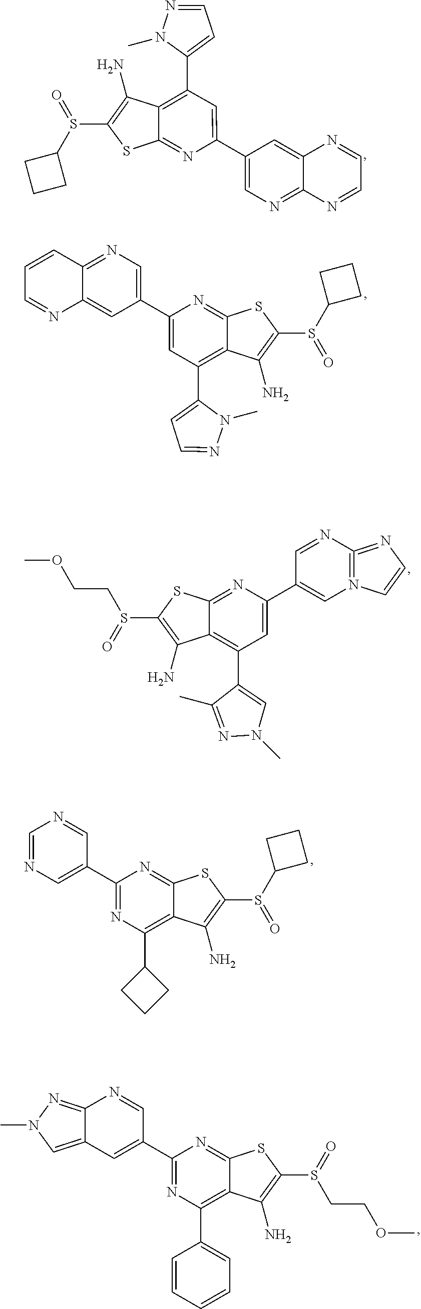

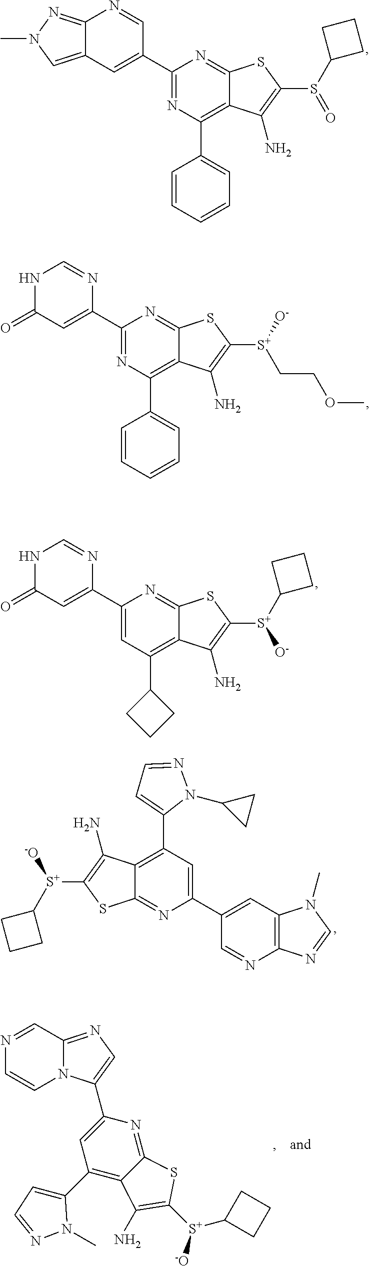

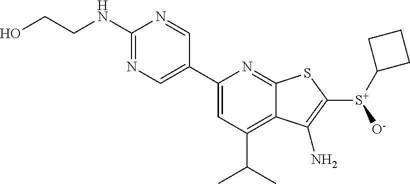 [SMILES: CC(C)c1cc(-c2cnc(NCCO)nc2)nc2sc([S@+]([O-])C3CCC3)c(N)c12]

or pharmaceutically acceptable salts thereof, a tautomers thereof, and a solvates thereof.

In other embodiments, the compound can include at least one of the formulas (IB) or (IIB), or a pharmaceutically acceptable salt thereof:

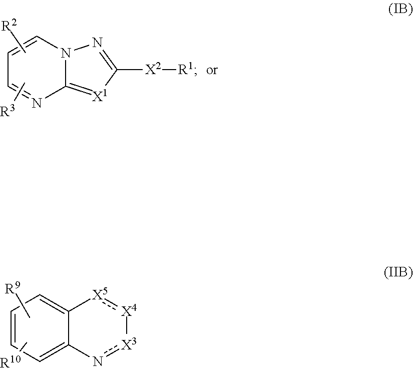

wherein X¹ is N or CR⁴;
X² is S, S═O, S(═O)₂, or C═O;
X³ is CR⁸, the compound forming a polycyclic heteroaryl with 10 ring atoms, or absent, the compound forming a polycyclic heteroaryl with 9 ring atoms;
X⁴ is N, NH, or CR⁷;
X⁵ is N, C═O, or CR¹⁶, and X⁵ is N if X⁴ is CR⁷, or X³ is absent, X⁴ is NH if X⁵ is C═O, and X⁵ is CR¹⁶ if X⁴ is N and X³ is CR⁸;
R¹, R², R³, R⁴, R⁹, R¹⁰, and R¹⁶ are the same or different and are independently selected from the group consisting of hydrogen, substituted or unsubstituted C₁-C₂₄ alkyl, C₂-C₂₄ alkenyl, C₂-C₂₄ alkynyl, C₃-C₂₀ aryl, heterocycloalkenyl containing from 5-7 ring atoms, (wherein from 1-3 of the ring atoms is independently selected from N, NH, N(C₁-C₆ alkyl), NC(O) (C₁-C₆ alkyl), O, and S), heteroaryl or heterocyclyl containing from 5-14 ring atoms, (wherein from 1-6 of the ring atoms is independently selected from N, NH, N(C₁-C₃ alkyl), O, and S), C₆-C₂₄ alkaryl, C₆-C₂₄ aralkyl, halo, silyl, hydroxyl, sulfhydryl, C₁-C₂₄ alkoxy, C₂-C₂₄ alkenyloxy, C₂-C₂₄ alkynyloxy, C₅-C₂₀ aryloxy, acyl (including C₂-C₂₄ alkylcarbonyl (—CO-alkyl) and C₆-C₂₀ arylcarbonyl (—CO-aryl)), acyloxy (—O-acyl), C₂-C₂₄ alkoxycarbonyl (—(CO)—O-alkyl), C₆-C₂₀ aryloxycarbonyl (—(CO)—O-aryl), C₂-C₂₄ alkylcarbonato (—O—(CO)—O-alkyl), C₆-C₂₀ arylcarbonato (—O—(CO)—O-aryl), carboxy (—COOH), carboxylato (—COO⁻), carbamoyl (—(CO)—NH₂), C₁-C₂₄ alkyl-carbamoyl (—(CO)—NH(C₁-C₂₄ alkyl)), arylcarbamoyl (—(CO)—NH-aryl), thiocarbamoyl (—(CS)—NH₂), carbamido (—NH—(CO)—NH₂), cyano (—CN), isocyano (—N⁺C⁻), cyanato (—O—CN), isocyanato (—O—N⁺═C⁻), isothiocyanato (—S—CN), azido (—N═N⁺═N⁻), formyl (—(CO)—H), thioformyl (—(CS)—H), amino (—NH₂), C₁-C₂₄ alkyl amino, C₅-C₂₀ aryl amino, C₂-C₂₄ alkylamido (—NH—(CO)-alkyl), C₆-C₂₀ arylamido (—NH—(CO)-aryl), sulfanamido (—SO₂N(R)₂ where R is independently H, alkyl, aryl or heteroaryl), imino (—CR═NH where R is hydrogen, C₁-C₂₄ alkyl, C₅-C₂₀ aryl, C₆-C₂₄ alkaryl, C₆-C₂₄ aralkyl, etc.), alkylimino (—CR═N(alkyl), where R=hydrogen, alkyl, aryl, alkaryl, aralkyl, etc.), arylimino (—CR═N(aryl), where R=hydrogen, alkyl, aryl, alkaryl, etc.), nitro (—NO₂), nitroso (—NO), sulfo (—SO₂—OH), sulfonato (—SO₂—O⁻), C₁-C₂₄ alkylsulfanyl (—S-alkyl; also termed “alkylthio”), arylsulfanyl (—S-aryl; also termed “arylthio”), C₁-C₂₄ alkylsulfinyl (—(SO)-alkyl), C₅-C₂₀ arylsulfinyl (—(SO)-aryl), C₁-C₂₄ alkylsulfonyl (—SO₂-alkyl), C₅-C₂₀ arylsulfonyl (—SO₂-aryl), sulfonamide (—SO₂—NH₂, —SO₂NY₂ (wherein Y is independently H, aryl or alkyl), phosphono (—P(O)(OH)₂), phosphonato (—P(O)(O⁻)₂), phosphinato (—P(O)(O⁻)), phospho (—PO₂), phosphino (—PH₂), polyalkyl ethers (—[(CH₂)ₙO]ₘ), phosphates, phosphate esters [—OP(O)(OR)₂ where R=H, methyl or other alkyl], groups incorporating amino acids or other moieties expected to bear positive or negative charge at physiological pH, and combinations thereof;
R⁷ and R⁸ are same or different and are each independently selected from the group consisting of H, a substituted or unsubstituted aryl, a substituted or unsubstituted heteroaryl, a substituted or unsubstituted cycloalkyl, and a substituted or unsubstituted heterocyclyl, and at least one of R⁷ or R⁸ is not H; and
wherein the compound is not

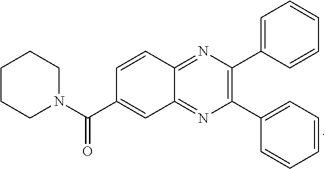 [SMILES: O=C(c1ccc2nc(-c3ccccc3)c(-c3ccccc3)nc2c1)N1CCCCC1]

In some embodiments, at least one of R² or R³ is not H, and at least one of R⁹ or R¹⁰ is not H.

In some embodiments, the 15-PGDH inhibitor can include a compound having at least one of the following formulas:

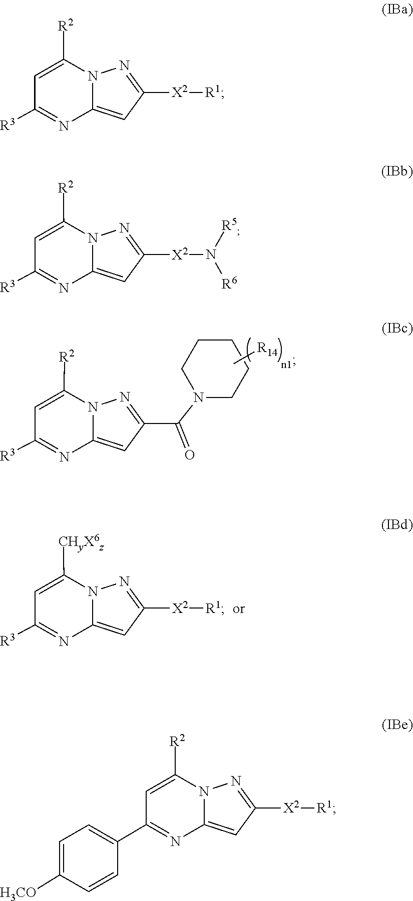

or a pharmaceutically acceptable salt thereof;
wherein X² is S, S═O, S(═O)₂, or C═O;
X⁶ is C₁, Br, or F, and y+z=3;
R¹, R², R³, R⁵, R⁶, and R¹⁴ are the same or different and are independently selected from the group consisting of hydrogen, substituted or unsubstituted C₁-C₂₄ alkyl, C₂-C₂₄ alkenyl, C₂-C₂₄ alkynyl, C₃-C₂₀ aryl, heterocycloalkenyl containing from 5-7 ring atoms, (wherein from 1-3 of the ring atoms is independently selected from N, NH, N(C₁-C₆ alkyl), NC(O) (C₁-C₆ alkyl), O, and S), heteroaryl or heterocyclyl containing from 5-14 ring atoms, (wherein from 1-6 of the ring atoms is independently selected from N, NH, N(C₁-C₃ alkyl), O, and S), C₆-C₂₄ alkaryl, C₆-C₂₄ aralkyl, halo, silyl, hydroxyl, sulfhydryl, C₁-C₂₄ alkoxy, C₂-C₂₄ alkenyloxy, C₂-C₂₄ alkynyloxy, C₅-C₂₀ aryloxy, acyl (including C₂-C₂₄ alkylcarbonyl (—CO-alkyl) and C₆-C₂₀ arylcarbonyl (—CO-aryl)), acyloxy (—O-acyl), C₂-C₂₄ alkoxycarbonyl (—(CO)—O-alkyl), C₆-C₂₀ aryloxycarbonyl (—(CO)—O-aryl), C₂-C₂₄ alkylcarbonato (—O—(CO)—O-alkyl), C₆-C₂₀ arylcarbonato (—O—(CO)—O-aryl), carboxy (—COOH), carboxylato (—COO⁻), carbamoyl (—(CO)—NH₂), C₁-C₂₄ alkyl-carbamoyl (—(CO)—NH(C₁-C₂₄ alkyl)), arylcarbamoyl (—(CO)—NH-aryl), thiocarbamoyl (—(CS)—NH₂), carbamido (—NH—(CO)—NH₂), cyano (—CN), isocyano (—N⁺C⁻), cyanato (—O—CN), isocyanato (—O—N⁺═C⁻), isothiocyanato (—S—CN), azido (—N═N⁺═N⁻), formyl (—(CO)—H), thioformyl (—(CS)—H), amino (—NH₂), C₁-C₂₄ alkyl amino, C₅-C₂₀ aryl amino, C₂-C₂₄ alkylamido (—NH—(CO)-alkyl), C₆-C₂₀ arylamido (—NH—(CO)-aryl), sulfanamido (—SO₂N(R)₂ where R is independently H, alkyl, aryl or heteroaryl), imino (—CR═NH where R is hydrogen, C₁-C₂₄ alkyl, C₅-C₂₀ aryl, C₆-C₂₄ alkaryl, C₆-C₂₄ aralkyl, etc.), alkylimino (—CR═N(alkyl), where R=hydrogen, alkyl, aryl, alkaryl, aralkyl, etc.), arylimino (—CR═N(aryl), where R=hydrogen, alkyl, aryl, alkaryl, etc.), nitro (—NO₂), nitroso (—NO), sulfo (—SO₂—OH), sulfonato (—SO₂—O⁻), C₁-C₂₄ alkylsulfanyl (—S-alkyl; also termed “alkylthio”), arylsulfanyl (—S-aryl; also termed “arylthio”), C₁-C₂₄ alkylsulfinyl (—(SO)-alkyl), C₅-C₂₀ arylsulfinyl (—(SO)-aryl), C₁-C₂₄ alkylsulfonyl (—SO₂-alkyl), C₅-C₂₀ arylsulfonyl (—SO₂-aryl), sulfonamide (—SO₂—NH₂, —SO₂NY₂ (wherein Y is independently H, aryl or alkyl), phosphono (—P(O)(OH)₂), phosphonato (—P(O)(O⁻)₂), phosphinato (—P(O)(O⁻)), phospho (—PO₂), phosphino (—PH₂), polyalkyl ethers (—[(CH₂)ₙO]ₘ), phosphates, phosphate esters [—OP(O)(OR)₂ where R=H, methyl or other alkyl], groups incorporating amino acids or other moieties expected to bear positive or negative charge at physiological pH, and combinations thereof; wherein R⁵ and R⁶ may be linked to form a cyclic or polycyclic ring, wherein the ring is a substituted or unsubstituted aryl, a substituted or unsubstituted heteroaryl, a substituted or unsubstituted cycloalkyl, and a substituted or unsubstituted heterocyclyl, n¹ is 0-4, and each R¹⁴ is the same or different.

In other embodiments, the 15-PGDH inhibitor can include a compound having at least one of the following formulas:

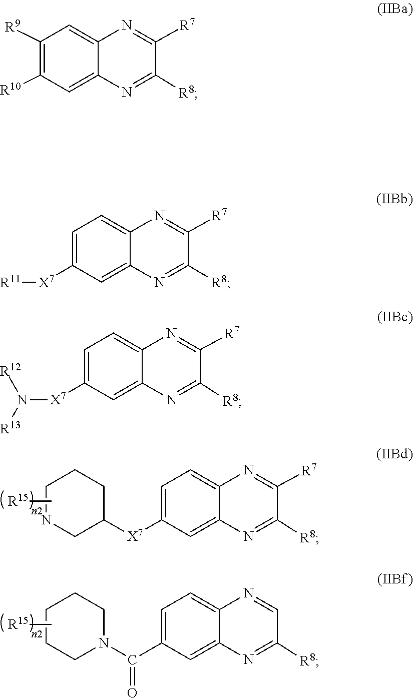

or a pharmaceutically acceptable salt thereof;
wherein X⁷ is S, S═O, S(═O)₂, or C═O;
R⁷ and R⁸ are same or different and are each independently selected from the group consisting of H, a substituted or unsubstituted aryl, a substituted or unsubstituted heteroaryl, a substituted or unsubstituted cycloalkyl, and a substituted or unsubstituted heterocyclyl, and at least one of R⁷ or R⁸ is not H;
R⁹, R¹⁰, R¹¹, R¹², R¹³, and R¹⁵ are the same or different and are independently selected from the group consisting of hydrogen, substituted or unsubstituted C₁-C₂₄ alkyl, C₂-C₂₄ alkenyl, C₂-C₂₄ alkynyl, C₃-C₂₀ aryl, heterocycloalkenyl containing from 5-7 ring atoms, (wherein from 1-3 of the ring atoms is independently selected from N, NH, N(C₁-C₆ alkyl), NC(O) (C₁-C₆ alkyl), O, and S), heteroaryl or heterocyclyl containing from 5-14 ring atoms, (wherein from 1-6 of the ring atoms is independently selected from N, NH, N(C₁-C₃ alkyl), O, and S), C₆-C₂₄ alkaryl, C₆-C₂₄ aralkyl, halo, silyl, hydroxyl, sulfhydryl, C₁-C₂₄ alkoxy, C₂-C₂₄ alkenyloxy, C₂-C₂₄ alkynyloxy, C₅-C₂₀ aryloxy, acyl (including C₂-C₂₄ alkylcarbonyl (—CO-alkyl) and C₆-C₂₀ arylcarbonyl (—CO-aryl)), acyloxy (—O-acyl), C₂-C₂₄ alkoxycarbonyl (—(CO)—O-alkyl), C₆-C₂₀ aryloxycarbonyl (—(CO)—O-aryl), C₂-C₂₄ alkylcarbonato (—O—(CO)—O-alkyl), C₆-C₂₀ arylcarbonato (—O—(CO)—O-aryl), carboxy (—COOH), carboxylato (—COO⁻), carbamoyl (—(CO)—NH₂), C₁-C₂₄ alkyl-carbamoyl (—(CO)—NH(C₁-C₂₄ alkyl)), arylcarbamoyl (—(CO)—NH-aryl), thiocarbamoyl (—(CS)—NH₂), carbamido (—NH—(CO)—NH₂), cyano (—CN), isocyano (—N⁺C⁻), cyanato (—O—CN), isocyanato (—O—N⁺═C⁻), isothiocyanato (—S—CN), azido (—N═N⁺═N⁻), formyl (—(CO)—H), thioformyl (—(CS)—H), amino (—NH₂), C₁-C₂₄ alkyl amino, C₅-C₂₀ aryl amino, C₂-C₂₄ alkylamido (—NH—(CO)-alkyl), C₆-C₂₀ arylamido (—NH—(CO)-aryl), sulfanamido (—SO₂N(R)₂ where R is independently H, alkyl, aryl or heteroaryl), imino (—CR═NH where R is hydrogen, C₁-C₂₄ alkyl, C₅-C₂₀ aryl, C₆-C₂₄ alkaryl, C₆-C₂₄ aralkyl, etc.), alkylimino (—CR═N(alkyl), where R=hydrogen, alkyl, aryl, alkaryl, aralkyl, etc.), arylimino (—CR═N(aryl), where R=hydrogen, alkyl, aryl, alkaryl, etc.), nitro (—NO₂), nitroso (—NO), sulfo (—SO₂—OH), sulfonato (—SO₂—O⁻), C₁-C₂₄ alkylsulfanyl (—S-alkyl; also termed “alkylthio”), arylsulfanyl (—S-aryl; also termed “arylthio”), C₁-C₂₄ alkylsulfinyl (—(SO)-alkyl), C₅-C₂₀ arylsulfinyl (—(SO)-aryl), C₁-C₂₄ alkylsulfonyl (—SO₂-alkyl), C₅-C₂₀ arylsulfonyl (—SO₂-aryl), sulfonamide (—SO₂—NH₂, —SO₂NY₂ (wherein Y is independently H, aryl or alkyl), phosphono (—P(O)(OH)₂), phosphonato (—P(O)(O⁻)₂), phosphinato (—P(O)(O⁻)), phospho (—PO₂), phosphino (—PH₂), polyalkyl ethers (—[(CH₂)ₙO]ₘ), phosphates, phosphate esters [—OP(O)(OR)₂ where R=H, methyl or other alkyl], groups incorporating amino acids or other moieties expected to bear positive or negative charge at physiological pH, and combinations thereof; wherein R¹² and R¹³ may be linked to form a cyclic or polycyclic ring, wherein the ring is a substituted or unsubstituted aryl, a substituted or unsubstituted heteroaryl, a substituted or unsubstituted cycloalkyl, and a substituted or unsubstituted heterocyclyl, n² is 0-4, and each R¹⁵ is the same or different

Examples of 15-PGDH inhibitors having formulas (IB), (IBa), (IBb), (IBc), (IBd), (IBe), (IIB), (IIBa), (IIBb), (IIBc), (IIBd), (IBe), or (IIBf) can include the following compounds:

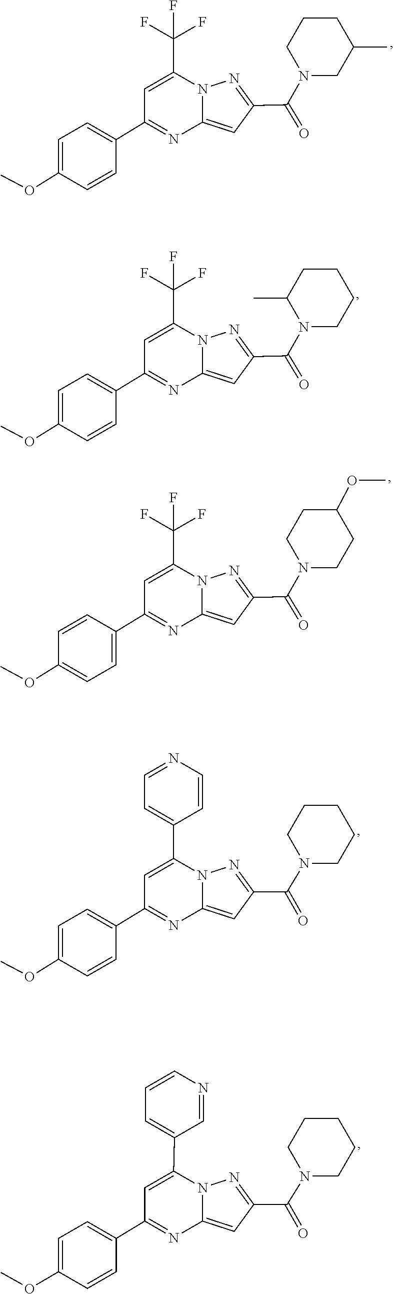

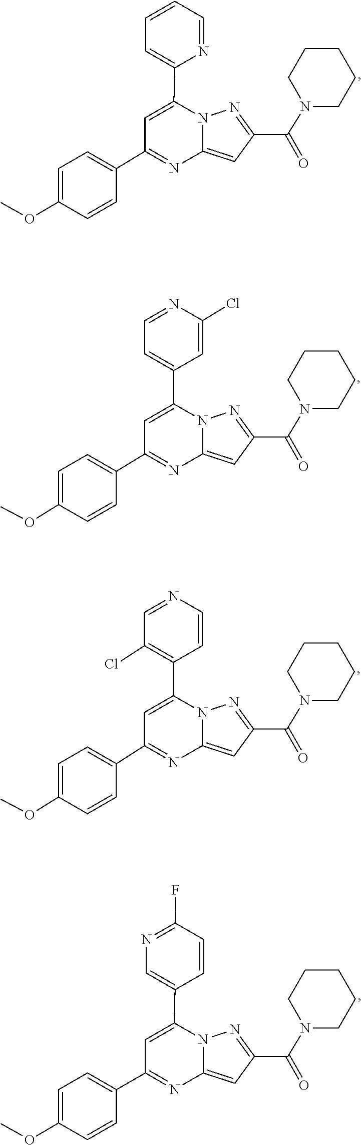 [SMILES: COc1ccc(-c2cc(-c3ccccn3)n3nc(C(=O)N4CCCCC4)cc3n2)cc1 ; COc1ccc(-c2cc(-c3ccnc(Cl)c3)n3nc(C(=O)N4CCCCC4)cc3n2)cc1 ; COc1ccc(-c2cc(-c3ccncc3Cl)n3nc(C(=O)N4CCCCC4)cc3n2)cc1 ; COc1ccc(-c2cc(-c3ccc(F)nc3)n3nc(C(=O)N4CCCCC4)cc3n2)cc1]

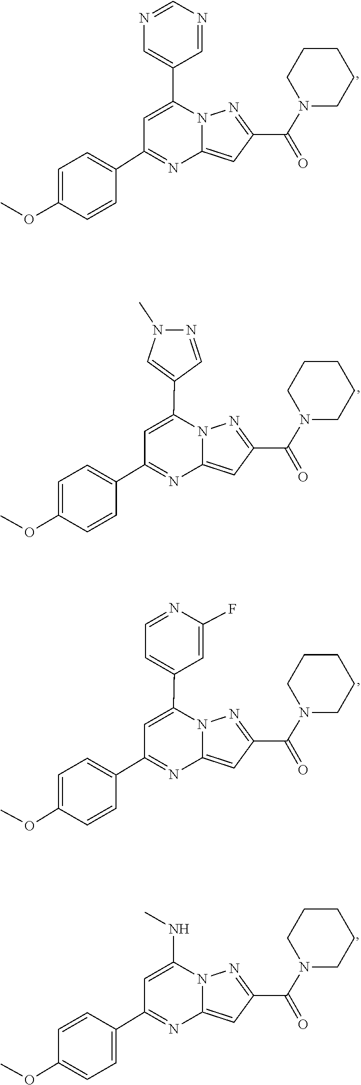 [SMILES: COc1ccc(-c2cc(-c3cncnc3)n3nc(C(=O)N4CCCCC4)cc3n2)cc1 ; COc1ccc(-c2cc(-c3cnn(C)c3)n3nc(C(=O)N4CCCCC4)cc3n2)cc1 ; COc1ccc(-c2cc(-c3ccnc(F)c3)n3nc(C(=O)N4CCCCC4)cc3n2)cc1 ; CNc1cc(-c2ccc(OC)cc2)nc2cc(C(=O)N3CCCCC3)nn12]

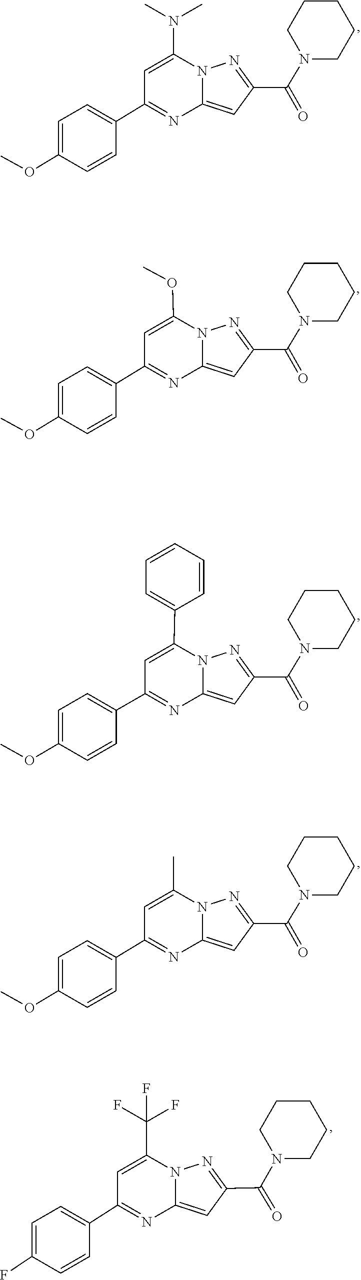 [SMILES: COc1ccc(-c2cc(N(C)C)n3nc(C(=O)N4CCCCC4)cc3n2)cc1 ; COc1ccc(-c2cc(OC)n3nc(C(=O)N4CCCCC4)cc3n2)cc1 ; COc1ccc(-c2cc(-c3ccccc3)n3nc(C(=O)N4CCCCC4)cc3n2)cc1 ; COc1ccc(-c2cc(C)n3nc(C(=O)N4CCCCC4)cc3n2)cc1 ; O=C(c1cc2nc(-c3ccc(F)cc3)cc(C(F)(F)F)n2n1)N1CCCCC1]

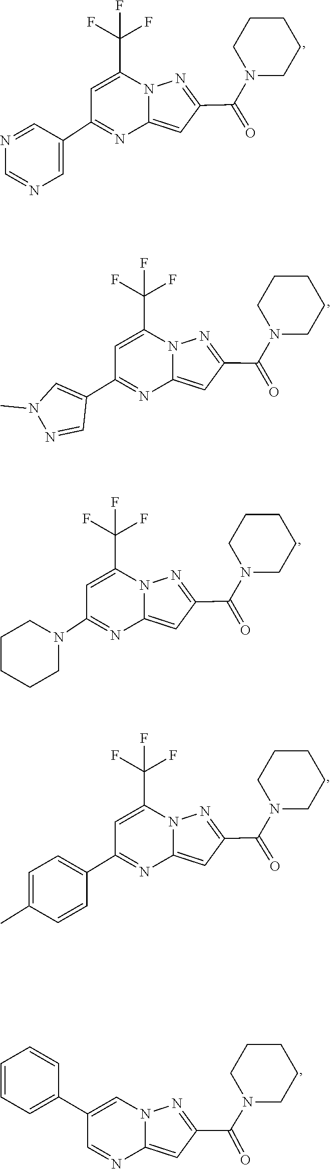 [SMILES: O=C(c1cc2nc(-c3cncnc3)cc(C(F)(F)F)n2n1)N1CCCCC1 ; Cn1cc(-c2cc(C(F)(F)F)n3nc(C(=O)N4CCCCC4)cc3n2)cn1 ; O=C(c1cc2nc(N3CCCCC3)cc(C(F)(F)F)n2n1)N1CCCCC1 ; Cc1ccc(-c2cc(C(F)(F)F)n3nc(C(=O)N4CCCCC4)cc3n2)cc1 ; O=C(c1cc2ncc(-c3ccccc3)cn2n1)N1CCCCC1]

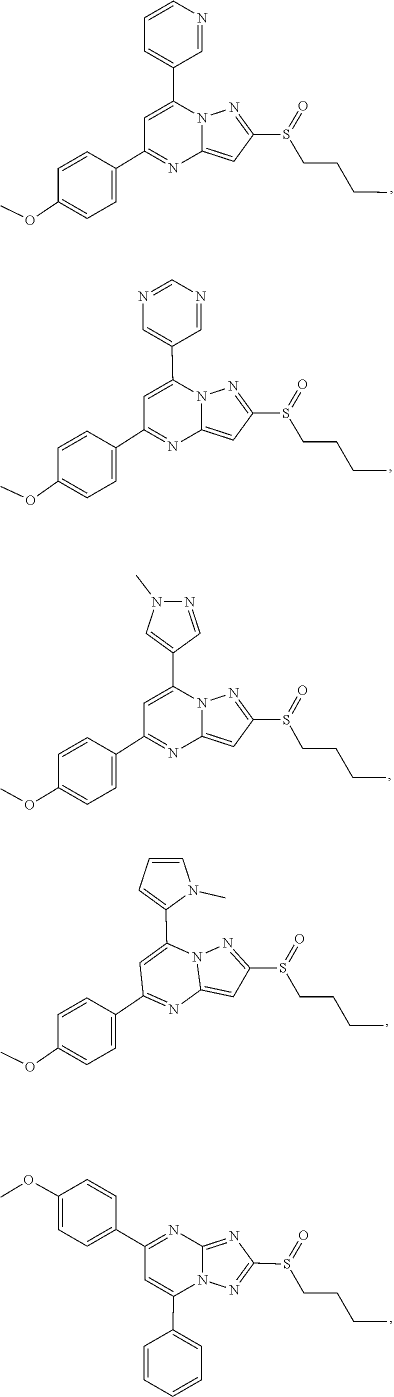

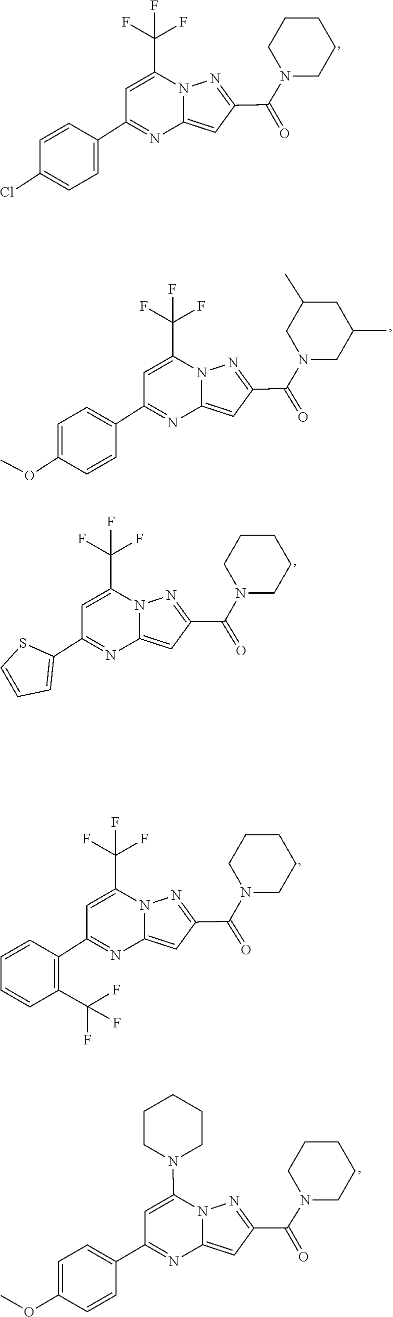

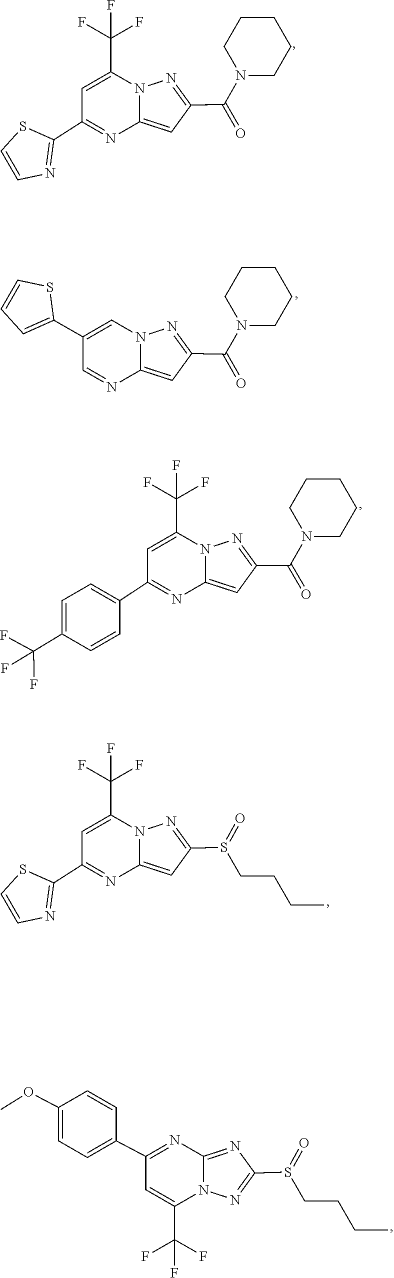

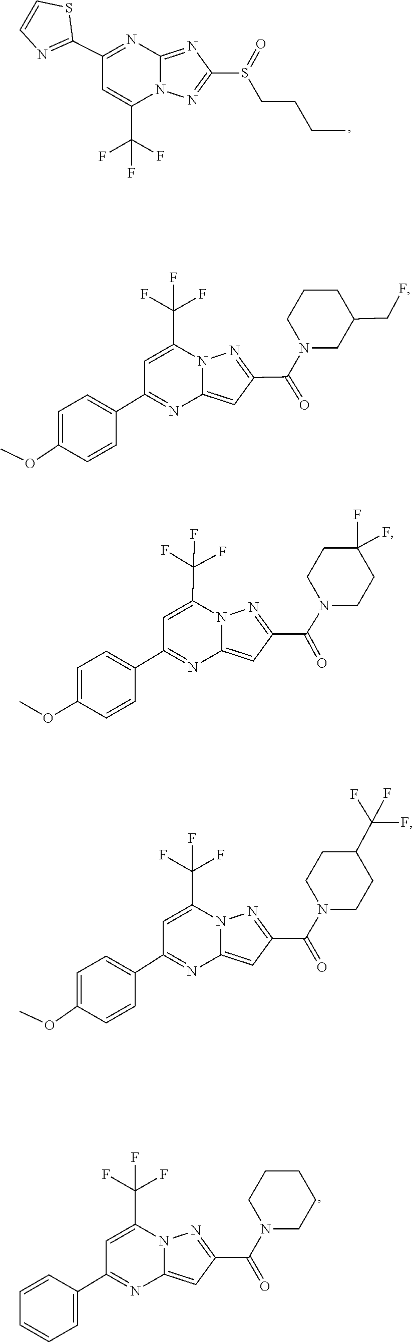

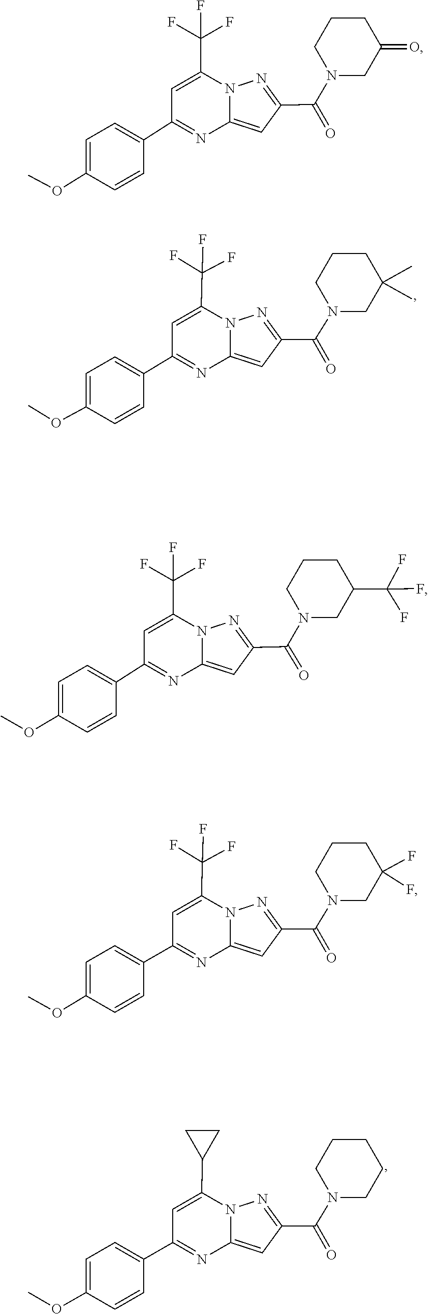

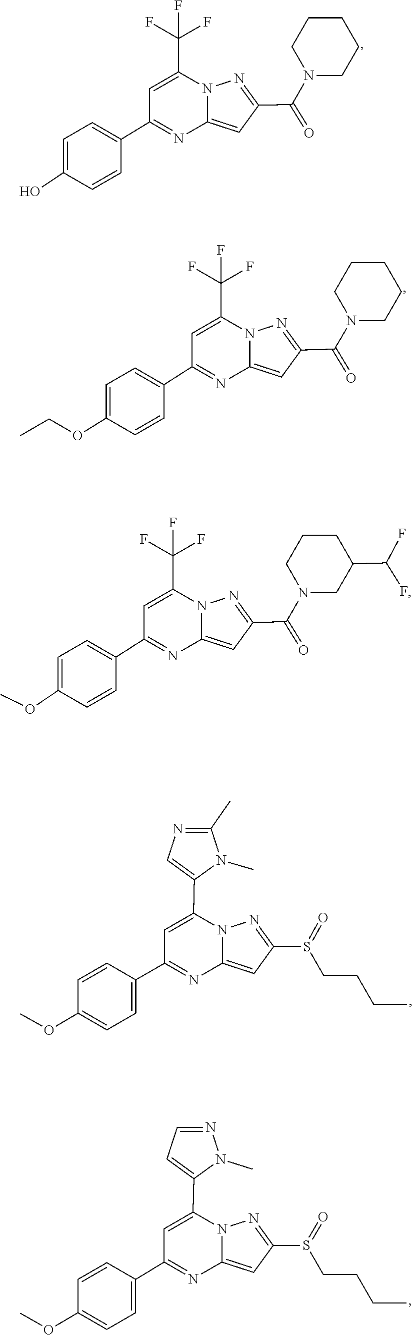

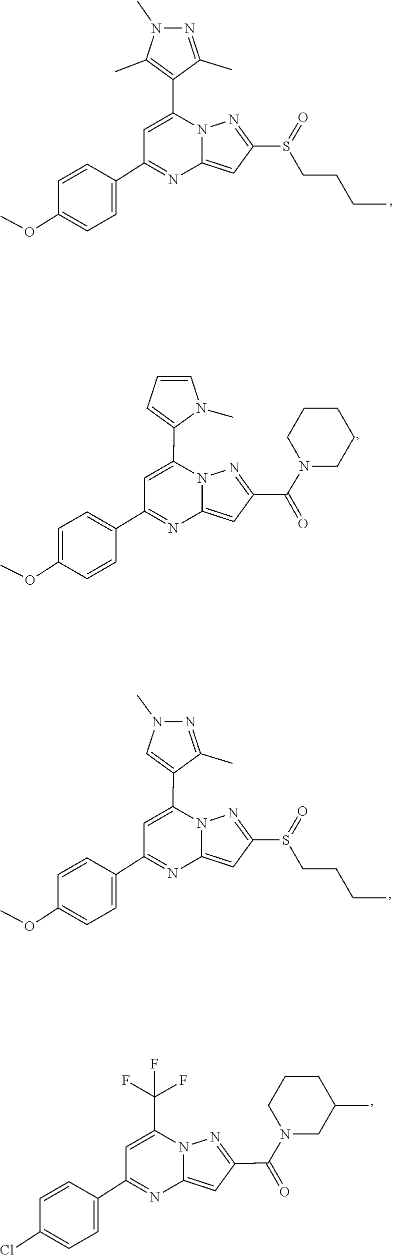

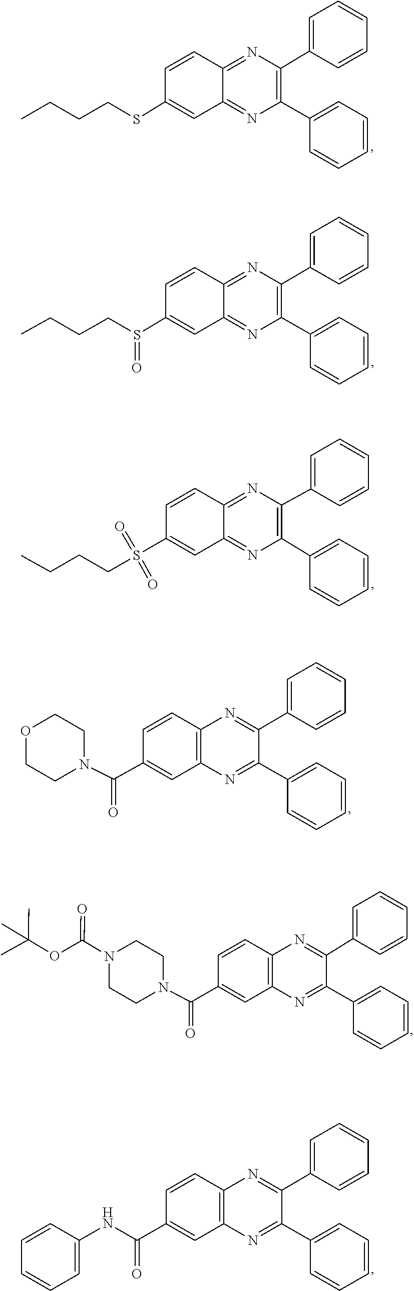 [SMILES: CCCCSc1ccc2nc(-c3ccccc3)c(-c3ccccc3)nc2c1 ; CCCCS(=O)c1ccc2nc(-c3ccccc3)c(-c3ccccc3)nc2c1 ; CCCCS(=O)(=O)c1ccc2nc(-c3ccccc3)c(-c3ccccc3)nc2c1 ; O=C(c1ccc2nc(-c3ccccc3)c(-c3ccccc3)nc2c1)N1CCOCC1 ; CC(C)(C)OC(=O)N1CCN(C(=O)c2ccc3nc(-c4ccccc4)c(-c4ccccc4)nc3c2)CC1 ; O=C(Nc1ccccc1)c1ccc2nc(-c3ccccc3)c(-c3ccccc3)nc2c1]

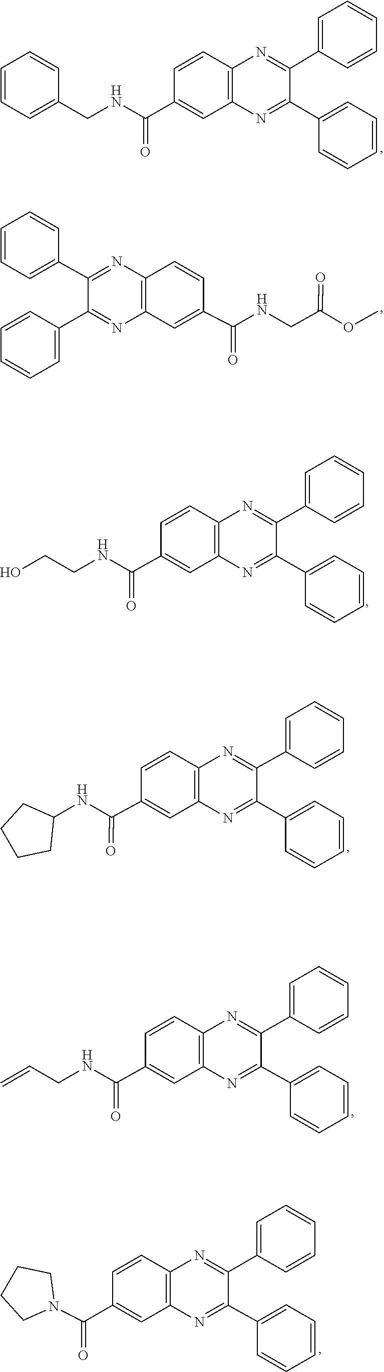

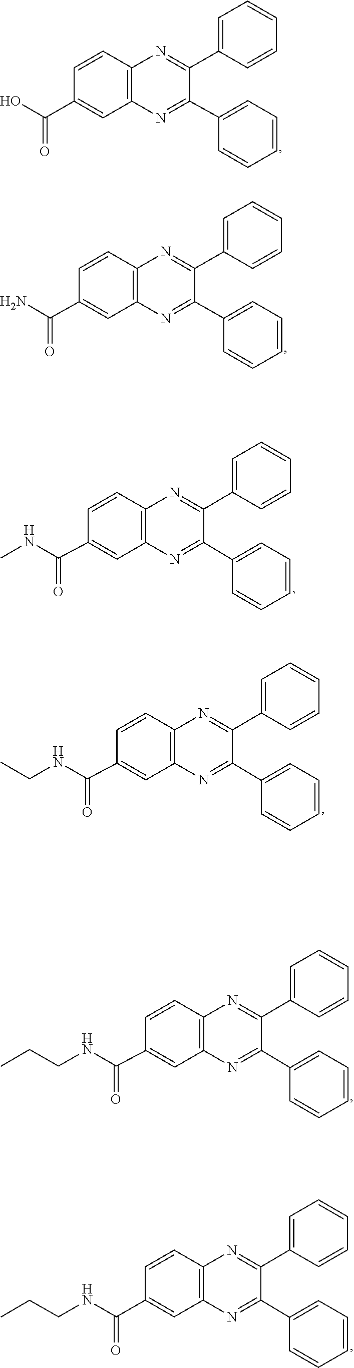

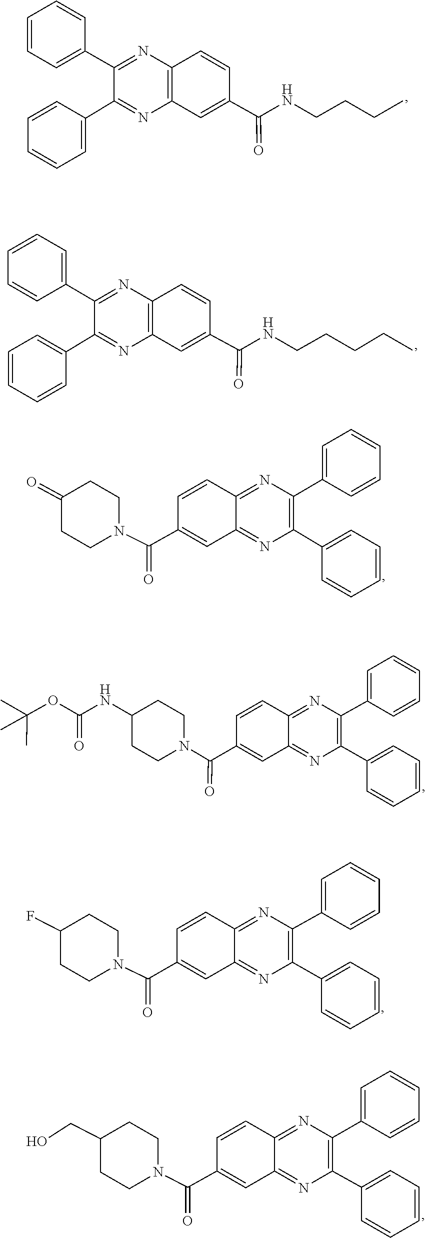

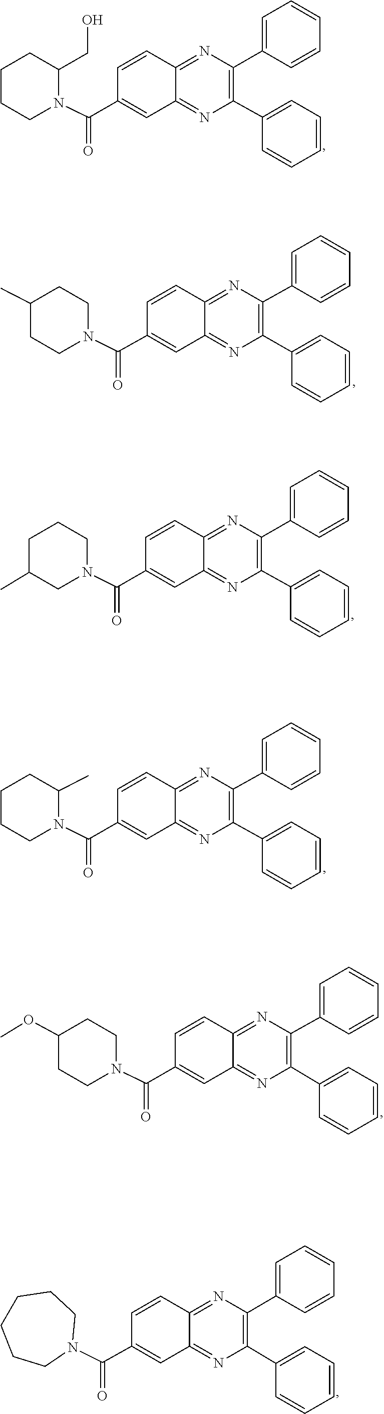 [SMILES: O=C(c1ccc2nc(-c3ccccc3)c(-c3ccccc3)nc2c1)N1CCCCC1CO ; CC1CCN(C(=O)c2ccc3nc(-c4ccccc4)c(-c4ccccc4)nc3c2)CC1 ; CC1CCCN(C(=O)c2ccc3nc(-c4ccccc4)c(-c4ccccc4)nc3c2)C1 ; CC1CCCCN1C(=O)c1ccc2nc(-c3ccccc3)c(-c3ccccc3)nc2c1 ; COC1CCN(C(=O)c2ccc3nc(-c4ccccc4)c(-c4ccccc4)nc3c2)CC1 ; O=C(c1ccc2nc(-c3ccccc3)c(-c3ccccc3)nc2c1)N1CCCCCC1]

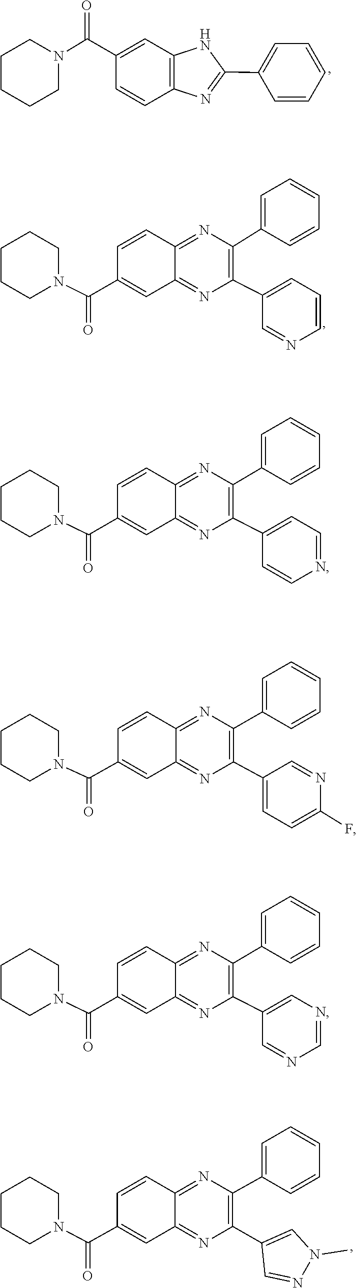 [SMILES: O=C(c1ccc2nc(-c3ccccc3)[nH]c2c1)N1CCCCC1 ; O=C(c1ccc2nc(-c3ccccc3)c(-c3cccnc3)nc2c1)N1CCCCC1 ; O=C(c1ccc2nc(-c3ccccc3)c(-c3ccncc3)nc2c1)N1CCCCC1 ; O=C(c1ccc2nc(-c3ccccc3)c(-c3ccc(F)nc3)nc2c1)N1CCCCC1 ; O=C(c1ccc2nc(-c3ccccc3)c(-c3cncnc3)nc2c1)N1CCCCC1 ; Cn1cc(-c2nc3cc(C(=O)N4CCCCC4)ccc3nc2-c2ccccc2)cn1]

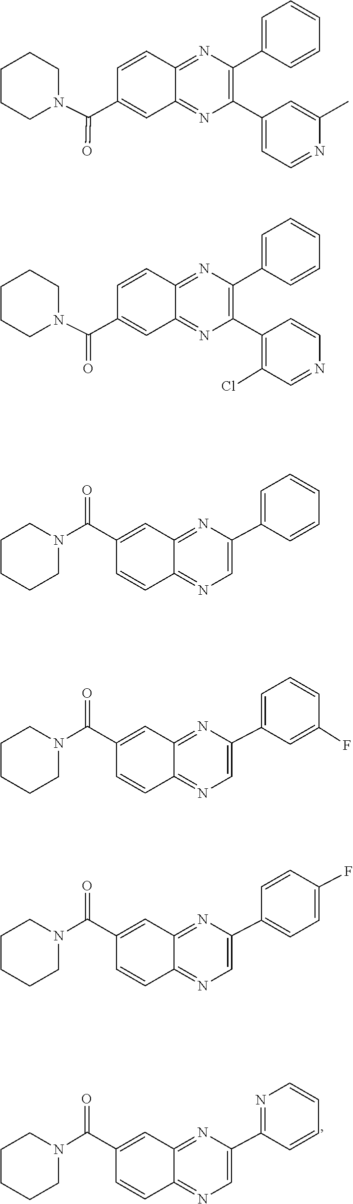 [SMILES: Cc1cc(-c2nc3cc(C(=O)N4CCCCC4)ccc3nc2-c2ccccc2)ccn1 ; O=C(c1ccc2nc(-c3ccccc3)c(-c3ccncc3Cl)nc2c1)N1CCCCC1 ; O=C(c1ccc2ncc(-c3ccccc3)nc2c1)N1CCCCC1 ; O=C(c1ccc2ncc(-c3cccc(F)c3)nc2c1)N1CCCCC1 ; O=C(c1ccc2ncc(-c3ccc(F)cc3)nc2c1)N1CCCCC1 ; O=C(c1ccc2ncc(-c3ccccn3)nc2c1)N1CCCCC1]

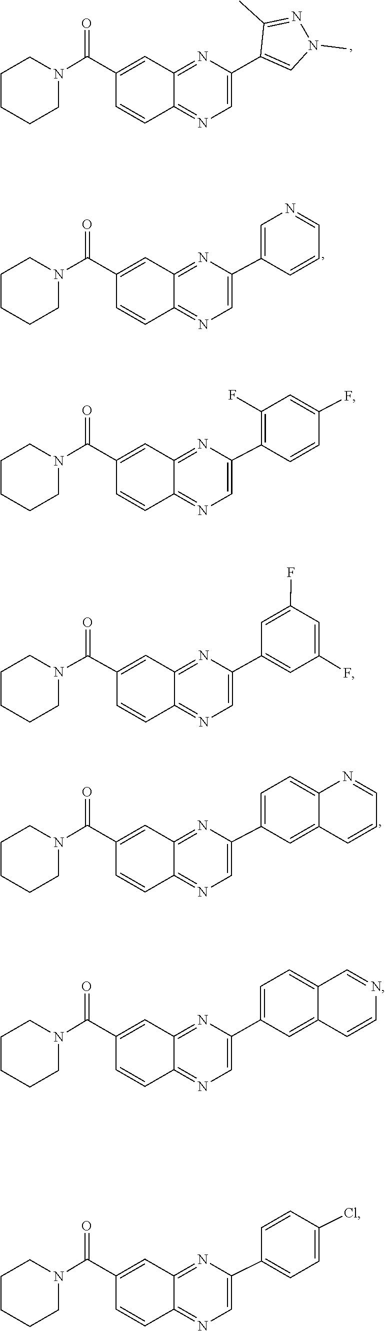

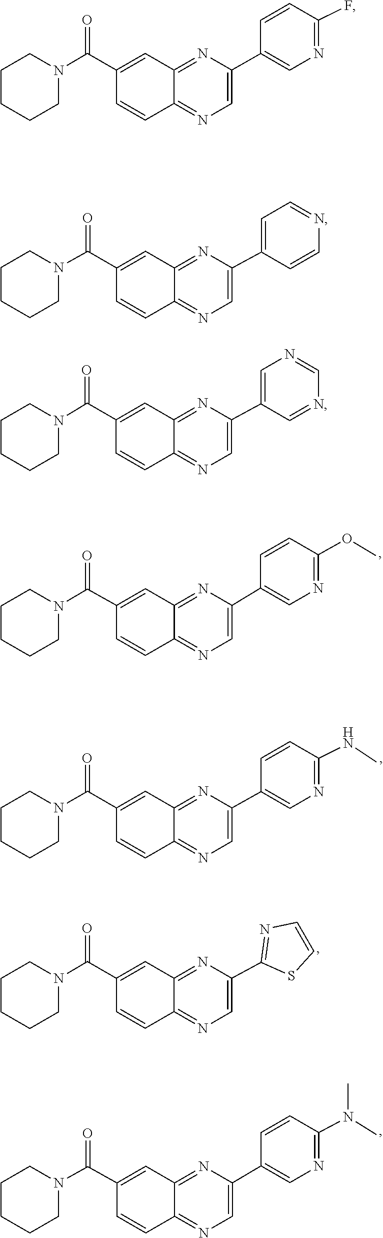

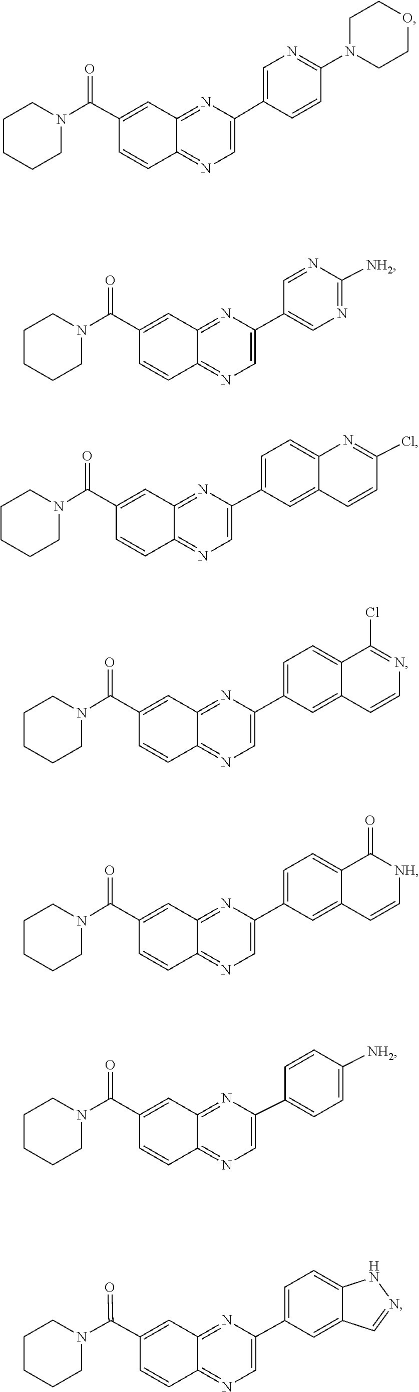

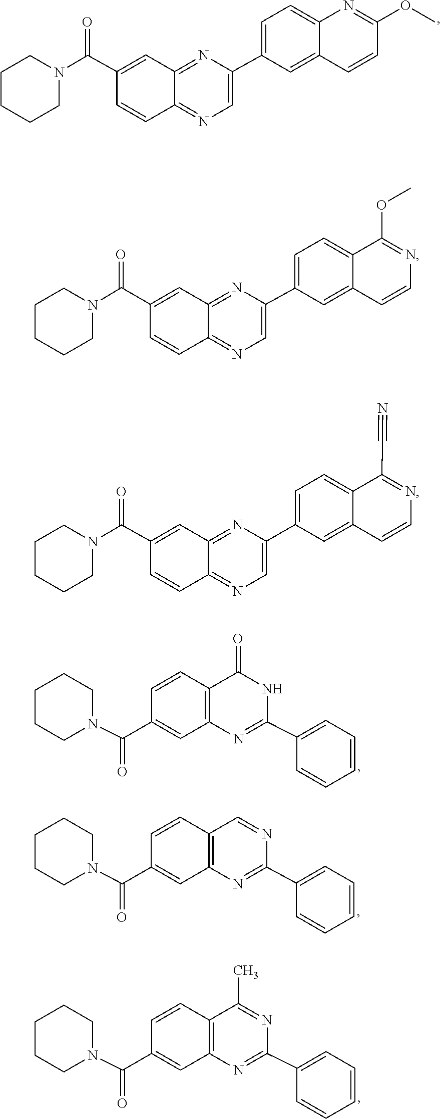 [SMILES: COc1ccc2cc(-c3cnc4ccc(C(=O)N5CCCCC5)cc4n3)ccc2n1 ; COc1nccc2cc(-c3cnc4ccc(C(=O)N5CCCCC5)cc4n3)ccc12 ; N#Cc1nccc2cc(-c3cnc4ccc(C(=O)N5CCCCC5)cc4n3)ccc12 ; O=C(c1ccc2c(=O)[nH]c(-c3ccccc3)nc2c1)N1CCCCC1 ; O=C(c1ccc2cnc(-c3ccccc3)nc2c1)N1CCCCC1 ; Cc1nc(-c2ccccc2)nc2cc(C(=O)N3CCCCC3)ccc12]

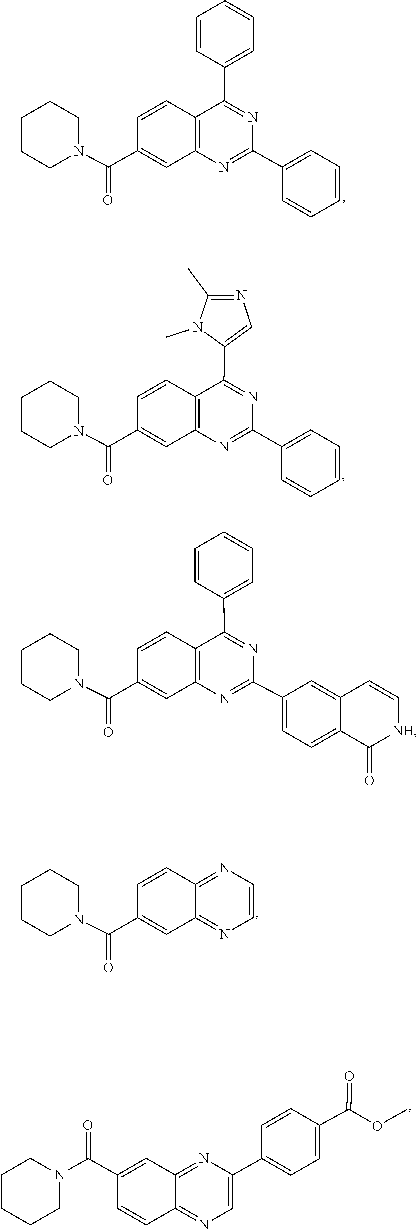 [SMILES: O=C(c1ccc2c(-c3ccccc3)nc(-c3ccccc3)nc2c1)N1CCCCC1 ; Cc1ncc(-c2nc(-c3ccccc3)nc3cc(C(=O)N4CCCCC4)ccc23)n1C ; O=C(c1ccc2c(-c3ccccc3)nc(-c3ccc4c(=O)[nH]ccc4c3)nc2c1)N1CCCCC1 ; O=C(c1ccc2nccnc2c1)N1CCCCC1 ; COC(=O)c1ccc(-c2cnc3ccc(C(=O)N4CCCCC4)cc3n2)cc1]

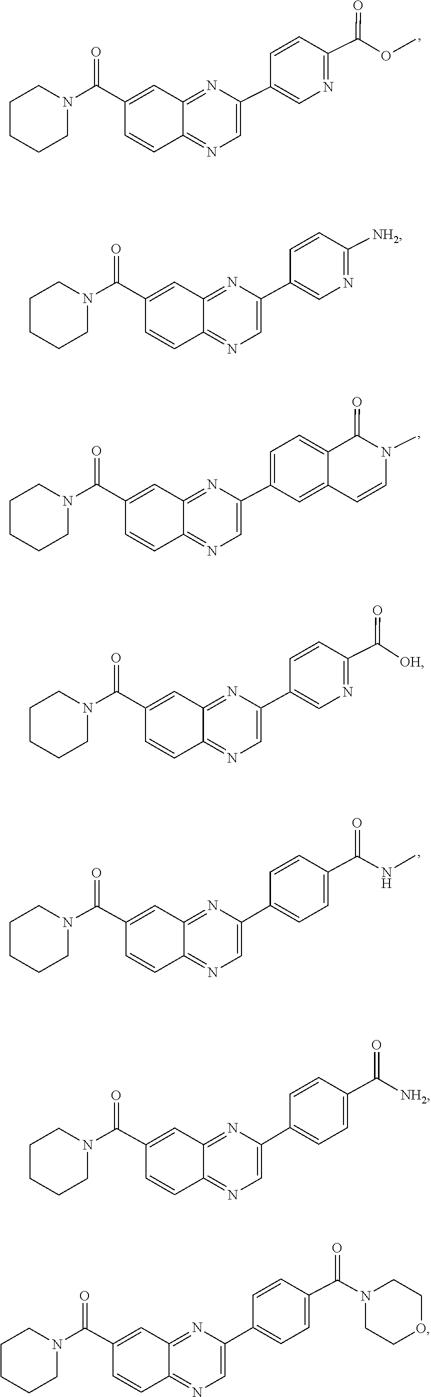

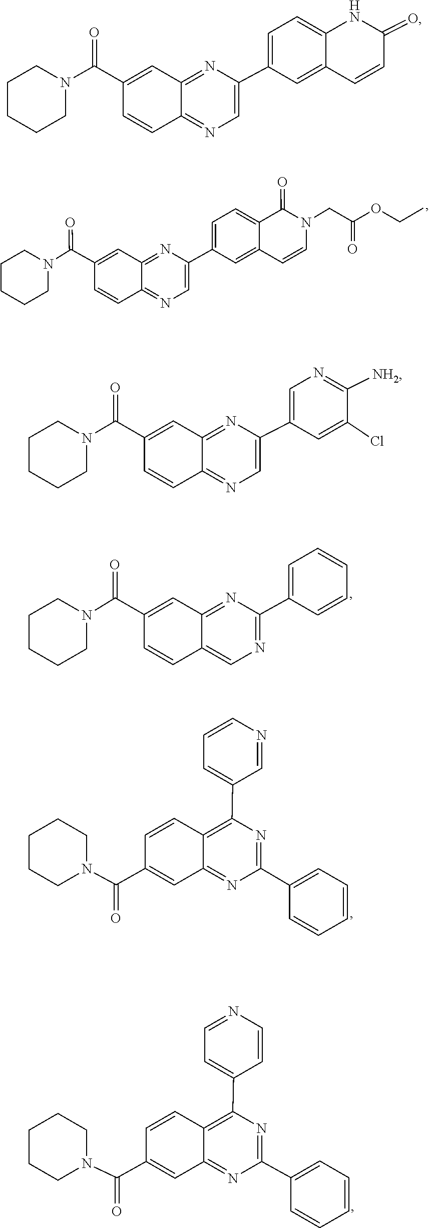 [SMILES: O=C(c1ccc2ncc(-c3ccc4[nH]c(=O)ccc4c3)nc2c1)N1CCCCC1 ; CCOC(=O)Cn1ccc2cc(-c3cnc4ccc(C(=O)N5CCCCC5)cc4n3)ccc2c1=O ; Nc1ncc(-c2cnc3ccc(C(=O)N4CCCCC4)cc3n2)cc1Cl ; O=C(c1ccc2cnc(-c3ccccc3)nc2c1)N1CCCCC1 ; O=C(c1ccc2c(-c3cccnc3)nc(-c3ccccc3)nc2c1)N1CCCCC1 ; O=C(c1ccc2c(-c3ccncc3)nc(-c3ccccc3)nc2c1)N1CCCCC1]

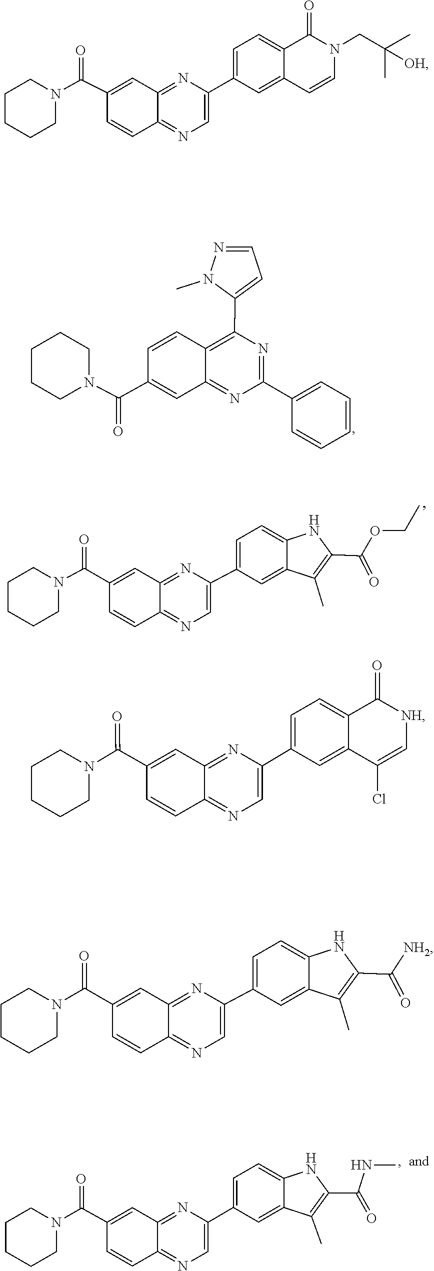 [SMILES: CC(C)(O)Cn1ccc2cc(-c3cnc4ccc(C(=O)N5CCCCC5)cc4n3)ccc2c1=O ; Cn1nccc1-c1nc(-c2ccccc2)nc2cc(C(=O)N3CCCCC3)ccc12 ; CCOC(=O)c1[nH]c2ccc(-c3cnc4ccc(C(=O)N5CCCCC5)cc4n3)cc2c1C ; O=C(c1ccc2ncc(-c3ccc4c(=O)[nH]cc(Cl)c4c3)nc2c1)N1CCCCC1 ; Cc1c(C(N)=O)[nH]c2ccc(-c3cnc4ccc(C(=O)N5CCCCC5)cc4n3)cc12 ; CNC(=O)c1[nH]c2ccc(-c3cnc4ccc(C(=O)N5CCCCC5)cc4n3)cc2c1C]

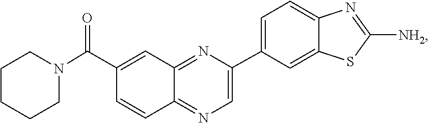 [SMILES: Nc1nc2ccc(-c3cnc4ccc(C(=O)N5CCCCC5)cc4n3)cc2s1]

or pharmaceutically acceptable salts thereof, a tautomers thereof, and a solvates thereof.

Still other example of 15-PGDH inhibitors include compounds described in WO2018/145080, which is incorporated by reference in its entirety.

In certain embodiments, the 15-PGDH inhibitor can be selected that can ia) at 2.5 μM concentration, stimulate a Vaco503 reporter cell line expressing a 15-PGDH luciferase fusion construct to a luciferase output level of greater than 70 (using a scale on which a value of 100 indicates a doubling of reporter output over baseline); iia) at 2.5 μM concentration stimulate a V9m reporter cell line expressing a 15-PGDH luciferase fusion construct to a luciferase output level of greater than 75; iiia) at 7.5 μM concentration stimulate a LS174T reporter cell line expressing a 15-PGDH luciferase fusion construct to a luciferase output level of greater than 70; and iva) at 7.5 μM concentration, does not activate a negative control V9m cell line expressing TK-Renilla luciferase reporter to a level greater than 20; and va) inhibits the enzymatic activity of recombinant 15-PGDH protein at an IC₅₀ of less than 1 μM.

In other embodiments, the 15-PGDH inhibitor can ib) at 2.5 μM concentration, stimulate a Vaco503 reporter cell line expressing a 15-PGDH luciferase fusion construct to increase luciferase output; iib) at 2.5 μM concentration stimulate a V9m reporter cell line expressing a 15-PGDH luciferase fusion construct to increase luciferase output; iiib) at 7.5 μM concentration stimulate a LS174T reporter cell line expressing a 15-PGDH luciferase fusion construct to increase luciferase output; ivb) at 7.5 μM concentration, does not activate a negative control V9m cell line expressing TK-Renilla luciferase reporter to a luciferase level greater than 20% above background; and vb) inhibits the enzymatic activity of recombinant 15-PGDH protein at an IC₅₀ of less than 1 μM.

In other embodiments, the 15-PGDH inhibitor can inhibit the enzymatic activity of recombinant 15-PGDH at an IC50 of less than 1 μM, or preferably at an IC50 of less than 250 nM, or more preferably at an IC₅₀ of less than 50 nM, or more preferably at an IC₅₀ of less than 10 nM, or more preferably at an IC₅₀ of less than 5 nM at a recombinant 15-PGDH concentration of about 5 nM to about 10 nM.

It will be appreciated that other 15-PGDH inhibitors can be used in the methods described herein. These other 15-PGDH inhibitors can include known 15-PGDH inhibitors including, for example, tetrazole compounds of formulas (I) and (II), 2-alkylideneaminooxyacetamide compounds of formula (I), heterocyclic compounds of formulas (VI) and (VII), and pyrazole compounds of formula (III) described in U.S. Patent Application Publication No. 2006/0034786 and U.S. Pat. No. 7,705,041; benzylidene-1,3-thiazolidine compounds of formula (I) described in U.S. Patent Application Publication No. 2007/0071699; phenylfurylmethylthiazolidine-2,4-dione and phenylthienylmethylthiazolidine-2,4-dione compounds described in U.S. Patent Application Publication No. 2007/0078175; thiazolidenedione derivatives described in U.S. Patent Application Publication No. 2011/0269954; phenylfuran, phenylthiophene, or phenylpyrrazole compounds described in U.S. Pat. No. 7,294,641, 5-(3,5-disubstituted phenylazo)-2-hydroxybenzene-acetic acids and salts and lactones described in U.S. Pat. No. 4,725,676, and azo compounds described in U.S. Pat. No. 4,889,846.

Still other examples of 15-PGDH inhibitors are described in the following publications: Seo S Y et al. Effect of 15-hydroxyprostaglandin dehydrogenase inhibitor on wound healing. Prostaglandins Leukot Essent Fatty Acids. 2015; 97:35-41. doi: 10.1016/j.plefa.2015.03.005. PubMed PMID: 25899574; Piao Y L et al. Wound healing effects of new 15-hydroxyprostaglandin dehydrogenase inhibitors. Prostaglandins Leukot Essent Fatty Acids. 2014; 91(6):325-32. doi: 10.1016/j.plefa.2014.09.011. PubMed PMID: 25458900; Choi D et al. Control of the intracellular levels of prostaglandin E(2) through inhibition of the 15-hydroxyprostaglandin dehydrogenase for wound healing. Bioorg Med Chem. 2013; 21(15):4477-84. doi: 10.1016/j.bmc.2013.05.049. PubMed PMID: 23791868; Wu Y et al. Synthesis and biological evaluation of novel thiazolidinedione analogues as 15-hydroxyprostaglandin dehydrogenase inhibitors. J Med Chem. 2011; 54(14):5260-4. Epub 2011/06/10. doi: 10.1021/jm200390u. PubMed PMID: 21650226; Duveau D Y et al. Structure-activity relationship studies and biological characterization of human NAD(+)-dependent 15-hydroxyprostaglandin dehydrogenase inhibitors. Bioorg Med Chem Lett. 2014; 24(2):630-5. doi: 10.1016/j.bmcl.2013.11.081. PubMed PMID: 24360556; PMCID: PMC3970110; Duveau D Y et al. Discovery of two small molecule inhibitors, ML387 and ML388, of human NAD+-dependent 15-hydroxyprostaglandin dehydrogenase. Probe Reports from the NIH Molecular Libraries Program. Bethesda (MD)2010; Wu Y et al. Synthesis and SAR of thiazolidinedione derivatives as 15-PGDH inhibitors. Bioorg Med Chem. 2010; 18(4):1428-33. doi: 10.1016/j.bmc.2010.01.016. PubMed PMID: 20122835; Wu Y et al. Synthesis and biological evaluation of novel thiazolidinedione analogues as 15-hydroxyprostaglandin dehydrogenase inhibitors. J Med Chem. 2011; 54(14):5260-4. Epub 2011/06/10. doi: 10.1021/jm200390u. PubMed PMID: 21650226; Jadhav A et al. Potent and selective inhibitors of NAD+-dependent 15-hydroxyprostaglandin dehydrogenase (HPGD). Probe Reports from the NIH Molecular Libraries Program. Bethesda (MD)2010; Niesen F H et al. High-affinity inhibitors of human NAD-dependent 15-hydroxyprostaglandin dehydrogenase: mechanisms of inhibition and structure-activity relationships. PLoS One. 2010; 5(11):e13719. Epub 2010/11/13. doi: 10.1371/journal.pone.0013719. PubMed PMID: 21072165; PMCID: 2970562; Michelet, J. et al. Composition comprising at least one 15-PGDH inhibitor. US20080206320 A1, 2008; and Rozot, R et al. Care/makeup compositions comprising a 2-alkylideneaminooxyacetamide compound for stimulating the growth of the hair or eyelashes and/or slowing loss thereof. U.S. Pat. No. 7,396,525 B2, 2008.

The 15-PGDH inhibitors described herein can be used to treat, prevent, or reduce the symptoms or severity of any neudegenerative disease, disorder, or condition associated with aberrant 15-PGDH activity. In some embodiments, a subject having the neudegenerative disease, disorder, or condition can have or be at risk of memory loss, cognitive decline, axonal degeneration, neuronal cell death, glia cell damage, and/or blood brain barrier permeability and the 15-PGDH inhibitor can be administered to the subject at an amount effective to treat or prevent the memory loss, cognitive decline, axonal degeneration, neuronal cell death, glia cell damage, and/or blood brain barrier permeability.

Subjects amenable to treatment by 15-PGDH inhibitors as disclosed herein include subjects at risk of a neurodegenerative condition, disease, or disorder but not showing symptoms (for example asymptomatic subjects), as well as subjects presently showing symptoms. In the case of dementia diseases, virtually anyone is at risk of suffering from dementia if he or she lives long enough. Therefore, the present methods can be administered prophylactically to the general population without any assessment of the risk of the subject patient. The methods as disclosed herein are especially useful for individuals who do have a known genetic risk of neurodegenative condition, disease, of disorder. Such individuals include those having relatives who have experienced this disease, and those whose risk is determined by analysis of genetic or biochemical markers, as disclosed herein.

Subjects can be screened for their likelihood of having or developing a neurodegenerative condition, disease, or disorder based on a number of biochemical and genetic markers. For example, one can also diagnose a subject with increased risk of developing Alzheimer's Disease using genetic markers for Alzheimer's Disease. Genetic abnormality in a few families has been traced to chromosome 21 (St. George-Hyslop et al., Science 235:885-890, 1987). One genetic marker is, for example mutations in the APP gene, particularly mutations at position 717 and positions 670 and 671 referred to as the Hardy and Swedish mutations respectively (see Hardy, TINS, supra). Other markers of risk are mutations in the presenilin genes, PS1 and PS2, and ApoE4, family history of Alzheimer's Disease, hypercholesterolemia or atherosclerosis. Subjects with APP, PS1 or PS2 mutations are highly likely to develop Alzheimer's disease. ApoE is a susceptibility gene, and subjects with the e4 isoform of ApoE (ApoE4 isoform) have an increased risk of developing Alzheimer's disease. Test for subjects with ApoE4 isoform are disclosed in U.S. Pat. No. 6,027,896, which is incorporated in its entirety herein by reference. Other genetic links have been associated with increased risk of Alzheimer's disease, for example variances in the neuronal sortilin-related receptor SORL1 may have increased likelihood of developing late-onset Alzheimer's disease (Rogaeva at al, Nat. Genet. 2007 February; 39(2):168-77). Other potential Alzheimer disease susceptibility genes, include, for example ACE, CHRNB2, CST3, ESR1, GAPDHS, IDE, MTHFR, NCSTN, PRNP, PSEN1, TF, TFAM and TNF and be used to identify subjects with increased risk of developing Alzheimer's disease (Bertram et al, Nat. Genet. 2007 January; 39(1):17-23), as well as variances in the alpha-T catenin (VR22) gene (Bertram et al, J Med. Genet. 2007 January; 44(1):e63) and Insulin-degrading enzyme (IDE) and Kim et al, J Biol. Chem. 2007; 282:7825-32).

Neurodegenative conditions, disease, or disorders associated with cognitive decline or memory loss can be diagnosed using standard practice and the progression can be monitored over an extended period of time. One such method includes at least one of the following; (i) a memory assessment, (ii) an extensive neuropsychological exam, (iii) an examination by a geriatric neurologist and (iv) MRI imaging of the brain. Disease progression can be documented by changes in these parameters over time. In some embodiments, changes in the parameters of at least one of these assessments can be used to assess the efficacy of 15-PGDH inhibitor in the subject over time.

Other methods to diagnose a patient at risk of or having a neurodegenerative condition, disease or disorder, such as vascular dementia or Alzheimer's Disease, includes measurement of 15-PGDH activity and/or expression in the neurotissue, such as brain tissue, wherein increased 15-PGDH activity and/or expression compared to a control (e.g., normal or healthy neurotissue) is indicative of the subject having or at increased risk of the neurodegenerative condition, disease, or disorder.

In some embodiments, where the subject has or is at risk of blood brain barrier breakdown, direct detection of BBB breakdown can be assessed using MRI and injection of contrasting agent. Improvements in the resolution of MRIs and in the use of special contrasting agents can be used to detect BBB permeability. In one method, subjects are administered a contrasting agent immediately prior to brain imaging, such as MRI imaging. In cases of intact BBB, the contrasting agents are confined to brain blood vessels whereas, in subjects with a disrupted BBB, the contrasting agent is “sprayed out” into the brain tissue, which can be visualized. Thus, the brain locations, the size of BBB breakdown and extent of BBB compromise, such as extent of vascular leak in subjects can be directly and quantitatively assessed as measurable parameters of BBB permeability. Furthermore, such methods can be used to assess any improvements in these parameters resulting from treatment of the subject with an agent that inhibits 15-PGDH activity. Direct visualization of BBB breakdown and its associated vascular leak into the brain is useful in the methods as disclosed herein for monitoring the beneficial effects of treating a subject with BBB permeability with a 15-PGDH inhibitor. An improvement in at least one measurable parameter of BBB permeability, such as location, size of BBB breakdown and extent of vascular leak in subjects administered a 15-PGDH inhibitor indicates a positive outcome from administration of an inhibitor of 15-PGDH. Parameters of BBB permeability can be monitored by direct visualization and quantified by MRI-associated image analysis and are useful in the methods as disclosed herein.

In some embodiments, the 15-PGDH inhibitors described herein are useful in preventing and treating neurodegenerative conditions, diseases and disorders, such as those associated with risk of memory loss, cognitive decline, axonal degeneration, neuronal cell death, glia cell damage, and/or blood brain barrier permeability as well as those with aberrant 15-PGDH activity. As discussed previously, such neurodegenerative conditions, diseases, or disorders can include subarachnoid hemorrhage, schizophrenia, major depression, bipolar disorder, normal aging, epilepsy, traumatic brain injury and/or a visual symptom associated therewith, post-traumatic stress disorder, Parkinson's disease, Parkinson Plus syndromes, Lewy Body Dementia, multiple system atrophy, corticobasal neurodegeneration, progressive supranuclear palsy, Alzheimer's disease, Alzheimer's disease related dementias, Down syndrome, spinocerebellar ataxia, amyotrophic lateral sclerosis, Huntington's disease, stroke, brain radiation therapy, chronic stress, abuse of a neuro-active drug, retinal degeneration, spinal cord injury, peripheral nerve injury, idiopathic peripheral neuropathy, cognitive decline and/or general frailty associated with normal aging and/or chemotherapy, chemotherapy induced neuropathy, concussive injury, peripheral nerve crush injury, peripheral neuropathy, diabetic neuropathy, post-traumatic headache, multiple sclerosis, retinal degeneration and dystrophy, Leber congenital amaurosis, retinitis pigmentosa, cone-rod dystrophy, microphthalmia, anophthalmia, myopia, and hyperopia, spinal cord injury, traumatic spinal cord injury, peripheral nerve injury, retinal neuronal death related diseases, retinal trauma, Autism, Stargardt disease, Kearns-Sayre syndrome, Pure neurosensory deafness, Hereditary hearing loss with retinal diseases, Hereditary hearing loss with system atrophies of the nervous system, Progressive spinal muscular atrophy, Progressive bulbar palsy, Primary lateral sclerosis, Hereditary forms of progressive muscular atrophy and spastic paraplegia, Frontotemporal dementia, Dementia with Lewy bodies, Corticobasal degeneration, Progressive supranuclear palsy, Prion disorders causing neurodegeneration, Multiple system atrophy, Hereditary spastic paraparesis, Friedreich ataxia, Non-Friedreich ataxia, Spinocerebellar atrophies, Amyloidoses, Metabolic-related neurodegenerative disorders, Toxin-related neurodegenerative disorders, Multiple sclerosis, Charcot Marie Tooth, Diabetic neuropathy, Metabolic neuropathies, Endocrine neuropathies, Creutzfeldt-Jacob Disease, Primary progressive aphasia, Frontotemporal Lobar Degeneration, Cortical blindness, Shy-Drager Syndrome, Diffuse cerebral cortical atrophy of non-Alzheimer type, Lewy-body dementia, Pick disease, Thalamic degeneration, Mesolimbocortical dementia of non-Alzheimer type, Nonhuntingtonian types of chorea and dementia, Cortical-striatal-spinal degeneration, Dementia-Parkinson-amyotrophic lateral sclerosis complex, Cerebrocerebellar degeneration, Cortico-basal ganglionic degeneration, Familial dementia with spastic paraparesis or myoclonus, Tourette syndrome, or viral infection.

Additional examples of neurodegenerative diseases or disorders include, for example, polyglutamine repeat disorders such as Spinocerebellar ataxias (e.g., types 1, 2, 3, 6, 7 and 17), Machado-Joseph disease, Spinal and Bulbar muscular atrophy (SBMA or Kennedy's disease), Dentatorubral Pallidoluysian Atrophy (DRPLA) and other neurological conditions arising from polyglutamine expansions, or disease arising from non-coding DNA repeat expansions such as Fragile X syndrome, Fragile XE mental retardation, Friedreich ataxia, myotonic dystrophy, Spinocerebellar ataxias (types 8, 10 and 12) or other neurodegenerative diseases such as spinal muscular atrophy (Werdnig-Hoffman disease, Kugelberg-Welander disease), and spongiform encephalopathies.

Additional neurodegenerative diseases for which agents inhibiting 15-PGDH can be useful include, for example, age-related memory impairment, agyrophilic grain dementia, Parkinsonism-dementia complex of Guam, auto-immune conditions (eg Guillain-Barre syndrome, Lupus), Biswanger's disease, brain and spinal tumors (including neurofibromatosis), cerebral amyloid angiopathies (Journal of Alzheimer's Disease vol 3, 65-73 (2001)), cerebral palsy, chronic fatigue syndrome, corticobasal degeneration, conditions due to developmental dysfunction of the CNS parenchyma, conditions due to developmental dysfunction of the cerebrovasculature, dementia—multi infarct, dementia—subcortical, dementia with Lewy bodies, dementia of human immunodeficiency virus (HIV), dementia lacking distinct histology, Dementia Pugilistica, diffies neurofibrillary tangles with calcification, diseases of the eye, ear and vestibular systems involving neurodegeneration (including macular degeneration and glaucoma), dyskinesias (Paroxysmal), dystonias, essential tremor, Fahr's syndrome, fronto-temporal dementia and Parkinsonism linked to chromosome 17 (FTDP-17), frontotemporal lobar degeneration, frontal lobe dementia, hepatic encephalopathy, hereditary spastic paraplegia, hydrocephalus, pseudotumor cerebri and other conditions involving CSF dysffunction, Gaucher's disease, Hallervorden-Spatz disease, Korsakoffs syndrome, mild cognitive impairment, monomeric amyotrophy, motor neuron diseases, multiple system atrophy, multiple sclerosis and other demyelinating conditions (e.g., leukodystrophies), myalgic encephalomyelitis, myoclonus, neurodegeneration induced by chemicals, drugs and toxins, neurological manifestations of AIDS including AIDS dementia, neurological/cognitive manifestations and consequences of bacterial and/or virus infections, including but not restricted to enteroviruses, Niemann-Pick disease, non-Guamanian motor neuron disease with neurofibrillary tangles, non-ketotic hyperglycinemia, olivo-ponto cerebellar atrophy, oculopharyugeal muscular dystrophy, neurological manifestations of Polio myelitis including non-paralytic polio and post-polio-syndrome, primary lateral sclerosis, prion diseases including Creutzfeldt-Jakob disease (including variant form), kuru, fatal familial insomnia, Gerstmann-Straussler-Scheinker disease and other transmissible spongiform encephalopathies, prion protein cerebral amyloid angiopathy, postencephalitic Parkinsonism, progressive muscular atrophy, progressive bulbar palsy, progressive subcortical gliosis, progressive supranuclear palsy, restless leg syndrome, Rett syndrome, Sandhoff disease, spasticity, sporadic fronto-temporal dementias, striatonigral degeneration, subacute sclerosing panencephalitis, sulphite oxidase deficiency, Sydenham's chorea, tangle only dementia, Tay-Sach's disease, Tourette's syndrome, vascular dementia, and Wilson disease.

Additional neurodegenerative diseases for which 15-PGDH inhibitors are useful include other dementias not listed above, such as but without limitation, other mixed dementia, frontotemporal dementia, progressive supranuclear palsy (PSP), Parkinson's Disease with associated dementia, corticobasal degeneration, multiple system atrophy, HIV-induced dementia, white matter disease-associated dementias, mild cognitive impairment (MCI).

In some embodiments, the 15-PGDH inhibitors described herein are useful in preventing and treating blood brain barrier (BBB) permeability in a subject. Additional examples of diseases and disorders where BBB permeability occurs include, for example, multiple sclerosis, cerebral amyloid angiopathy, diabetic retinopathy, prion disorders, amyotrophic lateral sclerosis (ALS), Stiff-person Syndrome, Spinocerebellar Ataxias, Friedreich Ataxia, Ataxia Telangiectasia, Bulbospinal Atrophy (Kennedy Syndrome), Spinal Muscular Atrophy, Neuronal storage diseases (lipofuscinoses), Mitochondrial encephalomyopathies, Leukodystrophies, Neural sequelae of spinal shock/blunt trauma, Hypertensive Cerebrovascular disease, such as Lacunar Infarcts, Slit hemorrhages, Hypertensive encephalopathy. BBB permeability also occurs in brain tumors such as those with ‘sinusoidal’ (aka high nutrient) vascular supply.

BBB permeability also occurs in neurological sequellae associated with Streptococcal infections; pediatric autoimmune neuropsychiatric disorders associated with streptococcal infections (PANDAS), Tourette's syndrome, obsessive compulsive disease (OCD). BBB permeability also occurs in the following diseases and disorders; post-anesthesia neuropsychological dysfunction and neuropsychiatric disorders associated with any vasculopathy (e.g., Lupus, hypertension, etc.). BBB permeability also occurs in subjects with infections (either entry into the CNS, propagation within the CNS, or exit from the CNS upon established infection), for example, HIV, Tertiary Syphilis, Neuroborreliosis (Lyme Disease), Herpes Simplex Virus Type 1 (HSV-1)/Herpes Simplex Virus Type 2 (HSV-2), Varicella-Zoster Virus (Herpes Zoster), Cytomegalovirus, Poliomyelitis, Rabies, Progressive Multifocal Leukoencephalopathy, Subacute Sclerosing Panencephalitis (post-measles), Protozoal diseases (toxoplasmosis, amebiasis, and trypanosomiasis), Rickettsial infections (typhus and Rocky Mountain spotted fever), Metazoal diseases, Malaria, and Encephalitis/Meningitis. In some embodiments, encephalitis/meningitis results from sepsis.

In some embodiments, 15-PGDH inhibitors described herein are useful in preventing and/or treating BBB permeability occurring in subjects with or at risk of autism. Autism is a largely heritable disorder that is hallmarked by the expression of social deficits, language abnormalities and stereotyped, repetitive behaviors (American Psychiatric Association, 1994). Neuropathological and neuroimaging studies have reported increased brain size and weight (Bailey et al., 1998; Kemper and Bauman, 1998; Sparks et al., 2002; Herbert et al., 2003; Palmen et al., 2004). Many studies of autistic brains have reported an overall reduction in cell size and an increased cell packing density, especially in the hippocampus, subiculum and amygdala (Kemper and Bauman, 1993), indicating resident neurons have small dendritic trees and possible incomplete maturation or arrested development.

In some embodiments, the 15-PGDH inhibitors described herein can be provided in a pharmaceutical composition. A pharmaceutical composition containing the 15-PGDH inhibitors described herein as an active ingredient may be manufactured by mixing the derivative with a pharmaceutically acceptable carrier(s) or an excipient(s) or diluting the 15-PGDH inhibitors with a diluent in accordance with conventional methods. The pharmaceutical composition may further contain fillers, anti-cohesives, lubricants, wetting agents, flavoring agents, emulsifying agents, preservatives and the like. The pharmaceutical composition may be formulated into a suitable formulation in accordance with the methods known to those skilled in the art so that it can provide an immediate, controlled or sustained release of the 15-PGDH inhibitors after being administered into a mammal.

In some embodiments, the pharmaceutical composition may be formulated into a parenteral or oral dosage form. The solid dosage form for oral administration may be manufactured by adding excipient, if necessary, together with binder, disintegrants, lubricants, coloring agents, and/or flavoring agents, to the 15-PGDH inhibitors and shaping the resulting mixture into the form of tablets, sugar-coated pills, granules, powder or capsules. The additives that can be added in the composition may be ordinary ones in the art. For example, examples of the excipient include lactose, sucrose, sodium chloride, glucose, starch, calcium carbonate, kaolin, microcrystalline cellulose, silicate and the like. Exemplary binders include water, ethanol, propanol, sweet syrup, sucrose solution, starch solution, gelatin solution, carboxymethylcellulose, hydroxypropyl cellulose, hydroxypropyl starch, methylcellulose, ethylcellulose, shellac, calcium phosphonate and polypyrrolidone. Examples of the disintegrant include dry starch, sodium arginate, agar powder, sodium bicarbonate, calcium carbonate, sodium lauryl sulfate, stearic monoglyceride and lactose. Further, purified talc, stearates, sodium borate, and polyethylene glycol may be used as a lubricant; and sucrose, bitter orange peel, citric acid, tartaric acid, may be used as a flavoring agent. In some embodiments, the pharmaceutical composition can be made into aerosol formulations (e.g., they can be nebulized) to be administered via inhalation.

The 15-PGDH inhibitors described herein may be combined with flavoring agents, buffers, stabilizing agents, and the like and incorporated into oral liquid dosage forms such as solutions, syrups or elixirs in accordance with conventional methods. One example of the buffers may be sodium citrate. Examples of the stabilizing agents include tragacanth, acacia and gelatin.

In some embodiments, the 15-PGDH inhibitors described herein may be incorporated into an injection dosage form, for example, for a subcutaneous, intramuscular or intravenous route by adding thereto pH adjusters, buffers, stabilizing agents, relaxants, topical anesthetics. Examples of the pH adjusters and the buffers include sodium citrate, sodium acetate and sodium phosphate. Examples of the stabilizing agents include sodium pyrosulfite, EDTA, thioglycolic acid and thiolactic acid. The topical anesthetics may be procaine HCl, lidocaine HCl and the like. The relaxants may be sodium chloride, glucose and the like.

In other embodiments, the 15-PGDH inhibitors described herein may be incorporated into suppositories in accordance with conventional methods by adding thereto pharmaceutically acceptable carriers that are known in the art, for example, polyethylene glycol, lanolin, cacao butter or fatty acid triglycerides, if necessary, together with surfactants such as Tween.

The pharmaceutical composition may be formulated into various dosage forms as discussed above and then administered through various routes including an oral, inhalational, transdermal, subcutaneous, intravenous or intramuscular route. The dosage can be a pharmaceutically or therapeutically effective amount.

Therapeutically effective dosages of 15-PDGH inhibitor is one that reduces activity and/or expression of 15-PGDH, generates the maximum protective effect in preventing a neurodegenerative disease or disorder, or reduces a symptom of a neurodegenerative disease or disorder.

In some embodiments, an optimum dosage of the 15-PGDH inhibitor is one generating the maximum beneficial effect on damaged tissue. An effective dosage causes at least a statistically or clinically significant attenuation of at least one marker, symptom, or histological evidence characteristic of neurodegenerative condition, disease, or disorder. Markers, symptoms and histological evidence characteristic of neurodegenerative condition, disease, or disorder include memory loss, confusion, disturbances in axonal transport, demyelination, induction of metalloproteinases (MMPs), activation of glial cells, infiltration of lymphocytes, edema and immunological reactions that lead to tissue damage and further vascular injury. Stabilization of symptoms or diminution of tissue damage, under conditions wherein control patients or animals experience a worsening of symptoms or tissue damage, is one indicator of efficacy of a suppressive treatment.

Therapeutically effective dosage amounts of the 15-PGDH inhibitor may be present in varying amounts in various embodiments. For example, in some embodiments, a therapeutically effective amount of the 15-PGDH inhibitor may be an amount ranging from about 10-1000 mg (e.g., about 20 mg-1,000 mg, 30 mg-1,000 mg, 40 mg-1,000 mg, 50 mg-1,000 mg, 60 mg-1,000 mg, 70 mg-1,000 mg, 80 mg-1,000 mg, 90 mg-1,000 mg, about 10-900 mg, 10-800 mg, 10-700 mg, 10-600 mg, 10-500 mg, 100-1000 mg, 100-900 mg, 100-800 mg, 100-700 mg, 100-600 mg, 100-500 mg, 100-400 mg, 100-300 mg, 200-1000 mg, 200-900 mg, 200-800 mg, 200-700 mg, 200-600 mg, 200-500 mg, 200-400 mg, 300-1000 mg, 300-900 mg, 300-800 mg, 300-700 mg, 300-600 mg, 300-500 mg, 400 mg-1,000 mg, 500 mg-1,000 mg, 100 mg-900 mg, 200 mg-800 mg, 300 mg-700 mg, 400 mg-700 mg, and 500 mg-600 mg). In some embodiments, the 15-PGDH inhibitor is present in an amount of or greater than about 10 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg. In some embodiments, the 15-PGDH inhibitor is present in an amount of or less than about 1000 mg, 950 mg, 900 mg, 850 mg, 800 mg, 750 mg, 700 mg, 650 mg, 600 mg, 550 mg, 500 mg, 450 mg, 400 mg, 350 mg, 300 mg, 250 mg, 200 mg, 150 mg, or 100 mg.

In other embodiments, a therapeutically effective dosage amount may be, for example, about 0.001 mg/kg weight to 500 mg/kg weight, e.g., from about 0.001 mg/kg weight to 400 mg/kg weight, from about 0.001 mg/kg weight to 300 mg/kg weight, from about 0.001 mg/kg weight to 200 mg/kg weight, from about 0.001 mg/kg weight to 100 mg/kg weight, from about 0.001 mg/kg weight to 90 mg/kg weight, from about 0.001 mg/kg weight to 80 mg/kg weight, from about 0.001 mg/kg weight to 70 mg/kg weight, from about 0.001 mg/kg weight to 60 mg/kg weight, from about 0.001 mg/kg weight to 50 mg/kg weight, from about 0.001 mg/kg weight to 40 mg/kg weight, from about 0.001 mg/kg weight to 30 mg/kg weight, from about 0.001 mg/kg weight to 25 mg/kg weight, from about 0.001 mg/kg weight to 20 mg/kg weight, from about 0.001 mg/kg weight to 15 mg/kg weight, from about 0.001 mg/kg weight to 10 mg/kg weight.

In still other embodiments, a therapeutically effective dosage amount may be, for example, about 0.0001 mg/kg weight to 0.1 mg/kg weight, e.g. from about 0.0001 mg/kg weight to 0.09 mg/kg weight, from about 0.0001 mg/kg weight to 0.08 mg/kg weight, from about 0.0001 mg/kg weight to 0.07 mg/kg weight, from about 0.0001 mg/kg weight to 0.06 mg/kg weight, from about 0.0001 mg/kg weight to 0.05 mg/kg weight, from about 0.0001 mg/kg weight to about 0.04 mg/kg weight, from about 0.0001 mg/kg weight to 0.03 mg/kg weight, from about 0.0001 mg/kg weight to 0.02 mg/kg weight, from about 0.0001 mg/kg weight to 0.019 mg/kg weight, from about 0.0001 mg/kg weight to 0.018 mg/kg weight, from about 0.0001 mg/kg weight to 0.017 mg/kg weight, from about 0.0001 mg/kg weight to 0.016 mg/kg weight, from about 0.0001 mg/kg weight to 0.015 mg/kg weight, from about 0.0001 mg/kg weight to 0.014 mg/kg weight, from about 0.0001 mg/kg weight to 0.013 mg/kg weight, from about 0.0001 mg/kg weight to 0.012 mg/kg weight, from about 0.0001 mg/kg weight to 0.011 mg/kg weight, from about 0.0001 mg/kg weight to 0.01 mg/kg weight, from about 0.0001 mg/kg weight to 0.009 mg/kg weight, from about 0.0001 mg/kg weight to 0.008 mg/kg weight, from about 0.0001 mg/kg weight to 0.007 mg/kg weight, from about 0.0001 mg/kg weight to 0.006 mg/kg weight, from about 0.0001 mg/kg weight to 0.005 mg/kg weight, from about 0.0001 mg/kg weight to 0.004 mg/kg weight, from about 0.0001 mg/kg weight to 0.003 mg/kg weight, from about 0.0001 mg/kg weight to 0.002 mg/kg weight. In some embodiments, the therapeutically effective dose may be 0.0001 mg/kg weight, 0.0002 mg/kg weight, 0.0003 mg/kg weight, 0.0004 mg/kg weight, 0.0005 mg/kg weight, 0.0006 mg/kg weight, 0.0007 mg/kg weight, 0.0008 mg/kg weight, 0.0009 mg/kg weight, 0.001 mg/kg weight, 0.002 mg/kg weight, 0.003 mg/kg weight, 0.004 mg/kg weight, 0.005 mg/kg weight, 0.006 mg/kg weight, 0.007 mg/kg weight, 0.008 mg/kg weight, 0.009 mg/kg weight, 0.01 mg/kg weight, 0.02 mg/kg weight, 0.03 mg/kg weight, 0.04 mg/kg weight, 0.05 mg/kg weight, 0.06 mg/kg weight, 0.07 mg/kg weight, 0.08 mg/kg weight, 0.09 mg/kg weight, or 0.1 mg/kg weight. The effective dose for a particular individual can be varied (e.g., increased or decreased) over time, depending on the needs of the individual.

In some embodiments, a therapeutically effective dosage may be a dosage of 10 μg/kg/day, 50 μg/kg/day, 100 μg/kg/day, 250 μg/kg/day, 500 μg/kg/day, 1000 μg/kg/day or more. In various embodiments, the amount of the 15-PGDH inhibitor or pharmaceutical salt thereof is sufficient to provide a dosage to a patient of between 0.01 μg/kg and 10 μg/kg; 0.1 μg/kg and 5 μg/kg; 0.1 μg/kg and 1000 μg/kg; 0.1 μg/kg and 900 μg/kg; 0.1 μg/kg and 900 μg/kg; 0.1 μg/kg and 800 μg/kg; 0.1 μg/kg and 700 μg/kg; 0.1 μg/kg and 600 μg/kg; 0.1 μg/kg and 500 μg/kg; or 0.1 μg/kg and 400 μg/kg.

Particular doses or amounts to be administered in accordance with the present invention may vary, for example, depending on the nature and/or extent of the desired outcome, on particulars of route and/or timing of administration, and/or on one or more characteristics (e.g., weight, age, personal history, genetic characteristic, lifestyle parameter, severity of cardiac defect and/or level of risk of cardiac defect, etc., or combinations thereof). Such doses or amounts can be determined by those of ordinary skill. In some embodiments, an appropriate dose or amount is determined in accordance with standard clinical techniques. For example, in some embodiments, an appropriate dose or amount is a dose or amount sufficient to reduce a disease severity index score by 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100% or more. For example, in some embodiments, an appropriate dose or amount is a dose or amount sufficient to reduce a disease severity index score by 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100%. Alternatively or additionally, in some embodiments, an appropriate dose or amount is determined through use of one or more in vitro or in vivo assays to help identify desirable or optimal dosage ranges or amounts to be administered.

Various embodiments may include differing dosing regimen. In some embodiments, the 15-PGDH inhibitor can be administered via continuous infusion. In some embodiments, the continuous infusion is intravenous. In other embodiments, the continuous infusion is subcutaneous. Alternatively or additionally, in some embodiments, the 15-PGDH inhibitor can be administered bimonthly, monthly, twice monthly, triweekly, biweekly, weekly, twice weekly, thrice weekly, daily, twice daily, or on another clinically desirable dosing schedule. The dosing regimen for a single subject need not be at a fixed interval, but can be varied over time, depending on the needs of the subject.

For topical application, the composition can be administered in the form of aqueous, alcoholic, aqueous-alcoholic or oily solutions or suspensions, or of a dispersion of the lotion or serum type, of emulsions that have a liquid or semi-liquid consistency or are pasty, obtained by dispersion of a fatty phase in an aqueous phase (O/W) or vice versa (W/O) or multiple emulsions, of a free or compacted powder to be used as it is or to be incorporated into a physiologically acceptable medium, or else of microcapsules or microparticles, or of vesicular dispersions of ionic and/or nonionic type. It may thus be in the form of a salve, a tincture, milks, a cream, an ointment, a powder, a patch, an impregnated pad, a solution, an emulsion or a vesicular dispersion, a lotion, aqueous or anhydrous gels, a spray, a suspension, a shampoo, an aerosol or a foam. It may be anhydrous or aqueous. It may also comprise solid preparations constituting soaps or cleansing cakes.

Pharmaceutical compositions including the 15-PGDH inhibitor described herein can additionally contain, for example, at least one compound chosen from prostaglandins, in particular prostaglandin PGE₁, PGE₂, their salts, their esters, their analogues and their derivatives, in particular those described in WO 98/33497, WO 95/11003, JP 97-100091, JP 96-134242, in particular agonists of the prostaglandin receptors. It may in particular contain at least one compound such as the agonists (in acid form or in the form of a precursor, in particular in ester form) of the prostaglandin F₂α receptor, such as for example latanoprost, fluprostenol, cloprostenol, bimatoprost, unoprostone, the agonists (and their precursors, in particular the esters such as travoprost) of the prostaglandin E₂ receptors such as 17-phenyl PGE₂, viprostol, butaprost, misoprostol, sulprostone, 16,16-dimethyl PGE₂, 11-deoxy PGE₁, 1-deoxy PGE₁, the agonists and their precursors, in particular esters, of the prostacycline (IP) receptor such as cicaprost, iloprost, isocarbacycline, beraprost, eprostenol, treprostinil, the agonists and their precursors, in particular the esters, of the prostaglandin D₂ receptor such as BW245C ((4S)-(3-[(3R,S)-3-cyclohexyl-3-isopropyl]-2,5-dioxo)-4-imidazolidinehept-anoic acid), BW246C ((4R)-(3-[(3R,S)-3-cyclohexyl-3-isopropyl]-2,5-dioxo)-4-imidazolidinehept-anoic acid), the agonists and their precursors, in particular the esters, of the receptor for the thromboxanes A2 (TP) such as I-BOP ([1S-[1a,2a(Z), 3b(1E,3S),4a]]-7-[3-[3-hydroxy-4-[4-(iodophenoxy)-1-butenyl]-7-oxabicyclo-[2.2.1]hept-2-yl]-5-heptenoic acid).

Advantageously, the composition can include at least one 15-PGDH inhibitor as defined above and at least one prostaglandin or one prostaglandin derivative such as for example the prostaglandins of series 2 including in particular PGF_{2α} and PGE₂ in saline form or in the form of precursors, in particular of the esters (example isopropyl esters), their derivatives such as 16,16-dimethyl PGE₂, 17-phenyl PGE₂ and 16,16-dimethyl PGF_{2α} 17-phenyl PGF_{2α}, prostaglandins of series 1 such as 11-deoxyprostaglandin E1, 1-deoxyprostaglandin E1 in saline or ester form, is their analogues, in particular latanoprost, travoprost, fluprostenol, unoprostone, bimatoprost, cloprostenol, viprostol, butaprost, misoprostol, their salts or their esters.

The invention is further illustrated by the following examples, which is not intended to limit the scope of the claims.

### Examples

### Summary of Eicosanoid Profiling in Brains of Mice in Different Experiment Conditions

Eicosanoids were measured by methods of liquid chromatography with tandem mass spectrometry with the use of spiked in deuterated internal standards. Eicosanoids measured included: the 6-keto-PGF1 alpha (6-keto-PGF1a, a hydrolysis product of and proxy for prostacyclin), thromboxane B2 (TXB2, a metabolite/product of and proxy for thromboxane A2), PGD2, PGE2, 15-keto-PGE2 (produced by 15-PGDH degradation of PGE2), tetranor PGE1 (TN-E, a metabolic product produced by beta-oxidation of PGE2), PGJ2, PGF2 alpha (PGF2a), LTB4, 15-HETE, 12-HETE, 8-HETE, 5-HETE, 17-HDHA, 12,13-DiHOME, 14,15-DHET, 11,12-DHET.

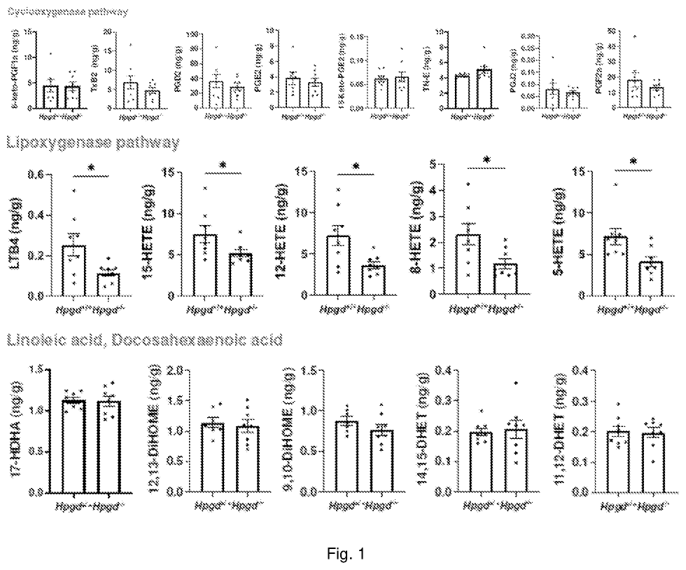

FIG. 1 illustrates graphs showing comparisons of eicosanoid values in brains of 15-PGDH wild-type (Hpgd^{+/+}) versus 15-PGDH knockout (Hpgd^{-/-}) male mice. 10-11 week old male mice on an FVB background were employed for study. The right hemispheres of the brains were snap frozen and powdered, and eicosanoids were extracted in organic solvent for analysis. Values were normalized to the wet weight of the companion left brain hemispheres and are recorded as ng eicosanoid per gm wet weight of tissue. Significant decreases were noted in the brains of knockout mice for LTB4, 15-HETE, 12-HETE, 8-HETE, 5-HETE.

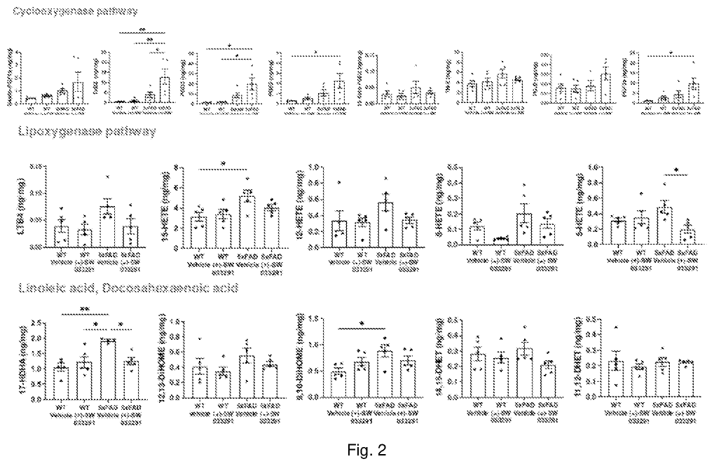

FIG. 2 illustrates graphs showing comparison of eicosanoid values in brain of 6 month old male mice that are either wild-type or that are the 5×FAD strain of mice that model Alzheimer's disease. Beginning at 5 months of age both genotypes of mice were injected twice daily with either 5 mk/kg (+)-SW033291 or with matched vehicle control as described in Zhang et al. Science. 2015; 348(6240):aaa2340. PMCID: PMC4481126. Treatment continued twice daily for 4 weeks. Mice were sacrificed at 3 hours following their final injection. After sacrifice the left hippocampus was dissected, flash frozen, and powdered, and eicosanoids were extracted in aqueous buffer for analysis. Protein concentration of the extracts were assayed, and eicosanoid values were recorded as ng eicosanoid per mg protein. Observations include that (+)-SW033291 treated mice wild-type mice showed trends for increases in 6-keto-PGF1a, PGD2, PGE2, PGF2a, and 9,10-DiHOME, versus vehicle treated mice. Additionally, versus wild-type mice, 5×FAD mice showed trends for increases in 6-keto-PGF1a, TXB2, PGD2, PGE2, 15-keto-PGE2, TN-E, PGF2a, LTB4, 15-HETE, 12-HETE, 8-HETE, 5-HETE, 12,13-DiHome, and showed significant increases in 17-HDHA and 9,10-DiHOME. Additionally, versus vehicle control treated 5×FAD mice, (+)-SW033291 treated mice showed a trend to increased 6-keto-PGF1a, PGD2, PGE2, PGJ2, PGF2a, and showed significant increase in TXB2.

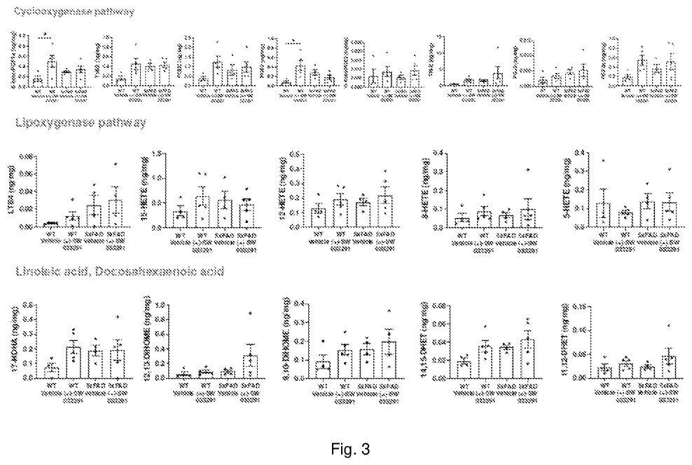

FIG. 3 illustrates graphs showing comparison of eicosanoid values in brain of 6 month old male mice that are either wild-type or that are the 5×FAD strain of mice that model Alzheimer's disease. Beginning at 5 months of age both genotypes of mice were injected twice daily with either 5 mk/kg (+)-SW033291 or with matched vehicle control as described in Zhang et al. Science. 2015; 348(6240):aaa2340. PMCID: PMC4481126. Treatment continued twice daily for 4 weeks. Mice were sacrificed at 3 hours following their final injection. After sacrifice the right hemisphere of the brain was dissected, flash frozen, and powdered, and eicosanoids were extracted in aqueous buffer for analysis. Protein concentration of the extracts were assayed, and eicosanoid values were recorded as ng eicosanoid per mg protein. Observations include that compared to vehicle treated wild-type mice, (+) SW033291 treated mice showed a trend to increased TXB2, PGD2, 15-keto-PGE2, TN-E, PGJ2, PGF2a, LTB4, 15-HETE, 12-HETE, 8-HETE, 17-HDHA, 9,10-DiHOME, 14,15-DHET, and showed significant increase in 6-keto-PGF1a, PGE2. Additionally, versus wild-type mice, 5×FAD mice showed trends for increases in 6-keto-PGF1a, TXB2, PGD2, PGE2, TN-E, PGJ2, PGF2a, LTB4, 15-HETE, 12-HETE, 17-HDHA, 9,10-DiHOME, 14,15-DHET. Additionally, versus vehicle control treated 5×FAD mice, (+)-SW033291 treated mice showed a trend to increased 15-keto-PGE2, TN-E, PGF2a, 12,13-DiHOME, and 11,12-DHET.

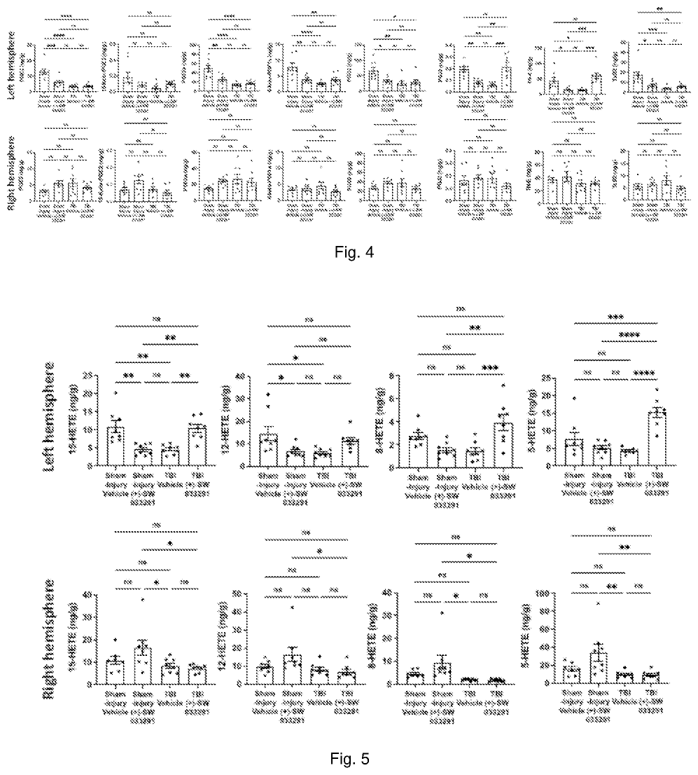

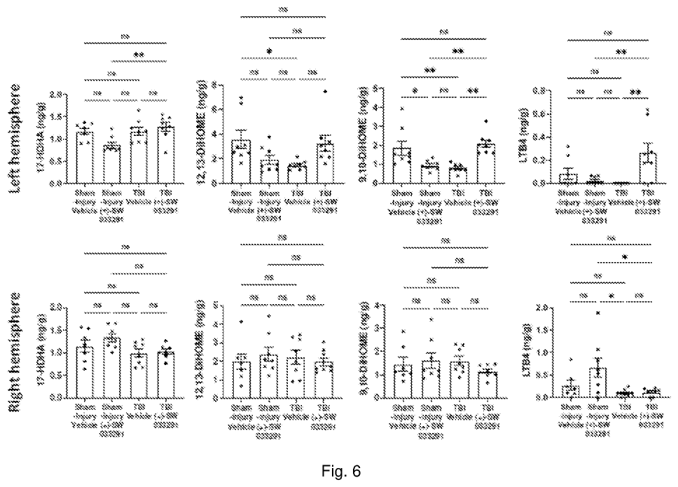

FIGS. 4-6 illustrate graphs showing comparisons of eicosanoid values in brain of wild-type male mice who at 8 weeks of age were subjected to sham or actual traumatic brain injury (TBI) delivered by blast wave to the left skull. 24 hours following sham or actual TBI treatment commenced by injection twice daily with either 5 mk/kg (+)-SW033291 or with matched vehicle control as described in Zhang et al. Science. 2015; 348(6240):aaa2340. PMCID: PMC4481126. Treatment continued for 21 days until mice reached 11 weeks of age. Three hours following the last injection, mice were sacrificed. After sacrifice the right or left hemisphere of the brain was dissected, flash frozen, and powdered, and eicosanoids were extracted in organic solvent for analysis. Values were normalized to the wet weight of the companion contralateral brain hemispheres and are recorded as ng eicosanoid per gm wet weight of tissue. Observations include that in the left hemisphere, compared to vehicle treated sham mice, (+) SW033291 treated sham mice showed significantly decreased PGE2, PGF2a, 6-keto-PGF1a, PGD2, PGJ2, TN-E, TxB2, 15-HETE, 12-HETE, 9,10-DiHOME, and trends of changes in other eicosonoids. Observations also include that in the right hemisphere, compared to vehicle treated sham mice, (+) SW033291 treated sham mice showed a trend toward increased PGE2, 15-keto-PGE2, PGF2a, TN-E, 15-HETE, 12-HETE, 8-HETE, 5-HETE, and LTB4.

Additionally, observations include that following TBI, compared to sham injury, that vehicle treated mice in the left hemisphere show significantly decreased PGE2, PGF2a, 6-keto-PGF1a, PGD2, PGJ2, TN-E, TxB2, 15-HETE, 12-HETE, 12,13-DiHOME, 9,10-DiHOME, and trends to changes in other eicosonoids. Observations also include that that following TBI, compared to sham injury, vehicle treated mice in the right hemisphere show a trend toward increased PGE2, increased PGF2a, increased PGD2, and increased TxB2.

Additionally, observations include that following TBI, (+)-SW033291 treated mice, compared to vehicle treated mice, in the left hemisphere show significantly increased PGJ2, TN-E, 15-HETE, 8-HETE, 5-HETE, 9,10-DiHOME, and LTB4, and trends to changes in other eicosonoids. Observations also include that following TBI, (+)-SW033291 treated mice, compared to vehicle treated mice, in the right hemisphere show trends toward decreased PGJ2, and TxB2.

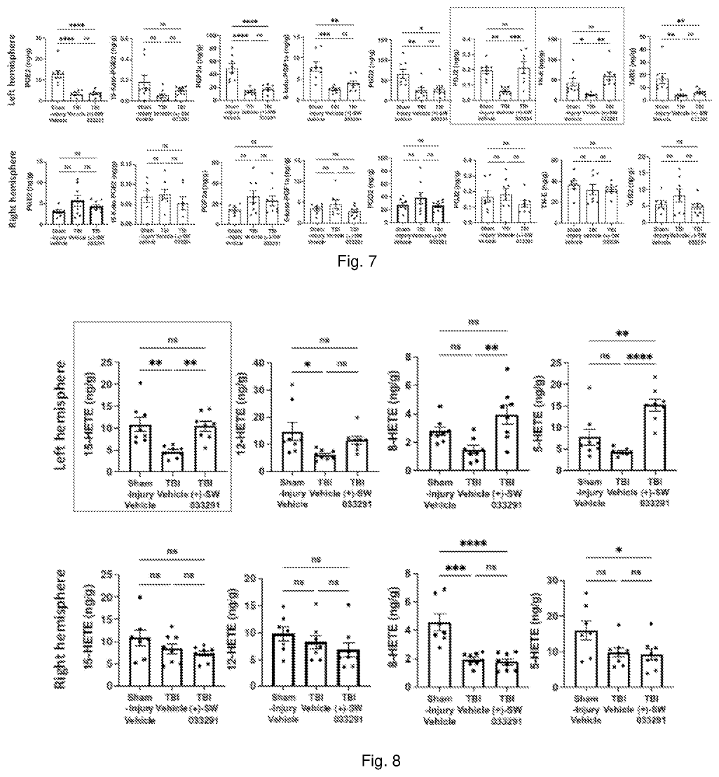

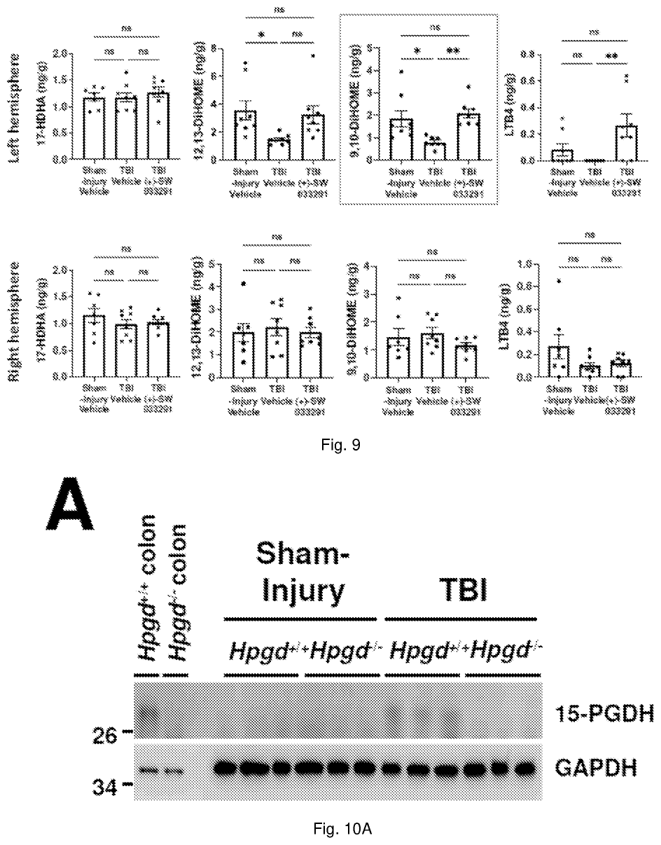

FIGS. 7-9 illustrate graphs that reprise the data of FIGS. 4-6 respectively, except highlighting comparison of post-TBI vehicle and (+)-SW033291 treated mice versus sham injury vehicle treated mice. Particularly highlighted are the decrease after TBI in vehicle treated mice, and the restoration by (+)-SW033291 treatment of TBI treated mice, of left hemisphere values of: PGJ2, TN-E, 15-HETE, and 9,10-DiHOME. It is noted that PGJ2 and 15-HETE are direct substrates of 15-PGDH.

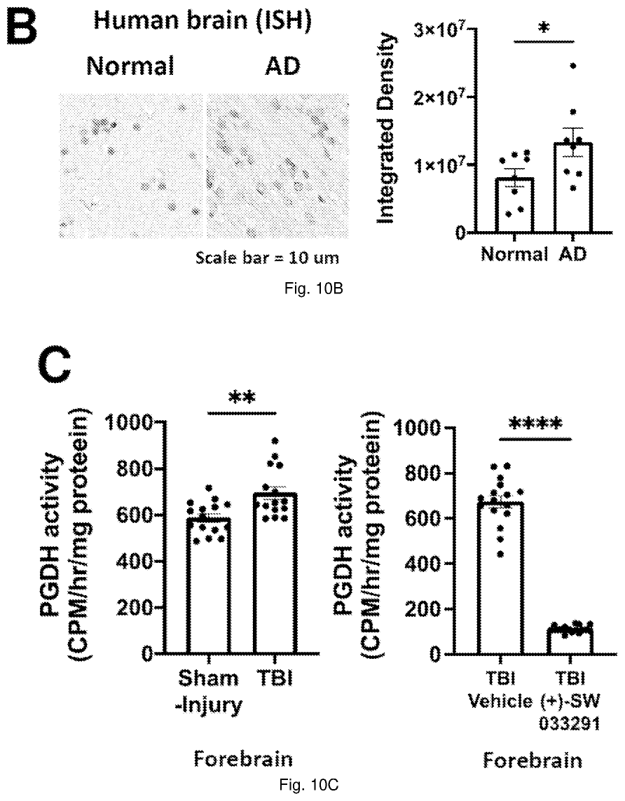

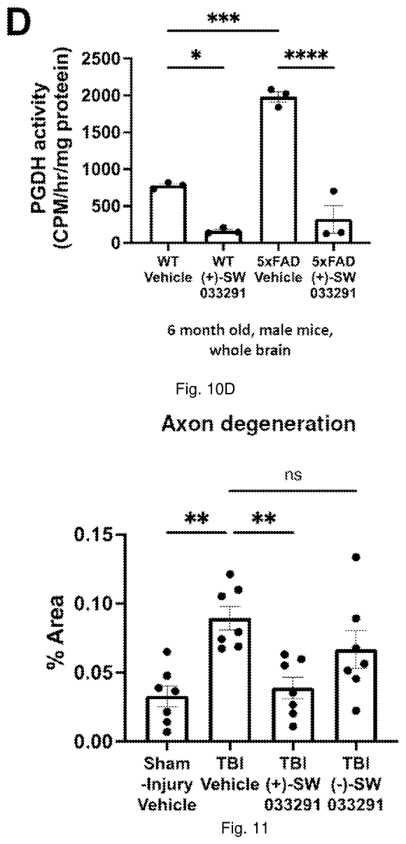

FIGS. 10(A-D) shows that 15-PGDH activity is pathologically elevated in mouse TBI and AD, and human AD, and in mouse TBI and AD is returned to normal by treatment with (+)-SW033291. (A) 8 week old 15-PGDH wild-type (Hpgd^{+/+}) and 15-PGDH knockout (Hpgd^{-/-}) mice were subjected to TBI, and brain tissue was dissected 2 weeks later for western blot. Hpgd^{+/+} mice showed TBI-dependent increase 15-PGDH expression in the brain, whereas 15-PGDH protein was absent in sham-injury and TBI Hpgd^{-/-} mice. (B) 15-PGDH expression was significantly elevated in human brain tissue from patients with Alzheimer's disease (AD), compared to age-matched non-AD patients. Individual human data points are shown on the graph. Data was analyzed with Student's t test. * p<0.05. All data is mean±SEM. (C) Hpgd^{+/+} mice were subjected to TBI at 8 weeks of age and then brain tissue was dissected 3 weeks later for biochemical determination of 15-PGDH enzymatic activity. 15-PGDH enzymatic activity in the forebrain was significantly increased by TBI, and restored to normal by treatment of mice with 5 mg/kg (+)-SW033291 started 24 hours after injury and continued throughout the duration of the experiment. Individual mouse data points are shown on the graph. Data was analyzed with Student's t test. ** p<0.01, **** p<0.0001. All data is mean±SEM. (D) 5×FAD mice and wild type littermates (WT) were administered either 5 mg/kg/day (+)-SW033291 twice daily (or vehicle) from 2 to 6 months of age, and then brains were dissected for biochemical assessment of 15-PGDH activity. Treatment of WT mice with (+)-SW033291 significantly lowered 15-PGDH activity in the whole brain, compared to WT mice treated with vehicle. 5×FAD mice treated with vehicle had significantly higher 15-PGDH activity than WT littermates treated with vehicle. Treatment of 5×FAD mice with (+)-SW033291 significantly lowered 15-PGDH activity in the whole brain, compared to 5×FAD mice treated with vehicle. Individual mouse data points are shown on the graph. Data was analyzed using two-way repeated measures ANOVA; * p<0.05, ** p<0.01, *** p<0.001. All data is mean±SEM.

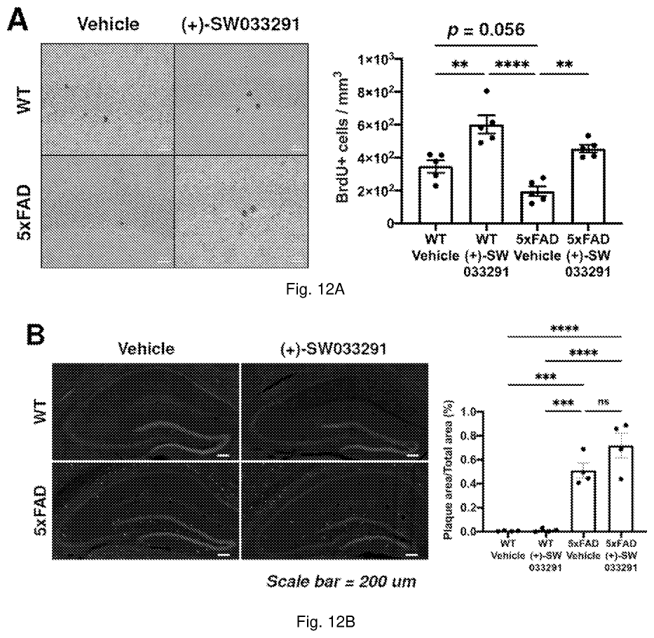

FIG. 11 illustrates treatment with (+)-SW033921 protects wild type mice from axon degeneration after TBI. 8 week old C57/B6 mice were exposed to sham-injury or TBI. Twice daily treatment with 5 mg/kg (+)-SW033291, 5 mg/kg (−)-SW033291, or vehicle was started 24 hours after injury and continued throughout the duration of the experiment. Animals were sacrificed by transcardial perfusion 3 weeks after TBI or sham-injury, and fixed brains were processed for silver staining. Increased silver staining indicates greater axon degeneration in TBI-vehicle compared to sham-injury-vehicle, while TBI-(+)-SW033291 showed reduced silver staining equivalent to sham-injury-vehicle, indicating protection from axonal degeneration. This protection was not noted in TBI-(−)-SW033291, consistent with the fact that this (−) enantiomer is inactive. Individual mouse data points are shown on the graph. Data was analyzed using two-way repeated measures ANOVA; * p<0.05, ** p<0.01. All data is mean±SEM.

FIGS. 12(A-B) illustrates treatment with (+)-SW033291 augments survival of newborn hippocampal neurons, thereby preserving hippocampal neurogenesis in 5×FAD mice, and does not affect accumulation of amyloid plaque in 5×FAD mice. Two month old 5×FAD and wild type (WT) littermates were treated with 5 mg/kg/day twice daily (+)-SW033291 (or vehicle) every day from 2 to 6 months. At 5 months of age, mice were injected with a single dose of 150 mg/kg bromodeoxyuridine (BrdU) to label replicating cells. At the age of 6 months (28 days after BrdU) mice were transcardially perfused and brains were dissected for examination of fixed tissue. (A) Immunohistochemical staining for BrdU showed that treatment with (+)-SW033291 augments the survival of newborn hippocampal neurons in both WT and 5×FAD mice. 5×FAD mice showed a strong trend towards decreased survival compared to WT, and 5×FAD mice treated with (+)-SW033291 showed survival of young hippocampal neurons to a comparable extent as WT-vehicle mice. (B) 5×FAD mice show accumulation of amyloid plaque (Congo Red staining), and WT mice do not show any amyloid accumulation. Treatment with (+)-SW033291 does not alter amyloid plaque accumulation. Individual mouse data points are shown on the graph. Data was analyzed using two-way repeated measures ANOVA; * p<0.05, ** p<0.01, ***p<0.001, ****p<0.0001. All data is mean±SEM.

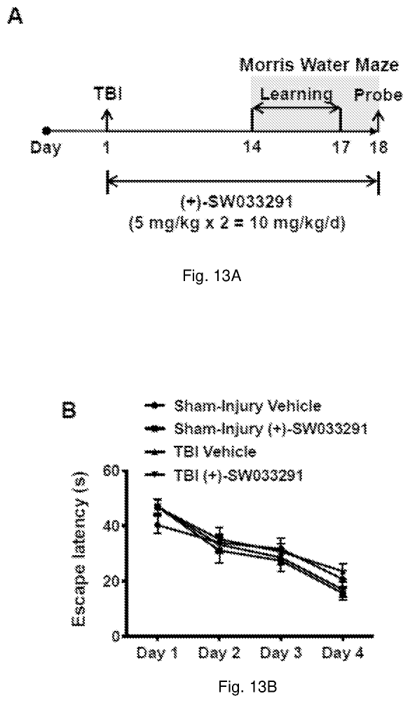

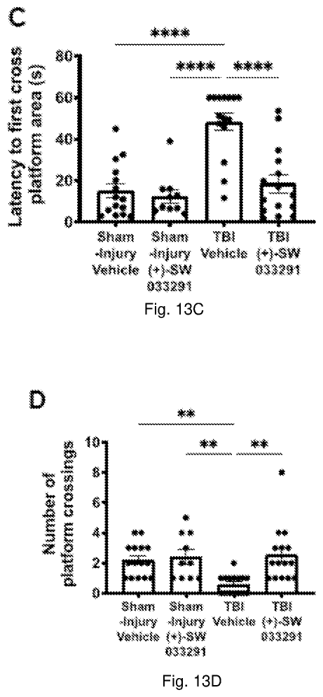

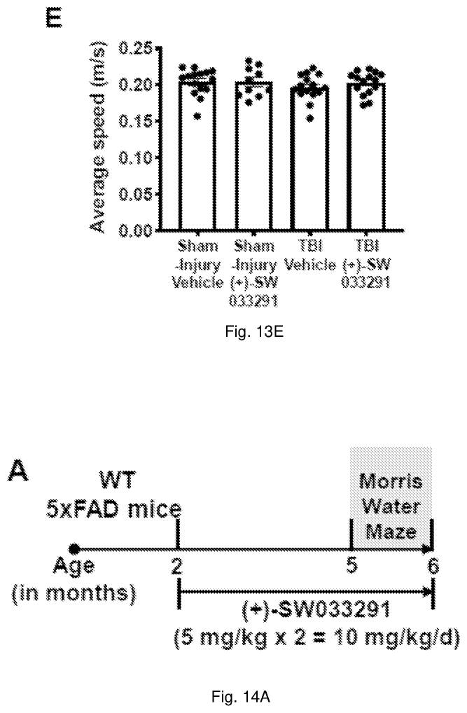

FIGS. 13(A-E) illustrate treatment with (+)-SW033921 protects wild type mice from cognitive impairment after TBI. (A) Experimental Timeline: 8 week old C57/B6 mice were exposed to sham-injury or TBI. Twice daily treatment with 5 mg/kg (+)-SW033291 or vehicle was started 24 hours after injury and continued throughout the duration of the experiment. Animals were tested for cognitive performance in the Morris water maze (MWM) 2 weeks after injury. (B) Animals were trained to find the hidden platform in the MWM for 4 consecutive days (4 trials/day), with performance measured as latency to find the hidden platform. TBI and sham-injured animals were able to learn the cognitive task over the 4-day learning period, as average (escape) latency to find the hidden platform was not different between groups and decreased steadily in successive sessions. (C, D) To probe for spatial memory of the learned location of the hidden platform, one day after the final learning day the animals were tested in the same MWM except the platform was removed. Memory was measured as latency to first cross the area where the platform was located and the number of times the animal crossed that area. TBI-injured animals treated with vehicle showed memory deficits compared to the (+)-SW033291-treated TBI group and sham-injured groups. Average latency to crossing the platform area significantly increased in the vehicle-treated TBI-injured group compared to all other groups, and average latency in the number of platform crosses significantly decreased in the vehicle-treated TBI-injured group compared to all other groups. (E) Average swim speed was not significantly different amongst groups, indicating that none of the mice experienced locomotor impairment. Individual mouse data points are shown on the graph. Data was analyzed using two-way repeated measures ANOVA; * p<0.05, ** p<0.01, *** p<0.001. All data is mean±SEM

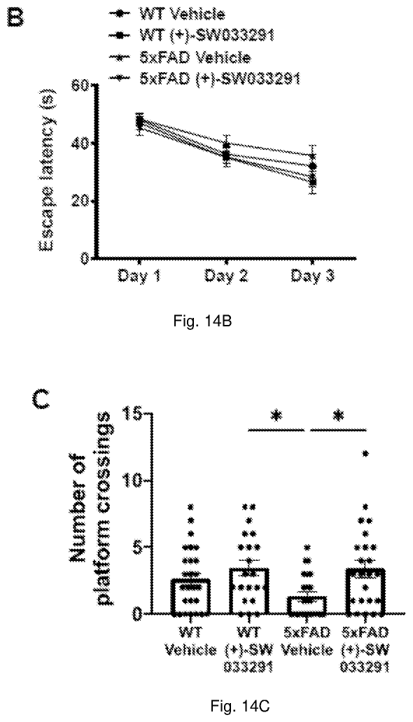

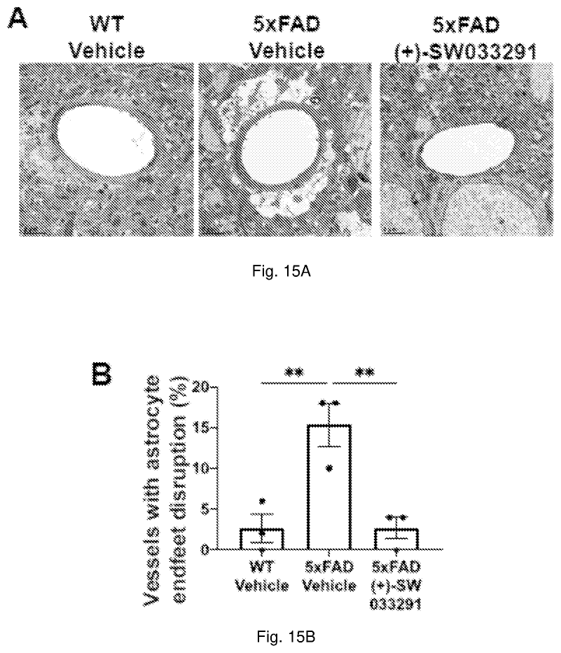

FIGS. 14(A-C) illustrate (+)-SW033921 protects cognitive function in 5×FAD mice. (A) Experimental Timeline: Two month old 5×FAD and wild type (WT) littermates were treated with 5 mg/kg/day twice daily (+)-SW033291 (or vehicle) every day from 2 to 6 months. Cognitive performance was tested using Morris water maze (MWM) as shown in the timeline. (B) Animals were trained to find the hidden platform in the MWM maze for 3 consecutive days (4 trials/day), and latency to find the hidden platform was measured. 5×FAD did not show any learning deficits when compared to WT mice. Average (escape) latency to find the hidden platform was not different between groups, and decreased steadily in line with the expected improved learning in successive sessions. (C) To probe for spatial memory of the hidden platform, one day after the last learning day the animals were tested in the same MWM, except the platform was removed. Memory was measured as latency to first cross the area where the platform was located and the number of times an animal crossed that area. 5×FAD mice treated with vehicle showed memory deficits, and treatment with (+)-SW033291 alleviated this deficit. The number of platform crosses was significantly decreased in the vehicle-treated 5×FAD group compared to the other groups. Individual mouse data points are shown on the graph. Data was analyzed using two-way repeated measures ANOVA; * p<0.05, ** p<0.01, *** p<0.001. All data is mean±SEM.

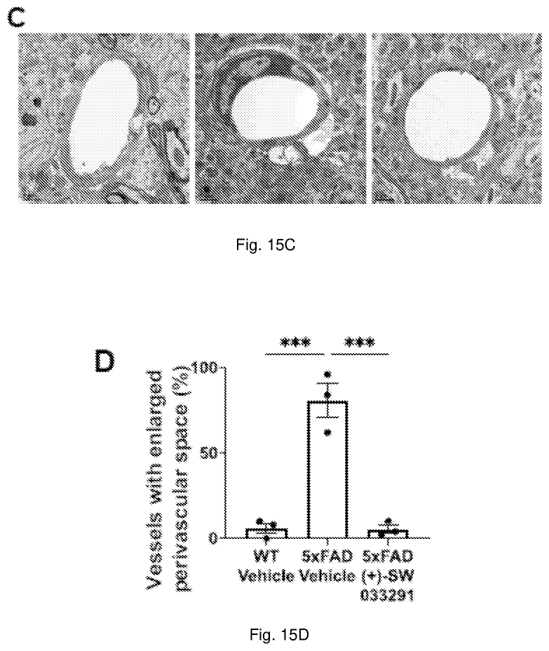

FIGS. 15(A-D) illustrate (+)-SW033921 protects the blood-brain barrier (BBB) in 5×FAD mice. BBB integrity was analyzed using transmission electron microscopy (TEM) in 6 month old 5×FAD mice. (A,B) Astrocyte end feet that envelope the BBB were disrupted in vehicle-treated 5×FAD mice (red arrows) while treatment with (+)-SW033291 prevented this disruption. (C,D) Enlargement of the perivascular space, another key feature of a damaged BBB, was observed in vehicle-treated 5×FAD mice (yellow arrow), while treatment with (+)-SW033291 prevented this disruption. Mice were treated with 5 mg/kg/day (+)-SW033291 (or vehicle) every day from 2 to 6 months of age, and then sacrificed for TEM analysis. Individual mouse data points are shown on the graph. Data was analyzed using one-way ANOVA; * p<0.05, ** p<0.01, *** p<0.001. All data is mean±SEM.

Thus, the data shows that inhibiting 15-PGDH stimulates hippocampal neurogenesis by increasing survival of new hippocampal neurons, as assessed by the persistence over time of new neurons labeled at the time of generations with BrdU. The data also shows that inhibiting 15-PGDH protects from TBI induced axonal degeneration, as assessed by reduced silver staining of degenerating neurons. The data shows that inhibiting 15-PGDH after TBI protects from TBI associated memory loss as assessed in the Morris water maze test, where post TBI the vehicle treated mice cross the old location of the prior safety platform fewer times and take longer before they find the old location, and where these defects are completely reversed in the treated mice. The data further shows that inhibiting 15-PGDH protects a genetic mouse model of Alzheimer's disease from loss of memory as assessed in the Morris water maze test and protects the blood brain barrier from disruption and increased permeability. Therefore, the data show the use of a 15-PGDH inhibitor can protect and treat memory and cognitive deficits consequent to neurodegenerative disease, and particularly support such use for protection from memory deficits in TBI and in Alzheimer's disease.

While this invention has been particularly shown and described with references to preferred embodiments thereof, it will be understood by those skilled in the art that various changes in form and details may be made therein without departing from the scope of the invention encompassed by the appended claims. All patents, publications and references cited in the foregoing specification are herein incorporated by reference in their entirety.

1-10. (canceled)

11: A method of treating and/or inhibiting memory loss and/or cognitive decline in a subject in need thereof, the method comprising:
administering to the subject a therapeutically effective amount of a 15-PGDH inhibitor.

12: The method of claim 11 wherein the subject has an enhanced or aberrant 15-PGDH activity in brain tissue.

13: The method of claim 11, wherein the memory loss and/or cognitive decline is associated with and/or caused by neurodegeneration and/or neurodegenerative condition, disease, or disorder and/or normal aging.

14: The method of claim 11, wherein the memory loss and/or cognitive decline is associated with a decrease or increase in the level of at least one of PGE2, 16-keto-PGE2, PGF2α, 6-keto-PGF1α, TXB2, PGD2, PGJ2, TN-E, 15-HETE, 12-HETE, 8-HETE, or 5-HETE in brain tissue of the subject.

15: The method of claim 11, wherein the memory loss and/or cognitive decline is associated is associated with a decrease in the level of at least one, at least two, or at least three or more of PGF2α, 6-keto-PGF1α, TXB2, PGD2, PGJ2, TN-E, 15-HETE, 12-HETE, 8-HETE, or 5-HETE in brain tissue of the subject.

16: The method of claim 14, wherein the brain tissue comprises the hippocampus of the subject.

17: The method of claim 11, wherein the 15-PGDH inhibitor can be administered at an amount effective to stimulate hippocampal neurogenesis.

18: The method of claim 11, wherein the memory loss and/or cognitive decline is associated with an abnormal blood brain barrier (BBB) in the subject.

19: The method of claim 18, wherein the abnormal BBB is a permeable blood brain barrier.

20: A method reducing blood brain barrier permeability in a subject in need thereof, the method comprising:
administering to the subject a therapeutically effective amount of a 15-PGDH inhibitor.

21: The method of claim 20, wherein the subject has or is at risk of a neurodegenerative condition, disorder, or disease.

22: The method of claim 20, wherein the subject has been identified with mild cognitive impairment, Alzheimer's disease, Lewy body dementia, Vascular dementia, Age-related dementia, Frontotemporal dementia, mixed dementia, Parkinson's disease, Huntington's disease, multiple sclerosis, diabetic retinopathy, prion disorders, or amyotrophic lateral sclerosis.

23: The method of claim 20, wherein the subject has a decrease or increase in the level of at least one of PGE2, 16-keto-PGE2, PGF2α, 6-keto-PGF1α, TXB2, PGD2, PGJ2, TN-E, 15-HETE, 12-HETE, 8-HETE, or 5-HETE brain tissue compared to a control.

24: The method of claim 20, wherein the subject has a decrease in the level of at least one, at least two, or at least three or more of PGF2α, 6-keto-PGF1α, TXB2, PGD2, PGJ2, TN-E, 15-HETE, 12-HETE, 8-HETE, or 5-HETE in brain tissue of the subject.

25: The method of claim 20, wherein the brain tissue comprises the hippocampus of the subject.

26: The method of claim 20, wherein the subject has memory loss and/or cognitive decline and the 15-PGDH inhibitor is administered at amount effective to improve memory and/or cognition.

27: The method of any of claims 1 to 26, wherein the 15-PGDH inhibitor can inhibit enzymatic activity of recombinant 15-PGDH at an IC50 of less than 1 μM.

28: The method of claim 11, wherein the 15-PGDH inhibitor has the following formula (V): 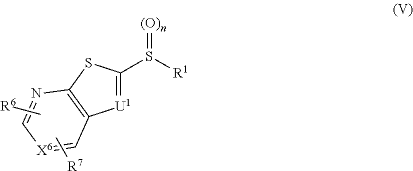
or a pharmaceutically acceptable salt, tautomer, or solvate thereof;
wherein n is 0-2
X⁶ is independently is N or CR^{c}
R¹, R⁶, R⁷, and R^{c} are the same or different each independently hydrogen or a substituted or unsubstituted group selected from C₁-C₂₄ alkyl, C₂-C₂₄ alkenyl, C₂-C₂₄ alkynyl, C₃-C₂₀ aryl, heteroaryl, heterocycloalkenyl containing from 5-6 ring atoms, C₆-C₂₄ alkaryl, C₆-C₂₄ aralkyl, halo, —Si(C₁-C₃ alkyl)₃, hydroxyl, sulfhydryl, C₁-C₂₄ alkoxy, C₂-C₂₄ alkenyloxy, C₂-C₂₄ alkynyloxy, C₅-C₂₀ aryloxy, acyl, acyloxy, C₂-C₂₄ alkoxycarbonyl, C₆-C₂₀ aryloxycarbonyl, C₂-C₂₄ alkylcarbonato, C₆-C₂₀ arylcarbonato, carboxy, carboxylato, carbamoyl, C₁-C₂₄ alkyl-carbamoyl, arylcarbamoyl, thiocarbamoyl, carbamido, cyano, isocyano, cyanato, isocyanato, isothiocyanato, azido, formyl, thioformyl, amino, C₁-C₂₄ alkyl amino, C₅-C₂₀ aryl amino, C₂-C₂₄ alkylamido, C₂-C₂₄ alkylamido substituted with a hydroxyl, C₆-C₂₀ arylamido, imino, alkylimino, arylimino, nitro, nitroso, sulfo, sulfonato, C₁-C₂₄ alkylsulfanyl, arylsulfanyl, C₁-C₂₄ alkylsulfinyl, C₅-C₂₀ arylsulfinyl, C₁-C₂₄ alkylsulfonyl, C₅-C₂₀ arylsulfonyl, sulfonamide, phosphono, phosphonato, phosphinato, phospho, phosphino, polyalkylethers, phosphates, and phosphate esters, groups incorporating amino acids or other moieties expected to bear positive or negative charge at physiological pH, and combinations thereof, and wherein R⁶ and R⁷ may be linked to form a cyclic or polycyclic ring, wherein the ring is a substituted or unsubstituted aryl, a substituted or unsubstituted heteroaryl, a substituted or unsubstituted cycloalkyl, and a substituted or unsubstituted heterocyclyl; and
U¹ is N, C—R², or C—NR³R⁴, wherein R² is selected from the group consisting of a H, a lower alkyl group, O, (CH₂)ₙ₁OR′ (wherein n1=1, 2, or 3), CF₃, CH₂—CH₂X, O—CH₂—CH₂X, CH₂—CH₂—CH₂X, O—CH₂—CH₂X, X, (wherein X=H, F, Cl, Br, or I), CN, (C═O)—R′, (C═O)N(R′)₂, O(CO)R′, COOR′ (wherein R′ is H or a lower alkyl group), and wherein R¹ and R² may be linked to form a cyclic or polycyclic ring, wherein R³ and R⁴ are the same or different and are each selected from the group consisting of H, a lower alkyl group, O, (CH₂)ₙ₁OR′ (wherein n1=1, 2, or 3), CF₃, CH₂—CH₂X, CH₂—CH₂—CH₂X, (wherein X=H, F, Cl, Br, or I), CN, (C═O)—R′, (C═O)N(R′)₂, COOR′ (wherein R′ is H or a lower alkyl group), and R³ or R⁴ may be absent.

29: The method of claim 20, wherein the 15-PGDH inhibitor has the following formula (V): 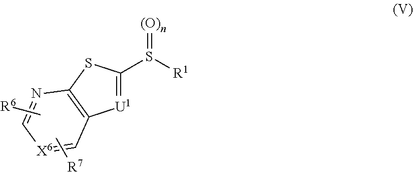
or a pharmaceutically acceptable salt, tautomer, or solvate thereof;
wherein n is 0-2
X⁶ is independently is N or CR^{c}
R¹, R⁶, R⁷, and R^{c} are the same or different each independently hydrogen or a substituted or unsubstituted group selected from C₁-C₂₄ alkyl, C₂-C₂₄ alkenyl, C₂-C₂₄ alkynyl, C₃-C₂₀ aryl, heteroaryl, heterocycloalkenyl containing from 5-6 ring atoms, C₆-C₂₄ alkaryl, C₆-C₂₄ aralkyl, halo, —Si(C₁-C₃ alkyl)₃, hydroxyl, sulfhydryl, C₁-C₂₄ alkoxy, C₂-C₂₄ alkenyloxy, C₂-C₂₄ alkynyloxy, C₅-C₂₀ aryloxy, acyl, acyloxy, C₂-C₂₄ alkoxycarbonyl, C₆-C₂₀ aryloxycarbonyl, C₂-C₂₄ alkylcarbonato, C₆-C₂₀ arylcarbonato, carboxy, carboxylato, carbamoyl, C₁-C₂₄ alkyl-carbamoyl, arylcarbamoyl, thiocarbamoyl, carbamido, cyano, isocyano, cyanato, isocyanato, isothiocyanato, azido, formyl, thioformyl, amino, C₁-C₂₄ alkyl amino, C₅-C₂₀ aryl amino, C₂-C₂₄ alkylamido, C₂-C₂₄ alkylamido substituted with a hydroxyl, C₆-C₂₀ arylamido, imino, alkylimino, arylimino, nitro, nitroso, sulfo, sulfonato, C₁-C₂₄ alkylsulfanyl, arylsulfanyl, C₁-C₂₄ alkylsulfinyl, 05-C₂₀ arylsulfinyl, C₁-C₂₄ alkylsulfonyl, C₅-C₂₀ arylsulfonyl, sulfonamide, phosphono, phosphonato, phosphinato, phospho, phosphino, polyalkylethers, phosphates, and phosphate esters, groups incorporating amino acids or other moieties expected to bear positive or negative charge at physiological pH, and combinations thereof, and wherein R⁶ and R⁷ may be linked to form a cyclic or polycyclic ring, wherein the ring is a substituted or unsubstituted aryl, a substituted or unsubstituted heteroaryl, a substituted or unsubstituted cycloalkyl, and a substituted or unsubstituted heterocyclyl; and
U¹ is N, C—R², or C—NR³R⁴, wherein R² is selected from the group consisting of a H, a lower alkyl group, O, (CH₂)ₙ₁OR′ (wherein n1=1, 2, or 3), CF₃, CH₂—CH₂X, O—CH₂—CH₂X, CH₂—CH₂—CH₂X, O—CH₂—CH₂X, X, (wherein X=H, F, Cl, Br, or I), CN, (C═O)—R′, (C═O)N(R′)₂, O(CO)R′, COOR′ (wherein R′ is H or a lower alkyl group), and wherein R¹ and R² may be linked to form a cyclic or polycyclic ring, wherein R³ and R⁴ are the same or different and are each selected from the group consisting of H, a lower alkyl group, O, (CH₂)ₙ₁OR′ (wherein n1=1, 2, or 3), CF₃, CH₂—CH₂X, CH₂—CH₂—CH₂X, (wherein X=H, F, Cl, Br, or I), CN, (C═O)—R′, (C═O)N(R′)₂, COOR′ (wherein R′ is H or a lower alkyl group), and R³ or R⁴ may be absent.

30: A method of treating Alzheimer's Disease in a subject in need thereof, the method comprising:
administering to the subject a therapeutically effective amount of a compound having the formulas (IB) or (IIB), or a pharmaceutically acceptable salt thereof: 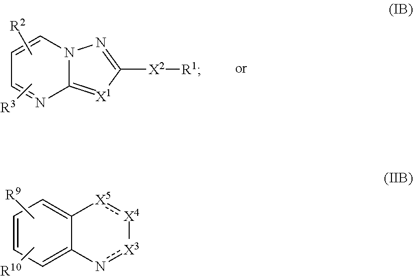
wherein X¹ is N or CR⁴;
X² is S, S═O, S(═O)₂, or C═O;
X³ is CR⁸, the compound forming a polycyclic heteroaryl with 10 ring atoms, or absent, the compound forming a polycyclic heteroaryl with 9 ring atoms;
X⁴ is N, NH, or CR⁷;
X⁵ is N, C═O, or CR¹⁶, and X⁵ is N if X⁴ is CR⁷, or X³ is absent, X⁴ is NH if X⁵ is C═O, and X⁵ is CR¹⁶ if X⁴ is N and X³ is CR⁸;
R¹, R², R³, R⁴, R⁹, R¹⁰, and R¹⁶ are the same or different and are independently selected from the group consisting of hydrogen, substituted or unsubstituted C₁-C₂₄ alkyl, C₂-C₂₄ alkenyl, C₂-C₂₄ alkynyl, C₃-C₂₀ aryl, heterocycloalkenyl containing from 5-7 ring atoms, heteroaryl or heterocyclyl containing from 5-14 ring atoms, C₆-C₂₄ alkaryl, C₆-C₂₄ aralkyl, halo, silyl, hydroxyl, sulfhydryl, C₁-C₂₄ alkoxy, C₂-C₂₄ alkenyloxy, C₂-C₂₄ alkynyloxy, C₅-C₂₀ aryloxy, acyl, acyloxy, C₂-C₂₄ alkoxycarbonyl, C₆-C₂₀ aryloxycarbonyl, C₂-C₂₄ alkylcarbonato, C₆-C₂₀ arylcarbonato, carboxy, carboxylato, carbamoyl, C₁-C₂₄ alkyl-carbamoyl, arylcarbamoyl, thiocarbamoyl, carbamido, cyano, isocyano, cyanato, isocyanato, isothiocyanato, azido, formyl, thioformyl, amino, C₁-C₂₄ alkyl amino, C₅-C₂₀ aryl amino, C₂-C₂₄ alkylamido, C₆-C₂₀ arylamido, sulfanamido, imino, alkylimino, arylimino, nitro, nitroso, sulfo, sulfonato, C₁-C₂₄ alkylsulfanyl, arylsulfanyl, C₁-C₂₄ alkylsulfinyl, C₅-C₂₀ arylsulfinyl, C₁-C₂₄ alkylsulfonyl, C₅-C₂₀ arylsulfonyl, sulfonamide, phosphono, phosphonato, phosphinato, phospho, phosphino, polyalkyl ethers, phosphates, phosphate esters, and combinations thereof;
R⁷ and R⁸ are same or different and are each independently selected from the group consisting of H, a substituted or unsubstituted aryl, a substituted or unsubstituted heteroaryl, a substituted or unsubstituted cycloalkyl, and a substituted or unsubstituted heterocyclyl, and at least one of R⁷ or R⁸ is not H.
